(19) United States
(12) Patent Application Publication
(10) Pub. No.: US 2023/0190796 A1
(43) Pub. Date: Jun. 22, 2023

(54) ENGINEERED CELLS EXPRESSING PROSTATE-SPECIFIC MEMBRANE ANTIGEN (PSMA) OR A MODIFIED FORM THEREOF AND RELATED METHODS

(71) Applicants: Juno Therapeutics, Inc., Seattle, WA (US); The Johns Hopkins University, Baltimore, MD (US)

(72) Inventors: David Jeffrey HUSS, Seattle, WA (US); Hyam I. LEVITSKY, Seattle, WA (US); Il MINN, Baltimore, MD (US); Martin G. POMPER, Baltimore, MD (US)

(73) Assignees: Juno Therapeutics, Inc., Seattle, WA (US); The Johns Hopkins University, Baltimore, MD (US)

(21) Appl. No.: 16/500,352

(22) PCT Filed: Apr. 7, 2018
(86) PCT No.: PCT/US2018/026619

§ 371 Date: Oct. 2, 2019

Related U.S. Application Data
(60) Provisional application No. 62/483,313, filed on Apr. 7, 2017.
(60) Provisional application No. 62/552,354, filed on Aug. 30, 2017.
(60) Provisional application No. 62/555,635, filed on Sep. 7, 2017.
(60) Provisional application No. 62/582,913, filed on Nov. 7, 2017.
(60) Provisional application No. 62/619,724, filed on Jan. 19, 2018.

(51) Int. Cl. A61K 35/17; C12N 15/86; C12N 5/0783; G01N 33/569; C07K 14/725; C07K 14/47; A61P 35/00

(52) U.S. Cl. CPC A61K 35/17; C12N 15/86; C12N 5/0636; G01N 33/56972; C07K 14/7051; C07K 14/4748; A61P 35/00; C12N 2740/15043; A61K 38/00

(57) ABSTRACT

Provided are cells, such as engineered cells, that express a prostate-specific membrane antigen (PSMA) or a modified form thereof. In some embodiments, the cell further contains a genetically engineered recombinant receptor, such as a chimeric antigen receptor, that specifically binds to an antigen. The present disclosure also provides methods of detecting, identifying, selecting or targeting cells expressing PSMA, such as in connection with administration of such cells to subjects, including methods of adoptive cell therapy, or in connection with methods of manufacturing engineered cells.

### CROSS-REFERENCE TO RELATED APPLICATIONS

This application is a National Stage application under 35 U.S.C. § 371 of International Application No. PCT/US2018/026619, filed on Apr. 7, 2018, which claims priority from U.S. provisional application No. 62/483,313, filed Apr. 7, 2017, entitled “ENGINEERED CELLS EXPRESSING PROSTATE-SPECIFIC MEMBRANE ANTIGEN (PSMA) OR A MODIFIED FORM THEREOF AND RELATED METHODS,” U.S. provisional application No. 62/552,354, filed Aug. 30, 2017, entitled “ENGINEERED CELLS EXPRESSING PROSTATE-SPECIFIC MEMBRANE ANTIGEN (PSMA) OR A MODIFIED FORM THEREOF AND RELATED METHODS,” U.S. provisional application No. 62/555,635, filed Sep. 7, 2017, entitled “ENGINEERED CELLS EXPRESSING PROSTATE-SPECIFIC MEMBRANE ANTIGEN (PSMA) OR A MODIFIED FORM THEREOF AND RELATED METHODS,” U.S. provisional application No. 62/582,913, filed Nov. 7, 2017, entitled “ENGINEERED CELLS EXPRESSING PROSTATE-SPECIFIC MEMBRANE ANTIGEN (PSMA) OR A MODIFIED FORM THEREOF AND RELATED METHODS,” and U.S. provisional application No. 62/619,724, filed Jan. 19, 2018, entitled “ENGINEERED CELLS EXPRESSING PROSTATE-SPECIFIC MEMBRANE ANTIGEN (PSMA) OR A MODIFIED FORM THEREOF AND RELATED METHODS,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042010600SeqList.txt, created Oct. 1, 2019 which is 59.4 kilobytes in size. The information in the electronic format of the Sequence Listing is incorporated by reference in its entirety.

### FIELD

The present disclosure relates in some aspects to engineered cells, such as engineered T cells, that express a prostate-specific membrane antigen (PSMA), typically a modified PSMA. The cells further contain a genetically engineered recombinant receptor, such as a chimeric antigen receptor, that specifically binds to an antigen. The present disclosure also provides methods of detecting, identifying, selecting or targeting cells expressing PSMA, such as in connection with administration of such cells to subjects, including methods of adoptive cell therapy, or in connection with methods of manufacturing engineered cells.

### BACKGROUND

Various strategies are available for treatment of diseases or conditions such as cancers or tumors, including the administration of cell therapies. Further, strategies are available for engineering immune cells to express genetically engineered recombinant receptors, such as chimeric antigen receptors (CARs), and administering compositions containing such cells to subjects. Improved strategies are needed, for example, to improve the ability to monitor, detect or modulate the engineered cells in connection with such therapies upon administration to subjects. Provided are compositions, cells, and methods that meet such needs.

### SUMMARY

Provided herein are engineered cells that include a prostate-specific membrane antigen (PSMA) or a modified form thereof; and a recombinant receptor. Also provided herein are engineered cells that include a nucleic acid encoding a prostate-specific membrane antigen (PSMA) or a modified form thereof; and a nucleic acid encoding a recombinant receptor. In some of any of the provided embodiments, the recombinant receptor is a chimeric receptor and/or a recombinant antigen receptor, such as a chimeric antigen receptor (CAR).

In some embodiments, the PSMA or modified form thereof is expressed on the surface of the cell. In some embodiments, the PSMA or modified form thereof includes an extracellular portion and a transmembrane domain. In some embodiments, the PSMA or modified form thereof, optionally the extracellular portion, is capable of being recognized by a PSMA-targeting molecule or a portion thereof

In some embodiments, the PSMA-targeting molecule or a portion thereof: is capable of binding to a PSMA and/or to the modified form thereof, and/or is capable of binding to the active site of a PSMA and/or of the modified form of PSMA, and/or is capable of being cleaved by a PSMA and/or by the modified form of PSMA; and/or is an antagonist, a selective antagonist, an inverse agonist, a selective inverse agonist, an agonist, a selective agonist, an inhibitor, and/or a selective inhibitor of a PSMA and/or of the modified form thereof.

In some embodiments, the PSMA or modified form thereof includes an N-acetylated-alpha-linked-acidic dipeptidase (NAALADase) domain and/or includes one or more active site residues and/or residues involved in PSMA substrate binding and/or PSMA catalytic activity, which optionally are residues at positions 210, 257, 269, 272, 377, 387, 387, 424, 424, 425, 433, 436, 453, 517, 518, 519, 552, 553, 534, 535, 536, 552, 553, 628, 666, 689, 699 and/or 700, with reference to positions in the sequence of amino acids set forth in SEQ ID NO:23.

In some embodiments, the PSMA or modified form thereof is a human PSMA or a modified form thereof. In some embodiments, the PSMA or modified form thereof is wild-type PSMA, optionally wild-type human PSMA. In some embodiments, the PSMA or modified form thereof includes the sequence of amino acids set forth in SEQ ID NO:23 or an extracellular and/or transmembrane domain thereof, or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NO:23 or an extracellular and/or transmembrane domain thereof.

In some embodiments, the PSMA or modified form thereof is a modified PSMA, the modified PSMA containing one or more amino acid modifications compared to a wild-type or unmodified PSMA.

In some embodiments, the wild-type or unmodified PSMA is human PSMA and/or includes the sequence of amino acids set forth in SEQ ID NO:23 or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity thereto.

In some embodiments, the one or more amino acid modification includes one or more amino acid substitutions, deletions and/or insertions. In some embodiments, the modified PSMA (i) exhibits reduced endogenous signaling; (ii) exhibits increased cell surface expression; and/or (iii) exhibits reduced cellular internalization compared to the wild-type or unmodified PSMA.

In some embodiments, the modified PSMA includes at least one amino acid substitution corresponding to W2G or does not comprise W2 or does not comprise any residue at position 2, with reference to positions in the sequence of amino acids set forth in SEQ ID NO:23. In some embodiments, the modified PSMA includes the sequence of amino acids set forth in SEQ ID NO:24 or a fragment thereof, or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NOS:24 or a fragment thereof and includes the at least one amino acid substitution.

In some embodiments, the modified PSMA includes a deletion of one or more N-terminal amino acid residues within the intracellular portion, compared to the wild-type or unmodified PSMA. In some embodiments, the modified PSMA includes a deletion of up to 2, 3, 4, 5, 6, 7, 8, 9, 10, 11, 12, 13, 14, 15, 16, 17 or 18 N-terminal amino acid residues compared to the wild-type or unmodified PSMA. In some embodiments, the modified PSMA comprises deletion of a contiguous amino acid sequence at the N-terminus starting from residue at position 2, 3, 4, or 5 and up to position 6, 7, 8, 9, 10, 11, 12, 13, 14, 15, 16, 17 18, or 19, compared to the wild-type or unmodified PSMA, with reference to positions in the sequence of amino acids set forth in SEQ ID NO:23. In some embodiments, the modified PSMA comprises a deletion of residues at positions 2-19, 2-18, 2-17, 2-16, 2-15, 2-14, 2-13, 2-12, 2-11, 2-10, 2-9, 2-8, 2-7, 2-6, 2-5, 2-4 or 2-3, with reference to positions in the sequence of amino acids set forth in SEQ ID NO:23.

In some embodiments, the PSMA or modified form thereof includes the sequence of amino acids set forth in SEQ ID NO:52 or a fragment thereof; or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NOS:52 or a fragment thereof and includes deletion of the one or more N-terminal amino acid residues, and, optionally, contains a methionine as the first residue or a methionine start codon.

In some embodiments, the PSMA or modified form thereof includes the sequence of amino acids set forth in SEQ ID NO:25 or a fragment thereof; or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NOS:25 or a fragment thereof and includes deletion of the one or more N-terminal amino acid residues.

In some embodiments, the PSMA or modified form thereof is encoded by the sequence of nucleic acids set forth in SEQ ID NO:26 or 53 or a fragment thereof; or a sequence of nucleic acids that exhibits at least or at least about 85%, 86%, 87%, 88%, 89%, 90%, 91%, 92%, 93%, 94%, 95%, 96%, 97%, 98%, 99% or more sequence identity to SEQ ID NO:26 or 53 or a fragment thereof and that, optionally, contains a nucleic acid encoding methionine as the first residue or a methionine start codon.

In some embodiments, the PSMA or modified form thereof is encoded by a sequence of nucleic acids that is modified to be CpG-free and/or is codon optimized. In some embodiments, the PSMA or modified form thereof is encoded by the sequence of nucleic acids set forth in SEQ ID NO:27 or a fragment thereof or a sequence of nucleic acids that exhibits at least or at least about 85%, 86%, 87%, 88%, 89%, 90%, 91%, 92%, 93%, 94%, 95%, 96%, 97%, 98%, 99% or more sequence identity to SEQ ID NO:27 or a fragment thereof, and optionally that contains a nucleic acids that encodes methionine as the first residue or a methionine start codon.

In some embodiments, the modified PSMA includes all or substantially all of the transmembrane domain of the wild-type or unmodified PSMA; or the modified PSMA includes a transmembrane domain with the same or at least the same number of amino acids as the transmembrane domain of the wild-type or unmodified PSMA.

In some embodiments, the PSMA-targeting molecule is or includes a small molecule, a ligand, an antibody or antigen-binding fragment thereof, an aptamer, a peptide, or a conjugate thereof.

In some embodiments, the PSMA-targeting molecule is or includes a small molecule. In some embodiments, the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N,N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE). In some embodiments, the PSMA-targeting molecule is or includes 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).

In some embodiments, the PSMA-targeting molecule is or includes an antibody or antigen-binding fragment thereof, optionally having a binding site that specifically binds to PSMA. In some embodiments, the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or includes a CDR3, a V_{H} and/or V_{L}, and/or competes for binding to PSMA or binds to the same PSMA epitope as any of the foregoing.

In some embodiments, the PSMA-targeting molecule is or includes an aptamer or a conjugate thereof. In some embodiments, the aptamer includes A9, A10, A10g, A10-3.2 or SZT101 or a conjugate thereof.

In some embodiments, the recombinant receptor is or includes a chimeric receptor and/or a recombinant antigen receptor. In some embodiments, the recombinant receptor is capable of binding to a target antigen that is associated with, specific to, and/or expressed on a cell or tissue of a disease, disorder or condition. In some embodiments, the disease, disorder or condition is an infectious disease or disorder, an autoimmune disease, an inflammatory disease, or a tumor or a cancer. In some embodiments, the target antigen is a tumor antigen.

In some embodiments, the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. In some embodiments, the target antigen is selected from among ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, G Protein Coupled Receptor 5D (GPCR5D), HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-A1, MAGE A1, HLA-A2, NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag.

In some embodiments, the recombinant receptor is or includes a functional non-TCR antigen receptor or a TCR or antigen-binding fragment thereof. In some embodiments, the recombinant receptor is a chimeric antigen receptor (CAR).

In some embodiments, the recombinant receptor includes an extracellular domain containing an antigen-binding domain. In some embodiments, the antigen-binding domain is or includes an antibody or an antibody fragment thereof, which optionally is a single chain fragment. In some embodiments, the fragment includes antibody variable regions joined by a flexible linker. In some embodiments, the fragment includes an scFv.

In some embodiments, the recombinant receptor also includes a spacer and/or a hinge region.

In some embodiments, the recombinant receptor includes an intracellular signaling region. In some embodiments, the intracellular signaling region includes an intracellular signaling domain. In some embodiments, the intracellular signaling domain is or includes a primary signaling domain, a signaling domain that is capable of inducing a primary activation signal in a T cell, a signaling domain of a T cell receptor (TCR) component, and/or a signaling domain containing an immunoreceptor tyrosine-based activation motif (ITAM). In some embodiments, the intracellular signaling domain is or includes an intracellular signaling domain of a CD3 chain, optionally a CD3-zeta (CD3ζ) chain, or a signaling portion thereof.

In some embodiments, the recombinant receptor also includes a transmembrane domain disposed between the extracellular domain and the intracellular signaling region.

In some embodiments, the intracellular signaling region also includes a costimulatory signaling region. In some embodiments, the costimulatory signaling region includes an intracellular signaling domain of a T cell costimulatory molecule or a signaling portion thereof. In some embodiments, the costimulatory signaling region includes an intracellular signaling domain of a CD28, a 4-1BB or an ICOS or a signaling portion thereof. In some embodiments, the costimulatory signaling region is between the transmembrane domain and the intracellular signaling region.

In some embodiments, the nucleic acid encoding the PSMA or modified form thereof and the nucleic acid encoding the recombinant receptor are comprised within one or more polynucleotide(s) comprised by the cell. In some embodiments, the one or more polynucleotide(s) is one polynucleotide and the nucleic acid encoding the PSMA or modified form thereof and the nucleic acid encoding the recombinant receptor are operably linked to the same promoter and are optionally separated by an internal ribosome entry site (IRES), or a nucleic acid encoding a self-cleaving peptide or a peptide that causes ribosome skipping, which optionally is a T2A, a P2A, a E2A or a F2A.

In some embodiments, the nucleic acid encoding the PSMA or modified form thereof and the nucleic acid encoding the CAR are comprised within one polynucleotide comprised by the cell, said polynucleotide comprising, in 5′ to 3′ order: i) a nucleic acid encoding a signal peptide; ii) a nucleic acid encoding the CAR said CAR comprising an scFv; a spacer; a transmembrane domain; an intracellular region comprising a costimulatory signaling region, and an intracellular signaling domain of a CD3-zeta (CD3ζ) chain, or a signaling portion thereof iii) a nucleic acid sequence encoding a self-cleaving peptide or a peptide that causes ribosome skipping, which optionally is a T2A, a P2A, a E2A or a F2A; and iv) a nucleic acid encoding the PSMA or modified form thereof, which optionally comprises the sequence of amino acids set forth in SEQ ID NO: 52 or a fragment thereof or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS: 52 or a fragment thereof and that comprises deletion of the one or more N-terminal amino acid residues.

In some embodiments, the nucleic acid encoding the PSMA or modified form thereof and the nucleic acid encoding the recombinant receptor are operably linked to two different promoters.

In some embodiments, the nucleic acid encoding the recombinant receptor is present downstream of the nucleic acid encoding the PSMA or modified form thereof.

In some embodiments, the one or more polynucleotides includes two different polynucleotides, the nucleic acid encoding the PSMA or modified form thereof and the nucleic acid encoding the recombinant receptor are operably linked to two different promoters, and/or the nucleic acid encoding the PSMA or modified form thereof and the nucleic acid encoding the recombinant receptor are present or inserted at different locations within the genome of the cell.

In some embodiments, the cell is an immune cell; the cell is a T cell, optionally selected from the group consisting of CD4+ T cells and subtypes thereof and CD8+ T cells and subtypes thereof; the cell is an NK cell; and/or the cell is derived from a multipotent or pluripotent cell, which optionally is an iPSC.

In some embodiments, the cell is a T cell selected from the group consisting of central memory T cells, effector memory T cells, naïve T cells, stem central memory T cells, effector T cells and regulatory T cells; and/or the cell includes a plurality of cells, the plurality containing at least 50% of a population of cells selected from the group consisting of CD4+ T cells, CD8+ T cells, central memory T cells, effector memory T cells, naïve T cells, stem central memory T cells, effector T cells and regulatory T cells. In some embodiments, the cell is a regulatory T cell.

In some embodiments, the engineered cells provided herein also include a recombinant FOXP3 or variant thereof.

Also provided are polynucleotides containing a first nucleic acid encoding a prostate-specific membrane antigen (PSMA) or modified form thereof and a second nucleic acid encoding a recombinant receptor. In some of any of the provided embodiments, the recombinant receptor is a chimeric receptor and/or a recombinant antigen receptor, such as a chimeric antigen receptor (CAR). In some embodiments, the nucleic acid encoding the PSMA or modified form thereof and the nucleic acid encoding the recombinant receptor are operably linked to the same promoter and are optionally separated by an internal ribosome entry site (IRES), or a nucleic acid encoding a self-cleaving peptide or a peptide that causes ribosome skipping, which optionally is a T2A, a P2A, a E2A or a F2A.

In some embodiments, the nucleic acid encoding the PSMA or modified form thereof and the nucleic acid encoding the recombinant receptor are operably linked to two different promoters.

In some embodiments, the nucleic acid encoding the recombinant receptor is present downstream of the nucleic acid encoding the PSMA or modified form thereof

Also provided are a set of polynucleotides that include a first polynucleotide containing a nucleic acid encoding a prostate-specific membrane antigen (PSMA) or modified form thereof and a second polynucleotide containing a nucleic acid encoding a recombinant receptor. Also provided are compositions that include such set of polynucleotides. In some of any of the provided embodiments, the recombinant receptor is a chimeric receptor and/or a recombinant antigen receptor, such as a chimeric antigen receptor (CAR).

In some embodiments of the polynucleotides, set of polynucleotides or compositions that include the set of polynucleotides provided herein, the nucleic acid encoding PSMA or a modified form thereof and the nucleic acid encoding the recombinant receptor each independently are operably linked to a promoter.

In some embodiments, the encoded PSMA or modified form thereof is capable of being expressed on the surface of a cell. In some embodiments, the encoded PSMA or modified form thereof includes an extracellular portion and a transmembrane domain.

In some embodiments, the PSMA or modified form thereof, optionally the extracellular portion, is capable of being recognized by a PSMA-targeting molecule or a portion thereof.

In some embodiments, the PSMA-targeting molecule or a portion thereof: is capable of binding to a PSMA and/or to the modified form thereof, and/or is capable of binding to the active site of a PSMA and/or of the modified form of PSMA, and/or is capable of being cleaved by a PSMA and/or by the modified form of PSMA; and/or is an antagonist, a selective antagonist, an inverse agonist, a selective inverse agonist, an agonist, a selective agonist, an inhibitor, and/or a selective inhibitor of a PSMA and/or of the modified form thereof

In some embodiments, the encoded PSMA or modified form thereof includes an N-acetylated-alpha-linked-acidic dipeptidase (NAALADase) domain and/or includes one or more active site residues and/or residues involved in PSMA substrate binding and/or PSMA catalytic activity, which optionally are residues at positions 210, 257, 269, 272, 377, 387, 387, 424, 424, 425, 433, 436, 453, 517, 518, 519, 552, 553, 534, 535, 536, 552, 553, 628, 666, 689, 699 and/or 700, with reference to positions in the sequence of amino acids set forth in SEQ ID NO:23.

In some embodiments, the encoded PSMA or modified form thereof is a human PSMA or a modified form thereof. In some embodiments, the encoded PSMA or modified form thereof is wild-type PSMA, optionally wild-type human PSMA. In some embodiments, the encoded PSMA or modified from thereof includes the sequence of amino acids set forth SEQ ID NO:23 or an extracellular and/or transmembrane domain thereof, or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NO:23 or an extracellular and/or transmembrane domain thereof.

In some embodiments, the encoded PSMA or modified form thereof is a modified PSMA, the modified PSMA containing one or more amino acid modifications compared to a wild-type or unmodified PSMA. In some embodiments, the wild-type or unmodified PSMA is human PSMA and/or includes the sequence of amino acids set forth in SEQ ID NO:23 or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity thereto.

In some embodiments, the one or more amino acid modification includes one or more amino acid substitutions, deletions and/or insertions. In some embodiments, the encoded modified PSMA (i) exhibits reduced endogenous signaling; (ii) exhibits increased cell surface expression; and/or (iii) exhibits reduced cellular internalization compared to the wild-type or unmodified PSMA.

In some embodiments, the encoded modified PSMA includes at least one amino acid substitution corresponding to W2G or does not comprise W2 or does not comprise any residue at position 2, with reference to positions in the sequence of amino acids set forth in SEQ ID NO:23. In some embodiments, the encoded modified PSMA includes the sequence of amino acids set forth in SEQ ID NO:24 or a fragment thereof or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NOS:24 or a fragment thereof and includes the at least one amino acid substitution.

In some embodiments, the encoded modified PSMA includes a deletion of one or more N-terminal amino acid residues within the intracellular portion, compared to the wild-type or unmodified PSMA. In some embodiments, the encoded modified PSMA includes a deletion of up to 2, 3, 4, 5, 6, 7, 8, 9, 10, 11, 12, 13, 14, 15, 16, 17 or 18 N-terminal amino acid residues compared to the wild-type or unmodified PSMA. In some embodiments, the encoded modified PSMA comprises deletion of a contiguous amino acid sequence at the N-terminus starting from residue at position 2, 3, 4, or 5 and up to position 6, 7, 8, 9, 10, 11, 12, 13, 14, 15, 16, 17, 18, or 19, with reference to positions in the sequence of amino acids set forth in SEQ ID NO:23. In some embodiments, the encoded modified PSMA comprises a deletion of residues at positions 2-19, 2-18, 2-17, 2-16, 2-15, 2-14, 2-13, 2-12, 2-11, 2-10, 2-9, 2-8, 2-7, 2-6, 2-5, 2-4 or 2-3, with reference to positions in the sequence of amino acids set forth in SEQ ID NO:23.

In some embodiments, the encoded PSMA or modified form thereof includes the sequence of amino acids set forth in SEQ ID NO:52 or a fragment thereof; or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NOS:52 or a fragment thereof and includes deletion of the one or more N-terminal amino acid residues and, optionally, contains methionine as the first codon or a methionine start codon.

In some embodiments, the encoded PSMA or modified form thereof includes the sequence of amino acids set forth in SEQ ID NO:25 or a fragment thereof or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NOS:25 or a fragment thereof and includes deletion of the one or more N-terminal amino acid residues.

In some embodiments, the PSMA or modified form thereof is encoded by the sequence of nucleic acids set forth in SEQ ID NO:26 or 53 or a fragment thereof; or a sequence of nucleic acids that exhibits at least or at least about 85%, 86%, 87%, 88%, 89%, 90%, 91%, 92%, 93%, 94%, 95%, 96%, 97%, 98%, 99% or more sequence identity to SEQ ID NO:26 or 53 or a fragment thereof and that, optionally, contains a nucleic acid encoding methionine as the first residue or a methionine start codon.

In some embodiments, the PSMA or modified form thereof is encoded by a sequence of nucleic acids that is modified to be CpG-free and/or is codon optimized. In some embodiments, the PSMA or modified form thereof is encoded by the sequence of nucleic acids set forth in SEQ ID NO:27 or a fragment thereof, or a sequence of nucleic acids that exhibits at least or at least about 85%, 86%, 87%, 88%, 89%, 90%, 91%, 92%, 93%, 94%, 95%, 96%, 97%, 98%, 99% or more sequence identity to SEQ ID NO:27 or a fragment thereof and that, optionally, contains a nucleic acid encoding a methionine as the first residue or a methionine start codon.

In some embodiments, the encoded modified PSMA includes all or substantially all of the transmembrane domain of the wild-type or unmodified PSMA; or the encoded modified PSMA includes a transmembrane domain with the same or at least the same number of amino acids as the transmembrane domain of the wild-type or unmodified PSMA.

In some embodiments, the PSMA-targeting molecule is or includes a small molecule, a ligand, an antibody or antigen-binding fragment thereof, an aptamer, a peptide, or a conjugate thereof.

In some embodiments, the PSMA-targeting molecule is or includes a small molecule. In some embodiments, the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N,N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE). In some embodiments, the PSMA-targeting molecule is or includes 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).

In some embodiments, the PSMA-targeting molecule is or includes an antibody or antigen-binding fragment thereof, optionally having a binding site that specifically binds to PSMA. In some embodiments, the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or includes a CDR3, a V_{H} and/or V_{L}, and/or competes for binding to PSMA or binds to the same PSMA epitope as any of the foregoing.

In some embodiments, the PSMA-targeting molecule is or includes an aptamer or a conjugate thereof. In some embodiments, the aptamer includes A9, A10, A10g, A10-3.2 or SZT101 or a conjugate thereof.

In some embodiments, the encoded recombinant receptor is or includes a chimeric receptor and/or a recombinant antigen receptor.

In some embodiments, the encoded recombinant receptor is capable of binding to a target antigen that is associated with, specific to, and/or expressed on a cell or tissue of a disease, disorder or condition. In some embodiments, the disease, disorder or condition is an infectious disease or disorder, an autoimmune disease, an inflammatory disease, or a tumor or a cancer. In some embodiments, the target antigen is a tumor antigen.

In some embodiments, target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. In some embodiments, the target antigen is selected from among ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, G Protein Coupled Receptor 5D (GPCR5D), HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-A1, MAGE A1, HLA-A2, NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag.

In some embodiments, the encoded recombinant receptor is or includes a functional non-TCR antigen receptor or a TCR or antigen-binding fragment thereof. In some embodiments, the encoded recombinant receptor is a chimeric antigen receptor (CAR).

In some embodiments, the encoded recombinant receptor includes an extracellular domain containing an antigen-binding domain.

In some embodiments, the antigen-binding domain is or includes an antibody or an antibody fragment thereof, which optionally is a single chain fragment. In some embodiments, the fragment includes antibody variable regions joined by a flexible linker. In some embodiments, the fragment includes an scFv.

In some embodiments, the encoded recombinant receptor also includes a spacer and/or a hinge region.

In some embodiments, the encoded recombinant receptor includes an intracellular signaling region. In some embodiments, the intracellular signaling region includes an intracellular signaling domain. In some embodiments, the intracellular signaling domain is or includes a primary signaling domain, a signaling domain that is capable of inducing a primary activation signal in a T cell, a signaling domain of a T cell receptor (TCR) component, and/or a signaling domain containing an immunoreceptor tyrosine-based activation motif (ITAM). In some embodiments, the intracellular signaling domain is or includes an intracellular signaling domain of a CD3 chain, optionally a CD3-zeta (CD3ζ) chain, or a signaling portion thereof

In some embodiments, the recombinant receptor also includes a transmembrane domain disposed between the extracellular domain and the intracellular signaling region.

In some embodiments, the intracellular signaling region also includes a costimulatory signaling region. In some embodiments, the costimulatory signaling region includes an intracellular signaling domain of a T cell costimulatory molecule or a signaling portion thereof. In some embodiments, the costimulatory signaling region includes an intracellular signaling domain of a CD28, a 4-1BB or an ICOS or a signaling portion thereof.

In some embodiments, the costimulatory signaling region is between the transmembrane domain and the intracellular signaling region.

In some embodiments, the polynucleotide comprises, in 5′ to 3′ order: i) a nucleic acid encoding a signal peptide; ii) a nucleic acid encoding the CAR said CAR comprising an scFv; a spacer; a transmembrane domain; an intracellular region comprising a costimulatory signaling region, and an intracellular signaling domain of a CD3-zeta (CD3ζ) chain, or a signaling portion thereof; iii) a nucleic acid sequence encoding a self-cleaving peptide or a peptide that causes ribosome skipping, which optionally is a T2A, a P2A, a E2A or a F2A; and iv) a nucleic acid encoding the PSMA or modified form thereof, which optionally comprises the sequence of amino acids set forth in SEQ ID NO: 52 or a fragment thereof; or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS: 52 or a fragment thereof and that comprises deletion of the one or more N-terminal amino acid residues.

Also provided are vectors, such as vectors that contain any of the polynucleotides described herein. In some embodiments, the vector is a viral vector. In some embodiments, the vector is a retroviral vector. In some embodiments, the vector is a lentiviral vector or a gammaretroviral vector.

Also provided are a set of vectors that include a first vector and a second vector. In some embodiments, the first vector includes any of the first polynucleotide described herein, and the second vector includes the second polynucleotide described herein. Also provided are compositions that include such set of vectors.

Also provided are methods of producing an engineered cell. In some embodiments, the methods include introducing any of the polynucleotides, polynucleotides in the set or compositions containing set of polynucleotides described herein, any of the vectors or vectors of the set or compositions containing set of vectors described herein, into a cell. Also provided are engineered cells produced using such method of production.

Also provided are engineered cells that contain any of the polynucleotides, polynucleotides in the set or compositions containing set of polynucleotides described herein, any of the vectors or vectors of the set or compositions containing set of vectors described herein.

Also provided are compositions containing any of the engineered cells described herein. In some embodiments, the compositions also contain a pharmaceutically acceptable excipient. In some embodiments, the compositions also contain a PSMA-targeting molecule, such as any PSMA binding molecule described herein.

Also provided are methods of treatment. In some embodiments, the methods of treatment involve administering any of the engineered cells or compositions described herein to a subject. In some embodiments, the methods also involve administering to the subject a PSMA-targeting molecule, or a composition containing a PSMA-targeting molecule. In some embodiments, the PSMA-targeting molecule is or includes a therapeutic agent, or also includes a therapeutic agent.

Also provided are method of treatment that involves administering to a subject: any of the engineered cells or compositions described herein and a PSMA-targeting molecule that is or includes or also includes a therapeutic agent, or a composition containing a PSMA-targeting molecule that is or includes or also includes a therapeutic agent.

Also provided are method of treatment that involves administering to a subject having been administered any of the engineered cells or compositions described herein, a PSMA-targeting molecule, said PSMA-targeting molecule that is or includes or also includes a therapeutic agent, or a composition containing a PSMA-targeting molecule that is or includes or also includes a therapeutic agent. In some of any of the provided embodiments, the recombinant receptor is a chimeric receptor and/or a recombinant antigen receptor, such as a chimeric antigen receptor (CAR).

In some embodiments, the PSMA-targeting molecule or the composition containing the PSMA-targeting molecule is administered simultaneously with or sequentially with, in any order, administration of the engineered cells or the composition containing the engineered cells. In some embodiments, the PSMA-targeting molecule or the composition containing the PSMA-targeting molecule is administered simultaneously with administration of the engineered cells or the composition containing the engineered cells, optionally in the same or different compositions. In some embodiments, the PSMA-targeting molecule or the composition containing the PSMA-targeting molecule is administered sequentially with, in any order, administration of the engineered cells or the composition containing the engineered cells.

In some embodiments, the PSMA-targeting molecule or a portion thereof: is capable of binding to a PSMA and/or to the modified form thereof, and/or is capable of binding to the active site of a PSMA and/or of the modified form of PSMA, and/or is capable of being cleaved by a PSMA and/or by the modified form of PSMA; and/or is an antagonist, a selective antagonist, an inverse agonist, a selective inverse agonist, an agonist, a selective agonist, an inhibitor, and/or a selective inhibitor of a PSMA and/or of the modified form thereof

In some embodiments, the PSMA or modified form thereof is expressed on one or more of the engineered cells.

In some embodiments, the subject has a disease, disorder or condition, optionally a cancer, a tumor, an autoimmune disease, disorder or condition, or an infectious disease.

In some embodiments, the PSMA-targeting molecule is or includes a small molecule, a ligand, an antibody or antigen-binding fragment thereof, an aptamer, a peptide, or a conjugate thereof.

In some embodiments, the PSMA-targeting molecule is or includes a small molecule. In some embodiments, the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE). In some embodiments, the PSMA-targeting molecule is or includes 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).

In some embodiments, the PSMA-targeting molecule is or includes an antibody or antigen-binding fragment thereof, optionally having a binding site that specifically binds to PSMA. In some embodiments, the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or includes a CDR3, a V_{H} and/or V_{L}, and/or competes for binding to PSMA or binds to the same PSMA epitope as any of the foregoing.

In some embodiments, the PSMA-targeting molecule is or includes an aptamer or a conjugate thereof. In some embodiments, the aptamer includes A9, A10, A10g, A10-3.2 or SZT101 or a conjugate thereof.

In some embodiments, the therapeutic agent is capable of modulating the tumor microenvironment (TME) or is cytotoxic to the tumor.

In some embodiments, the therapeutic agent is an immunomodulatory agent, a cytotoxic agent, an anti-cancer agent or a radiotherapeutic.

In some embodiments, the PSMA-targeting molecule is or includes a prodrug that is or includes or is capable of conversion into or unmasking of the therapeutic agent and/or the PSMA-targeting molecule is capable of being cleaved upon binding to the PSMA or modified form thereof, wherein cleavage results in at least one cleavage product containing the therapeutic agent. In some embodiments, the PSMA-targeting molecule is or includes Mipsagargin (G-202 (8-O-(12-aminododecanoyl)-8-O-debutanoyl thapsigargin)-Asp-γ-Glu-γ-Glu-γ-GluGluOH).

In some embodiments, the PSMA-targeting molecule is an antibody-drug conjugate (ADC).

In some embodiments, the PSMA-targeting molecule also includes a therapeutic agent, and the therapeutic agent is linked directly or indirectly, optionally via a linker, to a portion of the PSMA-targeting molecule capable of binding to the PSMA or to the modified form thereof.

In some embodiments, the linker is a peptide or a polypeptide or is a chemical linker. In some embodiments, the linker is a releasable linker or a cleavable linker. In some embodiments, the linker is capable of being cleaved upon binding the PSMA or modified form thereof by the PSMA-targeting molecule, wherein cleavage results in at least one cleavage product containing the therapeutic agent. In some embodiments, the releasable linker or the cleavable linker is released or cleaved in the presence of one or more conditions or factors present in the tumor microenvironment (TME), wherein cleavage results in at least one cleavage product containing the therapeutic agent. In some embodiments, the one or more conditions or factors present in the tumor microenvironment (TME) includes matrix metalloproteinase (MMP), hypoxic conditions or acidic conditions.

In some embodiments, the PSMA-targeting molecule induces killing or destruction of one or more of the engineered cells and/or of a cell or tissue present in the subject that is specifically recognized by the recombinant receptor.

In some embodiments, the therapeutic agent includes a cytotoxic agent. In some embodiments, the cytotoxic agent is or includes a toxin. In some embodiments, the toxin is a peptide toxin, ricin A chain toxin, Abrin A chain, Diphtheria Toxin (DT) A chain, Pseudomonas exotoxin, Shiga Toxin A chain, Gelonin, Momordin, Pokeweed Antiviral Protein, Saporin, Trichosanthin, proaerolysin or Barley Toxin.

In some embodiments, the therapeutic agent includes a photosensitizer. In some embodiments, the photosensitizer includes Pyropheophorbide-a (Ppa) or YC-9.

In some embodiments, the administration of the PSMA-targeting molecule does not, or does not substantially, induce killing or destruction of healthy tissue or healthy cells, of cells or tissues not containing the engineered cells and/or not expressing the antigen.

In some embodiments, the therapeutic agent is an immunomodulatory agent. In some embodiments, the immunomodulatory agent is an immune checkpoint inhibitor or modulator or a cytokine.

In some embodiments, the immunomodulatory agent is an immune checkpoint inhibitor capable of inhibiting or blocking a function of an immune checkpoint molecule or a signaling pathway involving an immune checkpoint molecule. In some embodiments, the wherein the immune checkpoint molecule is selected from among PD-1, PD-L1, PD-L2, CTLA-4, LAG-3, TIM3, VISTA, an adenosine receptor or extracellular adenosine, optionally an adenosine 2A Receptor (A2AR) or adenosine 2B receptor (A2BR), or adenosine or a pathway involving any of the foregoing.

In some embodiments, the methods also include detecting cells that express the PSMA or modified form thereof and/or detecting the binding of the PSMA-targeting molecule to the PSMA or modified form thereof and/or the presence of the PSMA-targeting molecule. In some embodiments, said detecting is performed in vivo and/or the detection is carried out via in vivo imaging.

Also provided are methods of detecting engineered cells. In some embodiments, the methods include contacting any of the engineered cells or compositions provided herein with a PSMA-targeting molecule; and detecting the binding of said PSMA-targeting molecule and/or the presence of said PSMA-targeting molecule to or with the PSMA or modified form thereof and/or the engineered cells. In some embodiments, the contacting includes administering, to a subject having been administered the engineered cells, the PSMA-targeting molecule.

Also provided are methods of detecting the presence or absence of engineered cells in a subject expressing a chimeric receptor and/or a recombinant antigen receptor and a PSMA or modified form thereof in a subject, said subject having been previously administered any of the provided engineered cells or any of the provided compositions, the method comprising: (a) administering to the a subject a PSMA-targeting molecule, said subject having been previously administered any of the engineered cells provided herein or any of the compositions provided herein, wherein the engineered cells express a chimeric receptor and/or a recombinant antigen receptor and a PSMA or modified form thereof in a subject; and (b) detecting the binding of the PSMA-targeting molecule to the PSMA or modified form thereof and/or to the engineered cells and/or the presence of the PSMA-targeting molecule in the subject.

Also provided are methods of detecting the presence or absence of engineered cells expressing a recombinant receptor and a PSMA or modified form thereof in a subject that has been previously administered any of the engineered cells or compositions provided herein. In some embodiments, the method includes administering to the subject a PSMA-targeting molecule; and detecting the binding of the PSMA-targeting molecule to the PSMA or modified form thereof and/or to the engineered cells and/or the presence of the PSMA-targeting molecule in the subject. In some embodiments, the detection is carried out via in vivo imaging. In some of any of the provided embodiments, the recombinant receptor is a chimeric receptor and/or a recombinant antigen receptor, such as a chimeric antigen receptor (CAR).

In some embodiments, the PSMA-targeting molecule or a portion thereof: is capable of binding to a PSMA and/or to the modified form thereof, and/or is capable of binding to the active site of a PSMA and/or of the modified form of PSMA, and/or is capable of being cleaved by a PSMA and/or by the modified form of PSMA; and/or is an antagonist, a selective antagonist, an inverse agonist, a selective inverse agonist, an agonist, a selective agonist, an inhibitor, and/or a selective inhibitor of a PSMA and/or of the modified form thereof. In some embodiments, the PSMA-targeting molecule is or includes a small molecule, a ligand, an antibody or antigen-binding fragment thereof, an aptamer, a peptide, or a conjugate thereof

In some embodiments, the PSMA-targeting molecule is or includes a small molecule. In some embodiments, the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE). In some embodiments, the PSMA-targeting molecule is or includes 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).

In some embodiments, the PSMA-targeting molecule is or includes an antibody or antigen-binding fragment thereof, optionally having a binding site that specifically binds to PSMA. In some embodiments, the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or includes a CDR3, a V_{H} and/or V_{L}, and/or competes for binding to PSMA or binds to the same PSMA epitope as any of the foregoing.

In some embodiments, the PSMA-targeting molecule is or includes an aptamer or a conjugate thereof. In some embodiments, the aptamer includes A9, A10, A10g, A10-3.2 or SZT101 or a conjugate thereof.

In some embodiments, the PSMA-targeting molecule provides a signal or induces a signal that is detectable or is capable of binding to a moiety that provides a signal or induces a signal that is detectable; and/or the PSMA-targeting molecule is or includes a moiety that provides a signal or induces a signal that is detectable. In some embodiments, wherein the detecting comprises identifying a signal in the subject or from a sample from the subject, wherein :the PSMA-targeting molecule provides the signal or induces the signal that is detectable or is capable of binding to a moiety that provides the signal or induces the signal that is detectable; and/or the PSMA-targeting molecule is or comprises a moiety that provides the signal or induces the signal that is detectable.

In some embodiments, the moiety includes a radioisotope, a bioluminescent compound, a chemiluminescent compound, a fluorescent compound, a chromogenic compound, a quantum dot, a nanoparticle, a metal chelate or an enzyme.

In some embodiments, the PSMA-targeting molecule is or includes an imaging probe or a detection reagent, which optionally is a radioligand.

In some embodiments, the PSMA-targeting molecule is or includes the moiety and/or the PSMA-targeting molecule is capable of being cleaved upon binding the PSMA or modified form thereof, wherein cleavage results in at least one cleavage product containing the moiety and/or is fluorescent and/or radioactive.

In some embodiments, the PSMA-targeting molecule also includes a moiety that provides a signal or induces a signal that is detectable, and the moiety is linked directly or indirectly, optionally via a linker, to a portion of the PSMA-targeting molecule capable of binding to the PSMA or to the modified form thereof.

In some embodiments, the linker is a releasable linker or a cleavable linker. In some embodiments, the linker is capable of being cleaved upon binding the PSMA or modified form thereof, wherein cleavage results in at least one cleavage product containing the moiety and/or is fluorescent and/or radioactive. In some embodiments, the releasable linker or the cleavable linker is released or cleaved in the presence of one or more conditions or factors present in the tumor microenvironment (TME), wherein cleavage results in at least one cleavage product containing the moiety and/or is fluorescent and/or radioactive. In some embodiments, the one or more conditions or factors present in the tumor microenvironment (TME) includes matrix metalloproteinase (MMP), hypoxic conditions or acidic conditions.

In some embodiments, the radiotherapeutic, radioisotope, radioligand or radioactive cleavage product includes ¹¹C, ¹⁸F, ⁶⁴Cu, ⁶⁸Ga, ⁶⁸Ge, ⁸⁶Y, ⁸⁹Zr, ⁹⁰Y, ^{99m}Tc, ¹¹¹In, ¹²³I, ¹²⁵I, ¹⁷⁷Lu and/or ²¹³Bi.

In some embodiments, the PSMA-targeting molecule is 2-(3-{1-carboxy-5-[(6-[¹⁸F]fluoro-pyridine-3-carbonyl)-amino]-pentyl}-ureido)-pentanedioic acid (¹⁸F-DCFPyL) or N-[N-[(S)-1,3-dicarboxypropyl]carbamoyl]-4-[¹⁸F]fluorobenzyl-L-cysteine (¹⁸F-DCFBC).

In some embodiments, the contacting and/or the detecting is performed in vivo and/or the detection is carried out via in vivo imaging.

In some embodiments, the in vivo imaging is selected from among magnetic resonance imaging (MRI), single-photon emission computed tomography (SPECT), computed tomography (CT), computed axial tomography (CAT), electron beam computed tomography (EBCT), high resolution computed tomography (HRCT), hypocycloidal tomography, positron emission tomography (PET), scintigraphy, gamma camera, a β+ detector, a γ detector, fluorescence imaging, low-light imaging, X-rays, bioluminescence imaging and near-infrared (NIR) optical tomography. In some embodiments, the in vivo imaging is positron emission tomography (PET), optionally coupled with computed tomography (CT).

In some embodiments, the methods are capable of detecting as low or few as, or as low or few as approximately 4,000, 5,000, 6,000, 7,000, 8,000, 9,000 or 10,000 cells or as low or as few as approximately 4,000, 5,000, 6,000, 7,000, 8,000, 9,000 or 10,000 cells present in a specified volume. In some embodiments, the specified volume is a volume of liquid, of a sample, and/or of an organ or tissue and/or is of between or between about 10 and about 100 μL.

In some embodiments, the contacting and/or the detecting is performed in vitro or ex vivo. In some embodiments, the contacting and/or the detecting includes immunohistochemistry (IHC), immunocytochemistry, or flow cytometry. In some embodiments, the methods are capable of detecting as low or few as, or as low or few as approximately 2,000, 3,000, 4,000, 5,000, 6,000, 7,000, 8,000, 9,000 or 10,000 cells or as low or as few as approximately 2,000, 3,000, 4,000, 5,000, 6,000, 7,000, 8,000, 9,000 or 10,000 cells present in a specified volume. In some embodiments, the specified volume is a volume of liquid, of a sample, and/or of an organ or tissue and/or is of between or between about 10 and about 100 μL.

In some embodiments, the method further includes determining the number or concentration of the administered engineered cells in the subject. In some embodiments, determining comprises comparing the signal to a standard curve. In some embodiments, the standard curve is generated from detection of the signal from a plurality of control samples containing a defined number of cells expressing the PSMA or modified form thereof, said plurality of control samples having been contacted with the PSMA-targeting molecule.

In some embodiments, the in vivo imaging is positron emission tomography (PET), optionally coupled with computed tomography (CT), and the PSMA-targeting molecule is 2-(3-{1-carboxy-5-[(6-[18F]fluoro-pyridine-3-carbonyl)-amino]-pentyl}-ureido)-pentanedioic acid (18F-DCFPyL).

Also provided herein are methods of selecting, isolating or separating cells expressing PSMA or a modified form thereof. In some embodiments, the methods include contacting a plurality of cells containing any of the engineered cells described herein with a PSMA-targeting molecule; and selecting, isolating or separating cells that are recognized or bound by the PSMA-targeting molecule. In some of any of the provided embodiments, the recombinant receptor is a chimeric receptor and/or a recombinant antigen receptor, such as a chimeric antigen receptor (CAR).

Also provided herein are methods of selecting, isolating or separating cells expressing PSMA or a modified form thereof. In some embodiments, the methods include selecting, isolating or separating cells that are recognized or bound by a PSMA-targeting molecule, from a plurality of cells containing any of the engineered cells herein that have been contacted with the PSMA-targeting molecule. In some of any of the provided embodiments, the recombinant receptor is a chimeric receptor and/or a recombinant antigen receptor, such as a chimeric antigen receptor (CAR).

In some embodiments, the PSMA-targeting molecule or a portion thereof: is capable of binding to a PSMA and/or to the modified form thereof, and/or is capable of binding to the active site of a PSMA and/or of the modified form of PSMA, and/or is capable of being cleaved by a PSMA and/or by the modified form of PSMA; and/or is an antagonist, a selective antagonist, an inverse agonist, a selective inverse agonist, an agonist, a selective agonist, an inhibitor, and/or a selective inhibitor of a PSMA and/or of the modified form thereof.

In some embodiments, the plurality of cells includes engineered cells containing any of the polynucleotides, set of polynucleotides or compositions containing set of polynucleotides described herein or vectors, set of vectors or compositions containing set of vectors described herein.

In some embodiments, the plurality of cells containing the engineered cells comprises peripheral blood leukocytes from a subject having been administered any of the engineered cells or compositions described herein.

In some embodiments, the PSMA-targeting molecule is or includes a small molecule, a ligand, an antibody or antigen-binding fragment thereof, an aptamer, a peptide, or a conjugate thereof.

In some embodiments, the PSMA-targeting molecule is or includes a small molecule. In some embodiments, the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE). In some embodiments, the PSMA-targeting molecule is or includes 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).

In some embodiments, the PSMA-targeting molecule is or includes an antibody or antigen-binding fragment thereof, optionally having a binding site that specifically binds to PSMA. In some embodiments, the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or includes a CDR3, a V_{H} and/or V_{L}, and/or competes for binding to PSMA or binds to the same PSMA epitope as any of the foregoing.

In some embodiments, the PSMA-targeting molecule is or includes an aptamer or a conjugate thereof. In some embodiments, the aptamer includes A9, A10, A10g, A10-3.2 or SZT101 or a conjugate thereof.

In some embodiments, the PSMA-targeting molecule is comprised in a matrix or immobilized on a solid support. In some embodiments, the solid support includes a magnetic particle.

Also provided herein are kits. In some embodiments, the kits contain a composition containing a therapeutically effective amount of any of the engineered cells provided herein; and a composition containing a PSMA-targeting molecule. In some embodiments, the PSMA-targeting molecule is or includes or also includes a therapeutic agent and/or the PSMA-targeting molecule provides a signal or induces a signal that is detectable or is capable of binding to a moiety that provides a signal or induces a signal that is detectable. In some of any of the provided embodiments, the recombinant receptor is a chimeric receptor and/or a recombinant antigen receptor, such as a chimeric antigen receptor (CAR). In some embodiments, wherein the detecting comprises identifying a signal in the subject or from a sample from the subject, wherein :the PSMA-targeting molecule provides the signal or induces the signal that is detectable or is capable of binding to a moiety that provides the signal or induces the signal that is detectable; and/or the PSMA-targeting molecule is or comprises a moiety that provides the signal or induces the signal that is detectable.

In some embodiments, the kit also includes instructions for administering, to a subject for treating a disease or condition, the engineered cell and the PSMA-targeting molecule in a combined therapy for treating the disease or condition. In some embodiments, the kit also includes instructions for administering the PSMA-targeting molecule to a subject receiving or having been administered the engineered cells for detecting the engineered cells.

Also provided herein are kits containing a composition containing a therapeutically effective amount of any of the engineered cells provided herein; and instructions for administering a PSMA-targeting molecule to a subject receiving or having been administered the engineered cells for detecting the engineered cells.

Also provided herein are kits containing a composition containing a PSMA-targeting molecule; and instructions for administering the PSMA-targeting molecule to a subject receiving or having been administered a therapeutically effective amount of any of the engineered cells provided herein for detecting the engineered cells. In some of any of the provided embodiments, the recombinant receptor is a chimeric receptor and/or a recombinant antigen receptor, such as a chimeric antigen receptor (CAR).

In some embodiments, instructions further specify determining the number or concentration of the administered engineered cells in the subject. In some embodiments, the instructions specify that determining comprises comparing the signal to a standard curve. In some embodiments, instructions further specify that the standard curve is generated from detection of the signal from a plurality of control samples containing a defined number of cells expressing the PSMA or modified form thereof, said plurality of control samples having been contacted with the PSMA-targeting molecule. In some embodiments, the instructions specify that detecting is carried out via positron emission tomography (PET), optionally coupled with computed tomography (CT), and the PSMA-targeting molecule is 2-(3-{1-carboxy-5-[(6-[¹⁸F]fluoro-pyridine-3-carbonyl)-amino]-pentyl}-ureido)-pentanedioic acid (¹⁸F-DCFPyL).

Also provided herein are kits containing a composition containing a therapeutically effective amount of any of the engineered cells provided herein; and instructions for administering, to a subject for treating a disease or condition, the engineered cells in a combined therapy with a PSMA-targeting molecule, said PSMA-targeting molecule that is or includes or also includes a therapeutic agent for treating the disease or condition. In some of any of the provided embodiments, the recombinant receptor is a chimeric receptor and/or a recombinant antigen receptor, such as a chimeric antigen receptor (CAR).

In some embodiments, the PSMA-targeting molecule or the therapeutic agent is capable of modulating the tumor microenvironment (TME) or is cytotoxic to the tumor. In some embodiments, the therapeutic agent is an immunomodulatory agent, a cytotoxic agent, an anti-cancer agent or a radiotherapeutic.

Also provided herein are kits containing a composition containing a PSMA-targeting molecule; and instructions for administering, to a subject for treating a disease or condition, the PSMA-targeting molecule in a combined therapy with a therapeutically effective amount of any of the engineered cells provided herein for treating the disease or condition. In some of any of the provided embodiments, the recombinant receptor is a chimeric receptor and/or a recombinant antigen receptor, such as a chimeric antigen receptor (CAR).

In some embodiments, the PSMA-targeting molecule is or includes a small molecule, a ligand, an antibody or antigen-binding fragment thereof, an aptamer, a peptide, or a conjugate thereof.

In some embodiments, the PSMA-targeting molecule is or includes a small molecule. In some embodiments, the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N,N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE). In some embodiments, the PSMA-targeting molecule is or includes 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).

In some embodiments, the PSMA-targeting molecule is or includes an antibody or antigen-binding fragment thereof, optionally having a binding site that specifically binds to PSMA. In some embodiments, the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or includes a CDR3, a V_{H} and/or V_{L}, and/or competes for binding to PSMA or binds to the same PSMA epitope as any of the foregoing.

In some embodiments, the PSMA-targeting molecule is or includes an aptamer or a conjugate thereof. In some embodiments, the aptamer includes A9, A10, A10g, A10-3.2 or SZT101 or a conjugate thereof.

Also provided herein are PSMA-targeting molecules. In some embodiments, the PSMA-targeting molecule includes a portion capable of binding to a PSMA or to a modified form thereof, wherein the PSMA-targeting molecule is or also includes an immunomodulatory agent. In some embodiments, the immunomodulatory agent is capable of modulating, optionally increasing, the activity of an immune cell or an immune response and/or is capable of modulating the tumor microenvironment (TME). In some of any of the provided embodiments, the recombinant receptor is a chimeric receptor and/or a recombinant antigen receptor, such as a chimeric antigen receptor (CAR).

In some embodiments, the PSMA-targeting molecule is or includes a prodrug that is or includes or is capable of conversion into or unmasking of the immunomodulatory agent and/or the PSMA-targeting molecule is capable of being cleaved upon binding to the PSMA or modified form thereof, wherein cleavage results in at least one cleavage product containing the immunomodulatory agent.

In some embodiments, the immunomodulatory agent is linked directly or indirectly, optionally via a linker, to a portion of the PSMA-targeting molecule capable of binding to the PSMA or to the modified form thereof. In some embodiments, the linker is a peptide or a polypeptide or is a chemical linker. In some embodiments, the linker is a releasable linker or a cleavable linker. In some embodiments, the linker is capable of being cleaved upon binding to the PSMA or modified form thereof by the binding molecule, wherein cleavage results in at least one cleavage product containing the immunomodulatory agent. In some embodiments, the releasable linker or the cleavable linker is released or cleaved in the presence of one or more conditions or factors present in the tumor microenvironment (TME), wherein cleavage results in at least one cleavage product containing the immunomodulatory agent. In some embodiments, the one or more conditions or factors present in the tumor microenvironment (TME) includes matrix metalloproteinase (MMP), hypoxic conditions or acidic conditions.

In some embodiments, the immunomodulatory agent is an immune checkpoint inhibitor or modulator or a cytokine. In some embodiments, the immunomodulatory agent is an immune checkpoint inhibitor capable of inhibiting or blocking a function of an immune checkpoint molecule or a signaling pathway involving an immune checkpoint molecule. In some embodiments, the immune checkpoint molecule is selected from among PD-1, PD-L1, PD-L2, CTLA-4, LAG-3, TIM3, VISTA, an adenosine receptor or extracellular adenosine, optionally an adenosine 2A Receptor (A2AR) or adenosine 2B receptor (A2BR), or adenosine or a pathway involving any of the foregoing.

Also provided herein are PSMA-targeting molecules. In some embodiments, the PSMA-targeting molecule includes a portion capable of binding to a PSMA or to a modified form thereof, wherein the PSMA-targeting molecule is or also includes a therapeutic agent capable of modulating the tumor microenvironment (TME), wherein the therapeutic agent is linked to the portion of the PSMA targeting molecule by a releasable or cleavable linker responsive to one or more conditions or factors present in the TME. In some embodiments, the one or more conditions or factors present in the tumor microenvironment (TME) includes matrix metalloproteinase (MMP), hypoxic conditions or acidic conditions. In some embodiments, the therapeutic agent is an immunomodulatory agent, a cytotoxic agent, an anti-cancer agent or a radiotherapeutic. In some of any of the provided embodiments, the recombinant receptor is a chimeric receptor and/or a recombinant antigen receptor, such as a chimeric antigen receptor (CAR).

In some embodiments, the PSMA-targeting molecule of is an antibody-drug conjugate (ADC).

In some embodiments, the PSMA-targeting molecule or a portion thereof: is capable of binding to a PSMA and/or to the modified form thereof, and/or is capable of binding to the active site of a PSMA and/or of the modified form of PSMA, and/or is capable of being cleaved by a PSMA and/or by the modified form of PSMA; and/or is an antagonist, a selective antagonist, an inverse agonist, a selective inverse agonist, an agonist, a selective agonist, an inhibitor, and/or a selective inhibitor of a PSMA and/or of the modified form thereof.

In some embodiments, the PSMA-targeting molecule is or includes a small molecule, a ligand, an antibody or antigen-binding fragment thereof, an aptamer, a peptide, or a conjugate thereof.

In some embodiments, the PSMA-targeting molecule is or includes a small molecule. In some embodiments, the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE). In some embodiments, the PSMA-targeting molecule is or includes 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).

In some embodiments, the PSMA-targeting molecule is or includes an antibody or antigen-binding fragment thereof, optionally having a binding site that specifically binds to PSMA. In some embodiments, the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or includes a CDR3, a V_{H} and/or V_{L}, and/or competes for binding to PSMA or binds to the same PSMA epitope as any of the foregoing.

In some embodiments, the PSMA-targeting molecule is or includes an aptamer or a conjugate thereof. In some embodiments, the aptamer includes A9, A10, A10g, A10-3.2 or SZT101 or a conjugate thereof.

Also provided herein are methods of treatment that includes administering any of the PSMA-targeting molecules described herein to a subject. In some of any of the provided embodiments, the recombinant receptor is a chimeric receptor and/or a recombinant antigen receptor, such as a chimeric antigen receptor (CAR).

Also provided herein are articles of manufacture. In some embodiments, the articles of manufacture includes any of the engineered cells, compositions, polynucleotides, set of polynucleotides, composition containing set of polynucleotides, vectors, set of vectors, composition containing set of vectors, kits or PSMA-targeting molecules provided herein. In some of any of the provided embodiments, the recombinant receptor is a chimeric receptor and/or a recombinant antigen receptor, such as a chimeric antigen receptor (CAR).

### BRIEF DESCRIPTION OF THE DRAWINGS

FIGS. 1A-1B depicts exemplary FACS plots, showing detected levels of binding of the anti-PSMA antibody (PSMA (mAb)) and CD4 in CAR+ T cell-enriched samples. The cells were engineered to express an anti-CD19 CAR and full-length wild-type PSMA (WT PSMA), N-terminally modified PSMA variant bearing an amino acid replacement at position 2 (PSMA^{(W2G)}) or a nine amino acid N-terminal deletion (PSMA^{(N9del)}), or as control, cells were transduced with a vector not encoding the CAR and/or not encoding PSMA (mock). FIG. 1C shows the geometric mean fluorescent intensities (gMFI) for expression of PSMA (or N-terminally modified variants) as determined by binding of the anti-PSMA antibody, for CD4+ and CD8+ T cells. FIG. 1D shows gMFI for expression of PSMA (or N-terminally modified variants) in CD4+ or CD8+ cells transduced to express one of the respective PSMA or variants, versus on T cells expressing a truncated EGFR (EGFRt) or that were subject to mock transduction. FIGS. 1E-1F depicts exemplary FACS plots, showing detected levels of binding of the YC-36-FITC (FITC-conjugated analog of DCFPyL) and CD8 in CAR+ T cell-enriched samples expressing anti-CD19 CAR and PSMA (or N-terminally modified variants). FIG. 1G depicts an exemplary FACS plot, showing co-expression of the modified PSMA^{(N9del)} on the surface of cells expressing the CAR, as determined by an anti-idiotype antibody specific for the binding domain of the CAR. FIGS. 1H-1L depict exemplary FACS plots showing the expression of PSMA and anti-CD19 CAR in CD4+ or CD8+ cells that had been transduced with WT PSMA or modified variants (PSMA^{(W2G)} and PSMA^{(N9del)}) FIG. 1M shows the gMFI for surface expression of the CAR by flow cytometry, in anti-CD19 CAR-expressing cells co-expressing the truncated PSMA variant (CD19-tPSMA; PSMA^{(N9del)}) or cells expressing a truncated receptor as a control surrogate marker (CD19-tReceptor).

FIG. 2A shows cytolytic activity of CAR+ cells expressing PSMA (or N-terminally modified variants), or CAR+ cells expressing the alternative EGFRt marker, as assessed by measuring the loss of viable NLR-labeled CD19-expressing target cells (NLR+ cells), at the 4:1 effector to target cell ratio. FIG. 2B shows a comparison of results for different killing index, at E:T ratios (4:1, 2:1, 1:1 and 1:2). FIG. 2C shows cytolytic activity of T cells expressing anti-CD19 CAR/PSMA^{(N9del)}, in the presence of 2-(3-{1-carboxy-5-[(6-fluoro-pyridine-3-carbonyl)-amino]-pentyl}-ureido)-pentanedioicacid (DCFPyL), a reagent that binds to the catalytic domain of PSMA, at an E:T ratio of 4:1. FIG. 2D shows the killing index, of T cells expressing anti-CD19 CAR/PSMA^{(N9del)}, in the presence of 2-(3-{1-carboxy-5-[(6-fluoro-pyridine-3-carbonyl)-amino]-pentyl}-ureido)-pentanedioicacid (DCFPyL), at E:T ratios of 4:1 or 1:1.

FIG. 3A (IFN-γ), FIG. 3B (TNF-α) and FIG. 3C (IL-2) depict the amount of respective cytokine production by T cells expressing anti-CD19 CAR/WT PSMA, anti-CD19 CAR/PSMA^{(W2G)}, anti-CD19 CAR/PSMA^{(N9del)} or mock-transduced cells (mock), in response to incubation with CD19-expressing target cells. FIG. 3D shows IFN-γ production in anti-CD19 CAR-expressing cells co-expressing the truncated PSMA variant (CD19-tPSMA; PSMA^{(N9del)}) or cells expressing a truncated receptor as a control surrogate marker (CD19-tReceptor). FIG. 3E depicts amount of IFN-γ produced in co-cultures of T cells expressing anti-CD19 CAR/PSMA^{(N9del)} and K562-CD19 target cells, at an E:T ratio of 4:1 and 1:1, cultured in the presence of varying concentrations of DCFPyL.

FIG. 4A shows tumor growth over time in mice with tumors, receiving a dose of engineered T cells expressing anti-CD19 CAR/PSMA^{(N9del)}, anti-CD19 CAR/EGFRt, or mock-transduced cells (mock). Tumor growth over time was determined by the average radiance (p/s/cm²/sr) determined bioluminescence imaging of mice model having a tumor expressing a bioluminescent protein. FIG. 4B shows the survival curve of the mice in each group.

FIG. 5A shows exemplary positron emission tomography—computed tomography (PET/CT) scans of mice injected with T cells containing 50% CAR+ cells and 50% non-CAR+ T cells, such that the mice had been injected with 500 (0.5k), 2,500 (2.5k), 5,000 (5k), 25,000 (25k), 50,000 (50k), 250,000 (250k), 500,000 (0.5M) or 2,500,000 (2.5M) engineered cells expressing anti-CD19 CAR/PSMA^{(Ndel)}. The mice also were injected with 400 μCi of 2-(3-{1-carboxy-5-[(6-[¹⁸F]fluoro-pyridine-3-carbonyl)-amino]-pentyl}-ureido)-pentanedioicacid ([¹⁸F]DCFPyL). A transverse view is shown for the exemplary animal having received 2.5M engineered cells expressing anti-CD19 CAR/PSMA^{(N9del)} and 400 μCi [¹⁸F]DCFPyL. FIG. 5B shows exemplary positron emission tomography—computed tomography (PET/CT) from an additional study, with 2,000 (2k), 4,000 (4k), 20,000 (20k),40,000 (40k), 200,000 (200k), 400,000 (400k) or 2,000,000 (2M) anti-CD19 CAR+ T cells expressing PSMA-N9del in 50 μL (50% Matrigel®) injected into the shoulders of NOD/Scid/gc−/− (NSG) mice (white arrows indicating the location of the injected cells). PET results were expressed in percentage of injected dose per cubic centimeter of tissue imaged (% ID/cc).

FIG. 6 shows the bioluminescence imaging and PET/CT scans of mice with disseminated tumors, who had received a dose of engineered T cells expressing anti-CD19 CAR/PSMA^{(N9del)}, anti-CD19 CAR/EGFRt, mock-transduced cells (mock) or no T cells. The top panels show bioluminescence imaging of tumors and spontaneous metastases developed in exemplary mice in each test group. Bottom panels show the corresponding PET/CT scans for detection of the modified PSMA-expressing CAR+ cells using the PSMA-targeting agent in each of the mice shown on the respective portions of the top panel.

FIG. 7 shows an in vitro detection of a varying number of engineered anti-CD19 CAR/PSMA^{(N9del)}-expressing T cells using PSMA-targeting radiolabeled agent ([¹⁸F]DCFPyL) and PET imaging. CAR+/PSMA^{(N9del)}-expressing T cell numbers in 20 μL PBS are depicted on the left panel; PET imaging results are depicted on the right panel.

FIG. 8 shows a survival curve representing survival over time of mice of a xenograft model with disseminated tumors, that had received a dose of engineered T cells expressing anti-CD19 CAR/PSMA^{(N9del)} or mock-transduced cells (mock), or without treatment (no treatment).

FIGS. 9A-9D depict exemplary bioluminescence imaging results, at day 0 and 11 after CAR+ T cell injection, and PET/CT imaging results, at days 5 and 12 after CAR+ T cell injection, in mice with disseminated tumors receiving a dose of engineered T cells expressing anti-CD19 CAR/PSMA^{(N9del)}. The first and third image panels show the results of bioluminescence imaging, at day 0 and 11, respectively, and the second and fourth image panels show the results of the PET/CT scans for detection of the modified PSMA-expressing CAR+ cells using the PSMA-targeting agent, at days 5 and 12, respectively. FIG. 9E depicts the bioluminescence imaging results, on day 0, and PET/CT imaging results, on day 9, of untreated mice (untreated) and the mock study group (mock treated). Arrows indicate location of the tumors.

FIG. 10 depicts the results of immunohistochemistry to determine the presence of tumor cells and PSMA-expressing CAR+ cells in mice with disseminated tumors that had received a dose of engineered T cells expressing anti-CD19 CAR/PSMA^{(N9del)}. Sections of tumor tissue were stained using an anti-GFP antibody to detect the Nalm6-GFP-ffluc tumor cells or an anti-PSMA antibody to detect the anti-CD19 CAR/PSMA^{(N9del)} T cells.

FIG. 11A shows the standard curve for determining the number of PSMA-expressing cells by PET/CT imaging using the PSMA-specific PET ligand [¹⁸F]DCFPyL. The standard curve was determined by plotting the total voxels (unit of graphic information in three-dimensional space) from the PET images (n=8) from an in vitro phantom imaging experiment, against corresponding cell numbers. The resulting linear regression equation was y=5×10⁻⁶x+0.0122 (R²=0.9989). The error bars in show the standard deviation. FIGS. 11B and 11C show exemplary bioluminescence imaging results and PET/CT imaging results for tumor model mice that had been administered anti-CD19 CAR/PSMA^{(N9del)}-expressing cells, on days 4, 5, 6, 7, 8, 9, 10, 11 and 12 after the initial BLI on day 0. The total number of CAR+ cells in the tumor were extrapolated based on the standard curve, and are listed in Table 1.

FIG. 12 shows the density of administered CAR+ T cells present in tumor biopsy samples obtained from tumor model mice that had been administered anti-CD19 CAR/PSMA^{(N9del)}-expressing cells between days 6 and 13 after administration was determined by counting the number of PSMA+ cells stained with an anti-PSMA antibody, and the density of CAR+ T cells from human tumor biopsy samples after administration of CAR+ T cells.

FIGS. 13A-13B depicts the exemplary results of immunohistochemistry to determine the presence of tumor cells and PSMA-expressing CAR+ cells in mice with disseminated tumors that had received engineered T cells expressing anti-CD19 CAR/PSMA^{(N9del)}, on days 4-11. Sections of tumor tissue were stained using an anti-GFP antibody to detect the Nalm6-GFP-ffluc tumor cells or an anti-PSMA antibody to detect the anti-CD19 CAR/PSMA^{(N9del)} T cells.

FIGS. 14A-14B show exemplary bioluminescence imaging results and PET/CT imaging results for tumor model mice that had been administered anti-CD19 CAR/PSMA^{(N9del)} expressing cells, on days 10, 11 and 12 after the initial BLI on day 0. FIGS. 14C-14D plot the total number of CAR+ cells in the tumor, extrapolated based on a standard curve, against the percentage of CAR+ T cells among live cells in peripheral blood (PPB) or within bone marrow (BM) samples determined using flow cytometry by staining with an anti-PSMA antibody, in each of the mice depicted in FIGS. 14A-14B.

### DETAILED DESCRIPTION

Provided herein are engineered cells that express a prostate-specific membrane antigen (PSMA) or a modified form thereof, e.g., a variant PSMA, and a recombinant molecule such as a recombinant receptor, e.g., a chimeric antigen receptor (CAR). Also provided are related methods and compositions including therapeutic methods and compositions. In some embodiments, the engineered cells also express a recombinant receptor, e.g., a recombinant antigen receptor and/or chimeric receptor. In some embodiments, the engineered cells co-express a PSMA, typically a modified PSMA, and a recombinant receptor (e.g. a CAR). Also provided are methods involving the detection and/or targeting of such cells using PSMA-targeting agents or PSMA-targeting molecules. Among the methods are detection and imaging methods and methods for combination therapy, e.g., of an engineered cell and additional therapeutic agent(s).

Among the provided methods are those in which the PSMA or modified PSMA expressed by the cells can be exploited, e.g., for a therapeutic, diagnostic, monitoring and/or modulatory outcome or approach. In some embodiments, the PSMA or modified PSMA is exploited for detection, e.g., imaging, of the cells, such as in vivo, in a subject to which the cells are or have been administered. In some embodiments, the PSMA or modified PSMA is exploited for targeting or delivery of a therapeutic agent, e.g., to an antigen, cell or tissue recognized by the cell or recombinant receptor. In some embodiments, the PSMA or modified PSMA is targeted for treating a disease or condition and/or for detecting, imaging, identifying, selecting, isolating, separating, engineering of cells, processing of cells and/or removing of cells, including in connection with genetically engineered cells, e.g. cells engineered with a recombinant receptor (e.g. a CAR). In some embodiments, the PSMA or modified form thereof acts as a marker, such as for confirmation of engineering of cells, e.g., transduction of cells and/or for confirming the presence or number of engineered cells. Also provided are polynucleotides and vectors encoding the PSMA and/or the recombinant receptor, methods for engineering such cells, and methods that employ such engineered cells, including methods for treating, detecting, selecting, isolating or separating such cells. Also provided are PSMA-targeting molecules that can target or bind PSMA, e.g., PSMA or modified form thereof expressed on the surface of the engineered cells. In some embodiments, the PSMA-targeting molecule includes a therapeutic agent and/or a moiety, e.g., a moiety that provides a signal or induces a signal that is detectable. In some embodiments, the PSMA-targeting molecules can be used in connection with the engineered cells in the methods provided herein.

Various strategies are available for producing and administering engineered cells for adoptive therapy. The cells generally are engineered by introducing one or more genetically engineered nucleic acid or product thereof,. Among the nucleic acids and products thereof are genetically engineered antigen receptors, including engineered T cell receptors (TCRs) and functional non-TCR antigen receptors, such as chimeric antigen receptors (CARs), including activating, stimulatory, and costimulatory CARs, and combinations thereof. For example, strategies are available for engineering cells, such as T cells, expressing chimeric receptors, such as CARs, and administering compositions containing such engineered cells to subjects.

Throughout the process of producing engineered cells, it can be beneficial to be able to identify, detect, locate, and/or select transduced cells and/or cells expressing the desired recombinant molecule such as the recombinant receptor. Likewise, subsequent to or substantially simultaneously with administration of the engineered cells for adoptive therapy, there can also be a need or desire to detect, monitor, observe, localize or identify the adoptively transferred cells. For example, it may be desired to determine or assess the presence or absence or degree of expansion, persistence, immunogenicity of, or to determine the biodistribution and/or pharmacokinetics of the adoptively transferred cells, and/or to provide a mechanism to deplete or reduce the number of adoptively transferred cells in a subject.

In some cases, certain available methods for determining the biodistribution and/or pharmacokinetics of administered cells, e.g., adoptively transferred cells, may not be entirely satisfactory. For example, it may be difficult to determine the whole-body spatial distribution of adoptively transferred cells, to determine specific location of the cells, e.g., within or around the site or location of a disease or disorder, e.g., tumor, persistence of the cells in the body and/or development of adverse effects, e.g., toxicities.

Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38), Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (e.g., encoding a CAR or TCR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the provided compositions and methods are advantageous in that they permit the detection or imaging of adoptively transferred cells with a high degree of sensitivity, which in some aspects may provide the ability for direct assessment of circulating engineered cells, e.g., CAR T cells, in vivo following infusion. In some aspects, provided embodiments are advantageous in their suitability for in vivo use, for example, by providing the ability to detect cells with reagents such as PSMA-targeting molecules, in a manner that targets, binds, and/or detects administered cells, such as CAR T cells, without impacting one or more functions of the engineered cells and/or for detection or separation during cell production. In some aspects, the provided embodiments are also advantageous in that they allow in vivo detection without changing the sequence or function of the engineered recombinant receptors. In some embodiments, the provided embodiments are based on the observation that the prostate-specific membrane antigen (PSMA) or modified form thereof is effectively expressed on the cell surface of the engineered cells for detection without altering the function of the engineered cells, e.g., as shown by assessing in vitro cytotoxicity and/or in vivo antitumor activity. In some embodiments, the provided embodiments also can be used to detect the presence, biodistribution and trafficking of administered cells engineered to express a recombinant receptor at primary and metastatic tumor sites in vivo.

In some embodiments, the provided agents, compositions, articles of manufacture, combinations and methods are useful and/or advantageous in the determining the biodistribution and/or pharmacokinetics of administered cell compositions. In some aspects, the methods are advantageous in providing safe, highly sensitive, minimally invasive, real-time, relatively non-immunogenic and/or in not impacting the functionality of the engineered cells. In some aspects, the provided embodiments include cell surface markers that can aid the production, monitoring, and/or post-administration stages involving transduced cell products. For example, in some aspects, provided are methods for efficient selection and isolation of cells positive for the transgene and for monitoring transgene-expressing cells in vivo and ex vivo. In some embodiments, the provided engineered cells, nucleic acids, vectors, compositions, PSMA-targeting molecules, and methods provide one or more advantages compared to existing markers or selections strategies used in connection with engineered cells.

In some embodiments, the prostate-specific membrane antigen (PSMA) or modified form thereof that is engineered to be expressed on the engineered cells provided herein, is a human PSMA. In some embodiments, the cells generally are engineered by introducing one or more genetically engineered nucleic acid or product thereof, such as those encoding recombinant receptors and/or PSMA or modified PSMA. Engineering cells to express PSMA or a modified form thereof, in particular human PSMA, for administering the engineered cells to human subjects, is advantageous because PSMA is not immunogenic. PSMA is an antigen that is expressed in the human body, in a highly tissue-specific manner. Thus, expression of PSMA or modified form thereof thus allows detection and targeting of particular cells, using PSMA-targeting molecules that specifically bind to and/or target PSMA, without eliciting an immune response or having immunogenic properties.

In some embodiments, the engineered cells expressing PSMA or a modified form thereof can be utilized to target a PSMA-targeting molecule that can include a portion capable of binding PSMA and a detectable moiety or agent. In some aspects, PSMA-targeting molecules can be utilized in connection with the engineered cells provided herein to permit delivery or targeting of therapeutic agents; detection, identification, or imaging of cells, selection; isolation or separation of cells; and to promote cell suicide or cell removal. Various known reagents that can specifically target PSMA may be used in connection with the embodiments for therapeutic and/or detection purposes.

In some aspects, targeting PSMA or modified form thereof can facilitate detection of the detectable moiety or agent can allow detection of the engineered cells, e.g., in a subject that has been administered the engineered cells. In particular, in some embodiments, the PSMA-targeting molecules and systems can be used for in vivo imaging of cells expressing PSMA. Using PSMA-targeting molecules for in vivo imaging in some aspects allows minimally invasive, real-time, accurate and/or rapid determination, assessment and/or confirmation or monitoring of biodistribution of the engineered cells.

In some embodiments, PSMA or modified form thereof as a marker renders the engineered cells compatible for detection using minimally invasive, rapid, accurate and/or sensitive methods. For example, the provided PSMA or a modified form thereof is capable of being recognized by and/or specifically bound by ligands, antibodies or antigen-binding fragment thereof, or other PSMA-targeting molecules used for real-time or in vivo imaging of cells, such as positron emission tomography (PET), computed tomography (CT) and single photon emission computed tomography (SPECT), using radionuclide-labeled ligands. PET is a minimally invasive, rapid, accurate and sensitive method for detecting particular cells in a body and provides whole body spatial resolution. In some aspects, the provided embodiments offer an advantage of accurately and rapidly providing quantitative assessment of the administered cells, e.g., biodistribution and/or pharmacokinetics of administered cells, without the use of an invasive method. In some aspects, the provided embodiments provide an advantage over existing methods of assessing biodistribution or pharmacokinetics, such as peripheral blood draws, which do not provide any spatial information or specific information at the tumor site, or biopsies, which are invasive and only provide information at the tumor site. Thus, in some aspects, engineering cells to express PSMA or a modified form thereof, such as one that can be recognized or targeted or bound by a PSMA-targeting molecule, can provide certain advantages. Further, the provided engineered cells, nucleic acids, vectors, compositions, PSMA-targeting molecules, and methods provide cell surface molecule target and reagents to facilitate the processing, production, and/or function of the cells, and/or to facilitate the selection, separation, killing and/or removal of the cells.

In some embodiments, the engineered cells expressing PSMA or a modified form thereof and PSMA-targeting molecule can be utilized to target therapeutic agents, including immunomodulatory agents or cytotoxic agents, to a certain site, location, microenvironment and/or to certain types of cells. For example, the PSMA or modified form thereof can be targeted for delivery of a PSMA-targeting molecule that can include a portion capable of binding PSMA or a modified form thereof and a therapeutic agent, to sites, locations and/or microenvironments that contain the engineered cells, e.g., tumor microenvironment (TME).

Also provided are methods for using cells expressing the PSMA or modified form thereof. Provided are methods for cell isolation and genetic engineering. Provided are nucleic acids, such as constructs, e.g., viral vectors encoding the PSMA or modified form thereof and/or encoding nucleic acids and/or proteins of the PSMA or modified form thereof, and methods for introducing such nucleic acids into the cells, such as by transduction. Also provided are PSMA-targeting molecules comprising a portion that is capable of binding PSMA or modified form thereof and an immunomodulatory agent. Also provided are compositions containing the engineered cells, and methods, kits, and articles of manufacture for administering and monitoring the cells and compositions to subjects, such as for adoptive cell therapy.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### I. ENGINEERED CELLS EXPRESSING PROSTATE-SPECIFIC MEMBRANE ANTIGEN (PSMA) AND RECOMBINANT RECEPTOR

Provided herein are engineered cells that express a prostate-specific membrane antigen (PSMA) or a modified form thereof, e.g., a variant of PSMA, and related compositions, articles of manufacture, combinations, methods and uses, including those involving administration of the cells and detection, binding or targeting thereof with PSMA-targeting agents or molecules. The engineered cells generally also express a recombinant molecule such as a recombinant receptor, e.g., a chimeric antigen receptor (CAR). In some embodiments, the methods, compositions and uses are in connection with the provided engineered cells.

A. Prostate-Specific Membrane Antigen (PSMA)

In some embodiments, the prostate-specific membrane antigen (PSMA; also known as glutamate carboxypeptidase 2 or folate hydrolase 1) or modified form thereof, is expressed in engineered cells, e.g. primary cells, such as immune cells, e.g. T cells, including cells also engineered with a recombinant receptor, e.g. CAR. In some embodiments, the PSMA is a human PSMA.

PSMA is a type II transmembrane protein, which contains a short cytoplasmic amino terminus, a single membrane-spanning domain, and a large extracellular domain. PSMA contains a sequence of amino acids that exhibit similarity to the peptidase family M28 proteins that include co-catalytic metallopeptidases. Wild-type, full-length human PSMA, is a 750-amino acid protein that includes an intracellular portion of 19 amino acid residues, a transmembrane portion of 24 amino acid residues, and an extracellular portion of 707 amino acid residue. In humans, PSMA is encoded by the FOLH1 gene, e.g., described in GenBank Accession No. DD461260 (set forth in SEQ ID NO:26), and isoforms and variants thereof. Exemplary human PSMA amino acid sequence is set forth in, e.g., UniProt Accession No. Q04609 (set forth in SEQ ID NO:23).

In some aspects, the intracellular (N-terminal) portion of PSMA contain amino acid residues involved in cellular internalization, e.g., clathrin-dependent endocytic internalization of the molecule. In some aspects, the cellular internalization is mediated by N-terminal amino acids, such as amino acid residues at positions 1-5 of the exemplary human PSMA amino acid sequence set forth in SEQ ID NO:23 (see, e.g., Rajasekaran et al. (2003) Mol. Biol. Cell. 14:4835-4845).

In some cases, the extracellular portion of PSMA folds into three distinct structural and functional domains: a protease domain (residues 56-116 and 352-590), an apical domain (residues 117-351) and a C-terminal helical domain (residues 592-750), with reference to positions a wild-type human PSMA sequence, e.g., the amino acid sequence set forth in SEQ ID NO:23 (see, e.g., Davis et al., (2005) Proc. Natl. Acad. Sci. 102(17): 5981-5986; Mesters et al., (2006) EMBO Journal 25:1375-1384). PSMA generally contains a binuclear zinc site and can act as glutamate carboxypeptidase or folate hydrolase, catalyzing the hydrolytic cleavage of glutamate from poly-γ-glutamated folates. PSMA also has N-acetylated-alpha-linked-acidic dipeptidase (NAALADase) activity and dipeptidyl-peptidase IV type activity. The enzymatic site contains two zinc ions, and is composed of two pockets, the glutamate-sensing pocket (S1′ pocket) and the non-pharmacophore pocket (S1 pocket). Amino acid residues from the three domains generally are involved in substrate recognition, binding, and/or catalytic activity. In some cases, active site residues and/or residues involved in substrate binding and/or catalytic activity in PSMA include amino acid residues at positions 210, 257, 269, 272, 377, 387, 387, 424,424,425,433,436,453,517,518,519,552,553,534,535,536,552,553,628,666,689, 699 and/or 700, with reference to positions a wild-type human PSMA sequence, e.g., the amino acid sequence set forth in SEQ ID NO:23. In some cases, active site residues include one more residues to coordinate the active zinc ions, such as one or more residues corresponding to His377, Asp387, Glu425, Asp453, and/or His553, with reference to position of a wild-type human PSMA sequence, e.g. the amino acid sequence set forth in SEQ ID NO:23. In some embodiments, the N-acetylated-alpha-linked-acidic dipeptidase (NAALADase) domain of PSMA can also be defined as including amino acid residues 274-587, with reference to positions a wild-type human PSMA sequence, e.g., the amino acid sequence set forth in SEQ ID NO:23 (Speno et al., (1999) Molecular Pharmacology 55:179-185).

In some embodiments, any of the PSMA or modified form thereof in connection with the provided disclosure is a recombinant PSMA. In some embodiments, the PSMA or modified form thereof in connection with the provided disclosure is expressed from a heterologous, recombinant, exogenous and/or transgenic nucleic acid molecule, i.e., a nucleic acid normally not present in a cell or sample obtained from the cell, such as one obtained from another organism or cell, which for example, is not ordinarily found in the cell being engineered and/or an organism from which such cell is derived.

In some embodiments, the PSMA or modified form thereof, e.g., recombinant PSMA or modified form thereof, in connection with the provided disclosure contains one more active site residues and/or residues involved in PSMA substrate binding and/or PSMA catalytic activity, such as one more amino acid residues corresponding to amino acid residues at positions 210, 257, 269, 272, 377, 387, 387, 424, 424, 425, 433, 436, 453, 517, 518, 519, 552, 553, 534, 535, 536, 552, 553, 628, 666, 689, 699 and/or 700 of wild-type human PSMA sequence, e.g., the amino acid sequence set forth in SEQ ID NO:23. In some embodiments, the PSMA or modified form thereof, e.g., recombinant PSMA or modified form thereof, in connection with the provided disclosure exhibits the same or substantially the same substrate recognition as wild-type human PSMA, e.g. the amino acid sequence set forth in SEQ ID NO:23 or an allelic variant or other variant thereof and/or a sequence that exhibits at least or about at least or at least about 85%, 86%, 87%, 88%, 89%, 90%, 91%, 92%, 93%, 94%, 95%, 96%, 97%, 98%, 99% or more sequence identity to SEQ ID NO:23.

In some embodiments, the PSMA or modified form thereof, e.g., recombinant PSMA or modified form thereof, in connection with the provided disclosure does not bind or recognize and/or exhibits reduced binding and/or recognition, e.g. reduced by greater than or greater than about 20%, 30%, 40%, 50%, 60%, 70%, 80%, 90% or more, of a substrate or ligand of wild-type human PSMA, e.g. the amino acid sequence set forth in SEQ ID NO:23. In some embodiments, the PSMA or modified form thereof, e.g., recombinant PSMA or modified form thereof, is modified in, e.g. by amino acid replacement, insertion or substitution, of one more residues involved in active site residues and/or residues involved in PSMA substrate binding and/or PSMA catalytic activity, such as one more amino acid residues corresponding to amino acid residues at positions 210, 257, 269, 272, 377, 387, 387, 424, 424, 425, 433, 436, 453, 517, 518, 519, 552, 553, 534, 535, 536, 552, 553, 628, 666, 689, 699 and/or 700 of wild-type human PSMA sequence, e.g., the amino acid sequence set forth in SEQ ID NO:23 or an allelic variant or other variant thereof and/or a sequence that exhibits at least or about at least or at least about 85%, 86%, 87%, 88%, 89%, 90%, 91%, 92%, 93%, 94%, 95%, 96%, 97%, 98%, 99% or more sequence identity to SEQ ID NO:23.

In some embodiment, the PSMA or modified form thereof, e.g., recombinant PSMA or modified form thereof, in connection with the provided disclosure contains the protease domain or a portion thereof and/or exhibits catalytic activity, such as activity to catalyze the hydrolytic cleavage of glutamate from poly-γ-glutamated folates. In some embodiments, the PSMA or modified form thereof, e.g., recombinant PSMA or modified form thereof, in connection with the provided disclosure contains one more residues associated with zinc binding and/or residues for catalytic activity, such as one or more residues His377, Asp387, Glu425, Asp453, and/or His553. In some embodiments, the PSMA or modified form thereof, e.g., recombinant PSMA or modified form thereof, in connection with the provided disclosure contains the NAALADase domain and/or includes amino acid residues 274-587, 274-700 or 247-750, with reference to positions a wild-type human PSMA sequence, e.g., the amino acid sequence set forth in SEQ ID NO:23 or an allelic variant or other variant thereof and/or a sequence that exhibits at least or about at least or at least about 85%, 86%, 87%, 88%, 89%, 90%, 91%, 92%, 93%, 94%, 95%, 96%, 97%, 98%, 99% or more sequence identity to SEQ ID NO:23.

In some embodiments, the PSMA or modified form thereof, e.g., recombinant PSMA or modified form thereof, is not catalytically active or exhibits reduced catalytic activity, e.g. reduced by greater than or greater than about 20%, 30%, 40%, 50%, 60%, 70%, 80%, 90% or more, such as does not exhibit and/or exhibits reduced catalytic activity to catalyze the hydrolytic cleavage of glutamate from poly-γ-glutamated folates. In some embodiments, the PSMA or modified form thereof, e.g., recombinant PSMA or modified form thereof, is modified, e.g. by amino acid replacement, insertion or substitution, of one or more amino acid residues associated with zinc binding and/or catalytic activity, such as one or more residues His377, Asp387, Glu425, Asp453, and/or His553 with reference to a wild-type human PSMA sequence, e.g. the amino acid sequence set forth in SEQ ID NO:23 or an allelic variant or other variant thereof and/or a sequence that exhibits at least or about at least or at least about 85%, 86%, 87%, 88%, 89%, 90%, 91%, 92%, 93%, 94%, 95%, 96%, 97%, 98%, 99% or more sequence identity to SEQ ID NO:23.

PSMA also exists on the membrane surface as a monomer but is enzymatically active only as a dimer. The C-terminal domain is involved in forming a dimerization interface with the protease domain and the apical domain of another PSMA monomer (see, e.g., Davis et al., (2005) Proc. Natl. Acad. Sci. 102(17): 5981-5986; Mesters et al., (2006) EMBO Journal 25:1375-1384). In some embodiments, the PSMA or modified form thereof in connection with the provided disclosure includes one or more residues involved in dimerization and/or is capable of forming a dimer. In some embodiments, the PSMA or modified form thereof is modified in, e.g. by amino acid replacement, insertion or substitution, or one or more amino acid residues involved in dimerization and/or is not capable of forming a dimer.

In some cases, PSMA or a modified form thereof in connection with the present disclosure is capable of being recognized by and/or specifically bound by ligands, antibodies or antigen-binding fragment thereof, or other PSMA-targeting molecules. In some cases, PSMA or a modified form thereof in connection with the present disclosure contains epitopes, e.g., epitopes capable of being recognized by antibodies or antigen-binding fragment thereof. In some aspects, the epitope is a linear epitope. In other aspects, the epitope is a conformational epitope. Exemplary epitopes that in some embodiments can be recognized by PSMA-targeting antibodies or antigen-binding fragment thereof include amino acid residues 57-134, 91-108, 100-104, 118-135, 271-288, 469-486, 638-657, 640-657 or 716-723, with reference to positions a wild-type human PSMA sequence, e.g., the amino acid sequence set forth in SEQ ID NO:23.

In some embodiments, the provided engineered cells contain or express a PSMA or a modified from thereof. In some embodiments, the PSMA is a mammalian PSMA or a modified form thereof. In some embodiments, the PSMA is a human PSMA or a modified form thereof. In some embodiments, the PSMA provided in connection with the present disclosure is wild-type PSMA, optionally wild-type human PSMA or an allelic variant or other variant thereof, e.g. alternative isoform or fragment thereof. An exemplary sequence encoding human PSMA is set forth in SEQ ID NO:26, which encodes a human PSMA set forth in SEQ ID NO:23. In some embodiments, the PSMA is encoded by a modified nucleic acid sequence, e.g., a nucleic acid sequence that is modified to be CpG-free and/or is codon optimized. In some embodiments, the modified nucleic acid sequence is codon optimized for expression in human cells. In some aspects, codon optimization involves balancing the percentages of codons selected with the published abundance of human transfer RNAs so that none is overloaded or limiting. In some embodiments, a CpG-free nucleic acid sequence encoding PSMA is or includes modified cDNA sequence that contains no CpG sequences. In some aspects, the CpG-free nucleic acid and/or codon optimized sequence does not change the protein sequence, compared to the wild-type or unmodified PSMA. An exemplary CpG-free nucleic acid sequence encoding PSMA is set forth in SEQ ID NO:27. In some aspects, the PSMA encoded by the CpG-free PSMA has substantial percent identity to the protein sequence set forth in SEQ ID NO:23.

In some embodiments, the PSMA or modified form thereof, e.g., recombinant PSMA or modified form thereof, is encoded by a nucleic acid sequence set forth in SEQ ID NO:26 or 27, or a sequence of nucleic acids that exhibits at least or at least about 85%, 86%, 87%, 88%, 89%, 89%, 90%, 91%, 92%, 93%, 94%, 95%, 96%, 97%, 98%, 99% or more sequence identity to any of SEQ ID NOS: 26 or 27, or a fragment thereof. In some embodiments, the PSMA or modified form thereof, e.g., recombinant PSMA or modified form thereof, is encoded by a nucleic acid sequence encoding a fragment containing the extracellular domain and/or transmembrane domain encoded by SEQ ID NO:26 or 27 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:26 or 27.

In some embodiments, the PSMA or modified form thereof, e.g., recombinant PSMA or modified form thereof, is encoded by a nucleic acid sequence set forth in SEQ ID NO:53, or a sequence of nucleic acids that exhibits at least or at least about 85%, 86%, 87%, 88%, 89%, 89%, 90%, 91%, 92%, 93%, 94%, 95%, 96%, 97%, 98%, 99% or more sequence identity to any of SEQ ID NOS: 53, or a fragment thereof. In some embodiments, the PSMA or modified form thereof, e.g., recombinant PSMA or modified form thereof, is encoded by a nucleic acid sequence encoding a fragment containing the extracellular domain and/or transmembrane domain encoded by SEQ ID NO:53 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:53.

In some embodiments, the wild-type or unmodified PSMA is human PSMA and/or comprises the sequence of amino acids set forth in SEQ ID NO:23, a sequence of amino acids that exhibits at least or at least about 85%, 86%, 87%, 88%, 89%, 90%, 91%, 92%, 93%, 94%, 95%, 96%, 97%, 98%, 99% or more sequence identity thereto, or is a fragment thereof In some embodiments, the fragment contains the extracellular domain and/or transmembrane domain of the sequence of amino acids set forth in SEQ ID NO:23 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:23.

In some embodiments, the PSMA or modified form thereof, e.g., recombinant PSMA or modified form thereof, comprises the sequence of amino acids set forth in SEQ ID NO:23 or a fragment thereof or an extracellular and/or transmembrane domain thereof or a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS:23 or a fragment thereof or an extracellular and/or transmembrane domain thereof. In some embodiments, the PSMA or modified form thereof, e.g., recombinant PSMA or modified form thereof, is a full-length PSMA. In some embodiments, the PSMA is a wild-type PSMA, or an unmodified PSMA. In some embodiments, the PSMA is a wild-type human PSMA. In some embodiments, the PSMA comprises or consists essentially of the sequence set forth in SEQ ID NO:23.

In some embodiments, the modified PSMA is a recombinant modified PSMA. In some embodiments, the modified PSMA in connection with the provided disclosure is expressed from a heterologous, recombinant, exogenous and/or transgenic nucleic acid molecule, i.e., a nucleic acid normally not present in a cell or sample obtained from the cell, such as one obtained from another organism or cell, which for example, is not ordinarily found in the cell being engineered and/or an organism from which such cell is derived.

In some embodiments, the PSMA or modified form thereof is a modified PSMA, e.g., recombinant modified PSMA, comprising one or more amino acid modifications compared to a wild-type or unmodified PSMA. In some embodiments, the one or more amino acid modification comprises one or more amino acid substitutions, deletions and/or insertions. In some embodiments, the one or more amino acid modification comprises a deletion or truncation of one or more contiguous amino acid residues, e.g., at, from or near the N- or C-terminal of the wild-type or unmodified PSMA. In some embodiments, the modified PSMA exhibits altered PSMA enzymatic activity and/or ligand binding ability and/or cellular internalization. In some embodiments, the modified PSMA (i) exhibits reduced endogenous signaling; (ii) exhibits increased cell surface expression; and/or (iii) exhibits reduced cellular internalization compared to the wild-type or unmodified PSMA.

In some embodiments, the PSMA or modified form thereof, e.g., recombinant PSMA or modified form thereof, contains one or more amino acid substitution. In some embodiments, the modified PSMA comprises at least one amino acid substitution, e.g., at the second amino acid residue, where the tryptophan is substituted by glycine, corresponding to W2G, with reference to positions in PSMA set forth in SEQ ID NO:23. In some embodiments, the modified PSMA comprises at least one amino acid substitution corresponding to W2G or does not comprise W2 or does not comprise any residue at position 2, with reference to positions in the PSMA sequence set forth in SEQ ID NO:23. For example, in some embodiments, the modified PSMA comprises the sequence of amino acids set forth in SEQ ID NO:24 or a fragment thereof, or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS:24 or a fragment thereof and comprises the at least one amino acid substitution.

In some embodiments, the modified PSMA comprises an amino acid substitution at one or more of amino acid residues at position 2, 3, 4, 5, 6, 7, 8, 9, 10 or 14 with reference to positions in the PSMA sequence set forth in SEQ ID NO:23, to alanine.

In some embodiments, the PSMA is a modified PSMA that comprises a deletion of one or more N-terminal amino acid residues within the intracellular portion, compared to the wild-type or unmodified PSMA. In some embodiments, the PSMA is a modified PSMA that comprises a deletion of one or more contiguous amino acid residues in the intracellular portion compared to the wild-type or unmodified PSMA. Wild-type, full-length human PSMA, is a 750-amino acid protein that includes an intracellular portion of 19 amino acid residues, a transmembrane portion of 24 amino acid residues, and an extracellular portion of 707 amino acid residue. In some embodiments, the modified PSMA contains a deletion at or near the N-terminus (corresponding to the 5′ end of the coding sequence in the nucleic acid sequence encoding PSMA or modified form thereof), the deletion being within the intracellular portion of PSMA. In some aspects, the modified PSMA containing one or more deletions, optionally contiguous amino acid residues, within the intracellular portion is also referred to as a truncated form of PSMA, a truncated PSMA or a tPSMA.

In some aspects, the truncated PSMA or tPSMA contains a deletion or truncation of one or more amino acid residues, optionally contiguous amino acid residues, at or near the N-terminal of the wild-type or unmodified PSMA. In some aspects, the modified PSMA contains a deletion or truncation of one or more amino acid residues, e.g., one or more contiguous amino acid residues, within an intracellular portion or domain of the PSMA. In some embodiments, the PSMA protein containing a deletion N-terminal amino acids allows the N-terminally modified PSMA to successfully localize to the cell membrane and centrosome and/or (i) exhibits reduced endogenous signaling; (ii) exhibits increased cell surface expression; and/or (iii) exhibits reduced cellular internalization compared to the wild-type or unmodified PSMA. In some embodiments, the modified PSMA exhibits reduced endogenous signaling or reduced cellular internalization, e.g. reduced by greater than or greater than about 20%, 30%, 40%, 50%, 60%, 70%, 80%, 90% or more. In some embodiments, the modified PSMA exhibits increased cell surface expression or increased localization to the cell membrane and centrosome, e.g. increased by greater than or greater than about 20%, 30%, 40%, 50%, 60%, 70%, 80%, 90% or more. In some aspects, cell surface expression and/or cellular internalization can be assessed using cell imaging techniques, such as confocal microscopy using labeled binding molecules, e.g., antibodies, that specifically bind to PSMA or variant thereof

In some embodiments, the modified PSMA, e.g. tPSMA, contains or retains a methionine as a first residue, which, in some cases, is required for translation. In some embodiments, the PSMA is a modified PSMA that comprises a deletion of one or more N-terminal amino acid residues, optionally contiguous amino acid residues, within the intracellular portion, compared to the wild-type or unmodified PSMA, but does not include a deletion of the initial methionine required for translation.

In some embodiments, the modified PSMA, e.g., the tPSMA, contains a deletion of up to 2, 3, 4, 5, 6, 7, 8, 9, 10, 11 or 12 N-terminal amino acid residues compared to the wild-type or unmodified PSMA. In some embodiments, the modified PSMA contains a deletion of up to 12, 13, 14, 15, 16, 17, 18, 19, 20 or 21 N-terminal amino acid residues compared to the wild-type or unmodified PSMA. In some aspects, the modified PSMA is a truncated PSMA (tPSMA) containing a deletion of up to 2, 3, 4, 5, 6, 7, 8, 9, 10, 11, 12, 13, 14, 15, 16, 17, 18, 19, 20 or 21 N-terminal amino acid residues compared to the wild-type or unmodified PSMA in which the first N-terminal methionine is retained or is not deleted.

In some embodiments, the modified PSMA contains deletion of a contiguous sequence of amino acids at the N-terminus starting from residues 2, 3, 4, or 5 and up to residues 6, 7, 8, 9, 10, 11, 12, 13, 14, 15, 16, 17, 18, 19, 20 or 21, with reference to positions in the PSMA sequence set forth in SEQ ID NO:23.. In some embodiments, the modified PSMA contains a deletion of residues 2-22, 2-21, 2-20, 2-19, 2-18, 2-17, 2-16, 2-15, 2-14, 2-13, 2-12, 2-11, 2-10, 2-9, 2-8, 2-7, 2-6, 2-5, 2-4 or 2-3, with reference to positions in the PSMA sequence set forth in SEQ ID NO:23

In some cases, the modified PSMA may contain deletion of the first N-terminal residue, e.g. a methionine. In some embodiments, the modified PSMA contains a deletion of residues 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3 or 1-2, with reference to positions in the PSMA sequence set forth in SEQ ID NO:23.

In some aspects, the nucleic acid sequences encoding the PSMA or modified form thereof is contained in a polynucleotide that also contains a nucleic acid sequence encoding another protein, such as a different protein, for example, in an expression construct. In some aspects, the nucleic acid sequence encoding the PSMA or modified form thereof is contained within a single open reading frame with nucleic acid sequences encoding a different molecule, e.g., a recombinant receptor. In some embodiments, the nucleic acid sequence encoding the PSMA or modified form thereof is separated from the nucleic acid sequence encoding a different molecule, e.g., a recombinant receptor, by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some embodiments of such polynucleotides, the nucleic acid encoding the PSMA may encode an initial methionine or need not encode an initial methionine. In some aspects, if the sequences encoding the PSMA are more downstream, e.g., closer to the 3′ end of the coding sequence, in relation to the sequences encoding another protein, for example, the PSMA is translated after another protein, the initial methionine may not be required to be retained. In some aspects, if the sequences encoding the PSMA are more upstream, e.g., closer to the 5′ end of the coding sequence, of or is present prior to the sequences encoding another protein, for example the PSMA is translated first, the initial methionine may be required to be retained.

In some aspects, the modified PSMA is a truncated PSMA (tPSMA) containing a deletion of 9 N-terminal amino acid residues (not including the N-terminal methionine required for translation) compared to the wild-type or unmodified PSMA, e.g., PSMA^{(N9del)}. In some embodiments, the modified PSMA is a truncated PSMA (tPSMA) that contains a deletion of residues 2-10 of the sequence set forth in SEQ ID NO:23, or a fragment thereof or of a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity thereto. In some embodiments, the modified PSMA comprises the sequence of amino acids set forth in SEQ ID NO:52, or a fragment thereof or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity thereto.

In some aspects, the modified PSMA is a truncated PSMA (tPSMA) containing a deletion of 9 N-terminal amino acid residues compared to the wild-type or unmodified PSMA. In some embodiments, the modified PSMA is a truncated PSMA (tPSMA) that contains a deletion of residues 1-9 of the sequence set forth in SEQ ID NO:23, or a fragment thereof or of a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity thereto. In some embodiments, the modified PSMA comprises the sequence of amino acids set forth in SEQ ID NO:25, or a fragment thereof or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity thereto.

In some embodiments, the PSMA or modified form thereof comprises the sequence of amino acids set forth in SEQ ID NO:25 or 52 or a fragment thereof or a sequence of amino acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NOS:25 or 52 or a fragment thereof and comprises deletion of the one or more N-terminal amino acid residues.

In some embodiments, the modified PSMA is encoded by a nucleic acid sequence set forth in SEQ ID NO:53, or a sequence of nucleic acids that exhibits at least or at least about 85%, 86%, 87%, 88%, 89%, 89%, 90%, 91%, 92%, 93%, 94%, 95%, 96%, 97%, 98%, 99% or more sequence identity to SEQ ID NOS:53, or is a fragment thereof, e.g., a fragment containing the extracellular domain and/or transmembrane domain. In some embodiments, the modified PSMA is encoded by a nucleic acid sequence that does not include a deletion of the methionine start codon required for translation, e.g., encodes a polypeptide that contains an initial methionine. In some embodiments, the modified PSMA is encoded by a modified nucleic acid sequence, e.g., a nucleic acid sequence that is modified to be CpG-free and/or is codon optimized.

In some embodiments, the modified PSMA comprises all or substantially all of the transmembrane domain of the wild-type or unmodified PSMA; or the modified PSMA comprises a transmembrane domain with the same or at least the same number of amino acids as the transmembrane domain of a wild-type or unmodified PSMA.

In some embodiments, the PSMA or modified form thereof comprises a sequence of amino acids bound by or recognized by a PSMA-targeting molecule, such as an antibody or an antigen-binding fragment thereof. In some embodiments, the PSMA or modified form thereof comprise an epitope recognized by an antibody or antigen-binding fragment thereof. In some embodiments, the PSMA or modified form thereof comprises a sequence of amino acids at residues 57-134, 91-108, 100-104, 118-135, 271-288, 469-486, 638-657, 640-657 or 716-723, with reference to positions in the sequence of amino acids set forth in SEQ ID NO:23, or a sequence of amino acids that exhibits at least or at least about 85%, 86%, 87%, 88%, 89%, 90%, 91%, 92%, 93%, 94%, 95%, 96%, 97%, 98%, 99% or more sequence identity thereto.

In some embodiments, the PSMA or modified form thereof comprises at least one or more of the extracellular domains of the PSMA or portions thereof. In some embodiments, the PSMA or modified form thereof comprises at least one or more extracellular domains selected from among a protease domain (residues 56-116 and 352-590), an apical domain (residues 117-351) and a C-terminal helical domain (residues 592-750), with reference to positions a wild-type human PSMA sequence, e.g., the amino acid sequence set forth in SEQ ID NO:23. In some embodiments, the modified PSMA contains at least two of the extracellular domains of PSMA.

In some embodiments, the PSMA or modified form thereof comprises an extracellular catalytic domain or an enzymatically active domain. In some embodiments, the PSMA or modified form thereof comprises an N-acetylated-alpha-linked-acidic dipeptidase (NAALADase) domain. In some embodiments, the PSMA or modified form thereof comprises a sequence of amino acids at residues 274-587, 247-700 or 247-750 with reference to positions in the sequence of amino acids set forth in SEQ ID NO:23, or a sequence of amino acids that exhibits at least or at least about 85%, 86%, 87%, 88%, 89%, 90%, 91%, 92%, 93%, 94%, 95%, 96%, 97%, 98%, 99% or more sequence identity thereto. In some embodiments, the PSMA or modified form thereof comprises active site residues and/or residues involved in substrate binding and/or catalytic activity. In some embodiments, the PSMA or modified form thereof comprises residues, e.g., conserved residues, at positions 210, 257, 269, 272, 377, 387, 387, 424, 424,425,433,436,453,517,518,519,552,553,534,535,536,552,553,628,666,689,699 and/or 700, with reference to positions in the sequence of amino acids set forth in SEQ ID NO:23.

In some embodiments, the PSMA or a modified form thereof is a modified PSMA that comprises one or more deletion of a domain or region of the extracellular portion. For example, in some embodiments, the deletion of a domain or region of the extracellular portion comprises a deletion of a sequence of amino acids at positions 44-273 or 588-750 with reference to positions in the sequence of amino acids set forth in SEQ ID NO:23. In some embodiments, the modified PSMA comprises a deletion of one or more amino acid residues, optionally one or more contiguous amino acid residues, in the extracellular portion that is not bound by or recognized by a PSMA-targeting molecule, e.g., a ligand or an antibody or an antigen-binding fragment thereof. In some embodiments, the PSMA or modified form thereof comprises a deletion of one or more C-terminal amino acid residues, optionally one or more contiguous amino acid residues. In some embodiments, the PSMA or modified form thereof comprises a deletion of amino acid residues 103-750, 626-750, 721-747 or 736-750, with reference to positions in PSMA set forth in SEQ ID NO:23. In some embodiments, the PSMA or modified form thereof comprises a deletion of 15 C-terminal amino acid residues, with reference to positions in PSMA set forth in SEQ ID NO:23.

In some embodiments, the modified PSMA includes a PSMA described in, e.g., International PCT Pub. No. WO2015143029, Rajasekaran et al. (2003) Mol. Biol. Cell. 14:4835-4845, Rajasekaran et al. (2008) Mol Cancer Ther. (2008) 7(7): 2142-2151, Barinka et al. (2004) Eur. J. Biochem. 271:2782-2790, and Davis et al. (2005) Proc. Natl. Acad. Sci. 102(17)-5981-5986.

In some embodiments, the PSMA or modified form thereof, optionally the extracellular portion, is capable of being recognized by a PSMA-targeting molecule or a portion thereof, such as any PSMA-targeting molecule or portion thereof described herein.

B. Recombinant Receptors

In some embodiments, provided are engineered cells, such as immune cells, such as T cells, that express a recombinant receptor and a PSMA or modified PSMA. Among the receptors are antigen receptors and receptors containing one or more component thereof. The recombinant receptors may include chimeric receptors, such as those containing ligand-binding domains or binding fragments thereof and intracellular signaling domains, functional non-TCR antigen receptors, chimeric antigen receptors (CARs), and T cell receptors (TCRs), such as transgenic TCRs, and components of any of the foregoing. In some embodiments, the provided engineered cells express the recombinant receptor and the PSMA or modified form thereof, typically a recombinant PSMA or modified PSMA. The chimeric receptor, such as a CAR, generally includes the extracellular antigen (or ligand) binding domain linked to one or more intracellular signaling components, in some aspects via linkers and/or transmembrane domain(s).

1. Chimeric Antigen Receptors (CARs)

In some embodiments, engineered cells, such as T cells, are provided that express a CAR with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as chimeric receptor, contains an intracellular signaling region, which includes a cytoplasmic signaling domain (also interchangeably called an intracellular signaling domain), such as a cytoplasmic (intracellular) region capable of inducing a primary activation signal in a T cell, for example, a cytoplasmic signaling domain of a T cell receptor (TCR) component (e.g. a cytoplasmic signaling domain of a zeta chain of a CD3-zeta (CD3ζ) chain or a functional variant or signaling portion thereof) and/or that comprises an immunoreceptor tyrosine-based activation motif (ITAM).

In some embodiments, the chimeric receptor further contains an extracellular ligand-binding domain that specifically binds to a ligand (e.g. antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb).

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MI-IC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g. CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the CAR contains an antibody or an antigen-binding fragment (e.g. scFv) that specifically recognizes an antigen, such as an intact antigen, expressed on the surface of a cell.

In some embodiments, the antigen (or a ligand) is a tumor antigen or cancer marker. In some embodiments, the antigen (or a ligand) is or includes orphan tyrosine kinase receptor ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), ephrine receptor A2 (EPHa2), Her2/neu (receptor tyrosine kinase erbB2), Her3 (erb-B3), Her4 (erb-B4), erbB dimers, type III epidermal growth factor receptor mutation (EGFR vIII), folate binding protein (FBP), FCRL5, Fc receptor like 5 (FCRL5; also known as Fc receptor homolog 5 or FCRH5), fetal acetylcholine receptor, ganglioside GD2, ganglioside GD3, G Protein Coupled Receptor 5D (GPCR5D), HMW-MAA, IL-22R-alpha, IL-13R-alpha2, kinase insert domain receptor (kdr), kappa light chain, Leucine Rich Repeat Containing 8 Family Member A (LRRC8A), Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, Human leukocyte antigen A1 (HLA-A1), MAGE A1, HLA-A2, NY-ESO-1, PSCA, folate receptor-a, CD44v6, CD44v7/8, αvβ6 integrin (avb6 integrin), 8H9, NCAM, VEGF receptors, 5T4, Foetal AchR, natural killer group 2 member D (NKG2D) ligands, CD44v6, dual antigen, a cancer-testes antigen, mesothelin, murine CMV, mucin 1 (MUC1), MUC16, prostate stem cell antigen (PSCA), NKG2D, a cancer-testis antigen cancer/testis antigen 1B (CTAG, also known as NY-ESO-1 and LAGE-2), MART-1, glycoprotein 100 (gp100), oncofetal antigen, ROR1, Trophoblast glycoprotein (TPBG also known as 5T4), TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, C-C Motif Chemokine Ligand 1 (CCL-1), CD138, a pathogen-specific antigen and an antigen associated with a universal tag, and/or biotinylated molecules, and/or molecules expressed by HIV, HCV, HBV or other pathogens.

In some embodiments, the antigen or ligand is a tumor antigen or cancer marker. In some embodiments, the antigen or ligand the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CDS, CD33, Igkappa, Iglambda, CD79a, CD79b or CD30.

In some embodiments, the antigen is a pathogen-specific or pathogen-expressed antigen. In some embodiments, the antigen is a viral antigen (such as a viral antigen from HIV, HCV, HBV, etc.), bacterial antigens, and/or parasitic antigens.

In some embodiments, the antigen is or includes a pathogen-specific or pathogen-expressed antigen, such as a viral antigen (e.g., a viral antigen from HIV, HCV, HBV), bacterial antigens, and/or parasitic antigens.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19.

In some embodiments the scFv and/or V_{H} domains is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the scFv and/or V_{H} domain is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302).

In some aspects, the CAR contains a ligand- (e.g., antigen-) binding domain that binds or recognizes, e.g., specifically binds, a universal tag or a universal epitope. In some aspects, the binding domain can bind a molecule, a tag, a polypeptide and/or an epitope that can be linked to a different binding molecule (e.g., antibody or antigen-binding fragment) that recognizes an antigen associated with a disease or disorder. Exemplary tag or epitope includes a dye (e.g., fluorescein isothiocyanate) or a biotin. In some aspects, a binding molecule (e.g., antibody or antigen-binding fragment) linked to a tag, that recognizes the antigen associated with a disease or disorder, e.g., tumor antigen, with an engineered cell expressing a CAR specific for the tag, to effect cytotoxicity or other effector function of the engineered cell. In some aspects, the specificity of the CAR to the antigen associated with a disease or disorder is provided by the tagged binding molecule (e.g., antibody), and different tagged binding molecule can be used to target different antigens. Exemplary CARs specific for a universal tag or a universal epitope include those described, e.g., in U.S. Pat. No. 9,233,125, WO 2016/030414, Urbanska et al., (2012) Cancer Res 72: 1844-1852, and Tamada et al., (2012). Clin Cancer Res 18:6436-6445.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g. US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US published application No. US20020150914, US2014/0294841; and Cohen CJ. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W. H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the recombinant receptor such as the CAR, such as the antibody portion thereof, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, Hudecek et al. (2015) Cancer Immunol Res. 3(2): 125-135 or international patent application publication number WO2014031687. In some embodiments, the spacer has the sequence set forth in SEQ ID NO: 1, and is encoded by the sequence set forth in SEQ ID NO: 2. In some embodiments, the spacer has the sequence set forth in SEQ ID NO: 3. In some embodiments, the spacer has the sequence set forth in SEQ ID NO: 4.

In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some aspects, the spacer is a polypeptide spacer such as one or more selected from: (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P (SEQ ID NO:66), where X₁ is glycine, cysteine or arginine and X₂ is cysteine or threonine. In some embodiments, the spacer comprises a sequence from an immunoglobulin. In some embodiments, the spacer comprises or consists of all or a portion of an immunoglobulin hinge, optionally an IgG4 hinge and/or IgG1 hinge. In some embodiments, the spacer comprises a portion of an immunoglobulin heavy chain constant region, optionally a C_{H}2 and/or C_{H}3 region.

The antigen recognition domain generally is linked to one or more intracellular signaling components, such as signaling components that mimic activation through an antigen receptor complex, such as a TCR complex, in the case of a CAR, and/or signal via another cell surface receptor. Thus, in some embodiments, the antigen binding component (e.g., antibody) is linked to one or more transmembrane and intracellular signaling regions. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding domain is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor γ and CD8, CD4, CD25 or CD16.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma or FcR beta. In some embodiments, cytoplasmic signaling molecule(s) in the CAR contain(s) a cytoplasmic signaling domain, portion thereof, or sequence derived from CD3 zeta.

In some embodiments, the CAR includes a signaling region and/or transmembrane portion of a costimulatory receptor, such as CD28, 4-1BB, OX40, DAP10, and ICOS. In some aspects, the same CAR includes both the signaling region and costimulatory components.

In some embodiments, the signaling region is included within one CAR, whereas the costimulatory component is provided by another CAR recognizing another antigen. In some embodiments, the CARs include activating or stimulatory CARs, and costimulatory CARs, both expressed on the same cell (see WO2014/055668).

In certain embodiments, the intracellular signaling region comprises a CD28 transmembrane and signaling domain linked to a CD3 (e.g., CD3-zeta) intracellular domain. In some embodiments, the intracellular signaling region comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing the antibody or fragment described herein. In some aspects, the chimeric antigen receptor includes an extracellular portion containing the antibody or fragment described herein and an intracellular signaling domain. In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain disposed between the extracellular domain and the intracellular signaling region.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about85%, 86%, 87%, 88%, 89%, 90%, 91%, 92%, 93%, 94%, 95%, 96%, 97%, 98%, 99% or more sequence identity to SEQ ID NO:8; in some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the chimeric antigen receptor contains an intracellular domain of a T cell costimulatory molecule. In some aspects, the T cell costimulatory molecule is CD28 or 4-1BB.

In some embodiments, the intracellular signaling region comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12. In some embodiments, the intracellular region comprises an intracellular costimulatory signaling domain of a human 4-1BB or functional variant or portion thereof.

In some embodiments, the intracellular signaling region comprises a human CD3 chain, optionally a CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. Nos. 7,446,190 or 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In some aspects, the spacer contains only a hinge region of an IgG, such as only a hinge of IgG4 or IgG1, such as the hinge only spacer set forth in SEQ ID NO:1. In other embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:3. In some embodiments, the spacer is an Ig hinge, e.g., an IgG4 hinge, linked to a C_{H}3 domain only, such as set forth in SEQ ID NO:4. In some embodiments, the spacer is or comprises a glycine-serine rich sequence or other flexible linker such as known flexible linkers.

In some embodiments, the CAR comprises:
(i) in order, an scFv specific for the antigen, a transmembrane domain, a cytoplasmic signaling domain derived from a costimulatory molecule, which optionally is or comprises a 4-1BB, and a cytoplasmic signaling domain derived from a primary signaling ITAM-containing molecule, which optionally is or comprises a CD3zeta signaling domain and optionally further comprises a spacer between the transmembrane domain and the scFv;
(ii) in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or
(iii) in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain; and:
the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 55, SEQ ID NO: 56, SEQ ID NO: 57, SEQ ID NO:58, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P (SEQ ID NO:66), where X₁ is glycine, cysteine or arginine and X₂ is cysteine or threonine; and/or
the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or
the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or
the scFv comprises a CDRL1 sequence of RASQDISKYLN (SEQ ID NO: 59), a CDRL2 sequence of SRLHSGV (SEQ ID NO:60), and/or a CDRL3 sequence of GNTLPYTFG (SEQ ID NO: 61) and/or a CDRH1 sequence of DYGVS (SEQ ID NO:62), a CDRH2 sequence of VIWGSETTYYNSALKS (SEQ ID NO:63), and/or a CDRH3 sequence of YAMDYWG (SEQ ID NO:64) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a V_{H}, a linker, optionally comprising SEQ ID NO: 65, and a V_{L}, and/or the scFv comprises a flexible linker and/or comprises the amino acid sequence set forth as SEQ ID NO: 65.

2. T Cell Receptors (TCRs)

In some embodiments, engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MI-IC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the genetically engineered antigen receptors include recombinant T cell receptors (TCRs) and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified by a skilled artisan. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPredl (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPredl (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)5-P- wherein P is proline, G is glycine and S is serine (SEQ ID NO:28). In some embodiments, the linker has the sequence

(SEQ ID NO: 29)
GSADDAKKDAAKKDGKS

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

3. Chimeric Auto-Antibody Receptor (CAAR)

In some embodiments, the recombinant receptor is a chimeric autoantibody receptor (CAAR). In some embodiments, the CAAR is specific for an autoantibody. In some embodiments, a cell expressing the CAAR, such as a T cell engineered to express a CAAR, can be used to specifically bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM). In some embodiments, the intracellular signaling region comprises a secondary or costimulatory signaling region (secondary intracellular signaling regions).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

4. Multi-Targeting

In some embodiments, the cells and methods include multi-targeting strategies, such as expression of two or more genetically engineered receptors on the cell, each recognizing the same of a different antigen and typically each including a different intracellular signaling component. Such multi-targeting strategies are described, for example, in International Patent Application Publication No: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating or stimulating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating or stimulating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-κB and/or AP-1, and/or induction of gene expression of factors such as cytokines, proliferation, and/or survival.

In some embodiments, the first and/or second receptor includes intracellular signaling domains or regions of costimulatory receptors such as CD28, CD137 (4-1BB), OX40, and/or ICOS. In some embodiments, the first and second receptors include an intracellular signaling domain of a costimulatory receptor that are different. In one embodiment, the first receptor contains a CD28 costimulatory signaling region and the second receptor contain a 4-1BB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating or stimulating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

C. Nucleic Acids and Vectors

Also provided are one or more polynucleotides (e.g., nucleic acid molecules) encoding the PSMA or modified form thereof and/or recombinant receptors, vectors for genetically engineering cells to express such the PSMA or modified form thereof and receptors and methods for producing the engineered cells.

In some embodiments, provided are polynucleotides that encode any of the PSMA or modified form thereof described herein. In some aspects, the polynucleotide contains a single coding sequence, such as only a coding sequence encoding the PSMA or modified form thereof. In other instances, the polynucleotide contains at least two different coding sequences, such as a first nucleic acid sequence encoding the PSMA or modified form thereof and a second nucleic acid sequence encoding a recombinant receptor. In some aspects, the recombinant receptor is or contains a chimeric antigen receptor (CAR). In some aspects, the recombinant receptor is or contains a T cell receptor (TCR), e.g., a transgenic TCR. In some embodiments, the polynucleotides and vectors are used for co-expression in cells of the PSMA or modified form thereof and the recombinant receptor.

Also provided are sets or combinations of polynucleotides. In some embodiments, the set or combination comprises a first polynucleotide comprising a nucleic acid encoding a prostate-specific membrane antigen (PSMA) or modified form thereof and a second polynucleotide comprising a nucleic acid encoding a recombinant receptor. Also provided are compositions containing such set or combination of polynucleotides. In some embodiments, the set or combination of polynucleotides, are used together for engineering of cells. In some embodiments, the first and the second polynucleotides in the set are introduced simultaneously or sequentially, in any order into a cell for engineering.

In some embodiments, the provided polynucleotide containing nucleic acids encoding PSMA or modified form thereof includes the nucleic acid sequence set forth in SEQ ID NO:26,27 or 53, or a nucleic acid sequence that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 26, 27 or 53 or a fragment thereof.

In some cases, the nucleic acid sequence encoding the PSMA or modified form thereof contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO: 31 and encoded by the nucleotide sequence set forth in SEQ ID NO:30. In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 31 and encoded by the nucleotide sequence set forth in SEQ ID NO:30, or the CD8 alpha signal peptide set forth in SEQ ID NO:32.

In some embodiments, the polynucleotide encoding the PSMA or modified form thereof and/or recombinant receptor contains at least one promoter that is operatively linked to control expression of the PSMA or modified form thereof and/or recombinant receptor. In some examples, the polynucleotide contains two, three, or more promoters operatively linked to control expression of the PSMA or modified form thereof and/or recombinant receptor.

In certain cases where nucleic acid molecules encode two or more different polypeptide chains, each of the polypeptide chains can be encoded by a separate nucleic acid molecule. For example, two separate nucleic acids are provided, and each can be individually transferred or introduced into the cell for expression in the cell.

In some embodiments, the nucleic acid encoding the recombinant receptor and the nucleic acid encoding the PSMA or modified form thereof are operably linked to the same promoter and are optionally separated by an internal ribosome entry site (IRES), or a nucleic acid encoding a self-cleaving peptide or a peptide that causes ribosome skipping, which optionally is a T2A, a P2A, an E2A or an F2A. In some embodiments, the nucleic acids encoding the PSMA or modified form thereof and the nucleic acid encoding the recombinant receptor are operably linked to two different promoters. In some embodiments, the nucleic acid encoding the PSMA or modified form thereof and the nucleic acid encoding the recombinant receptor are present or inserted at different locations within the genome of the cell. In some embodiments, the polynucleotide encoding the recombinant receptor is introduced into a composition containing cultured cells, such as by retroviral transduction, transfection, or transformation.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products ((e.g. encoding the PSMA or modified form thereof and encoding the recombinant receptor) by a message from a single promoter. Alternatively, in some cases, a single promoter may direct expression of an RNA that contains, in a single open reading frame (ORF), two or three genes (e.g. encoding the PSMA or modified form thereof and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 22), equine rhinitis A virus (E2A, e.g., SEQ ID NO: 21), Thosea asigna virus (T2A, e.g., SEQ ID NO: 6 or 17), and porcine teschovirus-1 (P2A, e.g., SEQ ID NO: 19 or 20) as described in U.S. Patent Publication No. 20070116690.

In some embodiments, the nucleic acid encoding the PSMA or modified form thereof and the nucleic acid encoding the recombinant receptor are operably linked to the same promoter and are optionally separated by an internal ribosome entry site (IRES), or a nucleic acid encoding a self-cleaving peptide or a peptide that causes ribosome skipping, which optionally is a T2A, a P2A, a E2A or a F2A. In some embodiments, the nucleic acid encoding the PSMA or modified form thereof and the nucleic acid encoding the recombinant receptor are operably linked to two different promoters. In some embodiments, the nucleic acid encoding the PSMA or modified form thereof and the nucleic acid encoding the recombinant receptor are present or inserted at different locations within the genome of the cell.

In some embodiments, also provided are polynucleotides containing nucleic acid sequences encoding any of the PSMA or modified form thereof and nucleic acid sequences encoding any of the chimeric receptors and/or recombinant antigen receptors, e.g., a CAR provided herein. In some aspects, the nucleic acid encoding the PSMA or modified form thereof and the nucleic acid encoding the CAR are comprised within one polynucleotide. In some embodiments, the provided polynucleotide comprises, in 5′ to 3′ order: i) a nucleic acid encoding a signal peptide;ii) a nucleic acid encoding the CAR said CAR comprising an scFv; a spacer; a transmembrane domain; an intracellular region comprising a costimulatory signaling region, and an intracellular signaling domain of a CD3-zeta (CD3ζ) chain, or a signaling portion thereof; iii) a nucleic acid sequence encoding a self-cleaving peptide or a peptide that causes ribosome skipping, which optionally is a T2A, a P2A, a E2A or a F2A; and iv) a nucleic acid encoding the PSMA or modified form thereof, which optionally comprises the sequence of amino acids set forth in SEQ ID NO: 52 or a fragment thereof; or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS: 52 or a fragment thereof and that comprises deletion of the one or more N-terminal amino acid residues.

In some embodiments, the encoded CAR comprises:
(i) in order, an scFv specific for the antigen, a transmembrane domain, a cytoplasmic signaling domain derived from a costimulatory molecule, which optionally is or comprises a 4-1BB, and a cytoplasmic signaling domain derived from a primary signaling ITAM-containing molecule, which optionally is or comprises a CD3zeta signaling domain and optionally further comprises a spacer between the transmembrane domain and the scFv;
(ii) in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or
(iii) in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain; and:
the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 55, SEQ ID NO: 56, SEQ ID NO: 57, SEQ ID NO:58, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P (SEQ ID NO:66), where X₁ is glycine, cysteine or arginine and X₂ is cysteine or threonine; and/or
the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or
the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or
the scFv comprises a CDRL1 sequence of RASQDISKYLN (SEQ ID NO: 59), a CDRL2 sequence of SRLHSGV (SEQ ID NO:60), and/or a CDRL3 sequence of GNTLPYTFG (SEQ ID NO: 61) and/or a CDRH1 sequence of DYGVS (SEQ ID NO:62), a CDRH2 sequence of VIWGSETTYYNSALKS (SEQ ID NO:63), and/or a CDRH3 sequence of YAMDYWG (SEQ ID NO:64) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a V_{H}, a linker, optionally comprising SEQ ID NO: 65, and a V_{L}, and/or the scFv comprises a flexible linker and/or comprises the amino acid sequence set forth as SEQ ID NO: 65.

In some embodiments, the polynucleotide encoding the PSMA or modified form thereof and/or recombinant receptor is introduced into a composition containing cultured cells, such as by retroviral transduction, transfection, or transformation.

Also provided are vectors or constructs containing such nucleic acids and/or polynucleotides. In some embodiments, the vectors or constructs contain one or more promoters operatively linked to the nucleic acid encoding the PSMA or modified form thereof and/or recombinant receptor to drive expression thereof. In some embodiments, the promoter is operatively linked to one or more than one nucleic acid molecules or polynucleotides. Thus, also provided are vectors, such as those that contain any of the polynucleotides provided herein. In some embodiments, the vector includes a first polynucleotide encoding PSMA or a modified form thereof and a second polynucleotide encoding a recombinant receptor, e.g., CAR.

In some cases, the vector is a viral vector, such as a retroviral vector, e.g., a lentiviral vector or a gammaretroviral vector. Also provided a set or combination of vectors. In some embodiments, the set or combination of vectors comprises a first vector and a second vector, wherein the first vector comprises the first polynucleotide, e.g., a first polynucleotide encoding PSMA or a modified form thereof, and the second vector comprises the second polynucleotide encoding a recombinant receptor, e.g., CAR. Also provided are compositions containing such set or combination of vectors. In some embodiments, the set or combination of vectors, are used together for engineering of cells. In some embodiments, the first and the second vectors in the set are introduced simultaneously or sequentially, in any order into a cell for engineering.

In some embodiments, the vectors include viral vectors, e.g., retroviral or lentiviral, non-viral vectors or transposons, e.g. Sleeping Beauty transposon system, vectors derived from simian virus 40 (SV40), adenoviruses, adeno-associated virus (AAV), lentiviral vectors or retroviral vectors, such as gamma-retroviral vectors, retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV) or adeno-associated virus (AAV).

Any of the PSMA or modified form thereof and/or recombinant receptors described herein can be encoded by polynucleotides containing one or more nucleic acid sequences encoding PSMA or modified form thereof and/or recombinant receptors, in any combinations or arrangements. For example, one, two, three or more polynucleotides can encode one, two, three or more different polypeptides, e.g., PSMA or modified form thereof and/or recombinant receptors. In some embodiments, one vector or construct contains a nucleic acid sequence encoding PSMA or modified form thereof, and a separate vector or construct contains a nucleic acid sequence encoding a recombinant receptor, e.g., CAR. In some embodiments, the nucleic acid encoding the PSMA or modified form thereof and the nucleic acid encoding the recombinant receptor are operably linked to two different promoters. In some embodiments, the nucleic acid encoding the recombinant receptor is present downstream of the nucleic acid encoding the PSMA or modified form thereof.

D. Cells and Preparation of Cells for Engineering

Provided herein are cells, such as engineered cells that contain the PSMA or modified form thereof and/or a recombinant receptor. Also provided are populations of such cells, compositions containing such cells and/or enriched for such cells, such as in which cells expressing the PSMA or modified form thereof and/or recombinant receptor, e.g. chimeric receptor, make up at least 50, 60, 70, 80, 90, 91, 92, 93, 94, 95, 96, 97, 98, 99, or more percent of the total cells in the composition or cells of a certain type such as T cells or CD8+ or CD4+ cells. Among the compositions are pharmaceutical compositions and formulations for administration, such as for adoptive cell therapy. Also provided are methods for engineering, producing or generating such cells, therapeutic methods for administering the cells and compositions to subjects, e.g., patients, and methods for detecting, selecting, isolating or separating such cells.

Thus, provided are genetically engineered cells expressing the PSMA or modified form thereof and/or recombinant receptors e.g., CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation. In some embodiments, the engineered cells are not cells in which PSMA is typically or normally expressed. In some embodiments, the engineered cells are not cells in which PSMA expression is increased during a diseased condition. In some embodiments, the cell is not a prostate cell. In some embodiments, the cells do not express PSMA prior to engineering by introduction of recombinant or heterologous nucleic acid sequences encoding PSMA or a variant thereof.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some embodiments, the cell is a regulatory T cell (Treg). In some embodiments, the cell further comprises a recombinant FOXP3 or variant thereof

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for engineering may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished in a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood.1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain aspects, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International PCT Publication No. WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood.1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10:1567-1573; and Godin et al. (2008) J Biophoton. 1(5):355-376). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of PSMA or modified form thereof and a recombinant receptor, e.g., CAR.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling region of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood.1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

E. Methods for Genetic Engineering

Various methods for the introduction of genetically engineered components, e.g., PSMA or modified form thereof and recombinant receptors, e.g., CARs or TCRs, are well known and may be used with the provided methods and compositions. Exemplary methods include those for transfer of nucleic acids encoding the polypeptides or receptors, including via viral vectors, e.g., retroviral or lentiviral, non-viral vectors or transposons, e.g. Sleeping Beauty transposon system. Methods of gene transfer can include transduction, electroporation or other method that results into gene transfer into the cell.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, it may be desired to safeguard against the potential that overexpression of a stimulatory factor (for example, a lymphokine or a cytokine) could potentially result in an unwanted outcome or lower efficacy in a subject, such as a factor associated with toxicity in a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 11 :223, 1977) which confers ganciclovir sensitivity; the cellular hypoxanthine phosphribosyltransferase (HPRT) gene, the cellular adenine phosphoribosyltransferase (APRT) gene, bacterial cytosine deaminase (Mullen et al., Proc. Natl. Acad. Sci. USA. 89:33 (1992)).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion, e.g. with nucleic acids encoding PSMA or modified form thereof and/or a recombinant receptor, e.g., a T cell receptor (TCR) or a chimeric antigen receptor (CAR). This transfection for the introduction of the gene of the desired polypeptide or receptor can be carried out with any suitable retroviral vector, for example. The genetically modified cell population can then be liberated from the initial stimulus (the CD3/CD28 stimulus, for example) and subsequently be stimulated with a second type of stimulus (e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

As described above, in some embodiments, the cells are incubated and/or cultured prior to or in connection with genetic engineering. The incubation steps can include culture, cultivation, stimulation, activation, propagation and/or freezing for preservation, e.g. cryopreservation.

### II. PSMA-TARGETING MOLECULES

In some embodiments, PSMA-targeting molecules are used in connection with the engineered cells provided herein, e.g., engineered cells expressing PSMA or modified form thereof and/or a recombinant receptor, e.g., CAR, and/or in the methods provided herein. In some embodiments, the PSMA-targeting molecule is capable of binding to PSMA or a modified form thereof, or comprises a portion capable of binding PSMA or modified form thereof. In some embodiments, the PSMA-targeting molecule is or also comprises one or more therapeutic agent and/or one or more moiety that provides a signal or induces a signal that is detectable, e.g., a detectable moiety. In some embodiments, the PSMA-targeting molecule comprises a portion that is capable of binding PSMA or modified form thereof and a therapeutic agent and/or a moiety that provides a signal or induces a signal that is detectable, e.g., detectable moiety.

In some embodiments, the PSMA-targeting molecule or a portion thereof is capable of binding to a PSMA and/or to the modified form thereof, and/or is capable of binding to the active site of a PSMA and/or of the modified form of PSMA, and/or is capable of being cleaved by a PSMA and/or by the modified form of PSMA; and/or is an antagonist, a selective antagonist, an inverse agonist, a selective inverse agonist, an agonist, a selective agonist, an inhibitor, and/or a selective inhibitor of a PSMA and/or of the modified form thereof

In some embodiments, the PSMA-targeting molecule is capable of binding to a moiety that provides a signal or induces a signal that is detectable. In some embodiments, the PSMA-targeting molecule contains a therapeutic moiety that is activated, cleaved and/or released upon binding to PSMA or modified form thereof, or activated, cleaved and/or released in the presence of a particular condition, e.g., a condition near the site, location or microenvironment of a disease or disorder, e.g., in the tumor microenvironment (TME).

In some embodiments, the PSMA-targeting molecule comprises a portion that is capable of binding PSMA or modified form thereof. In some embodiments, the portion that is capable of binding PSMA or modified form thereof is or comprises a small molecule, a ligand, an antibody or antigen-binding fragment thereof, an aptamer, a peptide, a nanoparticle or a conjugate thereof. In some embodiments, the PSMA-targeting molecule is or also comprises a therapeutic agent and/or a moiety that provides a signal or induces a signal that is detectable. In some embodiments, the PSMA-targeting molecule is or comprises a portion that is capable of binding PSMA or modified form thereof linked or conjugated to a therapeutic agent and/or a moiety, e.g., a detectable moiety.

In some embodiments, the PSMA-targeting molecule is a reagent known and available for detection and/or binding of PSMA, e.g. human PSMA. PSMA has been used as a target for imaging for prostate cancers, based on low background expression and tissue-specific expression, increased expression in later-stage prostate cancers, a large extracellular domain target, human biocompatibility, available probe diversity, and proven clinical utility and internalization and endosomal recycling. Several PSMA-binding ligands have been developed for the delivery of imaging and therapeutic agents for prostate cancer, including low-molecular-weight nuclear, fluorescent, and multi-modality imaging probes (Chen et al., Biochem. Biophys. Res. Comm. 2009, 390(3):624-629; Banerjee et al., Oncotarget 2011; 2(12): 1244-1253; see also Maurer et al. (2016) Nature Reviews Urology 13:226-235; Rowe et al. (2016) Prostate Cancer Prostatic Dis. 19(3):223-230; Mease et al., (2013) Curr Top Med Chem. 13(8):951-962). Exemplary other PSMA-targeting molecules are described below.

In some embodiments, the PSMA-targeting molecule provides a signal or induces a signal that is detectable or is capable of binding to a moiety that provides a signal or induces a signal that is detectable; and/or the PSMA-targeting molecule is or comprises a moiety that provides a signal or induces a signal that is detectable. In some embodiments, the PSMA-targeting molecule includes those known or available that bind to PSMA, such as those used in treatment and/or diagnosis of prostate cancer.

In some embodiments, also provided is a PSMA-targeting molecule comprising a portion that is capable of binding PSMA or modified form thereof and an immunomodulatory agent. In some embodiments, the immunomodulatory agent is capable of modulating, optionally increasing, the activity of an immune cell or an immune response and/or is capable of modulating the tumor microenvironment (TME).

A. PSMA-Binding Portion

I. Small Molecules

In some embodiments, the PSMA-targeting molecule is or comprises a ligand and/or small molecule. In some embodiments, the PSMA-targeting molecule comprises a portion that is capable of binding PSMA or modified form thereof, that is or comprises a ligand and/or small molecule. In some embodiments, the PSMA-targeting molecule is or comprises a small molecule that is capable of binding the active site or substrate binding site of PSMA.

In some embodiments, the PSMA-targeting molecule is or comprises is an antagonist, a selective antagonist, an inverse agonist, a selective inverse agonist, an agonist, a selective agonist, an inhibitor, and/or a selective inhibitor of a PSMA and/or of the modified form thereof. In some embodiments, the PSMA-targeting molecule is or comprises an inhibitor of PSMA. In some embodiments, the PSMA-targeting molecule is or comprises a small molecule, and/or a low molecular weight molecule and/or a low molecular weight inhibitor. In some embodiments, the PSMA-targeting molecule that is or comprises a portion that is capable of binding PSMA or modified form thereof that is a small molecule, and a therapeutic agent and/or detectable moiety.

As described above, PSMA possesses an enzymatic site in its extracellular domain that cleaves endogenous substrates such as N-aceylaspartylglutamate (NAAG), tri-alpha-glutamate peptides and poly-γ-glutamyl folic acid, and the enzymatic site contains two zinc ions, and is composed of two pockets, the glutamate-sensing pocket (S1′ pocket) and the non-pharmacophore pocket (S1 pocket). Some PSMA inhibitors contain a zinc binding moiety and glutamate or glutamate isostere with the glutamate or glutamate isostere residing in the S1′ pocket. The non-pharmacophore pocket contains an arginine rich region and can accommodate a moderately-sized lipophilic moiety.

In some cases, small molecule PSMA inhibitors are generally zinc binding compounds attached to a glutamate or glutamate isostere and fall into three families of scaffolds: (1) phosphonate-, phosphate-, and phosphoramidates; (2) thiols; and, (3) ureas. In some embodiments, the scaffold is hydroxamate. In some embodiments, the small molecule scaffolds share common features, including: a) a pentanedioic acid as a glutamate mimic to fit within the S1′ binding pocket of the PSMA active site; and b) a zinc-binding group to interact with the catalytic zinc atom at the PSMA active site. Due to their high binding affinity and synthetic simplicity, urea-based small molecules, e.g., urea-based PSMA inhibitors, have been developed for use in biological imaging, e.g., positron emission tomography (PET) and single photon emission computed tomography (SPECT), using radionuclides.

In some embodiments, PSMA-targeting molecule or portion thereof include any described in, e.g.; WO2015143029; WO2016/065142; US 201013257499; US 2012/0067162; US 201213566849; US 201214008715; US 201214126296; US 201313826079; US 2014/0060461; US 201414152864; US 201414277367; US 201414335055; US 2015/0021233; US 2015/0029504; US 2015/0054937; US 2015/0056914; US 201514937169; US 2016/0022309; US 2016/0046981; US 23913608; US 74498208; US 89753907; AU 2008/269094; AU 2009/276423; AU 2015/203742; EP 03703745; EP 2015001929; EP 2015069356; EP 2016069730; Dobrenkov et al. (2008) J Nucl Med. 49:1162-1170; Chen et al., Biochem. Biophys. Res. Comm. 2009, 390(3):624-629; Banerjee et al., Oncotarget 2011; 2(12): 1244-1253; Banerjee et al. (2011) Angew Chem Int Ed Engl. 50(39): 9167-9170; Maurer et al. (2016) Nature Reviews Urology 13:226-235; Rowe et al. (2016) Prostate Cancer Prostatic Dis. 19(3):223-230; Mease et al., (2013) Curr Top Med Chem. 13(8):951-962; Osborne et al., (2013) Urol Oncol. 31(2): 144-154; or Barinka et al., (2008) J Mol Biol. 2008 Mar. 7; 376(5): 1438-1450; Philipp Wolf (2011), Prostate Specific Membrane Antigen as Biomarker and Therapeutic Target for Prostate Cancer, Prostate Cancer—Diagnostic and Therapeutic Advances, Philippe E. Spiess (Ed.), Intech, pp.81-100.

In some embodiments, the PSMA-targeting molecule is or comprises one or more of a phosphonate, a phosphate, a phosphoramidate, a phosphinate, a hydroxamate, a thiol derivative, a urea, a pentanedioic acid or a derivative thereof.

In some embodiments, the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405, N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (25)-2-{[(2-carboxy-ethyl)-hydroxy-phosphinoyl]methyl}-pentanedioic acid (SPE).

In some embodiments, the PSMA-targeting molecule is or comprises 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC). In some embodiments, the PSMA-targeting molecule is or comprises 2-(3-{1-carboxy-5-[(6-fluoro-pyridine-3-carbonyl)-amino]-pentyl}-ureido)-pentanedioicacid (DCFPyL), a high-affinity positron-emitting ligand.

2. Antibodies

In some embodiments, the PSMA-targeting molecule is or comprises an antibody or antigen-binding fragment thereof. In some embodiments, the PSMA-targeting molecule comprises a portion that is capable of binding PSMA or modified form thereof, that is or comprises an antibody or antigen-binding fragment thereof. In some embodiments, the PSMA-targeting molecule or portion thereof is or comprises an antibody or antigen-binding fragment thereof that is capable of binding a part of the extracellular portion of PSMA. In some embodiments, the PSMA-targeting molecule or a portion thereof is or comprises an antibody or antigen-binding fragment thereof, and a therapeutic agent and/or detectable moiety. In some embodiments, the PSMA-targeting molecule is an antibody-drug conjugate and/or a radiolabeled antibody.

In some embodiments, the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5 and antigen-binding fragments and derivatives thereof, or comprises a CDR3, a V_{H} and/or V_{L}, and/or competes for binding to PSMA or binds to the same PSMA epitope as any of the foregoing. In some embodiments, the antibody or antigen-binding fragment thereof is a multi-specific antibody or antigen-binding fragment thereof, e.g., a bi-specific antibody, where one of the targets of the antibody is PSMA or a modified form thereof.

In some embodiments, the antibody or antigen-binding fragment thereof includes those described in, e.g., US 2002/0049712; US 2002/0147312; US 2003/0082187; US 2004/0136998; US 2005/0202020; US 2006/0088539; US 2007/0071759; US 2010/0297653; US 2011/0020273; US 2013/0225541; US 2013/0315830; US 2014/0099257; US 2014/0227180; US 2015/0168413; US 2016/0303253; US 2017/0051074; U.S. Pat. Nos. 6,572,856; 7,476,513; 8,470,330; 8,986,655; WO 2006/078892; WO 2010/135431; WO 2014/198223; WO 2015/177360; WO 2016/057917; WO 2016/130819; WO 2016/145139; WO 2016/201300; WO 2017/004144; WO 2017/023761; AU 2002/356844; AU 2006/204913; AU 2006/235421; AU 2006/262231; AU 2006/315500; AU 2010/325969; AU 2013/328619; AU 2015/205574; CA 2353267; EP 1390069; EP 1520588; EP 1581794; EP 1599228; EP 1610818; EP 2906250; Banerjee et al. (2011) Angew Chem Int Ed Engl. 50(39): 9167-9170; Maurer et al. (2016) Nature Reviews Urology 13:226-235; Rowe et al. (2016) Prostate Cancer Prostatic Dis. 19(3):223-230; Mease et al., (2013) Curr Top Med Chem. 13(8):951-962; Osborne et al., (2013) Urol Oncol. 31(2): 144-154; Philipp Wolf (2011), Prostate Specific Membrane Antigen as Biomarker and Therapeutic Target for Prostate Cancer, Prostate Cancer—Diagnostic and Therapeutic Advances, Dr. Philippe E. Spiess (Ed.), Intech, pp.81-100; Ruggiero et al., (2011) J Nucl Med. 52(10): 1608-1615; Liu et al., (1997) Cancer Research 57:3629-3634; Regino et al., (2009) Curr Radiopharm. January; 2(1): 9-17; Kampmeier et al. (2014) EJNMMI Research 4:13; Wolf et al., (2010) The Prostate 70:562-569; Tykvart et al. (2014) The Prostate 74:1674-1690; Jin et al., (2016) EMJ Urol. 4(1):62-69 and Tino et al. (2000) Hybridoma 19(3):24957, or a fragment thereof, a conjugate thereof or a derivative thereof.

In some embodiments, the antibody or antigen-binding fragment is a radiolabeled antibody or antigen-binding fragment thereof selected from among ¹¹¹In-J591, ^{99m}Tc-J591, ⁸⁹Zr-J591, ¹⁷⁷Lu-J591, ⁹⁰Y-J591, ⁶⁴Cu-J591, ⁶⁴Cu-3/A12 F(ab′)₂, ⁶⁴Cu-3/A12 Fab, ¹¹¹In-CYT356, ⁹⁰Y-CYT356 and ⁸⁹Zr-DFO-J591, or a derivative thereof.

In some embodiments, the PSMA-targeting molecule is or comprises an antibody-drug conjugate (ADC) selected from among J591-monomethyl auristatin E (MMAE) and A5-Pseudomonas Exotoxin A (PE40).

3. Aptamers

In some embodiments, the PSMA-targeting molecule is or comprises an aptamer. In some embodiments, the PSMA-targeting molecule comprises a portion that is capable of binding PSMA or modified form thereof, that is or comprises an aptamer. In some embodiments, the PSMA-targeting molecule or portion thereof is or comprises an aptamer that is capable of binding a part of the extracellular portion of PSMA. In some embodiments, the PSMA-targeting molecule or a portion thereof is or comprises an aptamer, and a therapeutic agent and/or detectable moiety. In some embodiments, the PSMA-targeting molecule is an antibody-drug conjugate and/or a radiolabeled antibody.

In some embodiments, the PSMA-targeting molecule or portion thereof is or comprises an aptamer or a conjugate thereof. Aptamers are either 8-15 kDa oligonucleotides or peptides isolated from combinatorial libraries, which can be selected for specific binding to target molecules, e.g., PSMA, through affinity maturation. Aptamers achieve high affinity and specificity for the targets by folding into a unique three-dimensional conformation that is complementary to the surface of the target.

Exemplary aptamers that can bind to PSMA or modified form thereof include A9, A10, A10g, A10-3.2 or SZT101 or a conjugate thereof. In some embodiments, the aptamer further comprises a nanoparticle, a quantum dot or a radioisotope.

In some embodiments, the PSMA-targeting molecule is or comprises an aptamer that is linked or conjugated to a therapeutic agent or a moiety, e.g., a detectable moiety. For example, in some embodiments, the PSMA-targeting molecule is or comprises aptamer-nanoparticle, aptamer-quantum dot, aptamer-doxorubicin, aptamer-cisplatin A10-doxorubicin, A10-shRNA, A10-nanoparticle, aptamer-polyamidoaminepolyethyleneglycol (PAMAM-PEG), ⁶⁴Cu-labeled DOTA-, NOTA-, and 3,6,9,15-tetraazabicyclo[9.3.1]pentadeca-1(15),11,13-triene-S-4-(4-nitrobenzyl-3,6,9-triacetic acid (PCTA)-A10, or docetaxelencapsulated nanoparticle formulated with biocompatible and biodegradable poly(D,L-lactic acid-co-glycolic acid)-block-poly(ethyleneglycol) copolymer (PLGA-b-PEG)-A10. In some embodiments, PSMA-targeting molecule or portion thereof include any described in, e.g., Maurer et al. (2016) Nature Reviews Urology 13:226-235; Rowe et al. (2016) Prostate Cancer Prostatic Dis. 19(3):223-230; Mease et al., (2013) Curr Top Med Chem. 13(8):951-962; Osborne et al., (2013) Urol Oncol. 31(2): 144-154; Philipp Wolf (2011), Prostate Specific Membrane Antigen as Biomarker and Therapeutic Target for Prostate Cancer, Prostate Cancer—Diagnostic and Therapeutic Advances, Dr. Philippe E. Spiess (Ed.), Intech, pp.81-100.

4. Peptides

In some embodiments, the PSMA-targeting molecule is or comprises peptide. In some embodiments, the PSMA-targeting molecule comprises a portion that is capable of binding PSMA or modified form thereof, that is or comprises peptide. In some embodiments, the PSMA-targeting molecule or portion thereof is or comprises peptide that is capable of binding a part of the extracellular portion of PSMA. In some embodiments, the PSMA-targeting molecule or a portion thereof is or comprises peptide, and a therapeutic agent and/or detectable moiety.

In some embodiments, the PSMA-targeting molecule is or comprises a peptide, e.g., a peptide that is capable of binding PSMA. Exemplary peptides that are capable of binding to PSMA include WQPDTAHHWATL (SEQ ID NO:35); HNAYWHWPPSMT (SEQ ID NO:36); GHLIPLRQPSH (SEQ ID NO:37); YTSPHHSTTGHL (SEQ ID NO:38); WTHHHSYPRPL (SEQ ID NO:39); NSFPLMLMHHHP (SEQ ID NO:40); KHMHWHPPALN (SEQ ID NO:41); SLDSMSPQWHAD (SEQ ID NO:42); SEFIHHWTPPPS (SEQ ID NO:43); NGFSHHAPLMRY (SEQ ID NO:44); HHEWTHHWPPP (SEQ ID NO:45); AWPENPSRRPF (SEQ ID NO:46); AGFQHHPSFYRF (SEQ ID NO:47); KSLSRHDHIHHH (SEQ ID NO:48); YRHWPIDYPPP (SEQ ID NO:49); MIHTNHWWAQD (SEQ ID NO:50) and QRSPMMSRIRLP (SEQ ID NO:51) (see, e.g., US 8258256).

B. Therapeutic Agents

In some embodiments, the PSMA-targeting molecule is or comprises one or more therapeutic agent(s). In some embodiments, the therapeutic agent is one that is used in connection with treatment of a disease or disorder or condition, e.g. a tumor. In some embodiments, the therapeutic agent can potentiate or enhance the effects of treatment with adoptive cell therapy, e.g., administration of engineered cells expressing PSMA or a modified form thereof. In some embodiments, the PSMA-targeting molecule or a portion thereof capable of binding PSMA or modified form thereof, can direct or target the therapeutic agent to the site of the disease or disorder or condition, e.g., tumor. In some embodiments, the therapeutic agent is an immunomodulatory agent, a cytotoxic agent, an anti-cancer agent, a radiotherapeutic agent and/or a photosensitizer.

In some embodiments, the PSMA-targeting molecule or a portion thereof is capable of binding to a PSMA and/or to the modified form thereof, and/or is capable of binding to the active site of a PSMA and/or of the modified form of PSMA, and/or is capable of being cleaved by a PSMA and/or by the modified form of PSMA. In some embodiments, the cleavage results in at least one cleavage product comprising the therapeutic agent.

In some embodiments, the therapeutic agent is or comprises a protein, a peptide, a nucleic acid, a small molecule agent, a cell, a toxin, a lipid, a carbohydrate or combinations thereof, or any other type of therapeutic agent, e.g. radiotherapeutic, or any conjugates or combinations thereof. In some embodiments, the therapeutic agent is a small molecule, an antibody or antigen-binding fragment thereof, an inhibitory nucleic acid, e.g., siRNA or shRNA, or any conjugates or combinations thereof.

In some embodiments, the PSMA-targeting molecule comprising a therapeutic agent can be used to remove or kill target cells, e.g., tumor cells, by targeting a cytotoxic agent to the tumor. In some embodiments, the PSMA-targeting molecule comprising a therapeutic agent can be used to remove or kill adoptively transferred cells, e.g., engineered cells expressing PSMA or a modified form thereof, from the sample or the body of the subject, e.g., targeting a cytotoxic agent to the adoptively transferred cells.

1. Immunomodulatory Agents

In some embodiments, the therapeutic agent is an immunomodulatory agent (also referred to herein as “immunomodulator”). In some aspects, immunomodulatory agents are substances that either, directly or indirectly, suppress or activate the body's immune response. For example, immunomodulatory agents that stimulate immune response to tumors and/or pathogens may be used in combination with the engineered cells.

In some embodiments, the PSMA-targeting molecule comprising an agent capable of binding PSMA or modified form thereof can contain one or more immunomodulatory agents. In some embodiments, the one or more immunomodulatory agents are the same or different. In some embodiments, the PSMA-targeting molecule can contain two or more different immunomodulatory agents.

In some embodiments, the therapeutic agent can be any immunomodulatory agent that can stimulate, amplify and/or otherwise enhance an anti-tumor immune response, such as by inhibiting immunosuppressive signaling or enhancing immunostimulant signaling. In some embodiments, the immunomodulatory agent is a peptide, protein or is a small molecule. In some embodiments, the protein can be a fusion protein or a recombinant protein. In some embodiments, the immunomodulatory agent binds to an immunologic target, such as a cell surface receptor expressed on immune cells, such a T cells, B cells or antigen-presenting cells. For example, in some embodiments, the immunomodulatory agent is an antibody or antigen-binding antibody fragment, a fusion protein, a small molecule or a polypeptide.

In some embodiments, the immunomodulatory agent inhibits or modulates an immune checkpoint pathway. The immune system has multiple inhibitory pathways that are involved in maintaining self-tolerance and for modulating immune responses. It is known that tumors can use certain immune-checkpoint pathways as a major mechanism of immune resistance, particularly against T cells that are specific for tumor antigens (Pardoll, 2012, Nature Reviews Cancer 12:252-264). Because many such immune checkpoints are initiated by ligand-receptor interactions, they can be readily blocked by antibodies against the ligands and/or their receptors.

Therefore, therapy with antagonistic molecules blocking an immune checkpoint pathway, such as small molecules, nucleic acid inhibitors (e.g., RNAi) or antibody molecules, are becoming promising avenues of immunotherapy for cancer and other diseases. In contrast to the majority of anti-cancer agents, checkpoint inhibitors do not necessarily target tumor cells directly, but rather target lymphocyte receptors or their ligands in order to enhance the endogenous antitumor activity of the immune system. (Pardoll, 2012, Nature Reviews Cancer 12:252-264).

As used herein, the term “immune checkpoint inhibitor” refers to molecules that totally or partially reduce, inhibit, interfere with or modulate one or more checkpoint proteins. Checkpoint proteins regulate T-cell activation or function. These proteins are responsible for co-stimulatory or inhibitory interactions of T-cell responses. Immune checkpoint proteins regulate and maintain self-tolerance and the duration and amplitude of physiological immune responses.

Immune checkpoint inhibitors include any agent that blocks or inhibits in a statistically significant manner, the inhibitory pathways of the immune system. In some embodiments, immune checkpoint inhibitor is capable of inhibiting or blocking a function of an immune checkpoint molecule or a signaling pathway involving an immune checkpoint molecule. Such inhibitors may include small molecule inhibitors or may include antibodies, or antigen binding fragments thereof, that bind to and block or inhibit immune checkpoint receptor ligands. Illustrative immune checkpoint molecules that may be targeted for blocking or inhibition include, but are not limited to, CD25, PD-1 (CD279), PD-L1 (CD274, B7-H1), PD-L2 (CD273, B7-DC), CTLA-4, LAG3 (CD223), TIM3, 4-1BB (CD137), 4-1BBL (CD137L), GITR (TNFRSF18, AITR), CD40, CD40L, ICOS, ICOS-L, OX40 (CD134, TNFRSF4), OX40L, CXCR2, tumor associated antigens (TAA), B7-H3, B7-H4, BTLA, HVEM, GAL9, B7H3, B7H4, CD28, VISTA, CD27, CD30, STING, A2A adenosine receptor, KIR, 2B4 (belongs to the CD2 family of molecules and is expressed on all NK, γδ, and memory CD8+ (αβ) T cells), CD160 (also referred to as BY55), CGEN-15049. Immune checkpoint inhibitors include antibodies, or antigen binding fragments thereof, or other binding proteins, that bind to and block or inhibit the activity of one or more of CD25, PD-1, PD-L1, PD-L2, CTLA-4, LAG3, TIM3, 4-1BB, 4-1BBL, GITR, CD40, CD40L, ICOS, ICOS-L, OX40, OX40L, CXCR2, TAA, B7-H3, B7-H4, BTLA, HVEM, GAL9, CD28, VISTA, CD27, CD30, STING, A2A adenosine receptor, KIR, 2B4, CD160, and CGEN-15049. Illustrative immune checkpoint inhibitors include Tremelimumab (CTLA-4 blocking antibody), anti-OX40, PD-L1 monoclonal antibody (Anti-B7-H1; MEDI4736), MK-3475 (PD-1 blocker), nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody) and Yervoy/ipilimumab (anti-CTLA-4 checkpoint inhibitor). In some embodiments, the immune checkpoint molecule is selected from among PD-1, PD-L1, PD-L2, CTLA-4, LAG-3, TIM3, VISTA, an adenosine receptor or extracellular adenosine, optionally an adenosine 2A Receptor (A2AR) or adenosine 2B receptor (A2BR), or adenosine or a pathway involving any of the foregoing.

In some embodiments, the immunomodulatory agent is an antibody or antigen-binding antibody fragment thereof. Exemplary of such antibodies include, but are not limited to, Daclizumab (Zenapax), Bevacizumab (Avastin®), Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXR0916, AMP-224, MSB0010718C (Avelumab), MEDI4736, PDR001, rHIgM12B7, Ulocuplumab, BKT140, Varlilumab (CDX-1127), ARGX-110, MGA271, lirilumab (BMS-986015, IPH2101), IPH2201, ARGX-115, Emactuzumab, CC-90002 and MNRP1685A or an antibody-binding fragment thereof.

In some embodiments, the immune-modulating agent is cytokine. In some embodiments, the immunomodulatory agent is a cytokine or is an agent that induces increased expression of a cytokine in the tumor microenvironment. By “cytokine” is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. For example, the immunomodulatory agent is a cytokine and the cytokine is IL-4, TNF-α, GM-CSF or IL-2. In some embodiments, the cytokine includes PDGF, TGF-β, VEGF, tumor necrosis factor-α (TNF-α), endothelin-1, or IL-10. In some embodiments, the cytokine is IL-12 or IL-2. In some embodiments, the immunomodulatory agent can contain one or more interleukins or other cytokines. For example, the interleukin can include leukocyte interleukin injection (Multikine), which is a combination of natural cytokines.

In some embodiments, the immunomodulatory agent can be one that enhances the immunogenicity of tumor cells such as patupilone (epothilone B), epidermal-growth factor receptor (EGFR)-targeting monoclonal antibody 7A7.27, histone deacetylase inhibitors (e.g., vorinostat, romidepsin, panobinostat, belinostat, and entinostat), the n3-polyunsaturated fatty acid docosahexaenoic acid, proteasome inhibitors (e.g., bortezomib), shikonin (the major constituent of the root of Lithospermum erythrorhizon,) and oncolytic viruses, such as TVec (talimogene laherparepvec). In some embodiments, the immunomodulatory agent activates immunogenic cell death of the cancer or tumor, such as antrhacyclins (doxorubicin, mitoxantron), BK channel agonists, bortezomib, botrtezomib plus mitocycin C plus hTert-Ad, Cardiac glycosides plus non-ICD inducers, cyclophosphamide, GADD34/PP1 inhibitors plus mitomycin, LV-tSMAC, and oxaliplatin. In some embodiments, the immunomodulatory agent can be an epigenetic therapy, such as DNA methyltransferase inhibitors (e.g., Decitabine, 5-aza-2′-deoxycytidine).

For example, in some embodiments, the immunomodulatory agent can be a DNA methyltransferase inhibitor, which can regulate expression of tumor associated antigens (TAA). TAAs are antigenic substances produced in tumor cells which trigger an immune response. TAAs are often down-regulated by DNA methylation in tumors to escape the immune system. Reversal of DNA methylation restores TAA expression, increasing the immunogencity of tumor cells. For example, demethylating agents such as decitabine (5-aza-2′-deoxycytidine) can upregulate expression of TAAs in tumor cells and increase immune recognition of the cancerous cells.

Exemplary immunomodulatory agents can include, but are not limited to, bevacizumab, cetuximab, panitumumab, zalutumumab, nimotuzumab, Tositumomab (Bexxar®)), Rituximab (Rittman, Mabthera), Ibritumomab tiuxetan (Zevalin), Daclizumab (Zenapax), Gemtuzumab (Mylotarg), Alemtuzumab, CEA-scan Fab fragment, OC125 monoclonal antibody, ab75705, B72.3, Bevacizumab (Avastin®), and Basiliximab, nivolumab, pembrolizumab, pidilizumab, MK-3475, BMS-936559, MPDL3280A, ipilimumab, tremelimumab, IMP321, BMS-986016, LAG525, urelumab, PF-05082566, TRX518, MK-4166, dacetuzumab, lucatumumab, SEA-CD40, CP-870, CP-893, MED16469, MEDI6383, MEDI4736, MOXR0916, AMP-224, PDR001, MSB0010718C, rHIgM12B7, Ulocuplumab, BKT140, Varlilumab (CDX-1127), ARGX-110, MGA271, lirilumab (BMS-986015, IPH2101), IPH2201, AGX-115, Emactuzumab, CC-90002 and MNRP1685A or is an antibody-binding fragment thereof.

In some embodiments, the therapeutic agent is an agent that modulates adenosine levels and/or modulates the activity or amount of an adenosine pathway component. Adenosine can function as an immunomodulatory agent in the body. For example, adenosine and some adenosine analogs that non-selectively activate adenosine receptor subtypes decrease neutrophil production of inflammatory oxidative products (Cronstein et al., Ann. N.Y. Acad. Sci. 451:291, 1985; Roberts et al., Biochem. J., 227:669, 1985; Schrier et al., J. Immunol. 137:3284, 1986; Cronstein et al., Clinical Immunol. Immunopath. 42:76, 1987). In some cases, concentration of extracellular adenosine or adenosine analogs can increase in specific environments, e.g., tumor microenvironment (TME). In some cases, adenosine or adenosine analog signaling depends on hypoxia or factors involved in hypoxia or its regulation, e.g., hypoxia inducible factor (HIF). In some embodiments, increase in adenosine signaling can increase in intracellular cAMP and cAMP-dependent protein kinase that results in inhibition of proinflammatory cytokine production, and can lead to the synthesis of immunosuppressive molecules and development of Tregs (Sitkovsky et al., Cancer Immunol Res (2014) 2(7):598-605). In some embodiments, the therapeutic agent can reduce or reverse immunosuppressive effects of adenosine, adenosine analogs and/or adenosine signaling. In some embodiments, the therapeutic agent can reduce or reverse hypoxia-driven A2-adenosinergic T cell immunosuppression. In some embodiments, the therapeutic agent is selected from among antagonists of adenosine receptors, extracellular adenosine-degrading agents, inhibitors of adenosine generation by CD39/CD73 ectoenzymes, and inhibitors of hypoxia-HIF-la signaling. In some embodiments, the therapeutic agent is an adenosine receptor antagonist or agonist.

In some embodiments, the therapeutic agent can inhibit or reduce extracellular adenosine or the adenosine receptor by virtue of an inhibitor of extracellular adenosine (such as an agent that prevents the formation of, degrades, renders inactive, and/or decreases extracellular adenosine), and/or an adenosine receptor inhibitor (such as an adenosine receptor antagonist) can enhance immune response, such as a macrophage, neutrophil, granulocyte, dendritic cell, T- and/or B cell-mediated response. In addition, inhibitors of the Gs protein mediated cAMP dependent intracellular pathway and inhibitors of the adenosine receptor-triggered Gi protein mediated intracellular pathways, can also increase acute and chronic inflammation.

In some embodiments, the therapeutic agent is an A2 receptor (A2R) antagonist. Exemplary A2R antagonists include, but are not limited to, KW6002 (istradefyline), SCH58261, caffeine, paraxanthine, 3,7-dimethyl-1-propargylxanthine (DMPX), 8-(m-chlorostyryl) caffeine (CSC), MSX-2, MSX-3, MSX-4, CGS-15943, ZM-241385, SCH-442416, preladenant, vipadenant (BII014), V2006, ST-1535, SYN-115, PSB-1115, ZM241365, FSPTP, and an inhibitory nucleic acid targeting A2R expression, e.g., siRNA or shRNA, or any antibodies or antigen-binding fragment thereof that targets an A2R. In some embodiments, the therapeutic agent is an A2R antagonist described in, e.g., Ohta et al., Proc Natl Acad Sci USA (2006) 103:13132-13137; Jin et al., Cancer Res. (2010) 70(6):2245-2255; Leone et al., Computational and Structural Biotechnology Journal (2015) 13:265-272; Beavis et al., Proc Natl Acad Sci USA (2013) 110:14711-14716; and Pinna, A., Expert Opin Investig Drugs (2009) 18:1619-1631; Sitkovsky et al., Cancer Immunol Res (2014) 2(7):598-605; U.S. Pat. No. 8,080,554; 8,716,301; US 20140056922; WO2008/147482; U.S. Pat. No. 8,883,500; US 20140377240; WO02/055083; U.S. Pat. Nos. 7,141,575; 7,405,219; 8,883,500; 8,450,329 and 8,987,279).

In certain embodiments, the therapeutic agent is an adenosine deaminase (ADA) or a modified form thereof, e.g., recombinant ADA and/or polyethylene glycol-modified ADA (ADA-PEG), to the subject. Adenosine deaminase can inhibit local tissue accumulation of extracellular adenosine. ADA-PEG has been used in treatment of patients with ADA SCID (Hershfield (1995) Hum Mutat. 5:107). In some embodiments, an agent that inhibits extracellular adenosine is administered to the subject that includes agents that prevent or decrease formation of extracellular adenosine, and/or prevent or decrease the accumulation of extracellular adenosine, thereby abolishing, or substantially decreasing, the immunosuppressive effects of adenosine. In some embodiments, an agent is administered to the subject that specifically inhibits enzymes and proteins that are involved in regulation of synthesis and/or secretion of pro-inflammatory molecules, including modulators of nuclear transcription factors. Suppression of adenosine receptor expression or expression of the Gₛ protein- or Gᵢ protein-dependent intracellular pathway, or the cAMP dependent intracellular pathway, can result in an increase/enhancement of immune response.

In some embodiments, the therapeutic agent is an agent that targets ectoenzymes that generate or produce extracellular adenosine. In some embodiments, the agent targets CD39 and CD73 ectoenzymes, which function in tandem to generate extracellular adenosine. CD39 (also called ectonucleoside triphosphate diphosphohydrolase) converts extracellular ATP (or ADP) to 5′AMP. Subsequently, CD73 (also called 5′nucleotidase) converts 5′AMP to adenosine. The activity of CD39 is reversible by the actions of NDP kinase and adenylate kinase, whereas the activity of CD73 is irreversible. CD39 and CD73 are expressed on tumor stromal cells, including endothelial cells and Tregs, and also on many cancer cells. For example, the expression of CD39 and CD73 on endothelial cells is increased under the hypoxic conditions of the tumor microenvironment. Tumor hypoxia can result from inadequate blood supply and disorganized tumor vasculature, impairing delivery of oxygen (Carroll and Ashcroft (2005), Expert. Rev. Mol. Med. 7(6):1-16). Hypoxia also inhibits adenylate kinase (AK), which converts adenosine to AMP, leading to very high extracellular adenosine concentration. Thus, adenosine is released at high concentrations in response to hypoxia, which is a condition that frequently occurs the tumor microenvironment (TME), in or around solid tumors. In some embodiments, the therapeutic agent is one or more of anti-CD39 antibody or antigen binding fragment thereof, anti-CD73 antibody or antigen binding fragment thereof, e.g., MEDI9447 or TY/23, α-β-methylene-adenosine diphosphate (ADP), ARL 67156, POM-3, IPH52 (see, e.g., Allard et al. Clin Cancer Res (2013) 19(20):5626-5635; Hausler et al., Am J Transl Res (2014) 6(2):129-139; Zhang, B., Cancer Res. (2010) 70(16):6407-6411).

2. Cytotoxic Agents

In some embodiments, the therapeutic agent is a cytotoxic agent. In some embodiments, the therapeutic agent can kill specific cells, e.g., cells targeted by the PSMA-targeting molecule or cells in the microenvironment targeted by the engineered cell and/or PSMA-targeting molecule. In some embodiments, the PSMA-targeting molecule induces killing or destruction of one or more of the engineered cells and/or of a cell or tissue present in the subject that is specifically recognized by the recombinant receptor, e.g., a tumor cell or a cancer cell.

In some embodiments, the PSMA-targeting molecule comprising a therapeutic agent that is a cytotoxic agent can be used in connection with the engineered cells provided herein, to trigger suicide of the engineered cell, e.g., after administration of the engineered cell to a subject, to remove or destroy the engineered cell. In some embodiments, the PSMA-targeting molecule comprises a portion capable of binding PSMA or modified form thereof, linked or conjugated to a cytotoxic agent.

In some embodiments, the PSMA-targeting molecule comprising a therapeutic agent that is a cytotoxic agent can be administered to target the cytotoxic agent to cells present in the subject that is in the microenvironment targeted by the engineered cell, and/or PSMA-targeting molecule, e.g., a cell or tissue present in the subject that is specifically recognized by the recombinant receptor. For example, in some embodiments, the cytotoxic agent can be targeted to the tumor microenvironment (TME).

In some embodiments, administration of the PSMA-targeting molecule comprising a therapeutic agent that is a cytotoxic agent does not, or does not substantially, induce killing or destruction of healthy tissue or healthy cells, of cells or tissues not containing the engineered cells and/or not expressing the antigen.

In some cases, the cytotoxic agent can be a toxin or a radiometal. Other cytotoxic agents include, but are not limited to cytotoxic components (e.g., chemotherapeutic drugs such as anti-mitotics (e.g., vindesine), antifolates, alkylating agents (e.g., temozolomide), bacterial toxins, ricin, anti-virals, radioisotopes, radiometals). Such PSMA-targeting molecule comprising cytotoxic agents can be useful for specific killing or disabling an engineered cell, for example, when activity of a recombinant receptor is not desired. In other embodiments, PSMA-targeting molecule comprising cytotoxic agents can be used to target cells involved in a disease or disorder or condition, e.g., tumor or cancer cells.

In some embodiments, the cell-toxic reagent is a bacterial toxin that belongs to a major class of bacterial toxins, termed AB toxins, which use a transporter protein (B or binding unit) that actively translocates enzymes (A unit) into cells. Examples of AB toxins include botulinum neurotoxin, anthrax toxin, diphtheria toxin, shiga toxin, shiga like toxin, exotoxin A, and cholera toxin. Due to the similar mechanism of action between all of these toxins, all these toxins are contemplated to work in the various aspects of the present invention. The A and B components of these and a variety of other toxins are well known.

Bacterial toxins frequently have two functionally distinct moieties, termed A and B. The “A” component is usually the “active” portion, and the “B” component is usually the “binding” portion. Thus, the A moiety or component contains the catalytic activity, while the B moiety or component possesses determinants needed for the cytoplasmic delivery of the A moieties into target cells. These delivery determinants include receptor binding activity, and often, but not always, membrane penetration activity. Many bacterial toxins, such as diphtheria toxin, contain both moieties within a single polypeptide. Anthrax toxin, by contrast, is a member of the so-called binary toxins, a class in which the A and B functions inhabit separate proteins. Although separate, the proteins having the A and B functions interact during the intoxication of cells. Anthrax toxin uses a single B moiety, protective antigen (PA; 83 kDa), for the delivery of two alternative A moieties, edema factor (EF; 89 kDa) and lethal factor (LF; 89 kDa) into the cytoplasm (see international patent application publication number WO2012096926 for examples of bacterial toxins).

In some aspects, the toxin is a peptide toxin, ricin A chain toxin, Abrin A chain, Diphtheria Toxin (DT) A chain, Pseudomonas exotoxin, Shiga Toxin A chain, Gelonin, Momordin, Pokeweed Antiviral Protein, Saporin, Trichosanthin, proaerolysin or Barley Toxin. In some embodiments, the peptide toxin comprises a sequence of amino acids set forth in SEQ ID NO:34.

In some embodiments, exemplary cytotoxic agent includes, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. In some embodiments, the cytotoxic agent is a hypomethylating agent, e.g., a DNA methyltransferase inhibitor, e.g., azacitidine or decitabine.

3. Anti-Cancer Agents

In some embodiments, the therapeutic agent is an anti-cancer agent. In some embodiments, an anti-cancer agent can include any agent whose use can reduce, arrest or prevent cancer in a subject. In some embodiments, the anti-cancer agent includes any agents, when used alone or in combination with other compounds, that can alleviate, reduce, ameliorate, prevent, or place or maintain in a state of remission of clinical symptoms or diagnostic markers associated with tumors and cancer, and can be used in combinations and compositions provided herein. In some embodiments, the anti-cancer agent is one whose therapeutic effect is generally associated with penetration or delivery of the anti-cancer agent into the tumor microenvironment or tumor space.

In some embodiments, the anti-cancer agent is the anti-cancer agent is an alkylating agent, a platinum drug, an antimetabolite, an anti-tumor antibiotic, a topoisomerase inhibitor, a mitotic inhibitor, a corticosteroid, a proteasome inhibitor, a kinase inhibitor, a histone-deacetylase inhibitor, an anti-neoplastic agent, or an antibody or antigen-binding antibody fragment thereof or a combination thereof. In some embodiments, the anti-cancer agent is a peptide, protein or small molecule drug.

In some embodiments, the anti-cancer agent is 5-Fluorouracil/leukovorin, oxaliplatin, irinotecan, regorafenib, ziv-afibercept, capecitabine, cisplatin, paclitaxel, toptecan, carboplatin, gemcitabine, docetaxel, 5-FU, ifosfamide, mitomycin, pemetrexed, vinorelbine, carmustine wager, temozolomide, methotrexate, capacitabine, lapatinib, etoposide, dabrafenib, vemurafenib, liposomal cytarabine, cytarabine, interferon alpha, erlotinib, vincristine, cyclophosphamide, lomusine, procarbazine, sunitinib, somastostatin, doxorubicin, pegylated liposomal encapsulated doxorubicin, epirubicin, eribulin, albumin-bound paclitaxel, ixabepilone, cotrimoxazole, taxane, vinblastine, temsirolimus, temozolomide, bendamustine, oral etoposide, everolimus, octreotide, lanredtide, dacarbazine, mesna, pazopanib, eribulin, imatinib, regorafenib, sorafenib, nilotinib, dasantinib, celecoxib, tamoxifen, toremifene, dactinomycin, sirolimus, crizotinib, certinib, enzalutamide, abiraterone acetate, mitoxantrone, cabazitaxel, fluoropyrimidine, oxaliplatin, leucovorin, afatinib, ceritinib, gefitinib, cabozantinib, oxoliplatin or auroropyrimidine.

In some embodiments, the anti-cancer agent is an antibody or antigen-binding antibody fragment. In some embodiments, the anti-cancer agent can be any one or more of bevacizumab, cetuximab, panitumumab, ramucirumab, ipilimumab, rituximab, trastuzumab, ado-trastuzumab emtansine, pertuzumab, nivolumab, lapatinib, dabrafenib, vemurafenib, erlotinib, sunitinib, pazopanib, imatinib, regorafenib, sorafenib, nilotinib, dasantinib, celecoxib, crizotinib, certinib, afatinib, axitinib, bevacizumab, bosutinib, cabozantinib, afatinib, gefitinib, temsirolimus, everolimus, sirolimus, ibrutinib, imatinib, lenvatinib, olaparib, palbociclib, ruxolitinib, trametinib, vandetanib or vismodegib, or an antigen-binding antibody fragment thereof.

In some embodiments, the anti-cancer agent is an alkylating agent. Alkylating agents are compounds that directly damage DNA by forming covalent bonds with nucleic acids and inhibiting DNA synthesis. Exemplary alkylating agents include, but are not limited to, mechlorethamine, cyclophosphamide, ifosamide, melphalan, chlorambucil, busulfan, and thiotepa as well as nitrosurea alkylating agents such as carmustine and lomustine.

In some embodiments, the anti-cancer agent is a platinum drug. Platinum drugs bind to and cause crosslinking of DNA, which ultimately triggers apoptosis. Exemplary platinum drugs include, but are not limited to, cisplatin, carboplatin, oxaliplatin, satraplatin, picoplatin, nedaplatin, triplatin, and lipoplatin.

In some embodiments, the anti-cancer agent is an antimetabolite. Antimetabolites interfere with DNA and RNA growth by substituting for the normal building blocks of RNA and DNA. These agents damage cells during the S phase, when the cell's chromosomes are being copied. In some cases, antimetabolites can be used to treat leukemias, cancers of the breast, ovary, and the intestinal tract, as well as other types of cancer. Exemplary antimetabolites include, but are not limited to, 5-fluorouracil (5-FU), 6-mercaptopurine (6-MP), capecitabine (Xeloda®), cytarabine (Ara-C®), floxuridine, fludarabine, gemcitabine (Gemzar®), hydroxyurea, methotrexate, and pemetrexed (Alimta®).

In some embodiments, the anti-cancer agent is an anti-tumor antibiotic. Anti-tumor antibiotics work by altering the DNA inside cancer cells to keep them from growing and multiplying. Anthracyclines are anti-tumor antibiotics that interfere with enzymes involved in DNA replication. These drugs generally work in all phases of the cell cycle. They can be widely used for a variety of cancers. Exemplary anthracyclines include, but are not limited to, daunorubicin, doxorubicin, epirubicin, and idarubicin. Other anti-tumor antibiotics include actinomycin-D, bleomycin, mitomycin-C, and mitoxantrone.

In some embodiments, the anti-cancer agent is a topoisomerase inhibitor. These drugs interfere with enzymes called topoisomerases, which help separate the strands of DNA so they can be copied during the S phase. Topoisomerase inhibitors can be used to treat certain leukemias, as well as lung, ovarian, gastrointestinal, and other cancers. Exemplary toposiomerase inhibitors include, but are not limited to, doxorubicin, topotecan, irinotecan (CPT-11), etoposide (VP-16), teniposide, and mitoxantrone.

In some embodiments, the anti-cancer agent is a mitotic inhibitor. Mitotic inhibitors are often plant alkaloids and other compounds derived from natural plant products. They work by stopping mitosis in the M phase of the cell cycle but, in some cases, can damage cells in all phases by keeping enzymes from making proteins needed for cell reproduction. Exemplary mitotic inhibitors include, but are not limited to, paclitaxel (Taxol®), docetaxel (Taxotere®), ixabepilone (Ixempra®), vinblastine (Velban®), vincristine (Oncovin®), vinorelbine (Navelbine®), and estramustine (Emcyt®).

In some embodiments, the anti-cancer agent is a corticosteroid. Corticosteroids, often simply called steroids, are natural hormones and hormone-like drugs that are useful in the treatment of many types of cancer. Corticosteroids can also be used before chemotherapy to help prevent allergic reactions as well as during and after chemotherapy to help prevent nausea and vomiting. Exemplary corticosteroids include, but are not limited to, prednisone, methylprednisolone (Solumedrol®), and dexamethasone (Decadron®).

In some embodiments, the anti-cancer agent is another type of chemotherapy drug, such as a proteosome inhibitor, a kinase inhibitor, or a histone-deacetylase inhibitor. In some embodiments, the therapeutic agent includes an anthracycline (e.g., doxorubicin, such as liposomal doxorubicin); a vinca alkaloid (e.g., vinblastine, vincristine, vindesine, vinorelbine); an alkylating agent (e.g., cyclophosphamide, decarbazine, melphalan, ifosfamide, temozolomide); an immune cell antibody (e.g., alemtuzamab, gemtuzumab, rituximab, tositumomab); an antimetabolite (including, e.g., folic acid antagonists, pyrimidine analogs, purine analogs and adenosine deaminase inhibitors such as fludarabine); a TNFR glucocorticoid induced TNFR related protein (GITR) agonist; a proteasome inhibitor (e.g., aclacinomycin A, gliotoxin or bortezomib); or an immunomodulatory agent such as thalidomide or a thalidomide derivative (e.g., lenalidomide).

4. Radiotherapeutic Agents

In some embodiments, the PSMA-targeting molecule is or comprises a radiotherapeutic agent. In some embodiments, the therapeutic agent is a radiotherapeutic agent and/or contains a radioisotope, radiometal and/or radionuclide. In some embodiments, the therapeutic agent that contains a radioisotope, radiometal and/or radionuclide can also be used for diagnosis as well as radiotherapy. Exemplary radioisotope, radiometal and/or radionuclide include ¹⁰³Pd, ¹⁰⁵Rh, ¹⁰⁸Ag, ¹¹¹In, ^{117m}Sn, ¹¹C, ¹²³I, ¹²⁴I, ¹²⁵I, ¹³¹I, ¹³¹C, ¹³³Xe, ¹³⁷Cs, ¹⁴⁹Pm, ¹⁵³Sm, ¹⁶⁵Dy, ¹⁶⁶Ho, ¹⁶⁹Er, ¹⁶⁹Yb, ¹⁷⁷Lu, ¹⁷⁷Yb, ¹⁸⁶Re, ¹⁸⁸Re, ¹⁸F, ¹⁹²Ir, ²⁰¹Tl, ²¹⁰Po, ²¹¹At, ²¹²Bi, ²¹²Pb, ²¹³Bi, ²²³Ra, ⁴⁷Sc, ⁵¹Cr, ⁵⁵Co, ⁶⁰Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁶⁸Ge, ⁷⁵Se, ⁸²Rb, ⁸⁶Y, ⁸⁷Y, ⁸⁹Sr, ⁸⁹Zr, ⁹⁰Y and ^{99m}Tc.

In some embodiments, the radiotherapeutic agent includes radioisotope, radiometal and/or radionuclide that are strong beta emitters. In some embodiments, the radiotherapaeutic agent comprises ¹⁷⁷Lu, a beta-particle emitting radionuclide that also decays by single-photon emission and can be imaged with a gamma camera, or ⁶⁸Ga. In some embodiments, the PSMA-targeting molecule comprises ¹⁷⁷Lu and/or ⁶⁸Ga.

In some embodiments, the PSMA-targeting molecule is a radiolabeled antibody, e.g., ¹⁷⁷Lu-J591, or a radioligand, e.g., ¹⁷⁷Lu-DKFZ-617. Other exemplary radiotherapeutic agent that can be comprised in the PSMA-targeting molecule include those described in, e.g., Maurer et al. (2016) Nature Reviews Urology 13:226-235; Rowe et al. (2016) Prostate Cancer Prostatic Dis. 19(3):223-230; Mease et al., (2013) Curr Top Med Chem. 13(8):951-962).

5. Photosensitizers

In some embodiments, the therapeutic agent comprises a photosensitizer. Photosensitizers include chemical compounds that can be excited by light of a specific wavelength. In some embodiments, by virtue of binding of the PSMA-targeting molecule comprising a therapeutic agent that is a photosensitizer, the effect of the photosensitizer and photodynamic therapies (PDT) can be directed or targeted to particular cells, sites, locations or microenvironments. In some embodiments, the photosensitizer comprises Pyropheophorbide-a (Ppa) or YC-9 (see, e.g., Chen et al. (2016) J Photochem Photobiol B 167:111-116; Liu et al. (2010) Cancer Lett, 296(1): 106-112; Liu et al. (2009) The Prostate 69(6): 585-594; Liu et al. (2010) Int J Oncol, 36(4):777-784).

C. Detectable Moiety

In some embodiments, the PSMA-targeting molecule is or comprises one or more moiety that provides a signal or induces a signal that is detectable. For example, in some embodiments, the PSMA-targeting molecule is or comprises a detectable moiety. In some embodiments, the PSMA-targeting molecule capable of binding to one or more moiety that provides a signal or induces a signal that is detectable, e.g., a detectable moiety. In some embodiments, PSMA-targeting molecule is linked or to a detectable moiety or is capable of producing a detectable signal. In some embodiments, the PSMA-targeting molecule comprising a moiety is capable of being cleaved upon binding the PSMA or modified form thereof, or cleaved in the presence of one or more conditions or factors present in the tumor microenvironment (TME), wherein cleavage results in at least one cleavage product comprising the moiety and/or is fluorescent and/or radioactive.

In some embodiments PSMA-targeting molecule is or comprises an imaging probe, a detection reagent, an imaging modality or a detectable label. In some embodiments, the detection reagent comprises a radioligand. In some embodiments, the imaging probe, detection reagent, imaging modality or detectable label comprises a radioisotope, a bioluminescent compound, a chemiluminescent compound, a fluorescent compound, a chromogenic compound, a quantum dot, a nanoparticle, a metal chelate, an enzyme, an iron-oxide nanoparticle or other known imaging agentsfor detection by X-ray, CT-scan, MRI-scan, PET-scan, ultrasound, flow-cytometry, near infrared imaging systems, or other imaging modalities (see, e.g., Yu et al., Theranostics (2012) 2:3).

In some embodiments, the moiety, e.g., detectable moiety, comprises a radioisotope, a bioluminescent compound, a chemiluminescent compound, a fluorescent compound, a chromogenic compound, a quantum dot, a nanoparticle, a metal chelate or an enzyme. In some embodiments, the PSMA-targeting molecule comprises an imaging probe or a detection reagent, which optionally is a radioligand. In some instances, the detectable moiety contains a fluorescent protein or a fluorescent label or a fluorophore. In some embodiments, the binding molecule is conjugated to an imaging modality.

In some embodiments, the detectable moiety comprises a fluorophore selected from among Alexa Fluor® 488 or related fluorophore; Alexa Fluor® 647 or related fluorophore; Alexa Fluor® 680 or 700 or related fluorophore; AmCyan or BD Horizon V500 or related fluorophore; APC or Alexa Fluor® 647 or related fluorophore; APC or related fluorophore; APC-Cy7 or APC-H7 or related fluorophore; BD Horizon PE-CF594 or related fluorophore; BD Horizon V450 or related fluorophore; Brilliant Violet 41 or related fluorophore; FITC or related fluorophore; PE or related fluorophore; PE-Cy7 or related fluorophore; PERCP-Cy5.5 or related fluorophore; PE-Texas Red® or related fluorophore.

Exemplary labels include radionuclides (e.g. ¹²⁵I, ¹³¹I, ³⁵S, ³H, or ³²P and/or chromium (⁵¹Cr), cobalt (⁵⁷Co), fluorine (¹⁸F), gadolinium (¹⁵³Gd, ¹⁵⁹Gd), germanium (⁶⁸Ge), holmium (¹⁶⁶Ho) indium (¹¹⁵In, ¹¹³In, ¹¹²In, ¹¹¹In), iodine (¹²⁵I, ¹²³I, ¹²¹I), lanthanum (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (186Re, 188Re), rhodium (105Rh), rutheroium (97Ru), samarium (¹⁵³Sm), scandium (⁴⁷Sc), selenium (⁷⁵Se), (⁸⁵Sr), sulphur (³⁵S), technetium (⁹⁹Tc), thallium (²⁰¹Ti), tin (¹¹³Sn, ¹¹⁷Sn), tritium (3H), xenon (¹³³Xe), ytterbium (¹⁶⁹Yb, ¹⁷⁵Yb), yttrium (⁹⁰Y)). In some embodiments, the PSMA-targeting molecule comprises radioisotope, radiometal and/or radionuclide, which optionally is selected from among ¹⁰³Pd, ¹⁰⁵Rh, ¹⁰⁸Ag, ¹¹¹In, ^{117m}Sn, ¹¹C, ¹²³I, ¹²⁴I, ¹²⁵I, ¹³¹I, ¹³¹Cs, ¹³³Xe, ¹³⁷Cs, ¹⁴⁹Pm, ¹⁵³Sm, ¹⁶⁵Dy, ¹⁶⁶Ho, ¹⁶⁹Er, ¹⁶⁹Yb, ¹⁷⁷Lu, ¹⁷⁷Yb, ¹⁸⁶Re, ¹⁸⁸Re, ¹⁸F, ¹⁹²Ir, ²⁰¹Tl, ²¹⁰Po, ²¹¹At, ²¹²Bi, ²¹²Pb, ²¹³Bi, ²²³Ra, ⁴⁷SC, ⁵¹Cr, ⁵⁵Co, ⁶⁰Co, ⁶⁰Cu, ⁶¹Cu, ⁶²Cu, ⁶⁴Cu, ⁶⁶Ga, ⁶⁷Cu, ⁶⁷Ga, 68Ga, ⁸⁶Ge, ⁷⁵Se, ⁸²Rb, ⁸⁶Y, ⁸⁷Y, ⁸⁹Sr, ⁸⁹Zr, ⁹⁰Y and ^{99m}Tc.

In some embodiments, exemplary detectable moieties or labels include various enzymes, prosthetic groups, fluorescent materials, luminescent materials, bioluminescent materials, and radioactive materials. Examples of suitable enzymes include horseradish peroxidase, alkaline phosphatase, beta-galactosidase, luciferase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorot[pi]azinylamine fluorescein, dansyl chloride, phycoerythrin, GFP, or BFP. Example of a luminescent material includes luminol and quantum dots (Qdot™ nanoparticles supplied by the Quantum Dot Corporation, Palo Alto, Calif). Examples of bioluminescent materials include luciferase, luciferin, and aequorin. In some embodiments, exemplary detectable moieties or labels include a light-emitting proteins or bioluminescent proteins, that can be detectable and can be monitored visually, or by using a spectrophotometer, luminometer, fluorometer or other related methods. In some embodiments, the reporter is a detectable moiety, such as an enzyme that produces bioluminescence, e.g., enzymes that can convert a substrate that emits light, e.g., luciferase or variants thereof. Non-limiting examples of light emitting proteins or enzymes that produce bioluminescence include, for example, luciferase, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS), and fluorescent protein and variants thereof, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. Luciferases and variants thereof can include luciferases from the firefly (Photinus pyralis), sea pansy (Renilla reniformis), Photobacterium species (Vibrio fischeri, Vibrio haweyi and Vibrio harveyi), dinoflagellates, marine copepod (Metridia longa), deep sea shrimp (Oplophorus) and Jack-O-Lantern mushroom (Omphalotus olearius), and variants thereof, including codon-optimized and/or enhanced variants. In some embodiments, the reporter molecule is a red fluorescent protein (RFP), optionally tdTomato.

D. Linkage of Components

In some embodiments, the PSMA-targeting molecule comprises a portion that is capable of binding PSMA or modified form thereof and a therapeutic agent and/or a moiety that provides a signal or induces a signal that is detectable, e.g., detectable moiety. In some embodiments, the PSMA-targeting molecule further comprises a therapeutic agent, and the therapeutic agent is linked directly or indirectly, optionally via a linker, to the PSMA-targeting molecule or portion thereof capable of binding PSMA or modified form thereof. In some embodiments, the PSMA-targeting molecule further comprises a moiety that provides a signal or induces a signal that is detectable, and the moiety is linked directly or indirectly, optionally via a linker, to the PSMA-targeting molecule or portion thereof capable of binding PSMA or modified form thereof.

In some embodiments, the PSMA-targeting molecule is capable of being cleaved, e.g., upon binding to the PSMA or modified form thereof and/or in the presence of particular conditions. In some embodiments, the components of the PSMA-targeting molecule, e.g., a portion thereof is capable of binding to a PSMA, and the therapeutic agent and/or the moiety that provides a signal or induces a signal that is detectable, are linked, and the linkage is capable of being cleaved, e.g., upon binding to the PSMA or modified form thereof and/or in the presence of particular conditions. In some embodiments, the components are linked indirectly via a linker, and the linker is capable of being cleaved, e.g., upon binding to the PSMA or modified form thereof and/or in the presence of particular conditions.

In some embodiments, the PSMA-targeting molecule or a portion thereof is capable of binding to a PSMA and/or to the modified form thereof, and/or is capable of binding to the active site of a PSMA and/or of the modified form of PSMA, and/or is capable of being cleaved by a PSMA and/or by the modified form of PSMA.

In some embodiments, the linkage between the portion capable of binding PSMA or modified form thereof and the therapeutic agent and/or the moiety, e.g., detectable moiety, can be cleaved or released under specific conditions. In some embodiments, the PSMA-targeting molecule capable of being cleaved upon binding the PSMA or modified form thereof, or cleaved in the presence of one or more conditions or factors present in the tumor microenvironment (TME), resulting in at least one cleavage product comprising the therapeutic agent and/or the moiety, e.g., detectable moiety. In some embodiments, the cleavage activates the therapeutic agent and/or releases the therapeutic agent from the portion capable of binding PSMA or modified form thereof, allowing targeting and/or delivery of the agent to the site, location or microenvironment. In some embodiments, the PSMA-targeting molecule comprising the moiety is capable of being cleaved upon binding the PSMA or modified form thereof, or cleaved in the presence of one or more conditions or factors present in the tumor microenvironment (TME), wherein cleavage results in at least one cleavage product comprising the moiety and/or is fluorescent and/or radioactive.

In some embodiments, the linker is a peptide or a polypeptide or is a chemical linker. In some embodiments, the linker is a releasable linker or a cleavable linker.

In some embodiments, the linker is capable of being cleaved upon binding the PSMA or modified form thereof by the PSMA-targeting molecule, wherein cleavage results in at least one cleavage product comprising the therapeutic agent and/or the moiety, e.g., detectable moiety. In some embodiments, the releasable linker or the cleavable linker is released or cleaved in the presence of one or more conditions or factors present in the tumor microenvironment (TME), wherein cleavage results in at least one cleavage product comprising the therapeutic agent and/or the moiety and/or is fluorescent and/or radioactive.

In some embodiments, the components of the PSMA-targeting molecules provided herein, which optionally can be or comprise a therapeutic agent or a moiety, e.g., detectable moiety, directly or indirectly, optionally via a linker. In some embodiments, the linkage is covalent or non-covalent.

In some embodiments, the linkage or attachment includes an indirect link, such as through a linker, binding moiety or domain or reactive group. In some embodiments, the linkage includes a direct interaction PSMA-targeting molecule and/or the therapeutic agent and/or the detectable moiety. In other embodiments, one or both or all of the components of the PSMA-targeting molecule are linked to one or more linkers, and the interaction is indirect, e.g., between a linker attached to one of the molecules and another molecule, or between two linkers, each attached to the PSMA-targeting molecule and/or the therapeutic agent and/or the detectable moiety. In some embodiments, the targeting molecule, the PSMA-targeting molecule and/or the therapeutic agent and/or the detectable moiety are non-covalently linked to or associated with the other components. In some examples, the non-covalent interaction include, for example, electrostatic interactions, van der Waals force, hydrophobic interactions, π-effects, ionic interactions, hydrogen bonding, halogen bonding and/or combinations thereof, or any interactions that depend on one or more of the forces. In some embodiments, the targeting molecule, the PSMA-targeting molecule and/or the therapeutic agent and/or the detectable moiety are linked using or using interactions that mimic non-covalent molecular interactions such as, for example, ligand-receptor interaction, antibody-antigen interaction, avidin-biotin interaction, streptavidin-biotin interaction, histidine-divalent metal ion interaction (e.g., Ni, Co, Cu, Fe), interactions between multimerization (e.g., dimerization) domains, glutathione S-transferase (GST)-glutathione interaction and/or any combination thereof.

In some embodiments, the PSMA-targeting molecule and/or the therapeutic agent and/or the detectable moiety are linked indirectly via a linker. For example, the linker can be a peptide, a polypeptide, or a chemical linker. Various peptide linkers, polypeptide linkers and chemical linkers may in some aspects be used in the PSMA-targeting molecules. For example, the linker may be a peptide linker, or a cleavable peptide linker. In some embodiments, the linker is a covalent linker, wherein the covalent linkage is linear or branched, cyclic or heterocyclic, saturated or unsaturated, having 1-60 atoms, such as selected from among C, N, P, O, and S. In some embodiments, the linkage, e.g., chemical linkage, may contain any combination of ether, thioether, amine, ester, carbamate, urea, thiourea, oxy or amide bonds. In some embodiments, the linkage, e.g., chemical linkage, may include single, double, triple or aromatic carbon-carbon bonds, phosphorus-oxygen, phosphorus-sulfur, nitrogen-nitrogen, nitrogen-oxygen, nitrogen-platinum bonds, or aromatic or heteroaromatic bonds. For example, in some embodiments, the linker can be a linker that has a reactive or activatable group, which is able to form a bond between the linker and the component being linked to. In some embodiments, the linker contains a reactive group.

In some embodiments, the PSMA-targeting molecule is linked to the therapeutic agent and/or detectable moiety via a releasable or cleavable linker. In some embodiments, the linker is not cleavable. In some embodiments, the release or cleavage of the linker permits release of the therapeutic agent from the PSMA-targeting molecule. Thus, the therapeutic agent can be targeted or delivered directly to the cells involved in a disease or condition and/or be released into the microenvironment of a disease, disorder or condition, by virtue of the PSMA-targeting molecule binding the surface-expressed PSMA or modified form thereof, e.g., on the engineered cells, in the microenvironment of the disease, disorder or condition.

In some embodiments, the linker is capable of being cleaved upon binding the PSMA or modified form thereof by the PSMA-targeting molecule, wherein cleavage results in at least one cleavage product comprising the therapeutic agent. As described above, PSMA exhibits glutamate carboxypeptidase activity and folate hydrolase activity. The enzymatic activity of PSMA can be exploited for cleavage of the linker, e.g., a linker that links PSMA-targeting molecule and/or the therapeutic agent and/or the detectable moiety. In some embodiments, the cleavage of the linker by PSMA can result in one or more cleavage products, wherein the cleavage product can comprise a therapeutic agent or a detectable moiety, such as a fluorescent or radioactive moiety (see, e.g., WO 2015143029). For example, in some embodiments, the linker can contain α-linked or γ-linked glutamic or aspartic acids and can be cleaved upon binding to PSMA.

The term “releasable linker” or “cleavable linker” as used herein, refers to a linker that includes at least one bond that can be broken under physiological conditions (e.g., a pH-labile, acid-labile, oxidatively-labile, or enzyme-labile bond). Physiological conditions resulting in breaking of the chemical bond can include standard chemical hydrolysis reactions that occur, for example, at physiological pH, or as a result of specific conditions present in a particular microenvironment, e.g., microenvironment of a lesion, such as the tumor microenvironment (TME).

In some embodiments, the releasable linker or the cleavable linker is released or cleaved in the microenvironment of the disease, disorder or condition. In some embodiments, the disease, disorder or condition is associated with specific microenvironment or physiological conditions. For example, in some embodiments, the disease, disorder or condition is a tumor, and the releasable linker or the cleavable linker is released or cleaved in the tumor microenvironment (TME), for example, under acidic or hypoxic conditions. In some embodiments, in the presence of one or more conditions or factors present in the tumor microenvironment (TME), wherein cleavage results in at least one cleavage product comprising the therapeutic agent. In some embodiments, the one or more conditions or factors present in the tumor microenvironment (TME) comprises matrix metalloproteinase (MMP), hypoxic conditions or acidic conditions.

A variety of exemplary linkers that can be used in connection with the cells, compositions and methods provided herein include those described in WO2004-010957, U.S. Publication Nos. 20060074008, 20050238649, and 20060024317.

In some embodiments, the releasable linker or the cleavable linker is released or cleaved in hypoxic conditions or acidic conditions. In some embodiments, the conditions in the TME are acidic or hypoxic. In some embodiments, the linker is acid-labile or cleavable in hypoxic conditions. In some embodiments, the cleavable linker is cleavable under acidic conditions, and the cleavable linker comprises one or more hydrazone, semicarbazone, thiosemicarbazone, cis-aconitic amide, orthoester, acetal, ketal, 4-(4′-acetylphenoxy) butanoic acid or thioether linkages. In some embodiments, the cleavable linker is cleavable under hypoxic conditions, and the linker comprises one or more disulfide linkages.

In some embodiments, the linker is cleavable by a cleaving agent that is present in the microenvironment of a lesion. The linker can be, e.g., a peptidyl linker that is cleaved by a peptidase or protease enzyme. For example, the releasable linker or the cleavable linker is released or cleaved by a matrix metalloproteinase (MMP) present in in the TME. In some embodiments, the cleavable linker comprises the sequence of amino acids Pro-Leu-Gly-Leu-Trp-Ala (set forth in SEQ ID NO:18). In some embodiments, the linker is cleavable by a cleaving agent that is overexpressed in the microenvironment of a lesion. In some embodiments, exemplary linkers include peptidyl linkers that are at least two amino acids long or at least three amino acids long. Exemplary linkers include a Phe-Leu linker, a Gly-Phe-Leu-Gly linker (SEQ ID NO:33), a Val-Cit linker or a Phe-Lys linker (see, e.g., U.S. Pat. No. 6,214,345). Other examples of such linkers are described, e.g., in U.S. Pat. No. 6,214,345 and Lu et al. (2016) Int. J. Mol. Sci. 17(4):561. In some embodiments, the linker is a linker cleavable by an enzyme that is overexpressed in the tumor interstitium, such as β-glucuronidase. In some embodiments, the linker is a β-glucuronide linker.

In other embodiments, the cleavable linker is pH-sensitive, i.e., sensitive to hydrolysis at certain pH values. Typically, the pH-sensitive linker hydrolyzable under acidic conditions, such as, for example, the microenvironment of a lesion. For example, an acid-labile linker that is hydrolyzable in acidic environments, e.g., a hydrazone, semicarbazone, thiosemicarbazone, cis-aconitic amide, orthoester, acetal or ketal linkage, can be used. In some embodiments, exemplary linkers include those described in e.g., U.S. Pat. Nos. 5,122,368; 5,824,805; 5,622,929; Dubowchik and Walker, 1999, Pharm. Therapeutics 83:67-123; Neville, et al., 1989, Biol. Chem. 264:14653-14661. Such linkers are relatively stable under neutral pH conditions, such as those in the blood, but are unstable in acidic conditions.

In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe, et al., 1987, Cancer Res. 47:5924-5931; Wawrzynczak, et al., In Immunoconjugates: Antibody Conjugates in Radioimagery and Therapy of Cancer (C. W. Vogel ed., Oxford U. Press, 1987). See also U.S. Pat. No. 4,880,935.).

In some embodiments, the linker is a malonate linker (Johnson, et al., 1995, Anticancer Res. 15:1387-93), a maleimidobenzoyl linker (Lau, et al., 1995, Bioorg-Med-Chem. 3(10):1299-1304), or a 3′-N-amide analog (Lau, et al., 1995, Bioorg-Med-Chem. 3(10):1305-12).

E. Prodrugs

In some embodiments, the PSMA-targeting molecule is or comprises a prodrug. In some embodiments, the PSMA-targeting molecule is or comprises or is capable of conversion into or unmasking of a therapeutic agent and/or the PSMA-targeting molecule is capable of being cleaved upon binding to the PSMA or modified form thereof, wherein cleavage results in at least one cleavage product comprising a therapeutic agent. In some embodiments, the cleavage results in at least one cleavage product comprising a therapeutic agent.

In some embodiments, the PSMA-targeting molecule is a prodrug that is activated in the microenvironment of the disease, disorder or condition. In some embodiments, the disease, disorder or condition is associated with specific microenvironment or physiological conditions. For example, in some embodiments, the disease, disorder or condition is a tumor, and prodrug is activated in the tumor microenvironment (TME), for example, under acidic or hypoxic conditions.

In some embodiments, as used herein the term “prodrug” refers to a pharmacologically inactive derivative of a parent “drug” molecule that requires biotransformation (e.g., either spontaneous or enzymatic) within the target physiological system to release, or to convert (e.g., enzymatically, mechanically, electromagnetically, etc.) the “prodrug” into the active “drug.” “Prodrugs” are designed to overcome problems associated with stability, toxicity, lack of specificity, or limited bioavailability. Exemplary “prodrugs” comprise an active “drug” molecule itself and a chemical masking group (e.g., a group that reversibly suppresses the activity of the “drug”). Some preferred “prodrugs” are variations or derivatives of compounds that have groups cleavable under metabolic conditions. Exemplary “prodrugs” become pharmaceutically active in vivo or in vitro when they undergo solvolysis under physiological conditions or undergo enzymatic degradation or other biochemical transformation (e.g., phosphorylation, hydrogenation, dehydrogenation, glycosylation, etc.). Prodrugs can offer advantages of solubility, tissue compatibility, or delayed release in the mammalian organism. Exemplary PSMA-targeting molecule that is a prodrug comprises a peptide that is cleaved by PSMA and a therapeutic agent.

Upon binding of a PSMA-targeting molecule that is a prodrug to the PSMA or modified form thereof, the glutamate carboxy peptidase activity of PSMA can activate the prodrugs to a therapeutically active agent. In some embodiments, the prodrug comprises methotrexate, thapsigargin or doxorubicin analogs. In some embodiments, the analogs contain α-linked or γ-linked glutamic or aspartic acids. In some embodiments, the PSMA-targeting molecule that is a prodrug includes those described in, e.g., Mhaka et al. (2006) Cancer Biology & Therapy 3(6):551-558; Mahalingam et al. (2016) British Journal of Cancer 114:986-994; U.S. Pat. No. 8,258,256). For example, in some embodiments, the PSMA-targeting molecule is Mipsagargin (G-202; (8-O-(12-aminododecanoyl)-8-O-debutanoyl thapsigargin)-Asp-γ-Glu-γ-Glu-γ-GluGluOH). In some embodiments, the prodrug is a derivative of a toxin.

F. Antibody-drug Conjugates (ADCs)

In some embodiments, the PSMA-targeting molecule is an antibody-drug conjugate (ADC). In some embodiments, a PSMA-targeting molecule that is an antibody or an antigen-binding fragment thereof, is conjugated or linked, directly or indirectly, to a therapeutic agent or a drug. In some embodiments, the therapeutic agent in the ADC comprises a cytotoxic agent, a toxin or an anti-cancer agent. In some embodiments, the therapeutic agent is an immunomodulatory agent. In some embodiments, the therapeutic agent of the ADC is a cytotoxic agent, a toxin or an anti-cancer agent, e.g., monomethyl auristatin E, maytansinoids DM1, Pseudomonas Exotoxin A (PE40) or a-amanitin.

Exemplary PSMA-targeting molecules that are ADCs include J591-monomethyl auristatin E (MMAE), A5-Pseudomonas Exotoxin A (PE40), anti-PSMA-a-amanitin, MLN2704, Progenics PSMA ADC, and those described in, e.g., US 2011/0165081; US 2011/0250216; US 2015/0110814; WO 2006/110745; WO 2007/002222; WO 2007/038658; and WO 2015/057250.

In some embodiments, the antibody or antigen-binding fragment thereof and the therapeutic agent of the ADC are linked or conjugated directly or indirectly. In some embodiments, the antibody or antigen-binding fragment thereof and the therapeutic agent of the ADC are linked indirectly, optionally via a linker, e.g. a cleavable or releasable. In some embodiments, the cleavable or releasable linker is capable of being cleaved upon binding to the PSMA or modified form thereof, or a condition in the microenvironment of the disease, disorder or condition. In some embodiments, the disease, disorder or condition is associated with specific microenvironment or physiological conditions. For example, in some embodiments, the disease, disorder or condition is a tumor, and prodrug is activated in the tumor microenvironment (TME), for example, under acidic or hypoxic conditions.

### III. COMPOSITIONS AND FORMULATIONS

Provided are compositions including cells, such as engineered cells containing the PSMA or modified form thereof and recombinant receptors, e.g., CAR, for administration. Also among the provided embodiments are those involving compositions containing PSMA-targeting molecules, such those described herein, for example, methods and uses thereof in connection with the administration of engineered cells as provided herein and combinations of such compositions and cell therapies. In some aspects, the pharmaceutical compositions and formulations are provided as unit dose form compositions including the number of cells for administration in a given dose or fraction thereof. In some aspects, the pharmaceutical compositions and formulations are provided as unit dose form compositions including a dose of PSMA-targeting molecules for administration in a given dose or fraction thereof. In some embodiments, the provided compositions can be used in connection with any of the methods described herein, e.g., methods of treatment, method of detection and/or method of selection. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredients useful for the particular indication, disease, or condition being treated with the cells, preferably those with activities complementary to the cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

In some embodiments, the pharmaceutical composition, such as the formulation or composition containing the PSMA-targeting molecule, in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains engineered cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The pharmaceutical compositions, such as those containing the engineered cells, may be administered using standard administration techniques, formulations, and/or devices. Provided are formulations and devices, such as syringes and vials, for storage and administration of the compositions. Administration of the engineered cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol(for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. In some aspects, the compositions, such as a composition containing the PSMA-targeting molecule, can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### IV. METHODS OF ADMINISTRATION, TREATMENT AND DETECTION

Also provided are methods of using and uses of the cells and compositions, such as those containing the engineered cells, in the treatment of diseases, conditions, and disorders in which the antigen recognized by the recombinant receptor (e.g. CAR) is expressed. In some embodiments, the methods of treatment involve administration of engineered cells expressing PSMA or a modified form thereof. In some embodiments, the administered cells also express a recombinant receptor, e.g., CAR. In some embodiments, the methods of treatment involve administering any of the engineered cells provided herein, or any of the compositions provided herein, to a subject. In some embodiments, the method also involves administering any of the PSMA-targeting molecule described herein, to a subject. In some embodiments, the PSMA-targeting molecule is capable of binding to PSMA or a modified form thereof, and therefore can be used to target specific cells or microenvironments for therapy, or to deliver therapeutic agents to specific cells or microenvironment. In some embodiments, such methods include diagnostic and prognostic methods as well as, in some cases, suicide or removal methods of the engineered cells. Included among such methods are methods of monitoring the administered engineered cells and methods of modulating the engineered cells, such as in connection with adoptive cell therapy.

In some embodiments, the method comprises administering to a subject any of the engineered cells described herein, and a PSMA-targeting molecule that is or further comprises a therapeutic agent.

In some embodiments, the PSMA-targeting molecule used in connection with the methods is capable of binding to PSMA or a modified form thereof, optionally to the active site of PSMA or the modified form, is cleaved by PSMA or a modified form thereof and/or is an antagonist or inhibitor of PSMA or a modified form thereof. In some embodiments, the PSMA-targeting molecule can bind part of the extracellular portion of the PSMA or modified form thereof, e.g., PSMA expressed on PSMA or modified form thereof is expressed on one or more of the engineered cells described herein. Thus, by virtue of the PSMA-targeting molecule binding to PSMA, the therapeutic agent can be targeted or delivered to specific cells or microenvironments that are associated with expression of PSMA or modified form thereof. In some embodiments, the method includes administering to a subject having been administered any of the engineered cells provided herein, a PSMA-targeting molecule, said PSMA-targeting molecule that is or further comprises a therapeutic agent. For example, in some embodiments, the PSMA-targeting molecule used in the methods of treatment provided herein, can be any that are described in Section III above.

Provided are methods of administering the engineered cells and compositions, and uses of such engineered cells and compositions to treat or prevent diseases, conditions, and disorders, including cancers. In some embodiments, the engineered cells and compositions are administered to a subject or patient having the particular disease or condition to be treated, e.g., via adoptive cell therapy, such as adoptive T cell therapy. In some embodiments, provided cells and compositions are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

The disease or condition that is treated in some aspects can be any in which expression of an antigen is associated with, specific to, and/or expressed on a cell or tissue of a disease, disorder or condition and/or involved in the etiology of a disease, condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the immunomodulatory polypeptide and/or recombinant receptor, e.g., the chimeric antigen receptor or TCR, specifically binds to an antigen associated with the disease or condition. In some embodiments, the subject has a disease, disorder or condition, optionally a cancer, a tumor, an autoimmune disease, disorder or condition, or an infectious disease.

In some embodiments, the disease, disorder or condition includes tumors associated with various cancers. The cancer can in some embodiments be any cancer located in the body of a subject, such as, but not limited to, cancers located at the head and neck, breast, liver, colon, ovary, prostate, pancreas, brain, cervix, bone, skin, eye, bladder, stomach, esophagus, peritoneum, or lung. For example, the anti-cancer agent can be used for the treatment of colon cancer, cervical cancer, cancer of the central nervous system, breast cancer, bladder cancer, anal carcinoma, head and neck cancer, ovarian cancer, endometrial cancer, small cell lung cancer, non-small cell lung carcinoma, neuroendocrine cancer, soft tissue carcinoma, penile cancer, prostate cancer, pancreatic cancer, gastric cancer, gall bladder cancer or espohageal cancer. In some cases, the cancer can be a cancer of the blood. In some embodiments, the disease, disorder or condition is a tumor, such as a solid tumor, lymphoma, leukemia, blood tumor, metastatic tumor, or other cancer or tumor type. In some embodiments, the disease, disorder or condition is selected from among cancers of the colon, lung, liver, breast, prostate, ovarian, skin, melanoma, bone, brain cancer, ovarian cancer, epithelial cancers, renal cell carcinoma, pancreatic adenocarcinoma, cervical carcinoma, colorectal cancer, glioblastoma, neuroblastoma, Ewing sarcoma, medulloblastoma, osteosarcoma, synovial sarcoma, and/or mesothelioma.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma,diffuse large B-cell lymphoma (DLBCL) and multiple myeloma (MM), a B cell malignancy is selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is selected from the group consisting of orphan tyrosine kinase receptor ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), ephrine receptor A2 (EPHa2), Her2/neu (receptor tyrosine kinase erb-B2), Her3 (erb-B3), Her4 (erb-B4), erbB dimers, type III epidermal growth factor receptor mutation (EGFR vIII), folate binding protein (FBP), FCRL5, Fc receptor like 5 (FCRL5; also known as Fc receptor homolog 5 or FCRH1), fetal acetylcholine receptor, ganglioside GD2, ganglioside GD3, G Protein Coupled Receptor 5D (GPCR5D), HMW-MAA, IL-22R-alpha, IL-13R-alpha2, kinase insert domain receptor (kdr), kappa light chain, Leucine Rich Repeat Containing 8 Family Member A (LRRC8A), Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, Human leukocyte antigen A1 (HLA-A1), MAGE A1, HLA-A2, NY-ESO-1, PSCA, folate receptor-a, CD44v6, CD44v7/8, αvβ6 integrin (avb6 integrin), 8H9, NCAM, VEGF receptors, 5T4, Foetal AchR, natural killer group 2 member D (NKG2D) ligands, CD44v6, dual antigen, a cancer-testes antigen, mesothelin, murine CMV, mucin 1 (MUC1), MUC16, prostate stem cell antigen (PSCA), NKG2D, a cancer-testis antigen cancer/testis antigen 1B (CTAG, also known as NY-ESO-1 and LAGE-2), MART-1, glycoprotein 100 (gp100), oncofetal antigen, ROR1, Trophoblast glycoprotein (TPBG also known as 5T4), TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, C-C Motif Chemokine Ligand 1 (CCL-1), CD138, a pathogen-specific antigen and an antigen associated with a universal tag, and/or biotinylated molecules, and/or molecules expressed by HIV, HCV, HBV or other pathogens.

In some embodiments, the antigen or ligand is a tumor antigen or cancer marker. In some embodiments, the antigen or ligand the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the antigen is a pathogen-specific or pathogen-expressed antigen. In some embodiments, the antigen is a viral antigen (such as a viral antigen from HIV, HCV, HBV, etc.), bacterial antigens, and/or parasitic antigens.

Methods for administration of engineered cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

As used herein, a “subject” is a mammal, such as a human or other animal, and typically is human. In some embodiments, the subject, e.g., patient, to whom the immunomodulatory polypeptides, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or engineered cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering the immunomodulatory polypeptides, engineered cells, or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The provided methods and uses include methods and uses for adoptive cell therapy. In some embodiments, the methods include administration of the engineered cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition, ameliorate one or more symptom of the disease or condition.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or super type as the first subject. The cells can be administered by any suitable means. Dosing and administration may depend in part on whether the administration is brief or chronic. Various dosing schedules include but are not limited to single or multiple administrations over various time-points, bolus administration, and pulse infusion.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸ or total such cells, or the range between any two of the foregoing values.

In some embodiments, the dose of genetically engineered cells comprises from or from about 1×10⁵ to 5×10⁸ total CAR-expressing T cells, 1×10⁵ to 2.5×10⁸ total CAR-expressing T cells, 1×10⁵ to 1×10⁸ total CAR-expressing T cells, 1×10⁵ to 5×10⁷ total CAR-expressing T cells, 1×10⁵ to 2.5×10⁷ total CAR-expressing T cells, 1×10⁵ to 1×10⁷ total CAR-expressing T cells, 1×10⁵ to 5×10⁶ total CAR-expressing T cells, 1×10⁵ to 2.5×10⁶ total CAR-expressing T cells, 1×10⁵ to 1×10⁶ total CAR-expressing T cells, 1×10⁶ to 5×10⁸ total CAR-expressing T cells, 1×10⁶ to 2.5×10⁸ total CAR-expressing T cells, 1×10⁶ to 1×10⁸ total CAR-expressing T cells, 1×10⁶ to 5×10⁷ total CAR-expressing T cells, 1×10⁶ to 2.5×10⁷ total CAR-expressing T cells, 1×10⁶ to 1×10⁷ total CAR-expressing T cells, 1×10⁶ to 5×10⁶ total CAR-expressing T cells, 1×10⁶ to 2.5×10⁶ total CAR-expressing T cells, 2.5×10⁶ to 5×10⁸ total CAR-expressing T cells, 2.5×10⁶ to 2.5×10⁸ total CAR-expressing T cells, 2.5×10⁶ to 1×10⁸ total CAR-expressing T cells, 2.5×10⁶ to 5×10⁷ total CAR-expressing T cells, 2.5×10⁶ to 2.5×10⁷ total CAR-expressing T cells, 2.5×10⁶ to 1×10⁷ total CAR-expressing T cells, 2.5×10⁶ to 5×10⁶ total CAR-expressing T cells, 5×10⁶ to 5×10⁸ total CAR-expressing T cells, 5×10⁶ to 2.5×10⁸ total CAR-expressing T cells, 5×10⁶ to 1×10⁸ total CAR-expressing T cells, 5×10⁶ to 5×10⁷ total CAR-expressing T cells, 5×10⁶ to 2.5×10⁷ total CAR-expressing T cells, 5×10⁶ to 1×10⁷ total CAR-expressing T cells, 1×10⁷ to 5×10⁸ total CAR-expressing T cells, 1×10⁷ to 2.5×10⁸ total CAR-expressing T cells, 1×10⁷ to 1×10⁸ total CAR-expressing T cells, 1×10⁷ to 5×10⁷ total CAR-expressing T cells, 1×10⁷ to 2.5×10⁷ total CAR-expressing T cells, 2.5×10⁷ to 5×10⁸ total CAR-expressing T cells, 2.5×10⁷ to 2.5×10⁸ total CAR-expressing T cells, 2.5×10⁷ to 1×10⁸ total CAR-expressing T cells, 2.5×10⁷ to 5×10⁷ total CAR-expressing T cells, 5×10⁷ to 5×10⁸ total CAR-expressing T cells, 5×10⁷ to 2.5×10⁸ total CAR-expressing T cells, 5×10⁷ to 1×10⁸ total CAR-expressing T cells, 1×10⁸ to 5×10⁸ total CAR-expressing T cells, 1×10⁸ to 2.5×10⁸ total CAR-expressing T cells, or 2.5×10⁸ to 5×10⁸ total CAR-expressing T cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷, 1×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the size of the dose is determined by the burden of the disease or condition in the subject. For example, in some aspects, the number of cells administered in the dose is determined based on the tumor burden that is present in the subject immediately prior to administration of the initiation of the dose of cells. In some embodiments, the size of the first and/or subsequent dose is inversely correlated with disease burden. In some aspects, as in the context of a large disease burden, the subject is administered a low number of cells. In other embodiments, as in the context of a lower disease burden, the subject is administered a larger number of cells.

In some embodiments, the methods include administration of a PSMA-targeting molecule. In some embodiments, the cells are administered simultaneously with or sequentially with, in any order, a PSMA-targeting molecule, e.g., a PSMA-targeting molecule that is or comprises a therapeutic agent and/or a detectable moiety.

In some embodiments, the PSMA-targeting molecule is administered sequentially, intermittently, or at the same time as or in the same composition as cells for adoptive engineered cells. For example, the PSMA-targeting molecule can be administered prior to, during, simultaneously with, or after administration of the engineered cells. In some embodiments, the PSMA-targeting molecule is administered simultaneously with the engineered cells, in the same composition or in different compositions. In some embodiments, the PSMA-targeting molecule is contacted with the engineered cells, e.g., contained in the same composition, prior to administration of both the engineered cells and the PSMA-targeting molecule. In some embodiments, the engineered cell and the PSMA-targeting molecule are administered independently, or in different compositions.

In some embodiments, the method involves administering the PSMA-targeting molecule prior to administration of the engineered cells. In some embodiments, the PSMA-targeting molecule is not further administered after initiation of the engineered cells. In other embodiments, the method further involves administering the PSMA-targeting molecule after administration of the engineered cells. In some cases, the dosage schedule comprises administering the PSMA-targeting molecule prior to and after initiation of the engineered cells. In some embodiments, the initiation of administration of the PSMA-targeting molecule is at a time point that is greater than or greater than about 1 hour, 2 hours, 6 hours, 12 hours, 24 hours, 3 days, 6 days, 12 days, 15 days, 30 days, 60 days or 90 days prior to initiation of the administration of the engineered cells. In some aspects, the initiation of administration of the PSMA-targeting molecule is more than 4 days before the administration of the engineered cells.

In some embodiments, the PSMA-targeting molecule is administered daily, every other day, once a week or only one time prior to initiation of administration of the engineered cells. In some aspects, the PSMA-targeting molecule is administered until a therapeutic effect of the PSMA-binding molecule that is or comprises a therapeutic agent is observed. In some embodiments, the PSMA-targeting molecule is administered for a time period up to 2 days, up to 7 days, up to 14 days, up to 21 days, up to 28 days, up to 35 days or up to 42 days after initiation of the administration of the engineered cells. In some embodiments, the PSMA-targeting molecule is administered daily, every other day, once a week or only one time after initiation of administration of the engineered cells for the time period. In some embodiments, the PSMA-targeting molecule can be administered greater than 4 hours, 5 hours, 6 hours, 7 hours, 8 hours, 9 hours, 10 hours, 11 hours, 12 hours, 18 hours, 24 hours, 36 hours, 2 days, 3 days, 4 days, or 5 days or more following administration of the engineered cells. In some of such embodiments, the inhibitor may be administered no later than 5 hours, 6 hours, 7 hours, 8 hours, 9 hours, 10 hours, 11 hours, 12 hours, 18 hours, 24 hours, 36 hours, 2 days, 3 days, 4 days, or 5 days or more following administration of the engineered cells.

In some embodiments, the PSMA-targeting molecule is independently administered at a dose determined based on one or more criteria such as type and characteristics of the PSMA-targeting molecule and/or therapeutic agent, response of the subject to prior treatment, dose of the administered engineered cells, disease burden in the subject, such as tumor load, bulk, size, or degree, extent, or type of metastasis, stage, and/or likelihood or incidence of the subject developing toxic outcomes.

In some embodiments, the PSMA-targeting molecule is independently administered in a dosage amount of from or from about 0.2 mg per kg body weight of the subject (mg/kg) to 200 mg/kg, 0.2 mg/kg to 100 mg/kg, 0.2 mg/kg to 50 mg/kg, 0.2 mg/kg to 10 mg/kg, 0.2 mg/kg to 1.0 mg/kg, 1.0 mg/kg to 200 mg/kg, 1.0 mg/kg to 100 mg/kg, 1.0 mg/kg to 50 mg/kg, 1.0 mg/kg to 10 mg/kg, 10 mg/kg to 200 mg/kg, 10 mg/kg to 100 mg/kg, 10 mg/kg to 50 mg/kg, 50 mg/kg to 200 mg/kg, 50 mg/kg to 100 mg/kg or 100 mg/kg to 200 mg/kg ; or the PSMA-targeting molecule is administered, or each administration of the PSMA-targeting molecule is independently administered, in a dosage amount of from or from about 25 mg to 2000 mg, 25 mg to 1000 mg, 25 mg to 500 mg, 25 mg to 200 mg, 25 mg to 100 mg, 25 mg to 50 mg, 50 mg to 2000 mg, 50 mg to 1000 mg, 50 mg to 500 mg, 50 mg to 200 mg, 50 mg to 100 mg, 100 mg to 2000 mg, 100 mg to 1000 mg, 100 mg to 500 mg, 100 mg to 200 mg, 200 mg to 2000 mg, 200 mg to 1000 mg, 200 mg to 500 mg, 500 mg to 2000 mg, 500 mg to 1000 mg or 1000 mg to 2000 mg, each inclusive. In some aspects, the PSMA-targeting molecule is administered, or each administration of the PSMA-targeting molecule is independently administered, in a dosage amount of at least or at least about or about 0.2 mg per kg body weight of the subject (mg/kg), 1 mg/kg, 3 mg/kg, 6 mg/kg, 10 mg/kg, 20 mg/kg, 30 mg/kg, 50 mg/kg, 100 mg/kg or 200 mg/kg; or the PSMA-targeting molecule is administered, or each administration of the PSMA-targeting molecule is independently administered, in a dosage amount of at least or at least about 25 mg, 50 mg, 100 mg, 200 mg, 400 mg, 500 mg, 600 mg, 800 mg, 1000 mg, 1200 mg, 1600 mg or 2000 mg. The PSMA-targeting molecule is administered daily in a dosage amount of at least or at least about 25 mg/day, 50 mg/day, 100 mg/day, 200 mg/day, 400 mg/day, 500 mg/day, 600 mg/day, 800 mg/day, 1000 mg/day, 1200 mg/day, 1600 mg/day or 2000 mg/day.

In some embodiments, the cells and/or PSMA-targeting molecules are administered as part of a further combination treatment, such as simultaneously with or sequentially with, in any order, another therapeutic intervention, such as an antibody or engineered cell or receptor or agent, such as a cytotoxic or therapeutic agent. For example, in some embodiments, an anti-cancer agent can be used in combination therapy with adoptive cell therapy with engineered cell expressing PSMA or modified form thereof together with an additional immunomodulatory agent, and/or in addition to administration of a PSMA-targeting molecule, e.g., a PSMA-targeting molecule that is capable of binding a PSMA or modified form thereof and is or comprises a therapeutic agent.

The cells in some embodiments are co-administered with one or more additional agents, a PSMA-targeting molecule, e.g., comprising a portion capable of binding PSMA and a therapeutic agent and/or a detectable moiety, and/or additional therapeutic agent or cytotoxic agent, in connection with another therapeutic intervention, either simultaneously or sequentially in any order. In some contexts, the cells are co-administered with PSMA-targeting molecules and/or another therapy sufficiently close in time such that the cell populations enhance the effect of the PSMA-targeting molecule and/or one or more additional therapeutic agents, or vice versa. In some embodiments, the cells are administered prior to the PSMA-targeting molecule and/or one or more additional therapeutic agents. In some embodiments, the cells are administered after the PSMA-targeting molecules and/or one or more additional therapeutic agents.

In some embodiments, the one or more additional therapeutic agents include a cytokine, such as IL-2, for example, to enhance persistence. In some embodiments, the methods comprise administration of a chemotherapeutic agent. In some embodiments, the one or more additional therapeutic agents include one or more lymphodepleting therapies, such as prior to or simultaneous with initiation of administration of the engineered cells. In some embodiments, the lymphodepleting therapy comprises administration of a phosphamide, such as cyclophosphamide. In some embodiments, the lymphodepleting therapy can include administration of fludarabine. In some embodiments, fludarabine is excluded in the lymphodepleting therapy. In some embodiments, a lymphodepleting therapy is not administered.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered recombinant receptor, such as CAR or TCR, expressed by the population can be conjugated either directly or indirectly through a linker to a targeting moiety. The practice of conjugating compounds, e.g., the CAR or TCR, to targeting moieties is known. See, for instance, Wadwa et al., J. Drug Targeting 3:111 (1995), and U.S. Pat. No. 5,087,616.

In some embodiments, the cell surface receptor conjugate is used for targeting engineered cells for suicide killing of engineered cells or removal of engineered cells. In some embodiments, provided are methods that can be used for ablation and/or depletion of engineered cells in vivo, for example, mediated via antibody-dependent cell-mediated cytotoxicity (ADCC) or via specific targeting of cells with a cytotoxic agent. In some aspects, killing of cells engineered to express the PSMA or modified form thereof uses PSMA-targeting molecules specific for the PSMA or modified form thereof. In other aspects, provided are methods of killing cells by targeting the agent of the PSMA or modified form thereof using a molecule comprising a PSMA-targeting molecule specific for the agent of the conjugate linked to a cytotoxic agent, such as a toxin.

In some embodiments, the PSMA-targeting molecule comprising a therapeutic agent that is a cytotoxic agent can be used in connection with the engineered cells provided herein, to trigger suicide of the engineered cell, e.g., after administration of the engineered cell to a subject, to remove or destroy the engineered cell. In some embodiments, the PSMA-targeting molecule comprises a portion that is capable of binding PSMA or modified form thereof, linked or conjugated to a cytotoxic agent. In some embodiments, the PSMA-targeting molecule comprising a cytotoxic agent is administered to a subject when the subject is known or suspected of having or likely having or developing an adverse side effect to the administered cells, such as associated with toxicity or immunogencitiy of the engineered cells.

In some embodiments, administration of the PSMA-targeting molecule comprising a therapeutic agent that is a cytotoxic agent does not, or does not substantially, induce killing or destruction of healthy tissue or healthy cells, of cells or tissues not containing the engineered cells and/or not expressing the antigen.

In some embodiments, the PSMA or modified form thereof may be used to induce cell suicide. For example, the cell surface molecule may be used as a suicide gene via antibody dependent cell mediated cytotoxicity (ADCC) pathways. ADCC refers to a cell-mediated reaction in which nonspecific cytotoxic cells that express Fc receptors, such as natural killer cells, neutrophils, and macrophages, recognize bound antibody on a target cell and cause lysis of the target cell. ADCC activity may be assessed using methods, such as those described in U.S. Pat. No. 5,821,337. In some embodiments, ADCC may be mediated by administering to a subject any antibody targeting the PSMA or modified form thereof. In some aspects, PSMA or modified form thereof, engineered to be expressed on the cell surface, may be used as a suicide gene via administration of a PSMA-targeting molecule that is an anti-PSMA antibody, such as any described herein.

In some embodiments, suicide killing of cells expressing the PSMA or modified form thereof is accomplished by employing a PSMA-targeting molecule that is or comprises a cytotoxic agent, e.g., a toxin. In some embodiments, by virtue of binding of the PSMA-targeting molecule that contains a cytotoxic agent, to the PSMA or modified form thereof, the toxin can be specifically targeted or delivered to the PSMA-expressing cell, e.g., the engineered cells described herein. In some embodiments, the binding of the PSMA-targeting molecule to the PSMA or modified form thereof on the cell surface of the engineered cell can result in internalization of the receptor and molecules bound thereto. Thus, by virtue of binding to the PSMA and internalizing, the PSMA-binding molecule containing the cytotoxic agent can be specifically targeted to the engineered cell to result in cell suicide or removal.

### V. METHODS OF DETECTION

In some embodiments, methods are provided for monitoring, such as detecting or identifying, engineered cells administered to the subject, such as for determining or assessing the presence, number or location of such cells in the subject. In some embodiments, detection is carried out in vivo is performed in vivo. In some embodiments, detection is carried out in vitro or ex vivo from a sample from the subject. Also provided are methods and uses for identification, detection or selection of cells and compositions, such as those containing the engineered cells, by recognition of the PSMA or modified form thereof expressed by the engineered cells.

In some embodiments, the method comprises detecting cells that express the PSMA or modified form thereof and/or detecting the binding of the PSMA-targeting molecule to the PSMA or modified form thereof and/or the presence of the PSMA-targeting molecule. In some embodiments, the PSMA-targeting molecule is or comprises one or more moiety that provides a signal or induces a signal that is detectable. For example, in some embodiments, the PSMA-targeting molecule is or comprises a detectable moiety. In some embodiments, the PSMA-targeting molecule used in connection with the methods of detection described herein can be any described in Section III above.

In some embodiments, the methods for detection include contacting any of the engineered cells described herein with a PSMA-targeting molecule; and detecting the binding of said PSMA-targeting molecule and/or the presence of said PSMA-targeting molecule to or with the PSMA or modified form thereof and/or the engineered cells. In some embodiments, the contacting comprises administering, to a subject having been administered the engineered cells, the PSMA-targeting molecule.

In some embodiments, methods are provided for detecting the presence or absence of engineered cells expressing a recombinant receptor and a PSMA or modified form thereof in a subject, said subject having been previously administered any of the engineered cells described herein. In some embodiments, the method involves administering to the subject a PSMA-targeting molecule; and detecting the binding of the PSMA-targeting molecule to the PSMA or modified form thereof and/or to the engineered cells and/or the presence of the PSMA-targeting molecule in the subject.

In some cases, a limit of detection (LOD) in any of the provided embodiments is, or the embodiments such as the methods or assays for detection provided herein, allow for, are useful in, or are or are or is capable of, detecting, as low or few as, or as low or few as approximately 500, 1,000, 2,000, 3,000, 4,000, 5,000, 6,000, 7,000, 8,000, 9,000 or 10,000 cells, such as engineered cells expressing the PSMA or modified form thereof such as a truncated form of PSMA. In some aspects of such embodiments, the limit of detection is, or the embodiments such as methods and assays, allow for, are useful in, or are or are or are capable of, detecting such cells, e.g., such number of cells or at least or as few as or as low as such number of cells, present in a specified volume or tissue or sample. In some aspects, the volume is between at or about 10 and at or about 200 μL, at or about 10 and at or about 200 μL, at or about 10 and at or about 100 μL, at or about 10 and at or about 90 μL, at or about 10 and at or about 80 μL, at or about 10 and at or about 70 μL, at or about 10 and at or about 60 μL, at or about 10 and at or about 50 μL, at or about 10 and at or about 40 μL, at or about 10 and at or about 30 μL, at or about 10 and at or about 20 μL, at or about 20 and at or about 100 μL, at or about 20 and at or about 90 μL, at or about 20 and at or about 80 μL, at or about 20 and at or about 70 μL, about at or 20 and at or about 60 μL, at or about 20 and at or about 50 μL, at or about 20 and at or about 40 μL, at or about 20 and at or about 30 μL, at or about 20 and at or about 20 μL, at or about 50 and at or about 100 μL, at or about 50 and at or about 90 μL, at or about 50 and at or about 80 μL, at or about 50 and at or about 70 μL, at or about 50 and at or about 60 μL, such as at or about 10, 20, 30, 40, 50, 60, 70, 80, 90 or 100 μL. In some aspects, the volume is a volume of liquid, buffer, medium, a volume of a sample such as a biological sample or biological fluid, and/or of an organ or tissue such as a tumor, such as a tumor or portion thereof corresponding to the disease or condition treated, and/or of an in vitro culture system, medium or buffer, and/or a mixture or a matrix, e.g., a matrigel.

In some of any of the embodiments, the limit of detection or number of cells per volume is as low or few as, or as low or few as approximately 10, 20, 30, 40, 50, 60, 70, 80, 90, 100, 150, 200, 250, 300, 400, 500 or 1,000 of the cells, such as the PSMA (e.g., tPSMA)-expressing cells or engineered cells, per μL. In some of any of the provided embodiments, the embodiments or methods are useful in or allow for or are capable of detecting, as low or few as, or as low or few as approximately 10, 20, 30, 40, 50, 60, 70, 80, 90, 100, 150, 200, 250, 300, 400, 500 or 1,000 of the cells/μL. In some of any of the embodiments, the limit of detection or number of cells per volume is as low or few as, or as low or few as approximately 500, 1,000, 2,000, 4,000, 5,000 or 10,000 cells in a 20 or 50 μL. In some embodiments, the limit of detection in any of the provided embodiments is, or the methods or assays for detection described herein is capable of detecting, as low or few as, or as low or few as approximately 2,000, 4,000, 5,000 or 10,000 cells in a 20 or 50 μL volume.

In some embodiments, a limit of detection is or the methods and assays are capable of detecting the cells present in a specific volume, e.g., volume of liquid, buffer, medium, of a sample, and/or of an organ or tissue, and/or of an in vitro culture system, medium or buffer, and/or a mixture or a matrix, e.g., a matrigel. In some embodiments, the limit of detection is a limit of detection in in vivo imaging. In some embodiments, the limit of detection is a limit of detection in in vitro or ex vivo imaging.

In some embodiments, the PSMA-targeting molecule used in connection with the methods is capable of binding to PSMA or a modified form thereof, optionally to the active site of PSMA or the modified form, is cleaved by PSMA or a modified form thereof and/or is an antagonist or inhibitor of PSMA or a modified form thereof.

In some embodiments, the method of detection involves assessment, such as quantitative assessment of the exposure, number, concentration, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the presence and/or characteristics of the T cells in vivo, in vitro or ex vivo, e.g., using any of the detection methods described below. In some embodiments, such assays can be used to determine or confirm the presence, proliferation, number, concentration and/or function of the T cells used for the immunotherapy, e.g. T cell therapy, before or after administering the provided cells or compositions, e.g., engineered T cells expressing PSMA or modified form thereof

In some aspects, the exposure, number, concentration, persistence and proliferation relate to pharmacokinetic parameters. In some embodiments, the pharmacokinetic parameters include the number or concentration of cells in a particular location of the body, e.g., in a particular organ or tissue, in a tumor, or in the plasma. In some embodiments, pharmacokinetic parameters also include assessing the change of number or concentration of the cells over time or determining the total exposure to the therapeutic, e.g., administered T cells, over a certain period of time. In some cases, pharmacokinetics can also be assessed by measuring such parameters as the maximum (peak) concentration (Cₘₐₓ), the peak time (i.e. when maximum concentration (Cₘₐₓ) occurs; Tₘₐₓ), the minimum plasma concentration (i.e. the minimum concentration between doses of a administered cells, e.g., engineered T cells expressing PSMA or variant thereof; Cₘᵢₙ), the elimination half-life (T_{1/2}) and area under the curve (i.e. the area under the curve generated by plotting time versus plasma concentration of the administered cells, e.g., CAR+ T cells expressing PSMA or a variant thereof; AUC), following administration. The concentration of a particular administered cells, e.g., engineered T cells expressing PSMA or variant thereof, in the plasma following administration can be measured using any detection methods for detecting the PSMA or variant thereof, e.g., by administering a PSMA-targeting molecule containing a detectable moiety. In some embodiments, the pharmacokinetic parameters can be determined using an in vivo detection method, e.g., positron emission tomography (PET), computed tomography (CT) and single photon emission computed tomography (SPECT), using radionuclide-labeled ligands. Other known in vivo, in vitro or ex vivo methods suitable for assessing concentrations of the administered cells, e.g., CAR+ T cells expressing PSMA or a variant thereof, in biological sample e.g., blood, or any methods described herein can be used to detect administered cells, e.g., CAR+ T cells expressing PSMA or a variant thereof. For example, nucleic acid-based methods, such as quantitative PCR (qPCR) or flow cytometry-based methods, or other assays, such as an immunoassay, ELISA, or chromatography/mass spectrometry-based assays can be used.

In some embodiments, “exposure” can refer to the body exposure of a administered cells, e.g., engineered T cells expressing PSMA or variant thereof in a particular location of the body, e.g., in a particular organ or tissue, in a tumor, or in the plasma (blood or serum), after administration of the administered cells, e.g., CAR+ T cells expressing PSMA or a variant thereof over a certain period of time. In some embodiments exposure can be set forth as the area under the administered cells, e.g., CAR+ T cells expressing PSMA or a variant thereof concentration-time curve (AUC) as determined by pharmacokinetic analysis after administration of a dose of the administered cells, e.g., engineered T cells expressing PSMA or variant thereof. In some cases, the AUC is expressed in cells*days/μL, for cells administered in cell therapy, or in corresponding units thereof. In some embodiments, the AUC is measured as an average AUC in a patient population, such as a sample patient population, e.g., the average AUC from one or more patient(s). In some embodiments, exposure refers to the area under the curve (AUC) within a certain period of time, e.g., from day 0 to day 1, 2, 3, 4, 5, 6, 7, 8, 9, 10, 11, 12, 13, 14, 21, 28 days or more, or week 1, 2, 3, 4, 5, 6, 7, 8, 9, 10, 11, 12, 13, 14, 15 or more, or month 1, 2, 3, 4, 5, 6, 7, 8, 9, 10, 11, 12, 18, 24, 48 or more. In some embodiments, the AUC is measured as an AUC from day 0 to day 28 (AUC₀₋₂₈) after administration of the administered cells, e.g., engineered T cells expressing PSMA or variant thereof, including all measured data and data extrapolated from measured pharmacokinetic (PK) parameters, such as an average AUC from a patient population, such as a sample patient population. In some embodiments, to determine exposure over time, e.g., AUC for a certain period of time, such as AUC₀₋₂₈, an administered cell concentration-time curve is generated, using multiple measurements or assessment of parameters, e.g., cell concentrations, over time, e.g., measurements taken every 1, 2, 3, 4, 5, 6, 7, 8, 9, 10, 11, 12, 13, 14, 21 or 28 days or more.

A. In Vivo Detection

In some embodiments, the method of detection, identification and/or monitoring is performed in vivo by administering to the subject a PSMA-targeting molecule that is capable of binding the PSMA or modified form thereof, and comprises one or more moiety that provides a signal or induces a signal that is detectable. In some embodiments, the PSMA-targeting molecule provides a signal or induces a signal that is detectable or is capable of binding to a moiety that provides a signal or induces a signal that is detectable; and/or the PSMA-targeting molecule is or comprises a moiety that provides a signal or induces a signal that is detectable. In some embodiments, imaging of cells, such as cells expressing the PSMA or modified form thereof and/or a recombinant receptor, in real time reveals the locations of transduced cells in vivo. In some embodiments, the method of detection can be used to monitor the biological distribution of the engineered cells, administered PSMA-targeting molecule or diagnose or assess outcomes associated with toxicity or immunogencitiy of the engineered cells.

In some aspects, in vivo detection is carried out using a PSMA-targeting molecule, such any described herein, that provides a signal or induces a signal that is detectable or is capable of binding to a moiety that provides a signal or induces a signal that is detectable; and/or comprises a moiety that provides a signal or induces a signal that is detectable, in vivo. In some embodiments PSMA-targeting molecule is or comprises an imaging probe, a detection reagent, an imaging modality or a detectable label. In some embodiments, the detection reagent comprises a radioligand. In some embodiments, the imaging probe, detection reagent, imaging modality or detectable label comprises a radioisotope, a bioluminescent compound, a chemiluminescent compound, a fluorescent compound, a chromogenic compound, a quantum dot, a nanoparticle, a metal chelate, an enzyme, an iron-oxide nanoparticle or other known imaging agents for detection by X-ray, CT-scan, MRI-scan, PET-scan, ultrasound, flow-cytometry, near infrared imaging systems, or other imaging modalities (see, e.g., Yu et al., Theranostics (2012) 2:3).

In some embodiments, the in vivo imaging, detection or diagnostic method for detecting cells can be magnetic resonance imaging (MRI), single-photon emission computed tomography (SPECT), computed tomography (CT), computed axial tomography (CAT), electron beam computed tomography (EBCT), high resolution computed tomography (HRCT), hypocycloidal tomography, positron emission tomography (PET), scintigraphy, gamma camera, a β+ detector, a γ detector, fluorescence imaging, low-light imaging, X-rays, bioluminescence imaging, near-infrared (NIR) optical tomography, and other imaging modalities.

Also provided are methods involving use of the provided PSMA-targeting molecules, e.g., small molecules, ligands or antibodies or antigen-binding fragment thereof, for detection, prognosis, diagnosis, staging, determining binding of a particular treatment to one or more tissues or cell types, and/or informing treatment decisions in a subject, such as by the detection of the presence of an epitope thereof recognized by the PSMA-targeting molecule. In some embodiments, the methods are diagnostic and/or prognostic methods in association with a disease or condition that is associated with expression of the target antigen specifically recognized by the recombinant receptor. The methods in some embodiments include incubating and/or probing a biological sample with the antibody and/or administering the antibody to a subject. In certain embodiments, a biological sample includes a cell or tissue or portion thereof, such as tumor or cancer tissue or biopsy or section thereof.

In some embodiments, the PSMA-targeting molecule or portion thereof that can be used for detection or diagnostic purposes include any described in, e.g.; WO2015143029; WO2016/065142; US 201013257499; US 2012/0067162; US 201213566849; US 201214008715; US 201214126296; US 201313826079; US 2014/0060461; US 201414152864; US 201414277367; US 201414335055; US 2015/0021233; US 2015/0029504; US 2015/0054937; US 2015/0056914; US 201514937169; US 2016/0022309; US 2016/0046981; U.S. Pat. No. 23,913,608; 74,498,208; 89,753,907; AU 2008/269094; AU 2009/276423; AU 2015/203742; EP 03703745; EP 2015001929; EP 2015069356; EP 2016069730; Dobrenkov et al. (2008) J Nucl Med. 49:1162-1170; Chen et al., Biochem. Biophys. Res. Comm. 2009, 390(3):624-629; Banerjee et al., Oncotarget 2011; 2(12): 1244-1253; Banerjee et al. (2011) Angew Chem Int Ed Engl. 50(39): 9167-9170; Maurer et al. (2016) Nature Reviews Urology 13:226-235; Rowe et al. (2016) Prostate Cancer Prostatic Dis. 19(3):223-230; Mease et al., (2013) Curr Top Med Chem. 13(8):951-962; Osborne et al., (2013) Urol Oncol. 31(2): 144-154; or Barinka et al., (2008) J Mol Biol. 2008 March 7; 376(5): 1438-1450; Philipp Wolf (2011), Prostate Specific Membrane Antigen as Biomarker and Therapeutic Target for Prostate Cancer, Prostate Cancer—Diagnostic and Therapeutic Advances, Philippe E. Spiess (Ed.), Intech, pp.81-100.

In some embodiments, the PSMA-targeting molecule or a portion thereof is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N,N′-diaceticacid, MIP-1072, MIP-1095, MIP-1404, MIP-1405, N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE).

In some embodiments, the method for in vivo imaging, detection or diagnostics for detecting engineered cells expressing a PSMA or a variant thereof is positron emission tomography (PET)/computed tomography (CT). In some embodiments, the method for in vivo imaging, detection or diagnostics for detecting engineered cells expressing a PSMA or a variant thereof is performed after administration to a subject who had received engineered cell therapy a PSMA-targeting molecule that can be used as a PET/CT ligand. In some embodiments, the PSMA-targeting molecule is or comprises radiolabeled PSMA-targeting molecule, e.g., 2-(3-{1-carboxy-5-[(6-fluoro-pyridine-3-carbonyl)-amino]-pentyl}-ureido)-pentanedioicacid (DCFPyL), such as 2-(3-{1-carboxy-5-[(6-[¹⁸F]fluoro-pyridine-3-carbonyl)-amino]-pentyl}-ureido)-pentanedioic acid (¹⁸F-DCFPyL), a high-affinity positron-emitting ligand.

In some embodiments, for PET/CT, 2-(3-{1-carboxy-5-[(6-[¹⁸F]fluoro-pyridine-3-carbonyl)-amino]-pentyl}-ureido)-pentanedioic acid (¹⁸F-DCFPyL) is administered to a subject at a dose based on the dose of the radiolabel. In some embodiments, for PET/CT, ¹⁸F-DCFPyL is administered to a subject at a dose that is safe or not harmful to humans, such as a dose that is with in regulatory requirements (e.g., guidelines set forth by International Commission on Radiological Protection (ICRP) or Code of Federal Regulations 21, part 361) for use of radiolabeled compounds in human subjects. In some embodiments, the dose of ¹⁸F-DCFPyL for administration is a dose that does not exceed a critical organ dose limit, for the organ with the highest mean absorbed dose unit per administered activity, based on absorbed dose in different organs (see, e.g., Chen et al., Clin Cancer Res. 2011 Dec. 15; 17(24): 7645-7653; Shan L. 2-(3-{1-Carboxy-5-[(6-[18F]fluoro-pyridine-3-carbonyl)-amino]-pentyl}-ureido)-pentanedioic acid. 2012 Nov. 10 [Updated 2012 Dec. 19]. In: Molecular Imaging and Contrast Agent Database (MICAD) [Internet]. Bethesda (MD): National Center for Biotechnology Information (US); 2004-2013. Available from: https://www.ncbi.nlm.nih.gov/books/NBK114904/).

In some embodiments, exemplary dose of ¹⁸F-DCFPyL for administration to a small animal, e.g., a mouse, includes between or between about 50 μCi (1.85 MBq) to about 10 mCi (370 Mbq), such as about 50 μCi to about 1 mCi, 100 μCi to 500 μCi, or 200 μCi to 400 μCi. In some embodiments, for a human subject a human equivalent dose based on a small animal experiment, e.g., mouse experiment, is used. In some embodiments, human dosimetry values can be extrapolated based on biodistribution results from a small animal experiment, e.g., mouse experiment (See, e.g., Stabin et al., (2005) J Nucl Med. 46:1023-7). In some embodiments, exemplary dose of ¹⁸F-DCFPyL for administration to a human is an equivalent dose of between or between about 50 μCi (1.85 MBq) to about 5 mCi (185 Mbq), such as about 50 μCi to about 1 mCi, 100 μCi to 500 μCi, or 200 μCi to 400 μCi, for a small animal, e.g., a mouse. In some embodiments, exemplary dose of ¹⁸F-DCFPyL for administration to human subject includes between or between about 50 μCi (1.85 MBq) to about 100 mCi (3.7 Gbq), such as about 50 μCi to 100 mCi, 100 μCi to 50 mCi, 200 μCi to 25 mCi, 500 μCi to 20 mCi, 1 mCi to 10 mCi, such as about 50 μCi, 100 μCi, 200 μCi, 400 μCi, 500 μCi, 750 μCi, 1 mCi, 2 mCi, 3 mCi, 4 mCi, 5 mCi, 6 mCi, 7 mCi, 8 mCi, 9 mCi, 10 mCi, 15 mCi, 20 mCi, 25 mCi, or 50 mCi. In some embodiments, the dose of ¹⁸F-DCFPyL for administration to human subject include suitable known doses for ¹⁸F-DCFPyL or related or similar compounds labeled with ¹⁸F for administration to human subject (see, e.g., Chen et al., Clin Cancer Res. 2011 Dec. 15; 17(24): 7645-7653; Shan L. 2-(3-{1-Carboxy-5-[(6-[18F]fluoro-pyridine-3-carbonyl)-amino]-pentyl}-ureido)-pentanedioic acid. 2012 Nov. 10 [Updated 2012 Dec. 19]. In: Molecular Imaging and Contrast Agent Database (MICAD) [Internet]. Bethesda (MD): National Center for Biotechnology Information (US); 2004-2013. Available from: https://www.ncbi.nlm.nih.gov/books/NBK114904/).

In some aspects, the provided embodiments, can be used to obtain information regarding spatial distribution of the administered cells. In some aspects, the provided embodiments, including methods of detection, can be used to assess the whole-body spatial distribution of adoptively transferred cells, to determine specific location of the cells, e.g., within or around the site or location of a disease or disorder, e.g., tumor, persistence of the cells in the body and/or development of adverse effects, e.g., toxicities. In some aspects, the provided embodiments can be used in connection with methods for in vivo imaging, detection or diagnosis, such as magnetic resonance imaging (MRI), single-photon emission computed tomography (SPECT), computed tomography (CT), computed axial tomography (CAT), electron beam computed tomography (EBCT), high resolution computed tomography (HRCT), hypocycloidal tomography, positron emission tomography (PET), scintigraphy, gamma camera, a β+ detector, a γ detector, fluorescence imaging, low-light imaging, X-rays, bioluminescence imaging, near-infrared (NIR) optical tomography, and other imaging modalities, to obtain distribution of spatial information.

In some embodiments, the method further includes determining the number or concentration of the administered engineered cells in the subject. In some embodiments, determining the number or concentration of the administered engineered cells comprises comparing a signal from the detection of binding or presence of the PSMA-targeting molecule in the subject or in a sample from the subject, to a standard curve. In some embodiments, the standard curve is generated from the signal from the detection of binding or presence of the PSMA-targeting molecule in a plurality of control samples containing a defined number of cells.

In some aspects, the provided embodiments, can be used to obtain quantitative information regarding the administered cells, e.g., to estimate the number of administered cells present throughout the body and/or present in a particular organ or tissue. In some aspects, the provided embodiments can be used to quantitatively assess the presence, biodistribution, trafficking and/or pharmacokinetics of the administered cells in real-time. In some embodiments, the method of detection includes assessment of the exposure, number, concentration, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the administered cells using in vivo methods. In some embodiments, such assays can be used to determine or confirm the function of the T cells used for the immunotherapy, e.g. T cell therapy, before or after administering the cell therapy provided herein.

In some embodiments, the provided methods include determining the number or concentration of the administered engineered cells in the subject. In some embodiments, determining the number or concentration of the administered engineered cells comprises comparing a signal from the detection of binding or presence of the PSMA-targeting molecule in the subject or in a sample from the subject, to a standard curve. In some embodiments, the standard curve is generated from the signal from the detection of binding or presence of the PSMA-targeting molecule in a plurality of control samples containing a defined number of cells.

In some aspects, the exposure, number, concentration, persistence and proliferation relate to pharmacokinetic parameters. In some embodiments, the pharmacokinetic parameters include the number or concentration of cells in a particular location of the body, e.g., in a particular organ or tissue, in a tumor, or in the plasma. In some embodiments, pharmacokinetic parameters also include assessing the change of number or concentration of the cells over time or determining the total exposure to the therapeutic, e.g., administered T cells, over a certain period of time. The concentration of the administered cells, e.g., CAR+ T cells expressing PSMA or variant thereof, in the plasma following administration can be measured using any detection methods for detecting the PSMA or variant thereof, e.g., by administering a PSMA-targeting molecule containing a detectable moiety. In some embodiments, the pharmacokinetic parameters can be determined using an in vivo detection method, e.g., positron emission tomography (PET), computed tomography (CT) and single photon emission computed tomography (SPECT), using radionuclide-labeled ligands.

In some aspects, the exposure, number, concentration, persistence and proliferation or other pharmacokinetic parameters of the administered engineered cells expressing PSMA or modified form thereof, can be determined using positron emission tomography (PET)/computed tomography (CT). In some embodiments, the number or concentration of cells in a particular location of the body, e.g., in a particular organ, tissue or tumor, and/or throughout the body. In some embodiments, the number or concentration of cells in a particular location and/or throughout the body can be quantitatively determined by extrapolation based on a standard curve determined using a particular detection method and known concentration of cells. For example, in some embodiments, a standard curve can be established based on imaging, e.g., by PET/CT, of several different known number or concentration of cells, after exposure to a PET ligand specific for the particular cells, e.g., engineered cells expressing PSMA or a modified form thereof, such as ¹⁸F-DCFPyL.

In some embodiments, the standard curve can be established using readouts or results, e.g., PET signal, from multiple known number or concentration of cells, e.g., of serial dilution of cells. In some embodiments, the standard curve can be established using multiple known number or concentration of cells that are over the limit of detection of the method, or within the range of detection of the method. For example, in some embodiments, the limit of detection of the method can be as low or few as approximately 500, 1,000, 2,000, 3,000, 4,000 or 5,000 cells in a particular condition, e.g., specific volume, and a standard curve can be generated using known cell concentrations above the limit of detection in the particular condition. In some embodiments, a range of cell concentrations used for standard curve can include any one or more concentrations selected from 1,000 (1k), 5,000 (5k), 10,000 (10k), 50,000 (50k), 100,000 (0.1M), 500,000 (0.5M), 1,000,000 (1M) or 5,000,000 (5M); 500 (0.5k), 2,500 (2.5k), 5,000 (5k), 25,000 (25k), 50,000 (50k), 250,000 (250k), 500,000 (0.5M) or 2,500,000 (2.5M); or 200 (0.2k), 400 (0.4k), 600 (0.6k), 800 (0.8k), 1000 (1k), 2000 (2k), 4000 (4k), 6000 (6k), 8000 (8k), 10000 (10k), 20000 (20k) or 40000 (40k), in a particular volume, e.g., between at or about 10 and at or about 200 μL, such as at or about 10, 20, 30, 40, 50, 60, 70, 80, 90 or 100 μL. In some embodiments, the standard curve can be generated using 5,000 (5k), 10,000 (10k), 20,000 (20k) and 40,000 (40k). In some embodiments, the readouts or results determined is PET signal, e.g., in voxels (unit of graphic information in three-dimensional space) or pixels. In some embodiments, the readouts or results determined is PET signal, e.g., in total voxels. In some embodiments, the standard curve is generated from detection of the signal from a plurality of control samples containing a defined number of cells expressing the PSMA or modified form thereof, said plurality of control samples having been contacted with the PSMA-targeting molecule.

In some embodiments, the standard curve can be generated using the same condition and methods as used for detection and quantitation, e.g., using PET/CT for in vivo imaging after administration of a PET ligand specific for the particular cells, e.g., engineered cells expressing PSMA or a modified form thereof, such as ¹⁸F-DCFPyL. In some embodiments, the standard curve can be generated in vitro, but using the same method as used for detection and quantitation, e.g., using PET/CT after exposure to a PET ligand specific for the particular cells, e.g., engineered cells expressing PSMA or a modified form thereof, such as ¹⁸F-DCFPyL. In some embodiments, the standard curve can be generated by regression methods based on the data points from known numbers or concentrations, e.g., by linear regression and/or logistic regression. In some embodiments, the standard curve can be generated by linear regression. In some embodiments, quantitation of the number or concentration of the administered cells in a test subject or sample can be determined based on the determined standard curve.

B. In Vitro or Ex Vivo Detection

In some embodiments, the method of monitoring is performed in vitro or ex vivo and includes detecting cells expressing the PSMA or modified form thereof by contacting a composition containing cells that express or are likely to express the PSMA or modified form thereof with a PSMA-targeting molecule capable of recognizing or binding to the PSMA or modified form thereof. In some aspects, a biological sample is obtained from the subject and contacted with a PSMA-targeting molecule, such as a small molecule, a ligand, an antibody or antigen-binding fragment thereof, including any as described herein. Biological samples include, but are not limited to, body fluids, such as blood, plasma, serum, cerebrospinal fluid, synovial fluid, urine and sweat, tissue and organ samples, including processed samples derived therefrom.

In some embodiments, any of the methods as described above can be employed for detecting or identifying cells expressing the PSMA or modified form thereof obtained from a sample from a subject. In certain embodiments, recombinant cells expressing the conjugate may be detected or tracked in vitro or ex vivo by using PSMA-targeting molecule, such as a small molecule, a ligand, an antibody or antigen-binding fragment thereof, e.g., an anti-PSMA antibody.

In some embodiments, method of detection that is carried out in vitro or ex vivo include, but are not limited to immunohistochemistry (IHC), immunocytochemistry, flow cytometry, immunoblotting and ex vivo fluorescence imaging. Various known methods for detecting specific antibody-antigen binding can be used. Other methods of detection that can be used include exemplary immunoassay, such as fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA).

Other methods of detection that can be used include exemplary nucleic acid amplification-based methods, e.g., such as quantitative PCR (qPCR), is used to assess the quantity of cells expressing the recombinant receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, the primers or probe used for qPCR or other nucleic acid-based methods are specific for binding, recognizing and/or amplifying nucleic acids encoding the provided PSMA or modified form thereof, and/or the nucleic acid encoding the recombinant receptor and/or other components or elements of the plasmid and/or vector, including regulatory elements, e.g., promoters, transcriptional and/or post-transcriptional regulatory elements or response elements. In some embodiments, the primers or probe used to detect nucleic acids are specific for nucleic acids encoding the provided PSMA or modified form thereof.

In some embodiments, the method of detection is carried out by flow cytometry-based methods. In some embodiments, a flow cytometry-based method comprises the step of providing the population of cells contacted with a ligand conjugated to a detectable moiety (e.g. fluorescent moiety) for analysis using a flow cytometer. In flow cytometry, cells bound by fluorescently labeled reagents are carried in a fluidic stream, are separated based on size and/or intensity of fluorescent signal and are subsequently analyzed and counted using a FACS software program (e.g., FlowJo software). In some embodiments, cells within the population of cells are passed by a source (e.g. laser beam or light source, which provides light of a particular wavelength or wavelengths) to generate the detectable signal (e.g. fluorescence).

Other exemplary in vitro or ex vivo detection methods, e.g., by flow cytometry-based or quantitative PCR-based methods, to extrapolate to total cell numbers using known methods. See, e.g., Brentjens et al., Sci Transl Med. 2013 5(177), Park et al, Molecular Therapy 15(4):825-833 (2007), Savoldo et al., JCI 121(5):1822-1826 (2011), Davila et al., (2013) PLoS ONE 8(4):e61338, Davila et al., Oncoimmunology 1(9):1577-1583 (2012), Lamers, Blood 2011 117:72-82, Jensen et al., Biol Blood Marrow Transplant 2010 September; 16(9): 1245-1256, Brentjens et al., Blood 2011 118(18):4817-4828.

### VI. METHOD OF SELECTION

Provided are methods of selecting, isolating or separating cells expressing PSMA or a modified form thereof, such as any of the engineered cells provided herein. In some embodiments of the methods, particular cells, e.g., cells engineered to express PSMA or modified form thereof and a recombinant receptor, are selected, isolated and/or separated from a plurality or a mixture of cells. In some embodiments, the method can be used in connection with manufacturing, such as preparing and processing, genetically engineered cells. In some embodiments, the engineered cells expressing PSMA or modified form thereof and a PSMA-targeting molecule can be used to detect, select or isolate and/or identify cells transduced with the PSMA or modified form thereof.

In some embodiments, the method of selecting, isolating or separating cells expressing PSMA or a modified form thereof involves contacting a plurality of cells comprising any of the engineered cells provided herein with a PSMA-targeting molecule; and selecting, isolating or separating cells that are recognized or bound by the PSMA-targeting molecule. In some embodiments, the plurality of cells comprises engineered cells comprising any of the polynucleotides, set of polynucleotides, vectors and/or set of vectors encoding the PSMA or modified form thereof and/or recombinant receptor, as described herein.

In some embodiments, the methods involve selecting, isolating or separating cells that are recognized or bound by a PSMA-targeting molecule, from a plurality of cells comprising any of the engineered cells provided herein that have been contacted with the PSMA-targeting molecule.

In some embodiments, the PSMA-targeting molecule used in connection with the methods is capable of binding to PSMA or a modified form thereof, optionally to the active site of PSMA or the modified form, is cleaved by PSMA or a modified form thereof and/or is an antagonist or inhibitor of PSMA or a modified form thereof. For example, in some embodiments, the PSMA-targeting molecule used in connection with the methods of selecting, isolating or separating cells, can be any that are described in Section III above.

In some aspects, provided are methods of detecting, selecting or isolating gene modified cells before, during or after one or more steps of gene transfer, cell processing, incubation, culture, and/or formulation steps of the methods of engineering cells, such as during any steps for engineering the cells as described above. In some aspects, during production and further processing of gene modified cells (e.g. T cells), it is of interest to specifically select and further process only those cells that are positive for the transgene. In the provided methods, detection and selection of gene modified cells is carried out by contacting with the PSMA-targeting molecule, such as any described herein. In some aspects, detection of the PSMA or modified form thereof is a surrogate marker for the recombinant receptor co-introduced and/or co-expressed with the PSMA or modified form thereof.

In some aspects, the plurality or mixture of cells used for the selection, isolation, separation, identification and/or detection include samples resulting from one or more processing steps, such as separation, centrifugation, genetic engineering (e.g. transduction with viral vector), washing, and/or incubation. In some embodiments, cells or a composition of cells obtained before, during or after one or more steps of gene transfer (e.g. transduction with a viral vector), cell processing, incubation, culture, washing and/or formulation steps of the methods of engineering cells, such as any described herein, are contacted with the PSMA-targeting molecule. In certain embodiments, the contacting is under conditions permissive for binding of the PSMA-targeting molecule to the PSMA or modified form thereof present in cells of the composition. In certain embodiments, the methods further include detecting whether a complex is formed between the PSMA-targeting molecule and PSMA or modified form thereof, and/or detecting the presence or absence or level of such binding.

In some embodiments, the methods can be used in connection with selecting, isolating or separating cells, e.g., cells engineered to express PSMA or modified form thereof and a recombinant receptor, from a biological sample containing a plurality or a mixture of cells. Biological samples include, but are not limited to, body fluids, such as blood, plasma, serum, cerebrospinal fluid, synovial fluid, urine and sweat, tissue and organ samples, including processed samples derived therefrom. In some embodiments, the biological sample can be obtained after administration of the engineered cells to the subject. For example, in some embodiment, the plurality of cells comprising the engineered cells comprises a biological sample, e.g., peripheral blood leukocytes from a subject having been administered any of the engineered cells described herein.

In some embodiments, the PSMA-targeting molecules, e.g., small molecules, ligands and/or antibodies, for use in connection with the methods, are not bound to a solid support, i.e., it is present in soluble form or is soluble. In some cases, the PSMA-targeting molecule is an oligomer or polymer of individual molecules or an oligomer or polymer of a complex of subunits that make up the individual molecule. In some embodiments, the PSMA-targeting molecule can be covalently coupled to a synthetic carrier such as a polyethylene glycol (PEG) molecule. In some embodiments, the PSMA-targeting molecule can be linked to, attached to or reacted with a carrier, such as an organic carrier. In some aspects, in addition to a reaction with a polysaccharide, it is also possible to use physiologically or pharmaceutically acceptable proteins such as serum albumin (for example human serum albumin (HSA) or bovine serum albumin (BSA)) as carrier protein.

In some embodiments, the PSMA-targeting molecule used in connection with the methods provided herein is comprised on a support, such as a solid support or surface, e.g., bead, or a stationary phase (chromatography matrix). In some such embodiments, the PSMA-targeting molecule is reversibly immobilized on the support. In some cases, the PSMA-targeting molecule is immobilized to the support via covalent bonds. In some aspects, the PSMA-targeting molecule is reversibly immobilized to the support non-covalently.

In some embodiments, the support is a solid support. In some embodiments, any solid support (surface) can be used for the immobilization of the PSMA-targeting molecule. Illustrative examples of solid supports on which the PSMA-targeting molecule can be immobilized include a magnetic bead, a polymeric bead, a cell culture plate, a microtiter plate, a membrane, or a hollow fiber. In some aspects, hollow fibers can be used as a bioreactor in the Quantum® Cell Expansion System, available from TerumoBCT Inc. (Lakewood, Colo., USA). In some embodiments, the PSMA-targeting molecule is covalently attached to the solid support. In other embodiments, non-covalent interactions can also be used for immobilization, for example on plastic substrates. Other illustrative examples that are readily commercially available are immobilized metal affinity chromatography (IMAC) resins such as the TALON® resins (Westburg, Leusden, The Netherlands) that can be used for the immobilization of oligo-histidine tagged (his-tagged) proteins, such as for the binding of an oligohistidine tag such as an penta- or hexa-histidine tag. Other examples include calmodulin sepharose available from GE Life Sciences which can be used for binding a conjugate in which the agent (affinity tag) is a calmodulin binding peptide. Further examples include sepharose to which glutathion is coupled, which can be used for binding a conjugate in which the agent (affinity tag) is glutathion-S-transferase.

In some embodiments, a solid support employed in the present methods may include magnetically attractable matter such as one or more magnetically attractable particles or a ferrofluid. A respective magnetically attractable particle may comprise a PSMA-targeting molecule with a binding site that is capable of binding a target cell. In some cases, magnetically attractable particles may contain diamagnetic, ferromagnetic, paramagnetic or superparamagnetic material. In general, superparamagnetic material responds to a magnetic field with an induced magnetic field without a resulting permanent magnetization. Magnetic particles based on iron oxide are for example commercially available as Dynabeads® from Dynal Biotech, as magnetic MicroBeads from Miltenyi Biotec, as magnetic porous glass beads from CPG Inc., as well as from various other sources, such as Roche Applied Science, BIOCLON, BioSource International Inc., micromod, AMBION, Merck, Bangs Laboratories, Polysciences, or Novagen Inc., to name only a few. Magnetic nanoparticles based on superparamagnetic Co and FeCo, as well as ferromagnetic Co nanocrystals have been described, for example by Hutten, A. et al. (J. Biotech. (2004), 112, 47-63). In some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher © Humana Press Inc., Totowa, N.J.).

In some embodiments, the support contains a stationary phase. Thus, in some embodiments, the PSMA-targeting molecule is comprised on a stationary phase (also called chromatography matrix). In some such embodiments, the PSMA-targeting molecule is reversibly immobilized on the stationary phase. In some cases, the PSMA-targeting molecule is reversibly immobilized to the stationary phase via covalent bonds. In some aspects, the PSMA-targeting molecule is reversibly immobilized to the stationary phase non-covalently.

Any material may in some aspects be employed as a chromatography matrix. In general, a suitable chromatography material is essentially innocuous, i.e. not detrimental to cell viability, such as when used in a packed chromatography column under desired conditions. In some embodiments, the stationary phase remains in a predefined location, such as a predefined position, whereas the location of the sample is being altered. Thus, in some embodiments the stationary phase is the part of a chromatographic system through which the mobile phase flows (either by flow through or in a batch mode) and where distribution of the components contained in the liquid phase (either dissolved or dispersed) between the phases occurs.

In some embodiments, the chromatography matrix has the form of a solid or semisolid phase, whereas the sample that contains the target cell to be isolated/separated is a fluid phase. The chromatography matrix can be a particulate material (of any suitable size and shape) or a monolithic chromatography material, including a paper substrate or membrane, such as any known and used. In one embodiment, the chromatography matrix/stationary phase is a non-magnetic material or non-magnetizable material. In other embodiments, a chromatography matrix employed in the present methods is void of any magnetically attractable matter. In some embodiments, non-magnetic or non-magnetizable chromatography stationary phases that are suitable in the present methods include derivatized silica or a crosslinked gel, such as a crosslinked gel based on natural polymers or synthetic polymers. Exemplary chromatography matrix/stationary phase are known, and can be used in connection with the methods provided herein.

In some embodiments, the solid support, such as a bead or chromatography matrix, can be used in enrichment and selection methods as described herein by contacting said solid support (e.g. matrix) with a sample, e.g., biological sample and/or a plurality or mixture of cells containing cells to be enriched or selected, e.g., cells expressing PSMA or modified form thereof, as described. In some embodiments, the selected cells are eluted or released from the solid support (e.g. matrix) by disrupting the interaction of the PSMA-targeting molecule and the PSMA or modified form thereof. In some embodiments, binding of the PSMA-targeting molecule to the PSMA or modified form thereof is reversible.

In some embodiments, the methods are carried out to select, isolate, separate or enrich cells that express the PSMA or modified form thereof based on detection by the PSMA-targeting molecule. In some aspects, the selected, isolated, separated or enriched cells represent cells that have been genetically engineered, such as by transduction, with one or more nucleic acid molecule(s) and/or vector(s) encoding the PSMA or modified form thereof, and, optionally, a co-expressed recombinant receptor, such as a CAR. In some embodiments, the provided methods produce or result in a cell composition containing cells enriched for cells expressing the PSMA or modified form thereof, and hence also cells expressing a recombinant receptor.

In some embodiments, the yield of cells expressing the PSMA or modified form thereof in the enriched composition, i.e., the number of enriched cells in the population compared to the number of the same population of cells in the starting sample, is 10% to 100%, such as 20% to 80%, 20% to 60%, 20% to 40%, 40% to 80%, 40% to 60%, or 60% to 80%.

In some embodiments, the percentage of the cells expressing the PSMA or modified form thereof in the enriched or isolated composition, i.e., the percentage of cells positive for the selected PSMA or modified form thereof versus total cells in the population of enriched or isolated cells, is at least 70%, 75%, 80%, 85%, 86%, 87%, 88%, 89%, 90%, 91%, 92%, 93%, 94%, and is generally at least 95%, 96%, 97%, 98%, 99% or greater.

### VII. KITS AND ARTICLES OF MANUFACTURE

Also provided are kits and articles of manufacture containing the provided engineered cells expressing a PSMA or modified form thereof and a recombinant receptor, and one or more PSMA-targeting molecule, and/or compositions thereof. Also provided herein are articles of manufacture. In some embodiments, the articles of manufacture includes any of the engineered cells, compositions, polynucleotides, set of polynucleotides, composition containing set of polynucleotides, vectors, set of vectors, composition containing set of vectors, kits and/or PSMA-targeting molecules provided herein.

In some embodiments, provided are kits that include a composition comprising a PSMA-targeting molecule; and instructions for administering and/or detecting the PSMA-targeting molecule to a subject receiving or having been administered a therapeutically effective amount of any of the engineered cells described herein, for detecting the engineered cells and/or the PSMA-targeting molecule. In some embodiments, the instructions specify steps for administration and/or detection according to any of the methods provided herein.

In some embodiments, provided are kits that include a composition comprising a therapeutically effective amount of any of the engineered cells described herein and a PSMA-targeting molecule described herein; and instructions for administering, to a subject for treating a disease or condition, the engineered cells and a PSMA-targeting molecule, said PSMA-targeting molecule that is, comprises or is linked to a one or more moiety that provides a signal or induces a signal that is detectable; and instructions for detecting the engineered cells and/or the PSMA-targeting molecule. In some embodiments, the instructions specify steps for administration and/or detection according to any of the methods provided herein.

In some embodiments, instructions further specify determining the number or concentration of the administered engineered cells in the subject. In some embodiments, the instructions specify that determining comprises comparing the signal to a standard curve. In some embodiments, instructions further specify that the standard curve is generated from detection of the signal from a plurality of control samples containing a defined number of cells expressing the PSMA or modified form thereof, said plurality of control samples having been contacted with the PSMA-targeting molecule. In some embodiments, the instructions specify that detecting is carried out via positron emission tomography (PET), optionally coupled with computed tomography (CT), and the PSMA-targeting molecule is 2-(3-{1-carboxy-5-[(6-[¹⁸F]fluoro-pyridine-3-carbonyl)-amino]-pentyl}-ureido)-pentanedioic acid (¹⁸F-DCFPyL).

In some embodiments, provided are kits that include a composition comprising a therapeutically effective amount of any of the engineered cells described herein, and instructions for administering, to a subject for treating a disease or condition, the engineered cells in a combined therapy with a PSMA-targeting molecule, said PSMA-targeting molecule that is, comprises or is linked to a therapeutic agent for treating the disease or condition. In some embodiments, the PSMA-targeting molecule or the therapeutic agent is capable of modulating the tumor microenvironment (TME) or is cytotoxic to the tumor. In some embodiments, the instructions specify steps for administration according to any of the methods provided herein.

In some embodiments, provided are kits that include a composition comprising a PSMA-targeting molecule; and instructions for administering, to a subject for treating a disease or condition, the PSMA-targeting molecule in a combined therapy with a therapeutically effective amount of any of the engineered cells described herein, for treating the disease or condition. . In some embodiments, the instructions specify steps for administration according to any of the methods provided herein.

In some embodiments, provided are kits that include a composition comprising a therapeutically effective amount of any of the engineered cells described herein and a composition comprising a PSMA-targeting molecule. In some embodiments, the PSMA-targeting molecule is or comprises or further comprises a therapeutic agent. In some embodiments, the kit further contains instructions for administering, to a subject for treating a disease or condition, the engineered cell and the PSMA-targeting molecule in a combined therapy for treating the disease or condition. In some embodiments, the kit further contains instructions for administering the PSMA-targeting molecule to a subject receiving or having been administered the engineered cells for detecting the engineered cells. In some embodiments, the therapeutic agent is an immunomodulatory agent, a cytotoxic agent, an anti-cancer agent or a radiotherapeutic. . In some embodiments, the instructions specify steps for administration according to any of the methods provided herein.

The kits and articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition. The article of manufacture may include (a) a first container with a composition contained therein, wherein the composition includes engineered cells expressing a PSMA or modified form thereof and a recombinant receptor; and (b) a second container with a composition contained therein, wherein the composition includes the second agent, such as one or more PSMA-targeting molecule. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VIII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g. : Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New.Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

### IX. EXEMPLARY EMBODIMENTS

Among the provided embodiments are:
1. An engineered cell, comprising
a prostate-specific membrane antigen (PSMA) or a modified form thereof and
a chimeric receptor and/or a recombinant antigen receptor.
2. An engineered cell, comprising
a nucleic acid encoding a prostate-specific membrane antigen (PSMA) or a modified form thereof; and
a nucleic acid encoding a chimeric receptor and/or a recombinant antigen receptor.
3. The engineered cell of embodiment 1 or embodiment 2, wherein the PSMA or modified form thereof is expressed on the surface of the cell.
4. The engineered cell of any of embodiments 1-3, wherein the PSMA or modified form thereof comprises an extracellular portion and a transmembrane domain.
5. The engineered cell of any of embodiments 1-4, wherein the PSMA or modified form thereof, optionally the extracellular portion, is capable of being recognized by a PSMA-targeting molecule or a portion thereof.
6. The engineered cell of embodiment 5, wherein the PSMA-targeting molecule or a portion thereof:
is capable of binding to a PSMA and/or to the modified form thereof, and/or
is capable of binding to the active site of a PSMA and/or of the modified form of PSMA, and/or
is capable of being cleaved by a PSMA and/or by the modified form of PSMA; and/or
is an antagonist, a selective antagonist, an inverse agonist, a selective inverse agonist, an agonist, a selective agonist, an inhibitor, and/or a selective inhibitor of a PSMA and/or of the modified form thereof.
7. The engineered cell of any of embodiments 1-6, wherein the PSMA or modified form thereof comprises an N-acetylated-alpha-linked-acidic dipeptidase (NAALADase) domain and/or comprises one or more active site residues and/or residues involved in PSMA substrate binding and/or PSMA catalytic activity, which optionally are residues at positions 210, 257, 269, 272,377,387,387,424,424,425,433,436,453,517,518,519,552,553,534,535,536,552, 553, 628, 666, 689, 699 and/or 700, with reference to positions in the sequence of amino acids set forth in SEQ ID NO:23.
8. The engineered cell of any of embodiments 1-7, wherein the PSMA or modified form thereof is a human PSMA or a modified form thereof.
9. The engineered cell of any of embodiments 1-8, wherein the PSMA or modified form thereof is wild-type PSMA, optionally wild-type human PSMA.
10. The engineered cell of any of embodiments 1-9, wherein the PSMA or modified form thereof comprises the sequence of amino acids set forth in SEQ ID NO:23 or an extracellular and/or transmembrane domain thereof, or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:23 or an extracellular and/or transmembrane domain thereof.
11. The engineered cell of any of embodiments 1-8 and 10, wherein the PSMA or modified form thereof is a modified PSMA, the modified PSMA comprising one or more amino acid modifications compared to a wild-type or unmodified PSMA.
12. The engineered cell of embodiment 11, wherein the wild-type or unmodified PSMA is human PSMA and/or comprises the sequence of amino acids set forth in SEQ ID NO:23 or a sequence of amino acids that exhibits at least or at least about 85%, 86%, 87%, 88%, 89%, 90%, 91%, 92%, 93%, 94%, 95%, 96%, 97%, 98%, 99% or more sequence identity thereto.
13. The engineered cell of embodiment 11 or embodiment 12, wherein the one or more amino acid modification comprises one or more amino acid substitutions, deletions and/or insertions.
14. The engineered cell of any of embodiments 11-13, wherein the modified PSMA (i) exhibits reduced endogenous signaling; (ii) exhibits increased cell surface expression; and/or (iii) exhibits reduced cellular internalization compared to the wild-type or unmodified PSMA.
15. The engineered cell of any of embodiments 1-8 and 10-14, wherein the modified PSMA comprises at least one amino acid substitution corresponding to W2G or does not comprise W2 or does not comprise any residue at position 2, with reference to positions in the sequence of amino acids set forth in SEQ ID NO:23.
16. The engineered cell of embodiment 15, wherein the modified PSMA comprises the sequence of amino acids set forth in SEQ ID NO:24 or a fragment thereof, or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS:24 or a fragment thereof and comprises the at least one amino acid substitution.
17. The engineered cell of any of embodiments 1-8 and 10-16, wherein the modified PSMA comprises a deletion of one or more N-terminal amino acid residues within the intracellular portion, compared to the wild-type or unmodified PSMA.
18. The engineered cell of embodiment 17, wherein the modified PSMA comprises a deletion of up to 2, 3, 4, 5, 6, 7, 8, 9, 10, 11, 12, 13, 14, 15, 16, 17 or 18 N-terminal amino acid residues compared to the wild-type or unmodified PSMA.
19. The engineered cell of any of embodiments 1-8 and 10-18, wherein the modified PSMA comprises deletion of a contiguous amino acid sequence at the N-terminus starting from residue at position 2, 3, 4, or 5 and up to position 6, 7, 8, 9, 10, 11, 12, 13, 14, 15, 16, 17 18 or 19, compared to the wild-type or unmodified PSMA, with reference to positions in the sequence of amino acids set forth in SEQ ID NO:23.
20. The engineered cell of any of embodiments 1-8 and 10-19, wherein the PSMA or modified form thereof comprises the sequence of amino acids set forth in SEQ ID NO:25 or 52 or a fragment thereof, or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS:25 or 52 or a fragment thereof and that comprises deletion of the one or more N-terminal amino acid residues and, optionally, contains a methionine start codon.
21. The engineered cell of any of embodiments 1-20, wherein the PSMA or modified form thereof is encoded by the sequence of nucleic acids set forth in SEQ ID NO:26 or 53 or a fragment thereof; or a sequence of nucleic acids that exhibits at least or at least about 85%, 86%, 87%, 88%, 89%, 90%, 91%, 92%, 93%, 94%, 95%, 96%, 97%, 98%, 99% or more sequence identity to SEQ ID NO:26 or 53 or a fragment thereof and that, optionally, contains a nucleic acid encoding a methionine start codon.
22. The engineered cell of any of embodiments 1-21, wherein the PSMA or modified form thereof is encoded by a sequence of nucleic acids that is modified to be CpG-free and/or is codon optimized.
23. The engineered cell of embodiment 22, wherein the PSMA or modified form thereof is encoded by the sequence of nucleic acids set forth in SEQ ID NO:27 or a fragment thereof; or a sequence of nucleic acids that exhibits at least or at least about 85%, 86%, 87%, 88%, 89%, 90%, 91%, 92%, 93%, 94%, 95%, 96%, 97%, 98%, 99% or more sequence identity to SEQ ID NO:27 or a fragment thereof, and optionally that contains a nucleic acids that encodes a methionine start codon.
24. The engineered cell of any of embodiments 1-8 and 10-23, wherein:
the modified PSMA comprises all or substantially all of the transmembrane domain of the wild-type or unmodified PSMA; or
the modified PSMA comprises a transmembrane domain with the same or at least the same number of amino acids as the transmembrane domain of the wild-type or unmodified PSMA.
25. The engineered cell of any of embodiments 5-24, wherein the PSMA-targeting molecule is or comprises a small molecule, a ligand, an antibody or antigen-binding fragment thereof, an aptamer, a peptide, or a conjugate thereof.
26. The engineered cell of any of embodiments 5-25, wherein the PSMA-targeting molecule is or comprises a small molecule.
27. The engineered cell of embodiment 26, wherein the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE).
28. The engineered cell of embodiment 27, wherein the PSMA-targeting molecule is or comprises 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).
29. The engineered cell of any of embodiments 5-25, wherein the PSMA-targeting molecule is or comprises an antibody or antigen-binding fragment thereof, optionally having a binding site that specifically binds to PSMA.
30. The engineered cell of embodiment 29, wherein the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or comprises a CDR3, a V_{H} and/or V_{L}, and/or competes for binding to PSMA or binds to the same PSMA epitope as any of the foregoing.
31. The engineered cell of any of embodiments 5-25, wherein the PSMA-targeting molecule is or comprises an aptamer or a conjugate thereof.
32. The engineered cell of embodiment 31, wherein the aptamer comprises A9, A10, A10g, A10-3.2 or SZT101 or a conjugate thereof.
33. The engineered cell of any of embodiments 1-32, wherein the chimeric receptor and/or the recombinant antigen receptor is capable of binding to a target antigen that is associated with, specific to, and/or expressed on a cell or tissue of a disease, disorder or condition.
34. The engineered cell of embodiment 33, wherein the disease, disorder or condition is an infectious disease or disorder, an autoimmune disease, an inflammatory disease, or a tumor or a cancer.
35. The engineered cell of embodiment 33 or embodiment 34, wherein the target antigen is a tumor antigen.
36. The engineered cell of any of embodiments 33-35, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.
37. The engineered cell of any of embodiments 33-36, wherein the target antigen is selected from among ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, G Protein Coupled Receptor 5D (GPCR5D), HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-A1, MAGE A1, HLA-A2, NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag.
38. The engineered cell of any of embodiments 1-37, wherein chimeric receptor and/or the recombinant antigen receptor is or comprises a functional non-TCR antigen receptor or a TCR or antigen-binding fragment thereof.
39. The engineered cell of any of embodiments 1-38, wherein chimeric receptor and/or the recombinant antigen receptor is a chimeric antigen receptor (CAR).
40. The engineered cell of any of embodiments 1-39, wherein chimeric receptor and/or the recombinant antigen receptor comprises an extracellular domain comprising an antigen-binding domain.
41. The engineered cell of embodiment 40, wherein the antigen-binding domain is or comprises an antibody or an antibody fragment thereof, which optionally is a single chain fragment.
42. The engineered cell of embodiment 41, wherein the fragment comprises antibody variable regions joined by a flexible linker.
43. The engineered cell of embodiment 41 or embodiment 42, wherein the fragment comprises an scFv.
44. The engineered cell of any of embodiments 1-43, wherein chimeric receptor and/or the recombinant antigen receptor further comprises a spacer and/or a hinge region.
45. The engineered cell of any of embodiments 1-44, wherein chimeric receptor and/or the recombinant antigen receptor comprises an intracellular signaling region.
46. The engineered cell of embodiment 45, wherein the intracellular signaling region comprises an intracellular signaling domain.
47. The engineered cell of embodiment 46, wherein the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).
48. The engineered cell of embodiment 47, wherein the intracellular signaling domain is or comprises an intracellular signaling domain of a CD3 chain, optionally a CD3-zeta (CD3ζ) chain, or a signaling portion thereof.
49. The engineered cell of any of embodiments 45-48, wherein chimeric receptor and/or the recombinant antigen receptor further comprises a transmembrane domain disposed between the extracellular domain and the intracellular signaling region.
50. The engineered cell of any of embodiments 45-49, wherein the intracellular signaling region further comprises a costimulatory signaling region.
51. The engineered cell of embodiment 50, wherein the costimulatory signaling region comprises an intracellular signaling domain of a T cell costimulatory molecule or a signaling portion thereof.
52. The engineered cell of embodiment 50 or embodiment 51, wherein the costimulatory signaling region comprises an intracellular signaling domain of a CD28, a 4-1BB or an ICOS or a signaling portion thereof.
53. The engineered cell of any of embodiments 50-52, wherein the costimulatory signaling region is between the transmembrane domain and the intracellular signaling region.
54. The engineered cell of any of embodiments 2-53, wherein the nucleic acid encoding the PSMA or modified form thereof and the nucleic acid encoding chimeric receptor and/or the recombinant antigen receptor are comprised within one or more polynucleotide(s) comprised by the cell.
55. The engineered cell of embodiment 54, wherein the one or more polynucleotide(s) is one polynucleotide and the nucleic acid encoding the PSMA or modified form thereof and the nucleic acid encoding the chimeric receptor and/or the recombinant antigen receptor are operably linked to the same promoter and are optionally separated by an internal ribosome entry site (IRES), or a nucleic acid encoding a self-cleaving peptide or a peptide that causes ribosome skipping, which optionally is a T2A, a P2A, a E2A or a F2A.
56. The engineered cell of any of embodiments 39-55, wherein the nucleic acid encoding the PSMA or modified form thereof and the nucleic acid encoding the CAR are comprised within one polynucleotide comprised by the cell, said polynucleotide comprising, in 5′ to 3′ order:
i) a nucleic acid encoding a signal peptide;
ii) a nucleic acid encoding the CAR said CAR comprising an scFv; a spacer; a transmembrane domain; an intracellular region comprising a costimulatory signaling region, and an intracellular signaling domain of a CD3-zeta (CD3ζ) chain, or a signaling portion thereof;
iii) a nucleic acid sequence encoding a self-cleaving peptide or a peptide that causes ribosome skipping, which optionally is a T2A, a P2A, a E2A or a F2A; and
iv) a nucleic acid encoding the PSMA or modified form thereof, which optionally comprises the sequence of amino acids set forth in SEQ ID NO: 52 or a fragment thereof; or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS: 52 or a fragment thereof and that comprises deletion of the one or more N-terminal amino acid residues.
57. The engineered cell of embodiment 55 or 56, wherein the nucleic acid encoding the PSMA or modified form thereof and the nucleic acid encoding the chimeric receptor and/or the recombinant antigen receptor are operably linked to two different promoters.
58. The engineered cell of any of embodiments 55-57, wherein the nucleic acid encoding the chimeric receptor and/or the recombinant antigen receptor is present downstream of the nucleic acid encoding the PSMA or modified form thereof.
59. The engineered cell of any of embodiments 54-58, wherein the one or more polynucleotides comprises two different polynucleotides, the nucleic acid encoding the PSMA or modified form thereof and the nucleic acid encoding the chimeric receptor and/or the recombinant antigen receptor are operably linked to two different promoters, and/or the nucleic acid encoding the PSMA or modified form thereof and the nucleic acid encoding the chimeric receptor and/or the recombinant antigen receptor are present or inserted at different locations within the genome of the cell.
60. The engineered cell of any of embodiments 1-59, wherein:
the cell is an immune cell;
the cell is a T cell, optionally selected from the group consisting of CD4+ T cells and subtypes thereof and CD8+ T cells and subtypes thereof;
the cell is an NK cell; and/or
the cell is derived from a multipotent or pluripotent cell, which optionally is an iPSC.
61. The engineered cell of embodiment 60, wherein:
the cell is a T cell selected from the group consisting of central memory T cells, effector memory T cells, naïve T cells, stem central memory T cells, effector T cells and regulatory T cells; and/or
the cell comprises a plurality of cells, the plurality comprising at least 50% of a population of cells selected from the group consisting of CD4+ T cells, CD8+ T cells, central memory T cells, effector memory T cells, naïve T cells, stem central memory T cells, effector T cells and regulatory T cells.
62. The engineered cell of embodiment 61, wherein the cell is a regulatory T cell.
63. The engineered cell of any of embodiments 60-62, further comprising a recombinant FOXP3 or variant thereof.
64. A polynucleotide comprising a first nucleic acid encoding a prostate-specific membrane antigen (PSMA) or modified form thereof and a second nucleic acid encoding a chimeric receptor and/or a recombinant antigen receptor.
65. The polynucleotide of embodiment 64, wherein the nucleic acid encoding the PSMA or modified form thereof and the nucleic acid encoding the chimeric receptor and/or the recombinant antigen receptor are operably linked to the same promoter and are optionally separated by an internal ribosome entry site (IRES), or a nucleic acid encoding a self-cleaving peptide or a peptide that causes ribosome skipping, which optionally is a T2A, a P2A, a E2A or a F2A.
66. The polynucleotide of embodiment 64, wherein the nucleic acid encoding the PSMA or modified form thereof and the nucleic acid encoding the chimeric receptor and/or the recombinant antigen receptor are operably linked to two different promoters.
67. The polynucleotide of any of embodiments 64-66, wherein the nucleic acid encoding the chimeric receptor and/or the recombinant antigen receptor is present downstream of the nucleic acid encoding the PSMA or modified form thereof.
68. A set of polynucleotides comprising a first polynucleotide comprising a nucleic acid encoding a prostate-specific membrane antigen (PSMA) or modified form thereof and a second polynucleotide comprising a nucleic acid encoding a chimeric receptor and/or a recombinant antigen receptor.
69. A composition, comprising the set of polynucleotides of embodiment 68.
70. The polynucleotide, set of polynucleotides or composition of any of embodiments 64-69 wherein the nucleic acid encoding PSMA or a modified form thereof and the nucleic acid encoding the chimeric receptor and/or the recombinant antigen receptor each independently are operably linked to a promoter.
71. The polynucleotide, set of polynucleotides or composition of any of embodiments 64-70, wherein the encoded PSMA or modified form thereof is capable of being expressed on the surface of a cell.
72. The polynucleotide, set of polynucleotides or composition of any of embodiments 64-71, wherein the encoded PSMA or modified form thereof comprises an extracellular portion and a transmembrane domain.
73. The polynucleotide, set of polynucleotides or composition of any of embodiments 64-72, wherein the PSMA or modified form thereof, optionally the extracellular portion, is capable of being recognized by a PSMA-targeting molecule or a portion thereof.
74. The polynucleotide, set of polynucleotides or composition of embodiment 73, wherein the PSMA-targeting molecule or a portion thereof:
is capable of binding to a PSMA and/or to the modified form thereof, and/or
is capable of binding to the active site of a PSMA and/or of the modified form of PSMA, and/or
is capable of being cleaved by a PSMA and/or by the modified form of PSMA; and/or
is an antagonist, a selective antagonist, an inverse agonist, a selective inverse agonist, an agonist, a selective agonist, an inhibitor, and/or a selective inhibitor of a PSMA and/or of the modified form thereof.
75. The polynucleotide, set of polynucleotides or composition of any of embodiments 64-74, wherein the encoded PSMA or modified form thereof comprises an N-acetylated-alpha-linked-acidic dipeptidase (NAALADase) domain and/or comprises one or more active site residues and/or residues involved in PSMA substrate binding and/or PSMA catalytic activity, which optionally are residues at positions 210, 257, 269, 272, 377, 387, 387, 424, 424, 425, 433, 436, 453, 517, 518, 519, 552, 553, 534, 535, 536, 552, 553, 628, 666, 689, 699 and/or 700, with reference to positions in the sequence of amino acids set forth in SEQ ID NO:23.
76. The polynucleotide, set of polynucleotides or composition of any of embodiments 64-75, wherein the encoded PSMA or modified form thereof is a human PSMA or a modified form thereof.
77. The polynucleotide, set of polynucleotides or composition of any of embodiments 64-76, wherein the encoded PSMA or modified form thereof is wild-type PSMA, optionally wild-type human PSMA.
78. The polynucleotide, set of polynucleotides or composition of any of embodiments 64-77, wherein the encoded PSMA or modified from thereof comprises the sequence of amino acids set forth SEQ ID NO:23 or an extracellular and/or transmembrane domain thereof, or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:23 or an extracellular and/or transmembrane domain thereof.
79. The polynucleotide, set of polynucleotides or composition of any of embodiments 64-76 and 78, wherein the encoded PSMA or modified form thereof is a modified PSMA, the modified PSMA comprising one or more amino acid modifications compared to a wild-type or unmodified PSMA.
80. The polynucleotide, set of polynucleotides or composition of embodiment 79, wherein the wild-type or unmodified PSMA is human PSMA and/or comprises the sequence of amino acids set forth in SEQ ID NO:23 or a sequence of amino acids that exhibits at least or at least about 85%, 86%, 87%, 88%, 89%, 90%, 91%, 92%, 93%, 94%, 95%, 96%, 97%, 98%, 99% or more sequence identity thereto.
81. The polynucleotide, set of polynucleotides or composition of embodiment 79 or embodiment 80, wherein the one or more amino acid modification comprises one or more amino acid substitutions, deletions and/or insertions.
82. The polynucleotide, set of polynucleotides or composition of any of embodiments 79-81, wherein the encoded modified PSMA (i) exhibits reduced endogenous signaling; (ii) exhibits increased cell surface expression; and/or (iii) exhibits reduced cellular internalization compared to the wild-type or unmodified PSMA.
83. The polynucleotide, set of polynucleotides or composition of any of embodiments 64-76 and 78-82, wherein the encoded modified PSMA comprises at least one amino acid substitution corresponding to W2G or does not comprise W2 or does not comprise any residue at position 2, with reference to positions in the sequence of amino acids set forth in SEQ ID NO:23.
84. The polynucleotide, set of polynucleotides or composition of embodiment 83, wherein the encoded modified PSMA comprises the sequence of amino acids set forth in SEQ ID NO:24 or a fragment thereof or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS:24 or a fragment thereof and comprises the at least one amino acid substitution.
85. The polynucleotide, set of polynucleotides or composition of any of embodiments 64-76 and 78-84, wherein the encoded modified PSMA comprises a deletion of one or more N-terminal amino acid residues within the intracellular portion, compared to the wild-type or unmodified PSMA.
86. The polynucleotide, set of polynucleotides or composition of embodiment 85, wherein the encoded modified PSMA comprises a deletion of up to 2, 3, 4, 5, 6, 7, 8, 9, 10, 11, 12, 13, 14, 15, 16, 17 or 18 N-terminal amino acid residues compared to the wild-type or unmodified PSMA.
87. The polynucleotide, set of polynucleotides or composition of any of embodiments 64-76 and 78-86, wherein the encoded modified PSMA comprises deletion of a contiguous amino acid sequence at the N-terminus starting from residue at position 2, 3, 4, or 5 and up to position 6, 7, 8, 9, 10, 11, 12, 13, 14, 15, 16, 17, 18 or 19, with reference to positions in the sequence of amino acids set forth in SEQ ID NO:23.
88. The polynucleotide, set of polynucleotides or composition of any of embodiments 64-76 and 78-87, wherein the encoded PSMA or modified form thereof comprises the sequence of amino acids set forth in SEQ ID NO:25 or 52 or a fragment thereof; or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS:25 or 52 or a fragment thereof and that comprises deletion of the one or more N-terminal amino acid residues and, optionally, contains a methionine start codon.
89. The polynucleotide, set of polynucleotides or composition of any of embodiments 64-88, wherein the PSMA or modified form thereof is encoded by the sequence of nucleic acids set forth in SEQ ID NO: 26 or 53 or a fragment thereof or a sequence of nucleic acids that exhibits at least or at least about 85%, 86%, 87%, 88%, 89%, 90%, 91%, 92%, 93%, 94%, 95%, 96%, 97%, 98%, 99% or more sequence identity to SEQ ID NO:26 or 53 or a fragment thereof and that, optionally, contains a nucleic acid encoding a methionine start codon.
90. The polynucleotide, set of polynucleotides or composition of any of embodiments 64-89, wherein the PSMA or modified form thereof is encoded by a sequence of nucleic acids that is modified to be CpG-free and/or is codon optimized.
91. The engineered cell of embodiment 90, wherein the PSMA or modified form thereof is encoded by the sequence of nucleic acids set forth in SEQ ID NO: 27 or a fragment thereof or a sequence of nucleic acids that exhibits at least or at least about 85%, 86%, 87%, 88%, 89%, 90%, 91%, 92%, 93%, 94%, 95%, 96%, 97%, 98%, 99% or more sequence identity to SEQ ID NO:27 or a fragment thereof and that, optionally, contains a nucleic acid encoding a methionine start codon.
92. The polynucleotide, set of polynucleotides or composition of any of embodiments 64-76 and 78-91, wherein:
the encoded modified PSMA comprises all or substantially all of the transmembrane domain of the wild-type or unmodified PSMA; or
the encoded modified PSMA comprises a transmembrane domain with the same or at least the same number of amino acids as the transmembrane domain of the wild-type or unmodified PSMA.
93. The polynucleotide, set of polynucleotides or composition of any of embodiments 73-91, wherein the PSMA-targeting molecule is or comprises a small molecule, a ligand, an antibody or antigen-binding fragment thereof, an aptamer, a peptide, or a conjugate thereof.
94. The polynucleotide, set of polynucleotides or composition of any of embodiments 73-93, wherein the PSMA-targeting molecule is or comprises a small molecule.
95. The polynucleotide, set of polynucleotides or composition of embodiment 94, wherein the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE).
96. The polynucleotide, set of polynucleotides or composition of embodiment 95, wherein the PSMA-targeting molecule is or comprises 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).
97. The polynucleotide, set of polynucleotides or composition of any of embodiments 73-93, wherein the PSMA-targeting molecule is or comprises an antibody or antigen-binding fragment thereof, optionally having a binding site that specifically binds to PSMA.
98. The polynucleotide, set of polynucleotides or composition of embodiment 97, wherein the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or comprises a CDR3, a V_{H} and/or V_{L}, and/or competes for binding to PSMA or binds to the same PSMA epitope as any of the foregoing.
99. The polynucleotide, set of polynucleotides or composition of any of embodiments 73-93, wherein the PSMA-targeting molecule is or comprises an aptamer or a conjugate thereof.
100. The polynucleotide, set of polynucleotides or composition of embodiment 99, wherein the aptamer comprises A9, A10, A10g, A10-3.2 or SZT101 or a conjugate thereof
101. The polynucleotide, set of polynucleotides or composition of any of embodiments 64-100, wherein the encoded chimeric receptor and/or the encoded recombinant antigen receptor is capable of binding to a target antigen that is associated with, specific to, and/or expressed on a cell or tissue of a disease, disorder or condition.
102. The polynucleotide, set of polynucleotides or composition of embodiment 101, wherein the disease, disorder or condition is an infectious disease or disorder, an autoimmune disease, an inflammatory disease, or a tumor or a cancer.
103. The polynucleotide, set of polynucleotides or composition of embodiment 101 or embodiment 102, wherein the target antigen is a tumor antigen.
104. The polynucleotide, set of polynucleotides or composition of any of embodiments 101-103, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRHS), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.
105. The polynucleotide, set of polynucleotides or composition of any of embodiments 101-104, wherein the target antigen is selected from among ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, G Protein Coupled Receptor 5D (GPCR5D), HMW-MAA, IL-22R-alpha, IL-13R-alpha2, kinase insert domain receptor (kdr), kappa light chain, Lewis Y, Ll-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-A1, MAGE A1, HLA-A2, NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag.
106. The polynucleotide, set of polynucleotides or composition of any of embodiments 64-104, wherein the encoded chimeric receptor and/or the encoded recombinant antigen receptor is or comprises a functional non-TCR antigen receptor or a TCR or antigen-binding fragment thereof.
107. The polynucleotide, set of polynucleotides or composition of any of embodiments 64-106, wherein the encoded chimeric receptor and/or the encoded recombinant antigen receptor is a chimeric antigen receptor (CAR).
108. The polynucleotide, set of polynucleotides or composition of any of embodiments 64-107, wherein the encoded chimeric receptor and/or the encoded recombinant antigen receptor comprises an extracellular domain comprising an antigen-binding domain.
109. The polynucleotide, set of polynucleotides or composition of embodiment 108, wherein the antigen-binding domain is or comprises an antibody or an antibody fragment thereof, which optionally is a single chain fragment.
110. The polynucleotide, set of polynucleotides or composition of embodiment 109, wherein the fragment comprises antibody variable regions joined by a flexible linker.
111. The polynucleotide, set of polynucleotides or composition of embodiment 109 or embodiment 110, wherein the fragment comprises an scFv.
112. The polynucleotide, set of polynucleotides or composition of any of embodiments 64-111, wherein the encoded chimeric receptor and/or the encoded recombinant antigen receptor further comprises a spacer and/or a hinge region.
113. The polynucleotide, set of polynucleotides or composition of any of embodiments 64-112, wherein the encoded chimeric receptor and/or the encoded recombinant antigen receptor comprises an intracellular signaling region.
114. The polynucleotide, set of polynucleotides or composition of embodiment 113, wherein the intracellular signaling region comprises an intracellular signaling domain.
115. The polynucleotide, set of polynucleotides or composition cell of embodiment 114, wherein the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).
116. The polynucleotide, set of polynucleotides or composition of embodiment 115, wherein the intracellular signaling domain is or comprises an intracellular signaling domain of a CD3 chain, optionally a CD3-zeta (CD3) chain, or a signaling portion thereof.
117. The polynucleotide, set of polynucleotides or composition of any of embodiments 113-116, wherein the chimeric receptor and/or the recombinant antigen receptor further comprises a transmembrane domain disposed between the extracellular domain and the intracellular signaling region.
118. The polynucleotide, set of polynucleotides or composition of any of embodiments 113-117, wherein the intracellular signaling region further comprises a costimulatory signaling region.
119. The polynucleotide, set of polynucleotides or composition of embodiment 118, wherein the costimulatory signaling region comprises an intracellular signaling domain of a T cell costimulatory molecule or a signaling portion thereof.
120. The polynucleotide, set of polynucleotides or composition of embodiment 118 or embodiment 119, wherein the costimulatory signaling region comprises an intracellular signaling domain of a CD28, a 4-1BB or an ICOS or a signaling portion thereof
121. The polynucleotide, set of polynucleotides or composition of any of embodiments 118-120, wherein the costimulatory signaling region is between the transmembrane domain and the intracellular signaling region.
122. The polynucleotide of any of embodiments 107-121, wherein the polynucleotide comprises, in 5′ to 3′ order:
i) a nucleic acid encoding a signal peptide;
ii) a nucleic acid encoding the CAR said CAR comprising an scFv; a spacer; a transmembrane domain; an intracellular region comprising a costimulatory signaling region, and an intracellular signaling domain of a CD3-zeta (CD3ζ) chain, or a signaling portion thereof;
iii) a nucleic acid sequence encoding a self-cleaving peptide or a peptide that causes ribosome skipping, which optionally is a T2A, a P2A, a E2A or a F2A; and
iv) a nucleic acid encoding the PSMA or modified form thereof, which optionally comprises the sequence of amino acids set forth in SEQ ID NO: 52 or a fragment thereof, or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NOS: 52 or a fragment thereof and that comprises deletion of the one or more N-terminal amino acid residues.
123. A vector, comprising the polynucleotide of any of embodiments 66-67 and 70-122.
124. The vector of embodiment 123 that is a viral vector.
125. The vector of embodiment 123 or embodiment 124 that is a retroviral vector.
126. The vector of any of embodiments 123-125 that is a lentiviral vector or a gammaretroviral vector.
127. A set of vectors, comprising a first vector and a second vector, wherein the first vector comprises the first polynucleotide of any of embodiments 66-122 and the second vector comprises the second polynucleotide of any of embodiments 66-122.
128. A composition comprising the set of vectors of embodiment 127.
129. A method of producing an engineered cell, comprising introducing the polynucleotide of, or polynucleotides of the set of, or polynucleotide in the composition of any of embodiments 64-121 or the vector of, or the vector of the set of or the vector in the composition of any of embodiments 123-128 into a cell.
130. An engineered cell produced by the method of embodiment 129.
131. An engineered cell, comprising polynucleotide of, or polynucleotides of the set of, or polynucleotide in the composition of any of embodiments 64-122 or the vector of, or the vector of the set of or the vector in the composition of any of embodiments 123-128.
132. The engineered cell of embodiment 130 and 131, wherein:
the cell is an immune cell;
the cell is a T cell, optionally selected from the group consisting of CD4+ T cells and subtypes thereof and CD8+ T cells and subtypes thereof;
the cell is an NK cell; and/or
the cell is derived from a multipotent or pluripotent cell, which optionally is an iPSC.
133. A composition comprising the engineered cell of any of embodiments 1-63 and 130-132.
134. The composition of embodiment 133, further comprising a pharmaceutically acceptable excipient.
135. The composition of embodiment 133 or embodiment 134, further comprising a PSMA-targeting molecule.
136. A method of treatment comprising administering the engineered cells of any of embodiments 1-63 and 130-132 or the composition of any of embodiments 133-135 to a subject.
137. The method of embodiment 136, further comprising:
administering to the subject a PSMA-targeting molecule, or a composition comprising a PSMA-targeting molecule.
138. The method of embodiment 137, wherein the PSMA-targeting molecule is or comprises a therapeutic agent, or further comprises a therapeutic agent.
139. A method of treatment, the method comprising administering to a subject:
(a) the engineered cells of any of embodiments 1-63 and 130-132 or the composition of any of embodiments 133-135, and
(b) a PSMA-targeting molecule that is or comprises or further comprises a therapeutic agent, or a composition comprising a PSMA-targeting molecule that is or comprises or further comprises a therapeutic agent.
140. A method of treatment, comprising administering to a subject having been administered the engineered cells of any of embodiments 1-63 and 130-132 or the composition of any of embodiments 133-135, a PSMA-targeting molecule, said PSMA-targeting molecule that is or comprises or further comprises a therapeutic agent, or a composition comprising a PSMA-targeting molecule that is or comprises or further comprises a therapeutic agent.
141. The method of any of embodiments 137-140, wherein the PSMA-targeting molecule or the composition comprising the PSMA-targeting molecule is administered simultaneously with or sequentially with, in any order, administration of the engineered cells or the composition comprising the engineered cells.
142. The method of any of embodiments 137-141, wherein the PSMA-targeting molecule or the composition comprising the PSMA-targeting molecule is administered simultaneously with administration of the engineered cells or the composition comprising the engineered cells, optionally in the same or different compositions.
143. The method of any of embodiments 137-141, wherein the PSMA-targeting molecule or the composition comprising the PSMA-targeting molecule is administered sequentially with, in any order, administration of the engineered cells or the composition comprising the engineered cells.
144. The method of any of embodiments 137-143, wherein the PSMA-targeting molecule or a portion thereof:
is capable of binding to a PSMA and/or to the modified form thereof, and/or
is capable of binding to the active site of a PSMA and/or of the modified form of PSMA, and/or
is capable of being cleaved by a PSMA and/or by the modified form of PSMA; and/or
is an antagonist, a selective antagonist, an inverse agonist, a selective inverse agonist, an agonist, a selective agonist, an inhibitor, and/or a selective inhibitor of a PSMA and/or of the modified form thereof.
145. The method of any of embodiments 136-144, wherein the PSMA or modified form thereof is expressed on one or more of the engineered cells.
146. The method of any of embodiments 136-145, wherein the subject has a disease, disorder or condition, optionally a cancer, a tumor, an autoimmune disease, disorder or condition, or an infectious disease.
147. The method of any of embodiments 137-146, wherein the PSMA-targeting molecule is or comprises a small molecule, a ligand, an antibody or antigen-binding fragment thereof, an aptamer, a peptide, or a conjugate thereof.
148. The method of any of embodiments 137-147, wherein the PSMA-targeting molecule is or comprises a small molecule.
149. The method of embodiment 148, wherein the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE).
150. The method of embodiment 149, wherein the PSMA-targeting molecule is or comprises 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).
151. The method of any of embodiments 137-147, wherein the PSMA-targeting molecule is or comprises an antibody or antigen-binding fragment thereof, optionally having a binding site that specifically binds to PSMA.
152. The method of embodiment 151, wherein the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or comprises a CDR3, a V_{H} and/or V_{L}, and/or competes for binding to PSMA or binds to the same PSMA epitope as any of the foregoing.
153. The method of any of embodiments 137-152, wherein the PSMA-targeting molecule is or comprises an aptamer or a conjugate thereof, which optionally is or comprises A9, A10, A10g, A10-3.2 or SZT101 or a conjugate thereof.
154. The method of any of embodiments 138-153, wherein the therapeutic agent is capable of modulating the tumor microenvironment (TME) or is cytotoxic to the tumor.
155. The method of any of embodiments 138-154, wherein the therapeutic agent is an immunomodulatory agent, a cytotoxic agent, an anti-cancer agent or a radiotherapeutic.
156. The method of any of embodiments 138-155, wherein the PSMA-targeting molecule is or comprises a prodrug that is or comprises or is capable of conversion into or unmasking of the therapeutic agent and/or the PSMA-targeting molecule is capable of being cleaved upon binding to the PSMA or modified form thereof, wherein cleavage results in at least one cleavage product comprising the therapeutic agent.
157. The method of embodiment 156, wherein the PSMA-targeting molecule is or comprises Mipsagargin (G-202 (8-O-(12-aminododecanoyl)-8-O-debutanoyl thapsigargin)-Asp-γ-Glu-γ-Glu-γ-GluGluOH).
158. The method of any of embodiments 138-155, wherein the PSMA-targeting molecule is an antibody-drug conjugate (ADC).
159. The method of any of embodiments 138-158, wherein the PSMA-targeting molecule further comprises a therapeutic agent, and the therapeutic agent is linked directly or indirectly, optionally via a linker, to a portion of the PSMA-targeting molecule capable of binding to the PSMA or to the modified form thereof.
160. The method of embodiment 159, wherein the linker is a peptide or a polypeptide or is a chemical linker.
161. The method of embodiment 159 or embodiment 160, wherein the linker is a releasable linker or a cleavable linker.
162. The method of any of embodiments 159-161, wherein the linker is capable of being cleaved upon binding the PSMA or modified form thereof by the PSMA-targeting molecule, wherein cleavage results in at least one cleavage product comprising the therapeutic agent.
163. The method of embodiment 161 or embodiment 162, wherein the releasable linker or the cleavable linker is released or cleaved in the presence of one or more conditions or factors present in the tumor microenvironment (TME), wherein cleavage results in at least one cleavage product comprising the therapeutic agent.
164. The method of embodiment 163, wherein the one or more conditions or factors present in the tumor microenvironment (TME) comprises matrix metalloproteinase (MMP), hypoxic conditions or acidic conditions.
165. The method of any of embodiments 138-164, wherein the PSMA-targeting molecule induces killing or destruction of one or more of the engineered cells and/or of a cell or tissue present in the subject that is specifically recognized by the chimeric receptor and/or the recombinant antigen receptor.
166. The method of any of embodiments 138-165, wherein the therapeutic agent comprises a cytotoxic agent.
167. The method of embodiment 166, wherein the cytotoxic agent is or comprises a toxin.
168. The method of embodiment 167, wherein the toxin is a peptide toxin, ricin A chain toxin, Abrin A chain, Diphtheria Toxin (DT) A chain, Pseudomonas exotoxin, Shiga Toxin A chain, Gelonin, Momordin, Pokeweed Antiviral Protein, Saporin, Trichosanthin, proaerolysin or Barley Toxin.
169. The method of any of embodiments 138-168, wherein the therapeutic agent comprises a photosensitizer, which optionally is or comprises Pyropheophorbide-a (Ppa) or YC-9.
170. The method of any of embodiments138-169, wherein the administration of the PSMA-targeting molecule does not, or does not substantially, induce killing or destruction of healthy tissue or healthy cells, of cells or tissues not containing the engineered cells and/or not expressing the antigen.
171. The method of any of embodiments 138-157 and 159-165, wherein the therapeutic agent is an immunomodulatory agent.
172. The method of embodiment 171, wherein the immunomodulatory agent is an immune checkpoint inhibitor or modulator or a cytokine.
173. The method of embodiment 172, wherein the immunomodulatory agent is an immune checkpoint inhibitor capable of inhibiting or blocking a function of an immune checkpoint molecule or a signaling pathway involving an immune checkpoint molecule.
174. The method of embodiment 173, wherein the immune checkpoint molecule is selected from among PD-1, PD-L1, PD-L2, CTLA-4, LAG-3, TIM3, VISTA, an adenosine receptor or extracellular adenosine, optionally an adenosine 2A Receptor (A2AR) or adenosine 2B receptor (A2BR), or adenosine or a pathway involving any of the foregoing.
175. The method of any of embodiments 136-174, the method further comprising detecting cells that express the PSMA or modified form thereof and/or detecting the binding of the PSMA-targeting molecule to the PSMA or modified form thereof and/or the presence of the PSMA-targeting molecule.
176. The method of embodiment 175, wherein said detecting is performed in vivo and/or the detection is carried out via in vivo imaging.
177. A method of detecting engineered cells, comprising:
(a) contacting the engineered cells of any of embodiments 1-63 and 130-132 or the composition of embodiment 133 or embodiment 134 with a PSMA-targeting molecule; and
(b) detecting the binding of said PSMA-targeting molecule and/or the presence of said PSMA-targeting molecule to or with the PSMA or modified form thereof and/or the engineered cells.
178. The method of embodiment 177, wherein the contacting comprises administering, to a subject having been administered the engineered cells, the PSMA-targeting molecule.
179. A method of detecting the presence or absence of engineered cells in a subject, the method comprising:
(a) administering to a subject a PSMA-targeting molecule, said subject having been previously administered the engineered cells of any of embodiments 1-63 and 130-132 or composition of any of embodiments 133-135, wherein the engineered cells express a chimeric receptor and/or a recombinant antigen receptor and a PSMA or modified form thereof in a subject; and
(b) detecting the binding of the PSMA-targeting molecule to the PSMA or modified form thereof and/or to the engineered cells and/or the presence of the PSMA-targeting molecule in the subject.
180. The method of embodiment 179, wherein the detection is carried out via in vivo imaging.
181. The method of any of embodiments 177-180, wherein the PSMA-targeting molecule or a portion thereof:
is capable of binding to a PSMA and/or to the modified form thereof, and/or
is capable of binding to the active site of a PSMA and/or of the modified form of PSMA, and/or
is capable of being cleaved by a PSMA and/or by the modified form of PSMA; and/or
is an antagonist, a selective antagonist, an inverse agonist, a selective inverse agonist, an agonist, a selective agonist, an inhibitor, and/or a selective inhibitor of a PSMA and/or of the modified form thereof.
182. The method of any of embodiments 177-181, wherein the PSMA-targeting molecule is or comprises a small molecule, a ligand, an antibody or antigen-binding fragment thereof, an aptamer, a peptide, or a conjugate thereof.
183. The method of any of embodiments 177-182, wherein the PSMA-targeting molecule is or comprises a small molecule.
184. The method of embodiment 183, wherein the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE).
185. The method of embodiment 184, wherein the PSMA-targeting molecule is or comprises 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).
186. The method of any of embodiments 177-182, wherein the PSMA-targeting molecule is or comprises an antibody or antigen-binding fragment thereof.
187. The method of embodiment 186, wherein the antibody or antigen-binding fragment thereof is selected from among J591, DFO-J591, CYT-356, J415, 3/A12, 3/F11, 3/E7, D2B, 164-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or comprises a CDR3, a V_{H} and/or V_{L}, and/or competes for binding to PSMA or binds to the same PSMA epitope as any of the foregoing.
188. The method of any of embodiments 177-182, wherein the PSMA-targeting molecule is or comprises an aptamer or a conjugate thereof.
189. The method of embodiment 188, wherein the aptamer comprises A9, A10, A10g, A10-3.2 or SZT101 or a conjugate thereof.
190. The method of any of embodiments 156-189, wherein the detecting comprises identifying a signal in the subject or from a sample from the subject, wherein:
the PSMA-targeting molecule provides the signal or induces the signal that is detectable or is capable of binding to a moiety that provides the signal or induces the signal that is detectable; and/or
the PSMA-targeting molecule is or comprises a moiety that provides the signal or induces the signal that is detectable.
191. The method of embodiment 190, wherein the moiety comprises a radioisotope, a bioluminescent compound, a chemiluminescent compound, a fluorescent compound, a chromogenic compound, a quantum dot, a nanoparticle, a metal chelate or an enzyme.
192. The method of embodiment 190 or embodiment 191, wherein the PSMA-targeting molecule is or comprises an imaging probe or a detection reagent, which optionally is a radioligand.
193. The method of any of embodiments 190-192, wherein the PSMA-targeting molecule is or comprises the moiety and/or the PSMA-targeting molecule is capable of being cleaved upon binding the PSMA or modified form thereof, wherein cleavage results in at least one cleavage product comprising the moiety and/or is fluorescent and/or radioactive.
194. The method of any of embodiments 190-193, wherein the PSMA-targeting molecule further comprises a moiety that provides a signal or induces a signal that is detectable, and the moiety is linked directly or indirectly, optionally via a linker, to a portion of the PSMA-targeting molecule capable of binding to the PSMA or to the modified form thereof.
195. The method of embodiment 194, wherein the linker is a releasable linker or a cleavable linker.
196. The method of embodiment 194 or embodiment 195, wherein the linker is capable of being cleaved upon binding the PSMA or modified form thereof, wherein cleavage results in at least one cleavage product comprising the moiety and/or is fluorescent and/or radioactive.
197. The method of embodiment 195 or embodiment 196, wherein the releasable linker or the cleavable linker is released or cleaved in the presence of one or more conditions or factors present in the tumor microenvironment (TME), wherein cleavage results in at least one cleavage product comprising the moiety and/or is fluorescent and/or radioactive.
198. The method of embodiment 197, wherein the one or more conditions or factors present in the tumor microenvironment (TME) comprises matrix metalloproteinase (MMP), hypoxic conditions or acidic conditions.
199. The method of any of embodiments 156 and 191-198, wherein the radiotherapeutic, radioisotope, radioligand or radioactive cleavage product comprises ¹¹C, ¹⁸F, ⁶⁴Cu, ⁶⁸Ga, ⁶⁸Ge, ⁸⁶Y, ⁸⁹Zr, ⁹⁰Y, ^{99m}Tc, ¹¹¹In, ¹²³I, ¹²⁵I, ¹⁷⁷Lu and/or ²¹³Bi.
200. The method of embodiment 199, wherein the PSMA-targeting molecule is 2-(3-{1-carboxy-5-[(6-[¹⁸F]fluoro-pyridine-3-carbonyl)-amino]-pentyl}-ureido)-pentanedioic acid (¹⁸F-DCFPyL) or N-[N-[(S)-1,3-dicarboxypropyl]carbamoyl]-4-[¹⁸F]fluorobenzyl-L-cysteine (¹⁸F-DCFBC).
201. The method of any of embodiments 177-200, wherein the contacting and/or the detecting is performed in vivo and/or the detection is carried out via in vivo imaging.
202. The method of any of embodiments 176 and 180-201, wherein the in vivo imaging is selected from among magnetic resonance imaging (MRI), single-photon emission computed tomography (SPECT), computed tomography (CT), computed axial tomography (CAT), electron beam computed tomography (EBCT), high resolution computed tomography (HRCT), hypocycloidal tomography, positron emission tomography (PET), scintigraphy, gamma camera, a ¹³⁺detector, a y detector, fluorescence imaging, low-light imaging, X-rays, bioluminescence imaging and near-infrared (NIR) optical tomography.
203. The method of any of embodiments 176 and 180-202, wherein the in vivo imaging is positron emission tomography (PET), optionally coupled with computed tomography (CT).
204. The method of any of embodiments 175 and 177-203, wherein the method is capable of detecting as low or few as, or as low or few as approximately 4,000, 5,000, 6,000, 7,000, 8,000, 9,000 or 10,000 cells or as low or as few as approximately 4,000, 5,000, 6,000, 7,000, 8,000, 9,000 or 10,000 cells present in a specified volume.
205. The method of embodiment 204, wherein the specified volume is a volume of liquid, of a sample, and/or of an organ or tissue and/or is of between or between about 10 and about 100 μL.
206. The method of any of embodiments 177-200, wherein the contacting and/or the detecting is performed in vitro or ex vivo.
207. The method of any of embodiments 177-200 and 206, wherein the contacting and/or the detecting comprises immunohistochemistry (IHC), immunocytochemistry, or flow cytometry.
208. The method of any of embodiments 177-200, 206 and 207, wherein the method is capable of detecting as low or few as, or as low or few as approximately 2,000, 3,000, 4,000, 5,000, 6,000, 7,000, 8,000, 9,000 or 10,000 cells or as low or as few as approximately 2,000, 3,000, 4,000, 5,000, 6,000, 7,000, 8,000, 9,000 or 10,000 cells present in a specified volume.
209. The method of embodiment 208, wherein the specified volume is a volume of liquid, of a sample, and/or of an organ or tissue and/or is of between or between about 10 and about 100 μL.
210. The method of any of embodiments 190-209, the method further comprising determining the number or concentration of the administered engineered cells in the subject.
211. The method of embodiment 210, wherein determining comprises comparing the signal to a standard curve.
212. The method of embodiment 211, wherein the standard curve is generated from detection of the signal from a plurality of control samples containing a defined number of cells expressing the PSMA or modified form thereof, said plurality of control samples having been contacted with the PSMA-targeting molecule.
213. The method of any of embodiments 176 and 180-185, 192, 199-205 and 210-212, wherein the in vivo imaging is positron emission tomography (PET), optionally coupled with computed tomography (CT), and the PSMA-targeting molecule is 2-(3-{1-carboxy-5-[(6-[¹⁸F]fluoro-pyridine-3-carbonyl)-amino]-pentyl}-ureido)-pentanedioic acid (¹⁸F-DCFPyL).
214. A method of selecting, isolating or separating cells expressing PSMA or a modified form thereof, comprising:
(a) contacting a plurality of cells comprising the engineered cells of any of embodiments 1-63 and 130-132 with a PSMA-targeting molecule; and
(b) selecting, isolating or separating cells that are recognized or bound by the PSMA-targeting molecule.
215. A method of selecting, isolating or separating cells expressing PSMA or a modified form thereof, comprising selecting, isolating or separating cells that are recognized or bound by a PSMA-targeting molecule, from a plurality of cells comprising the engineered cells of any of embodiments 1-63 and 130-132 that have been contacted with the PSMA-targeting molecule.
216. The method of embodiment 214 or embodiment 215, wherein the PSMA-targeting molecule or a portion thereof:
is capable of binding to a PSMA and/or to the modified form thereof, and/or
is capable of binding to the active site of a PSMA and/or of the modified form of PSMA, and/or
is capable of being cleaved by a PSMA and/or by the modified form of PSMA; and/or
is an antagonist, a selective antagonist, an inverse agonist, a selective inverse agonist, an agonist, a selective agonist, an inhibitor, and/or a selective inhibitor of a PSMA and/or of the modified form thereof.
217. The method of any of embodiments 214-216, wherein the plurality of cells comprises engineered cells comprising the polynucleotide, set of polynucleotides or composition of any of embodiments 58-110 or the vector, set of vectors or composition of any of embodiments 111-116.
218. The method of any of embodiments 214-218, wherein the plurality of cells comprising the engineered cells comprise peripheral blood leukocytes from a subject having been administered the engineered cells of any of embodiments 1-63 and 130-132 or composition of any of embodiments embodiment 133-135.
219. The method of any of embodiments 214-218, wherein the PSMA-targeting molecule is or comprises a small molecule, a ligand, an antibody or antigen-binding fragment thereof, an aptamer, a peptide, or a conjugate thereof.
220. The method of any of embodiments 214-219, wherein the PSMA-targeting molecule is or comprises a small molecule.
221. The method of embodiment 220, wherein the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE).
222. The method of embodiment 221, wherein the PSMA-targeting molecule is or comprises 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).
223. The method of any of embodiments 214-219, wherein the PSMA-targeting molecule is or comprises an antibody or antigen-binding fragment thereof.
224. The method of embodiment 223, wherein the antibody or antigen-binding fragment thereof is selected from among J591, DFO-J591, CYT-356, J415, 3/A12, 3/F11, 3/E7, D2B, 196-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or comprises a CDR3, a V_{H} and/or V_{L}, and/or competes for binding to PSMA or binds to the same PSMA epitope as any of the foregoing.
225. The method of any of embodiments 214-219, wherein the PSMA-targeting molecule is or comprises an aptamer or a conjugate thereof.
226. The method of embodiment 225, wherein the aptamer comprises A9, A10, A10g, A10-3.2 or SZT101 or a conjugate thereof.
227. The method of any of embodiments 224-226, wherein the PSMA-targeting molecule is comprised in a matrix or immobilized on a solid support.
228. The method of embodiment 227, wherein the solid support comprises a magnetic particle.
229. A kit, comprising:
(a) a composition comprising a therapeutically effective amount of the engineered cells of any of embodiments 1-63 and 130-132; and
(b) a composition comprising a PSMA-targeting molecule.
230. The kit of embodiment 229, wherein the detecting comprises identifying a signal in the subject or from a sample from the subject, wherein:
the PSMA-targeting molecule provides the signal or induces the signal that is detectable or is capable of binding to a moiety that provides the signal or induces the signal that is detectable; and/or
the PSMA-targeting molecule is or comprises a moiety that provides the signal or induces the signal that is detectable.
231. The kit of embodiment 229 or embodiment 230, further comprising instructions for administering, to a subject for treating a disease or condition, the engineered cell and the PSMA-targeting molecule in a combined therapy for treating the disease or condition.
232. The kit of embodiment 229 or embodiment 230, further comprising instructions for administering the PSMA-targeting molecule to a subject receiving or having been administered the engineered cells for detecting the engineered cells.
233. A kit, comprising:
(a) a composition comprising a therapeutically effective amount of the engineered cells of any of embodiments 1-63 and 130-132; and
(b) instructions for administering a PSMA-targeting molecule to a subject receiving or having been administered the engineered cells for detecting the engineered cells.
234. A kit, comprising:
(a) a composition comprising a PSMA-targeting molecule; and
(b) instructions for administering the PSMA-targeting molecule to a subject receiving or having been administered a therapeutically effective amount of the engineered cells of any of embodiments 1-63 and 130-132 for detecting the engineered cells.
235. The kit of embodiment any of embodiments 232-234, wherein the instructions further specify determining the number or concentration of the administered engineered cells in the subject.
236. The kit of embodiment 235, wherein the instructions further specify that determining comprises comparing the signal to a standard curve.
237. The kit of any of embodiments 232-236, wherein the instructions specify that detecting is carried out via positron emission tomography (PET), optionally coupled with computed tomography (CT), and the PSMA-targeting molecule is 2-(3-{1-carboxy-5-[(6-[¹⁸F]fluoro-pyridine-3-carbonyl)-amino]-pentyl}-ureido)-pentanedioic acid (¹⁸F-DCFPyL).
238. A kit, comprising:
(a) a composition comprising a therapeutically effective amount of the engineered cells of any of embodiments 1-63 and 130-132; and
(b) instructions for administering, to a subject for treating a disease or condition, the engineered cells in a combined therapy with a PSMA-targeting molecule, said PSMA-targeting molecule that is or comprises or further comprises a therapeutic agent for treating the disease or condition.
239. The kit of embodiment 238, wherein the PSMA-targeting molecule or the therapeutic agent is capable of modulating the tumor microenvironment (TME) or is cytotoxic to the tumor.
240. The kit of any of embodiments 230, 238 and 239, wherein the therapeutic agent is an immunomodulatory agent, a cytotoxic agent, an anti-cancer agent or a radiotherapeutic.
241. A kit, comprising:
(a) a composition comprising a PSMA-targeting molecule; and
(b) instructions for administering, to a subject for treating a disease or condition, the PSMA-targeting molecule in a combined therapy with a therapeutically effective amount of the engineered cells of any of embodiments 1-63 and 130-132 for treating the disease or condition.
242. The kit of any of embodiments 229-241, wherein the PSMA-targeting molecule is or comprises a small molecule, a ligand, an antibody or antigen-binding fragment thereof, an aptamer, a peptide, or a conjugate thereof.
243. The kit of any of embodiments 229-242, wherein the PSMA-targeting molecule is or comprises a small molecule.
244. The kit of embodiment 243, wherein the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE).
245. The kit of embodiment 244, wherein the PSMA-targeting molecule is or comprises 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).
246. The kit of any of embodiments 229-242, wherein the PSMA-targeting molecule is or comprises an antibody or antigen-binding fragment thereof, optionally having a binding site that specifically binds to PSMA.
247. The kit of embodiment 246, wherein the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or comprises a CDR3, a V_{H} and/or V_{L}, and/or competes for binding to PSMA or binds to the same PSMA epitope as any of the foregoing.
248. The kit of any of embodiments 229-242, wherein the PSMA-targeting molecule is or comprises an aptamer or a conjugate thereof.
249. The kit of embodiment 248, wherein the aptamer comprises A9, A10, A10g, A10-3.2 or SZT101 or a conjugate thereof.
250. A PSMA-targeting molecule comprising a portion capable of binding to a PSMA or to a modified form thereof, wherein the PSMA-targeting molecule is or further comprises an immunomodulatory agent.
251. The PSMA-targeting molecule of embodiment 250, wherein the immunomodulatory agent is capable of modulating, optionally increasing, the activity of an immune cell or an immune response and/or is capable of modulating the tumor microenvironment (TME).
252. The PSMA-targeting molecule of embodiment 250 or embodiment 251, wherein the PSMA-targeting molecule is or comprises a prodrug that is or comprises or is capable of conversion into or unmasking of the immunomodulatory agent and/or the PSMA-targeting molecule is capable of being cleaved upon binding to the PSMA or modified form thereof, wherein cleavage results in at least one cleavage product comprising the immunomodulatory agent.
253. The PSMA-targeting molecule of any of embodiments 250-252, wherein the immunomodulatory agent is linked directly or indirectly, optionally via a linker, to a portion of the PSMA-targeting molecule capable of binding to the PSMA or to the modified form thereof.
254. The PSMA-targeting molecule of embodiment 253, wherein the linker is a peptide or a polypeptide or is a chemical linker.
255. The PSMA-targeting molecule of embodiment 253 or embodiment 254, wherein the linker is a releasable linker or a cleavable linker.
256. The PSMA-targeting molecule of any of embodiments 253-255, wherein the linker is capable of being cleaved upon binding to the PSMA or modified form thereof by the binding molecule, wherein cleavage results in at least one cleavage product comprising the immunomodulatory agent.
257. The PSMA-targeting molecule of embodiment 255 or embodiment 256, wherein the releasable linker or the cleavable linker is released or cleaved in the presence of one or more conditions or factors present in the tumor microenvironment (TME), wherein cleavage results in at least one cleavage product comprising the immunomodulatory agent.
258. The PSMA-targeting molecule of embodiment 257, wherein the one or more conditions or factors present in the tumor microenvironment (TME) comprises matrix metalloproteinase (MMP), hypoxic conditions or acidic conditions.
259. The PSMA-targeting molecule of any of embodiments 250-258, wherein the immunomodulatory agent is an immune checkpoint inhibitor or modulator or a cytokine.
260. The PSMA-targeting molecule of any of embodiments 250-259, wherein the immunomodulatory agent is an immune checkpoint inhibitor capable of inhibiting or blocking a function of an immune checkpoint molecule or a signaling pathway involving an immune checkpoint molecule.
261. The PSMA-targeting molecule of embodiment 260, wherein the immune checkpoint molecule is selected from among PD-1, PD-L1, PD-L2, CTLA-4, LAG-3, TIM3, VISTA, an adenosine receptor or extracellular adenosine, optionally an adenosine 2A Receptor (A2AR) or adenosine 2B receptor (A2BR), or adenosine or a pathway involving any of the foregoing.
262. A PSMA-targeting molecule comprising a portion capable of binding to a PSMA or to a modified form thereof, wherein the PSMA-targeting molecule is or further comprises a therapeutic agent capable of modulating the tumor microenvironment (TME), wherein the therapeutic agent is linked to the portion of the PSMA targeting molecule by a releasable or cleavable linker responsive to one or more conditions or factors present in the TME.
263. The PSMA-targeting molecule of embodiment 262, wherein the one or more conditions or factors present in the tumor microenvironment (TME) comprises matrix metalloproteinase (MMP), hypoxic conditions or acidic conditions.
264. The PSMA-targeting molecule of embodiment 262 or embodiment 263, wherein the therapeutic agent is an immunomodulatory agent, a cytotoxic agent, an anti-cancer agent or a radiotherapeutic.
265. The PSMA-targeting molecule of any of embodiments 250-264 that is an antibody-drug conjugate (ADC).
266. The PSMA-targeting molecule of any of embodiments 250-265, wherein the PSMA-targeting molecule or a portion thereof:
is capable of binding to a PSMA and/or to the modified form thereof, and/or
is capable of binding to the active site of a PSMA and/or of the modified form of PSMA, and/or
is capable of being cleaved by a PSMA and/or by the modified form of PSMA; and/or
is an antagonist, a selective antagonist, an inverse agonist, a selective inverse agonist, an agonist, a selective agonist, an inhibitor, and/or a selective inhibitor of a PSMA and/or of the modified form thereof.
267. The PSMA-targeting molecule of any of embodiments 250-264 and 266, wherein the PSMA-targeting molecule is or comprises a small molecule, a ligand, an antibody or antigen-binding fragment thereof, an aptamer, a peptide, or a conjugate thereof.
268. The PSMA-targeting molecule of any of embodiments 250-264, 266 and 267, wherein the PSMA-targeting molecule is or comprises a small molecule and the small molecule is selected from among 2-(3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE).
269. The PSMA-targeting molecule of embodiment 268, wherein the PSMA-targeting molecule is or comprises 2-(3-{1-carboxy-5-[(6-fluoro-pyridine-3-carbonyl)-amino]-pentyl}-ureido)-pentanedioicacid (DCFPyL) or N-[N-[(S)-1,3-dicarboxypropyl]carbamoyl]-4-fluorobenzyl-L-cysteine (DCFBC).
270. The PSMA-targeting molecule of any of embodiments 250-267, wherein the PSMA-targeting molecule is or comprises an antibody or antigen-binding fragment thereof.
271. The PSMA-targeting molecule of embodiment 270, wherein the antibody or antigen-binding fragment thereof is selected from among J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments and derivatives thereof, or comprises a CDR3, a V_{H} and/or V_{L}, and/or competes for binding to PSMA or binds to the same PSMA epitope as any of the foregoing.
272. The PSMA-targeting molecule of any of embodiments 250-264, 266 and 267, wherein the PSMA-targeting molecule is or comprises an aptamer or a conjugate thereof.
273. The PSMA-targeting molecule of embodiment 272, wherein the aptamer comprises A9, A10, A10g, A10-3.2 or SZT101 or a conjugate thereof.
274. A method of treatment comprising administering the PSMA-targeting molecule of any of embodiments 250-273 to a subject.
275. An article of manufacture, comprising the engineered cell of any of embodiments 1-63 and 130-132, the composition of any of embodiments 133-135, the polynucleotide, set of polynucleotides or composition of any of embodiments 64-122 or the vector, set of vectors or composition of any of embodiments 123-128, the kit of any of embodiments 229-249 or the PSMA-targeting molecule of any of embodiments 250-273.

### I. EXAMPLES

The following examples are included for illustrative purposes only and are not intended to limit the scope of the invention.

### Example 1: Engineering of Cells Expressing a Chimeric Antigen Receptor (CAR) and Prostate-Specific Membrane Antigen (PSMA) or Variants Thereof

Nucleic acid constructs were generated that encoded a chimeric antigen receptor (CAR) and a wild-type human prostate-specific membrane antigen (PSMA) or a modified variant thereof. The nucleic acid encoding the PSMA or modified variant was separated from that encoding the CAR by a sequence encoding a self-cleaving T2A sequence, for co-expression of the PSMA or modified variant and the CAR, under the control of a single promoter in engineered cells.

Specifically, the CAR encoded by each nucleic acid construct contained, in order: an anti-CD19 scFv (VH-linker-VL orientation), an Ig-derived spacer; a human CD28-derived transmembrane domain; a human 4-1BB-derived intracellular signaling domain; and a human CD3 zeta-derived signaling domain. Each nucleic acid molecule also included a nucleotide sequence encoding a self-cleaving P2A (set forth in SEQ ID NO:19, encoded by the nucleic acid sequence set forth in SEQ ID NO:53) or T2A sequence (set forth in SEQ ID NO:17) followed by a sequence encoding either full-length wild-type PSMA (WT PSMA, with the amino acid sequence set forth in SEQ ID NO:23), or a modified variant thereof. The modified variants included: (1) an N-terminally modified PSMA variant bearing an amino acid substitution at position 2, with reference to the sequence of SEQ ID NO:23, observed to inhibit receptor cycling (PSMA^{(W2G)}, having the amino acid sequence set forth in SEQ ID NO:24) or (2) an N-terminal deletion of the 9 most N-terminal amino acid residues of SEQ ID NO:23, observed to inhibit receptor cycling and signaling, which for clarity does not include deletion of the initial methionine required for translation (PSMA^{(N9del)} containing a deletion of 9 N-terminal amino acid residues; with the amino acid sequence set forth in SEQ ID NO:52, encoded by the nucleic acid sequence set forth in SEQ ID NO:53).

For comparison, a corresponding nucleic acid molecule was generated including the sequence encoding the anti-CD19 CAR and, in place of the PSMA (or modified PSMA)-encoding sequence, included a sequence encoding a truncated EGFR (EGFRt; set forth in SEQ ID NO:16) sequence.

Individual lentiviral vectors encoding each of the nucleic acid molecules were used to transduce primary human T cells isolated by immunoaffinity-based enrichment from human donor peripheral blood samples. As a control, cells were transduced with a vector not encoding the CAR and/or not encoding PSMA (mock).

After enrichment for CAR-expressing T cells, surface expression of WT PSMA or N-terminally modified PSMA variants (PSMA^{(W2G)} or PSMA^{(N9del)}) was assessed by flow cytometry using an anti-PSMA antibody recognizing an extracellular region of PSMA. Also assessed by flow cytometry was binding of a non-antibody PSMA-targeting reagent (a FITC-conjugated analog (YC-36-FITC) of a small molecule compound (DCFPyL) that specifically binds to PSMA).

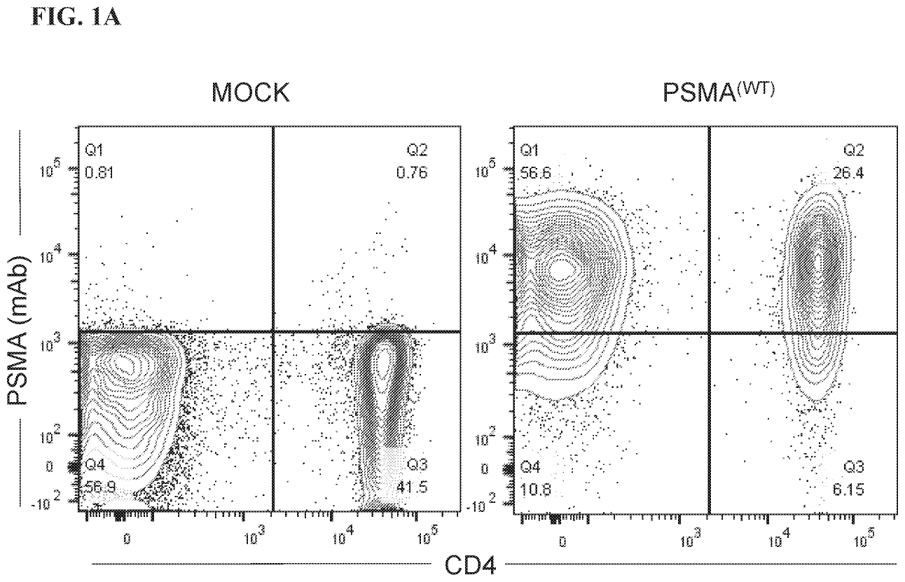

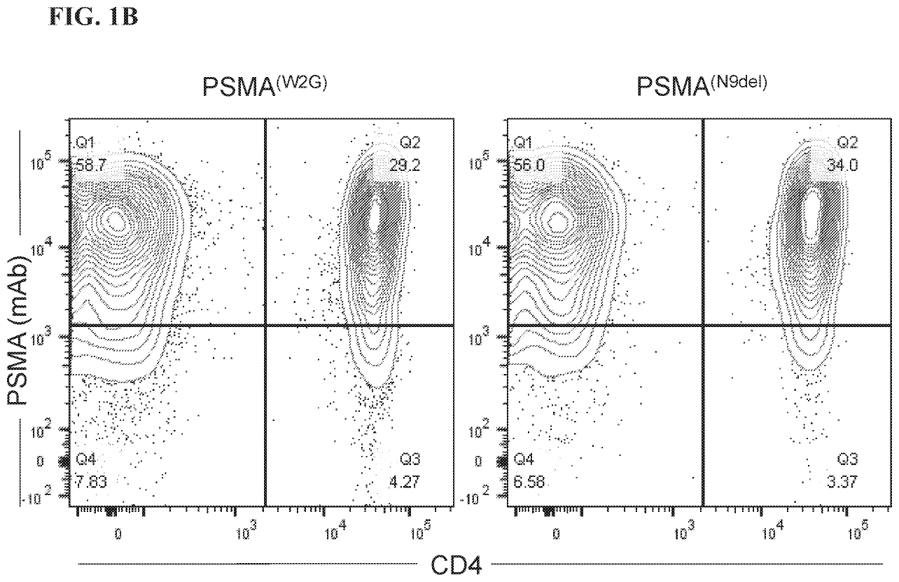

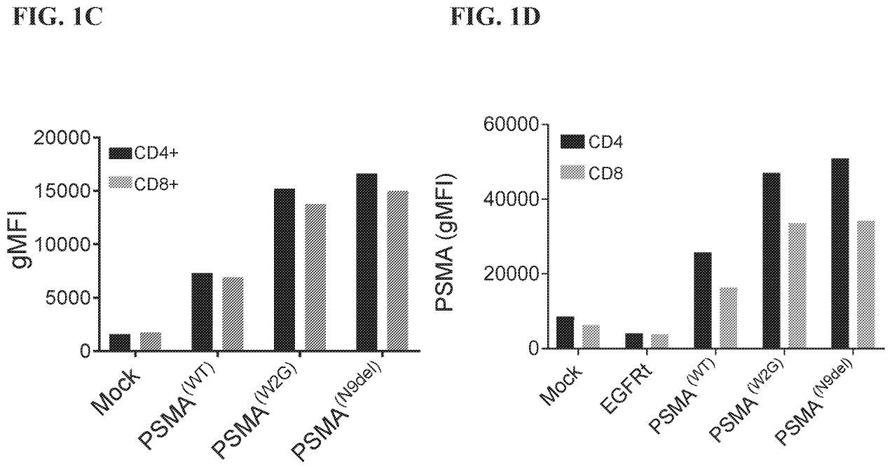

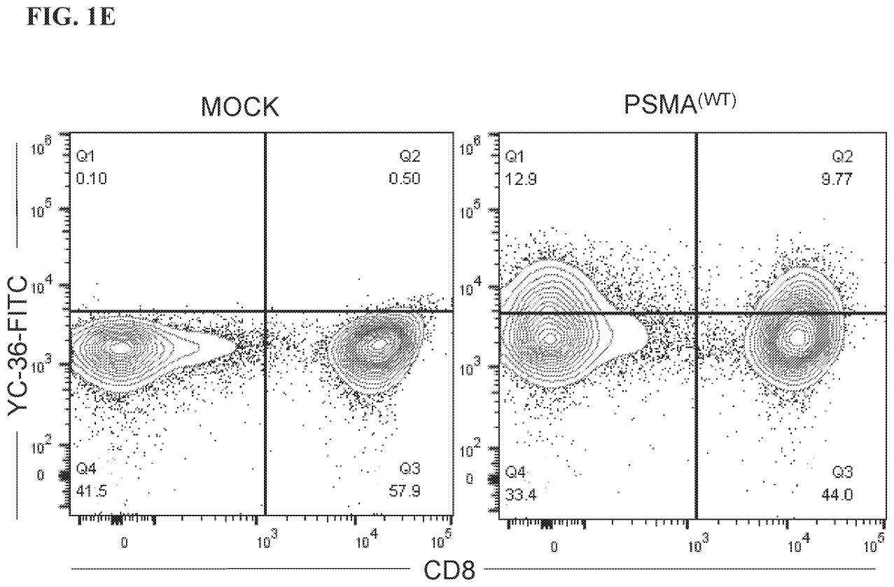

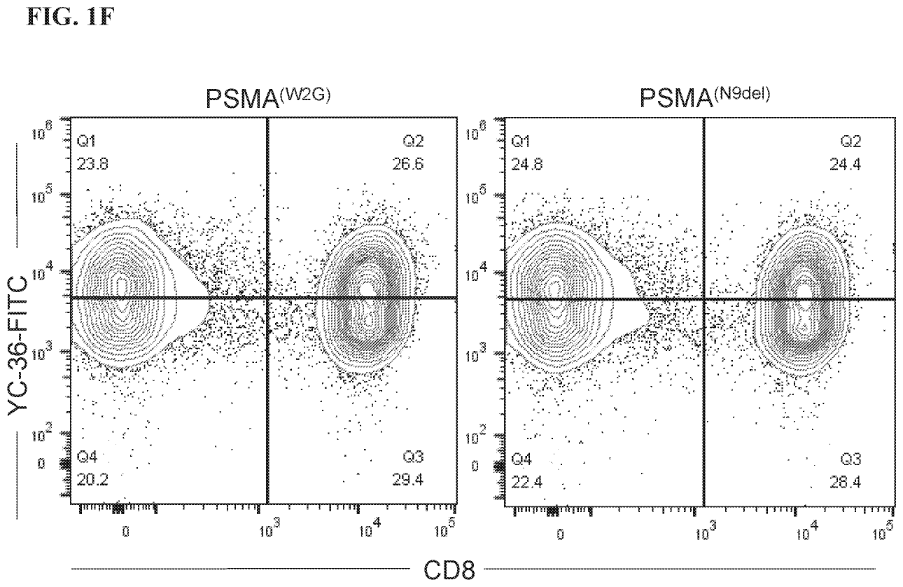

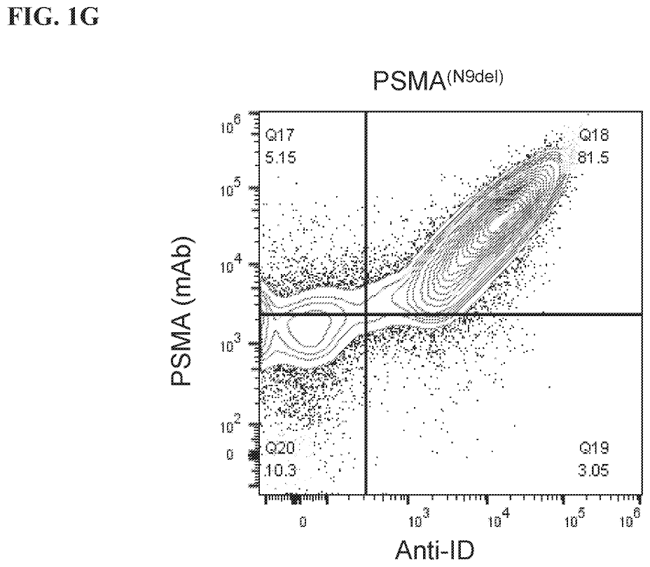

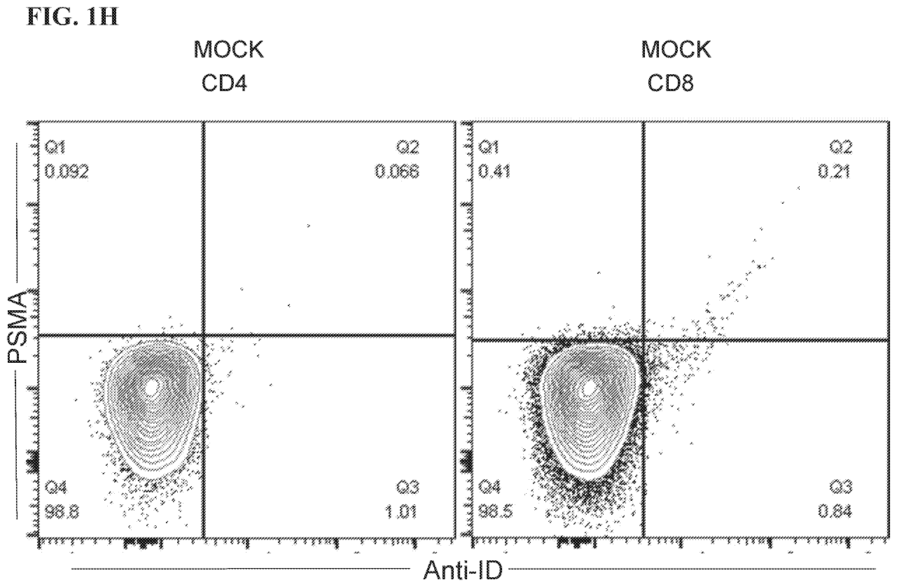

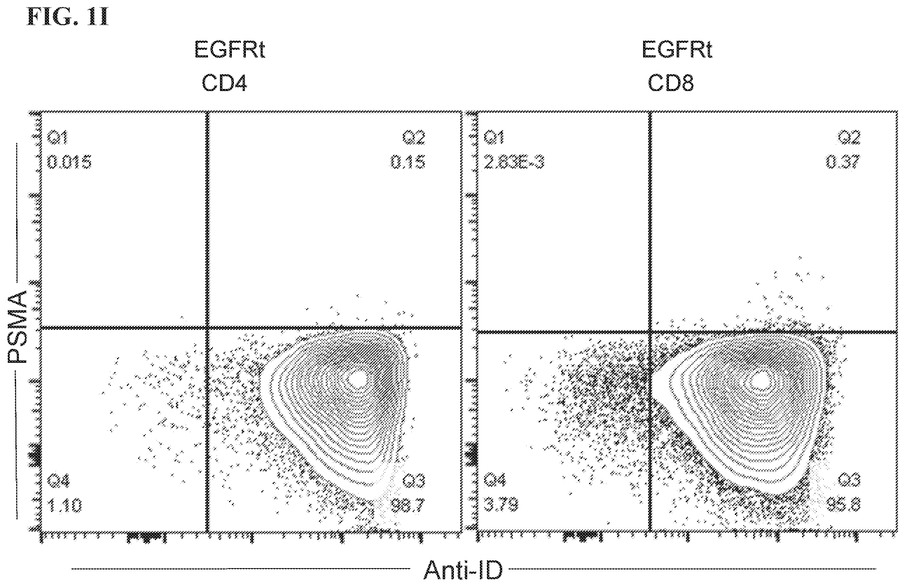

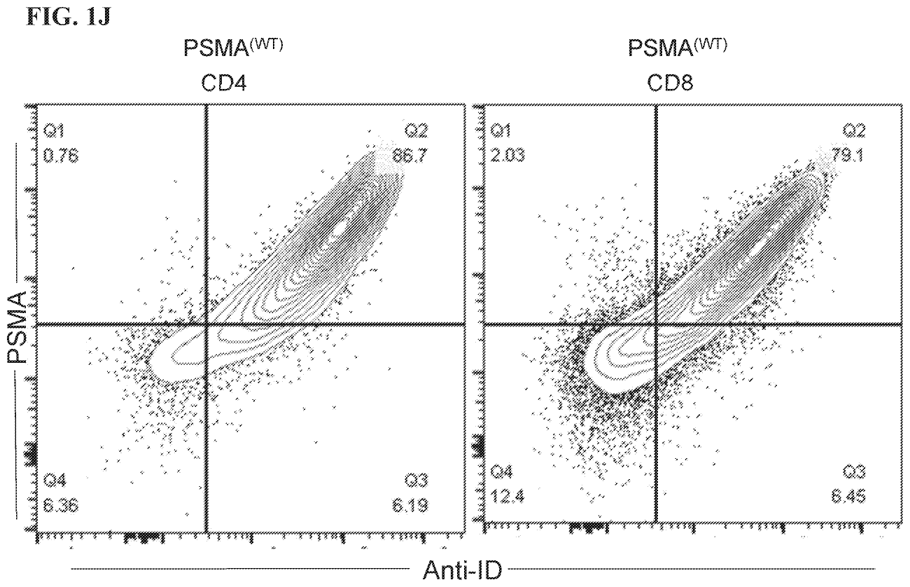

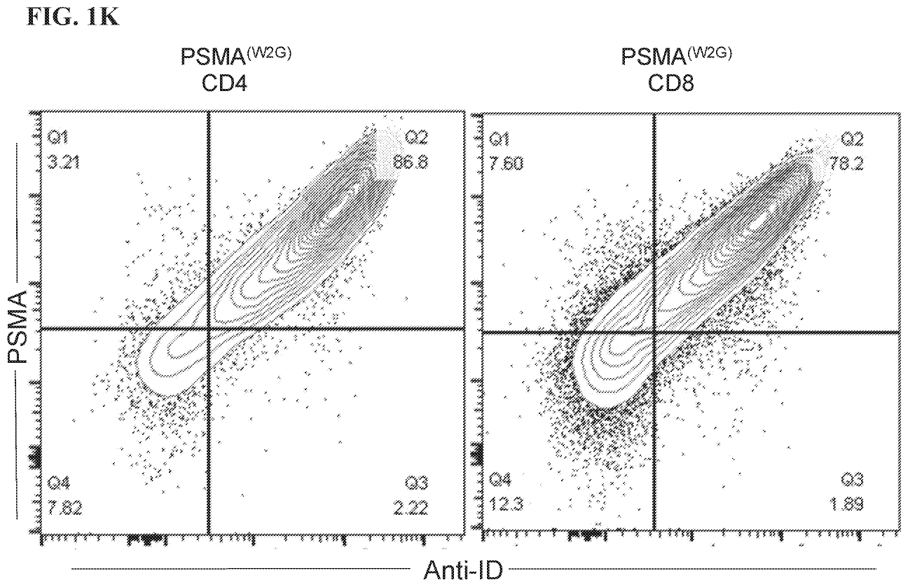

FIG. 1A-1B set forth exemplary FACS plots showing detected levels of binding of the anti-PSMA antibody and CD4 in CAR+ T cell-enriched samples from each of the transduced populations. FIG. 1C sets forth geometric mean fluorescent intensities (gMFI) for expression of PSMA (or N-terminally modified variants) as determined by antibody binding in this study for CD4+ and CD8+ T cells. As shown, PSMA and N-terminally modified variants were detected on the surface of transduced CD4+ and CD8+ cells. In this study, N-terminally modified PSMA^{(W2G)} and PSMA^{(N9del)} were expressed at higher levels. Similarly, as shown in FIG. 1D, the expression of the N-terminally modified PSMA^{(W2G)} and PSMA^{(N9del)} was greater than expression WT PSMA in CD4+ and CD8+ transduced cells, when gating for PSMA+ cells (FIG. 1D). No expression of PSMA was observed in non-transduced cells (mock) or cells transduced with the control recombinant protein truncated EGFR (EGFRt).

FIGS. 1E-1F shows levels of YC-36-FITC binding and CD8 expression observed by flow cytometry in the CAR-enriched cells that had been transduced with the PSMA or respective modified variants (PSMA^{(W2G)} and PSMA^{(N9del)}. FIG. 1G shows co-expression of the modified (N9del) PSMA on the surface of cells expressing the CAR, as determined by an anti-idiotype antibody specific for the binding domain of the CAR. Similar results were observed for the other PSMA constructs (FIGS. 1H-1L). FIG. 1M shows the gMFI for surface expression of the CAR as determined using an anti-idiotype antibody specific for the binding domain of the CAR by flow cytometry, in anti-CD19 CAR-expressing cells co-expressing the truncated PSMA variant (CD19-tPSMA; PSMA^{(N9del)}) or cells expressing a truncated receptor as a control surrogate marker (CD19-tReceptor), from an additional experiment with engineered cells generated from 2 different donors. The results confirmed the ability of the wild-type and modified PSMA variants to be expressed on the CAR-T cells in a manner that retained their ability to bind to a PSMA-targeting small molecule and a PSMA-specific antibody.

### Example 2: In Vitro Assessment of Engineered T cells Expressing Anti-CD19 CAR and PSMA or N-Terminally Modified PSMA Variants

Engineered human T cells expressing anti-CD19 CAR and/or PSMA or N-terminally modified variants thereof, produced as described in Example 1, were assessed in vitro.

A. Cytolytic Activity

CD19-expressing target cells (K562 cells transduced with CD19, designated K562-CD19) were labeled with NucLight Red (NLR) and incubated in triplicate with various engineered T cells generated as described in Example 1 at an effector to target (E:T) ratio of 4:1, 2:1, 1:1 and 1:2.

Cytolytic activity was assessed by measuring the loss of viable target cells over a period of three days, as determined by red fluorescent signal (using the IncuCyte® Live Cell Analysis System, Essen Bioscience). The number of NLR+ target cells was determined every 2 hours over time from 0 hours to 66 hours. Where indicated, a killing index was determined as 1/area under the curve (AUC) of the NLR+ target cell count over time, from 0 hours to 66 hours.

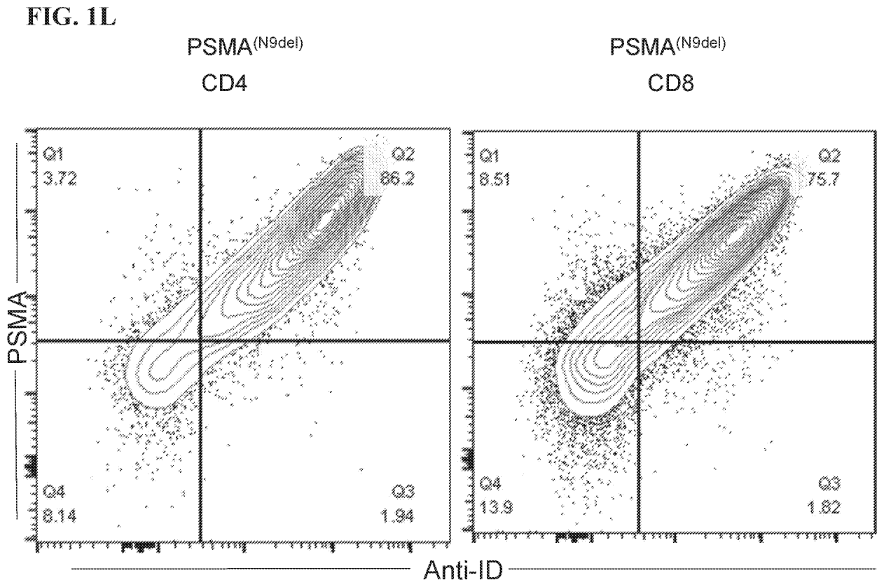

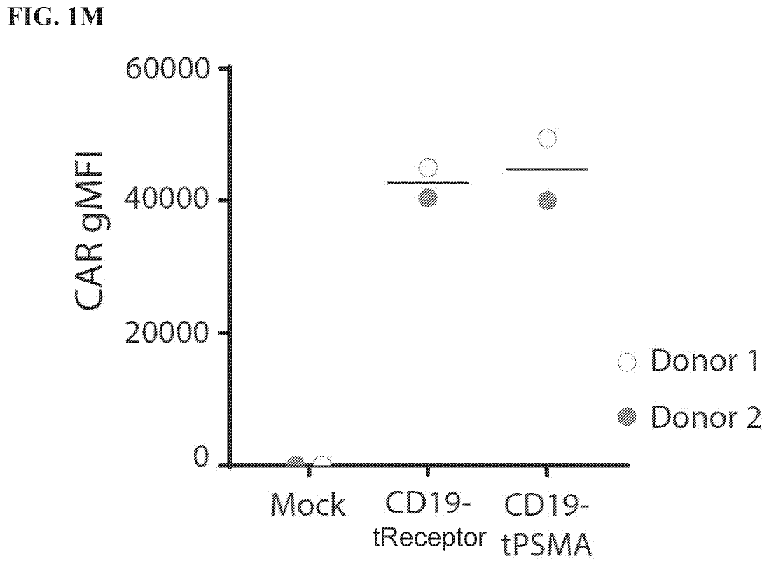

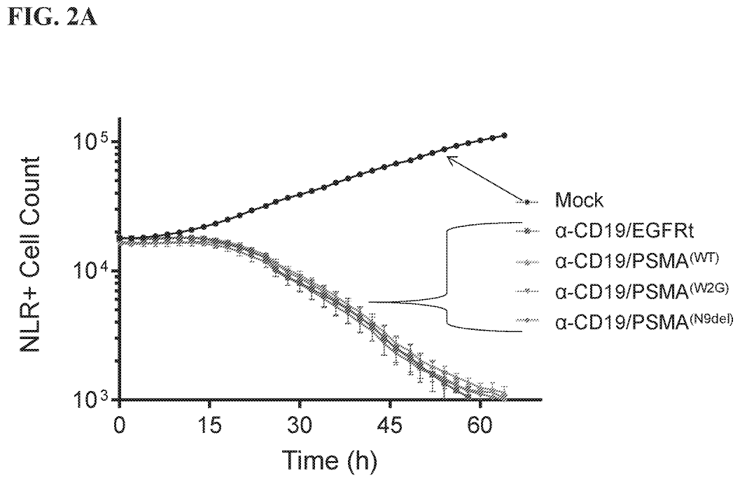

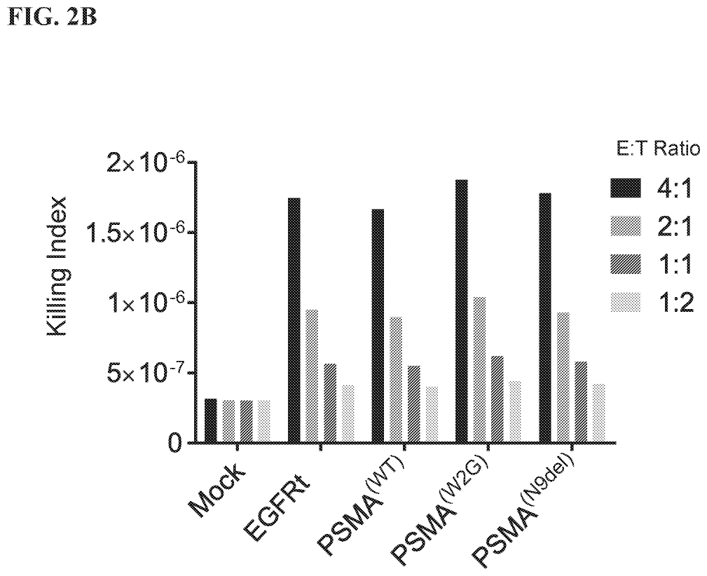

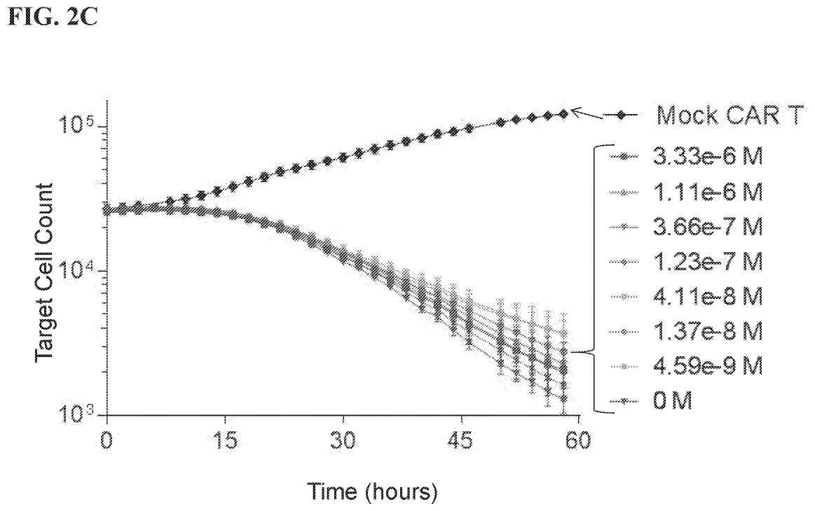

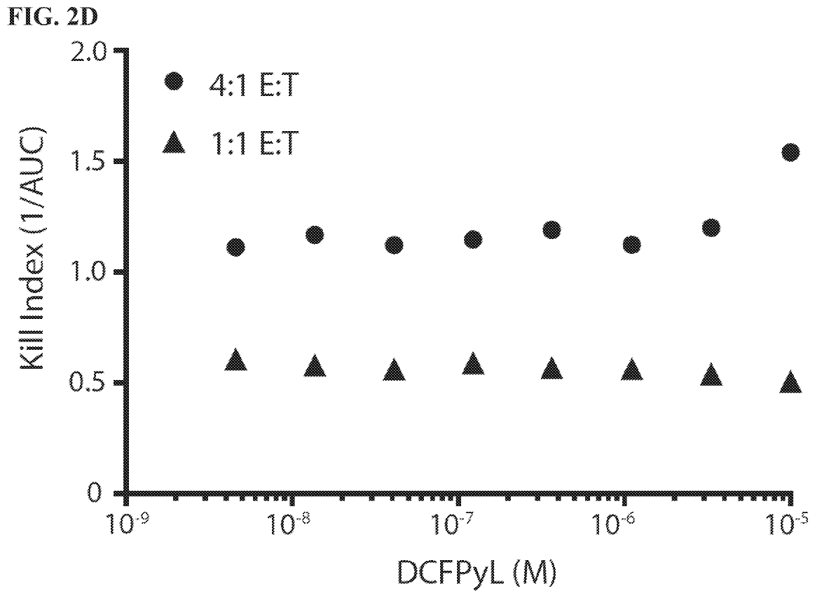

FIG. 2A shows results for the 4:1 effector to target cell ratio. FIG. 2B shows a comparison of results for killing index from different E:T ratios (4:1, 2:1, 1:1 and 1:2). The results showed that CAR-expressing cells that co-expressed PSMA (WT, PSMA^{(W2G)} or PSMA^{(N9del)}) were observed to exhibit antigen-specific killing activity of CAR-expressing cells not expressing PSMA (and expressing the alternative EGFRt marker).

A similar cytolytic assay as described above was performed by incubation of engineered T cells expressing anti-CD19 CAR/PSMA^{(N9del)} and K562-CD19 target cells at an E:T ratio of 4:1, except that the incubation was further in the presence of 2-(3-{1-carboxy-5-[(6-fluoro-pyridine-3-carbonyl)-amino]-pentyl}-ureido)-pentanedioicacid (DCFPyL), a reagent that binds to the catalytic domain of PSMA (see, e.g., Chen et al., Clin Cancer Res. 2011, 17(24):7645-7653; WO 2015/143029). Specifically, co-cultures were incubated with DCFPyL at one of seven (7) 3-fold serial dilutions of DCFPyL starting from 3.33×10⁻⁶ M, or were incubated without DCFPyL (0 M). As shown in FIG. 2C, incubation with DCFPyL did not affect the cytolytic activity of T cells expressing anti-CD19 CAR/PSMA^{(N9del)}.

Cytolytic activity was also assessed at an E:T ratio of 4:1 and 1:1, and a DFPCyL concentration ranging from 1×10⁻⁵M to 4.59×10⁻⁹ M. As shown in FIG. 2D, similar results were observed with no impact of DFPCyL on the cytolytic activity of T cells expressing anti-CD19 CAR/PSMA^{(N9del)}.

The result was consistent with a conclusion that engaging PSMA^{(N9del)} with a PSMA-targeting agent such as DCFPyL did not impact CAR antigen-specific cytotoxic function of CAR-expressing T cells.

B. Cytokine Release

Cytokine levels were assessed in supernatants following co-cultures of various engineered T cells generated as described in Example 1 with CD19-expressing target cells (K562-CD19). Specifically, accumulated amounts (pg/mL) of cytokine(s) (IL-2, IFN-gamma, TNF-alpha) were assessed following incubation of the target cells and the various populations of engineered cells, for a period of approximately 22-24 hours. Such assays provided a measure of antigen-specific cytokine secretion per CAR+ cell in the dose. In other assays, cytolytic activity against CD19-expressing target cells was assessed after incubation with the T cells.

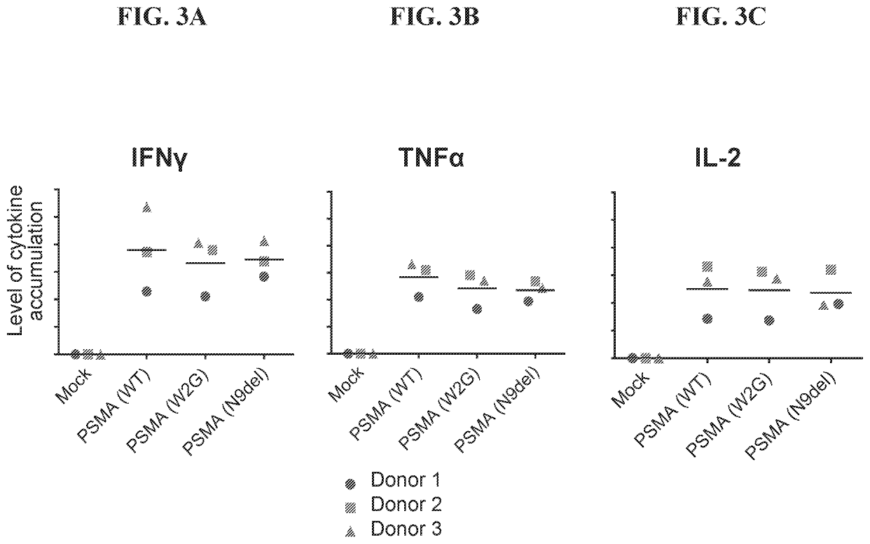

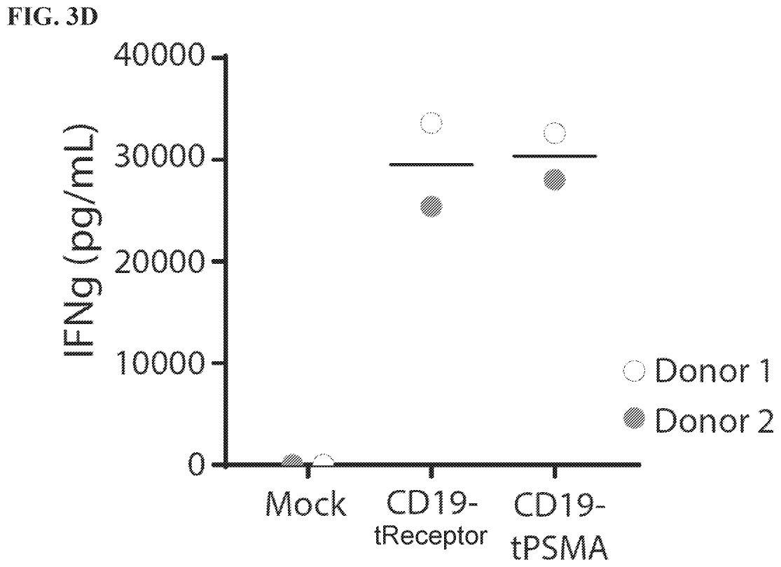

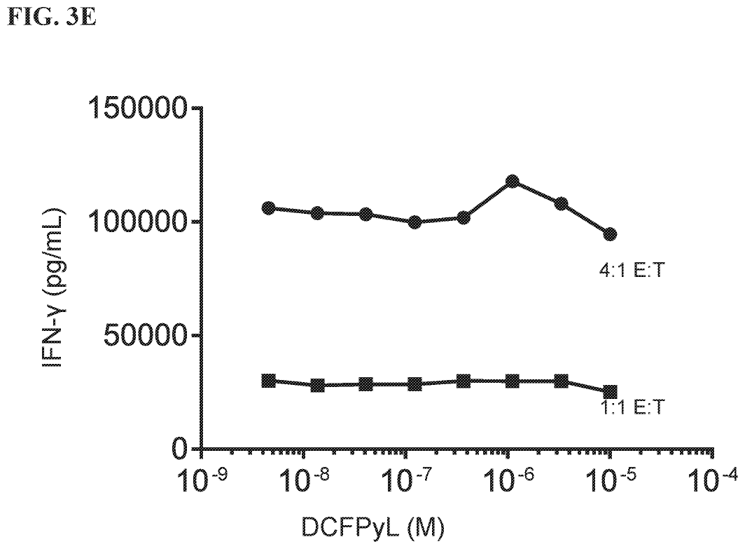

Cytokine amounts in culture supernatants were determined using a multiplex cytokine immunoassay (Luminex®). As shown in FIG. 3A (IFN-γ), FIG. 3B (TNF-α) and FIG. 3C (IL-2), T cells expressing anti-CD19 CAR/WT PSMA, anti-CD19 CAR/PSMA^{(W2G)}, or anti-CD19 CAR/PSMA^{(N9del)} but not mock-transduced cells, produced cytokine in response to incubation with CAR antigen-specific cells. FIG. 3D shows IFN-γ production in anti-CD19 CAR-expressing cells co-expressing the truncated PSMA variant (CD19-tPSMA; PSMA^{(N9del)}) or cells expressing a truncated receptor as a control surrogate marker (CD19-tReceptor), from an additional experiment with engineered cells generated from 2 different donors.

Cytokine levels were also assessed in co-cultures of engineered T cells expressing anti-CD19 CAR/PSMA^{(N9del)} and K562-CD19 target cells, at an E:T ratio of 4:1 and 1:1, cultured in the presence of DCFPyL. Co-cultures were incubated with DCFPyL at one of eight (8) 3-fold serial dilutions of DCFPyL starting from 1×10⁻⁵ M. As shown in FIG. 3E, incubation with a PSMA-targeting agent DCFPyL did not substantially affect IFN-γ production at either the 4:1 or 1:1 E:T ratio. Similar results were obtained for TNF-α and IL-2. The result was consistent with a conclusion that engaging PSMA^{(N9del)} with a PSMA-targeting agent such as DCFPyL did not substantially impact cytokine production by CAR-expressing T cells. The results showed that CAR+ T cells expressing a modified PSMA variant (e.g., truncated PSMA, tPSMA) exhibits in vitro CAR+ T cell function similar to that of CAR+ T cells expressing an alternative marker.

### Example 3: Assessment of in Vivo Anti-Tumor Activity of PSMA-Expressing CAR+ T Cells in a Tumor Model

A tumor xenograft mouse model was generated by injecting NOD/Scid/gc−/− (NSG) mice with cells of a CD19+ Nalm-6 disseminated tumor line. Specifically, on day zero (0), NSG mice were injected intravenously (i.v.) with 5×10⁵Nalm6 human B cell precursor leukemia cell line transfected with green fluorescence protein and firefly luciferase (Nalm6-GFP-ffluc). Tumor engraftment was allowed to occur for 4 days and verified using bioluminescence imaging. On day 4, mice were grouped into two study groups receiving a single intravenous (i.v.) injection of a sub-optimal dose of engineered cells (1×10⁶ CAR+ T cells) generated as described in Example 1 as follows: (1) T cells engineered with anti-CD19 CAR/PSMA^{(N9del)} or (2) T cells engineered with anti-CD19 CAR/EGFRt. Two additional study groups were added as controls, specifically, a study group in which mice were not injected with any cells (no treatment) and a study group in which mice were injected with 1×10⁶ T cells that did not express a CAR (mock study group).

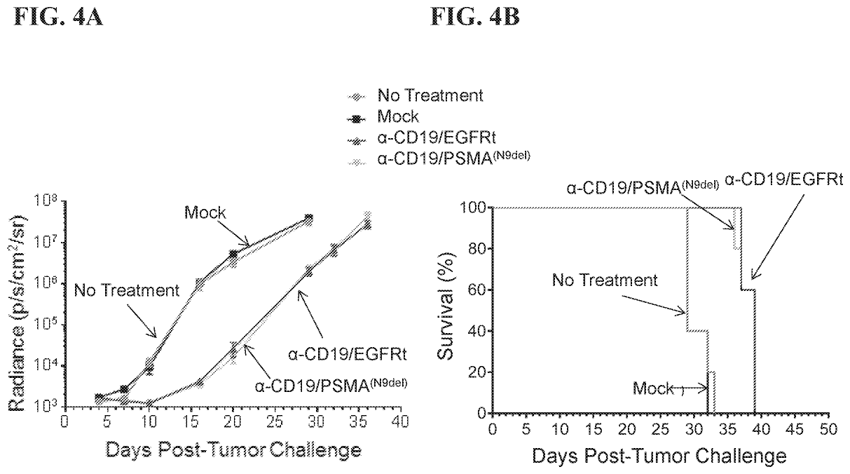

Following treatment, tumor growth over time was monitored by bioluminescence imaging and the average radiance (p/s/cm²/sr) was measured on days 4, 7, 10, 17, 21, 29, 32 and 36. For bioluminescence imaging, mice received intraperitoneal (i.p.) injections of luciferin substrate (CaliperLife Sciences, Hopkinton, Mass.) resuspended in PBS (15 μg/g body weight). As shown in FIG. 4A, tumors in negative control mice continued to grow over the course of the study following adoptive transfer of control T cells. Compared to the control mice, mice having been administered adoptive transfer of engineered T cells expressing anti-CD19 CAR/PSMA^{(N9del)} or anti-CD19 CAR/EGFRt were observed to have a reduction in the amount of average radiance at all post-treatment time points tested. The results indicate that the anti-CD19 CAR/PSMA^{(N9del)} T cells maintained similar anti-tumor activities compared to anti-CD19 CAR/EGFRt T cells.

The survival of the mice in each group was also monitored for up to 40 days after injection of the CD19+ Nalm-6 cells. The results are shown in FIG. 4B, survival of mice that received adoptive transfer of anti-CD19 CAR/PSMA^{(N9del)} T cells or anti-CD19 CAR/EGFRt cells, was observed to be similar, and in each case greater compared to the mice administered mock transduced cells or left untreated. The results showed that CAR+ T cells expressing a modified PSMA variant (e.g., truncated PSMA, tPSMA) exhibits in vivo CAR+ T cell function similar to that of CAR+ T cells expressing an alternative marker.

### Example 4: In Vivo PET/CT Imaging of Modified PSMA-Expressing CAR+ T Cells Using [¹⁸F]DCFPyL

Varying numbers of anti-CD19 CAR+ T cells expressing PSMA^{(N9del)} were injected into a mouse tumor model and imaged using a radiolabeled PSMA-specific (positron emission tomography) PET reagent for in vivo detection of modified PSMA-expressing CAR+ T cells in vivo using positron emission tomography-computed tomography (PET/CT).

Four- to six-week-old athymic male nude mice were injected subcutaneously with 1,000 (1k), 5,000 (5k), 10,000 (10k), 50,000 (50k), 100,000 (0.1M), 500,000 (0.5M), 1,000,000 (1M) or 5,000,000 (5M) T cells, of which half were engineered anti-CD19 CAR+ T cells expressing PSMA^{(N9del)}. The cells were injected in the upper flanks in a 50 μL suspension with Matrigel (BD Biosciences, Bedford, Mass.) at a 2:1 (cell:Matrigel) ratio. The number of anti-CD19 CAR/PSMA^{(N9del)}-expressing T cells in each 50 μLMatrigel suspension was 500 (0.5k), 2,500 (2.5k), 5,000 (5k), 25,000 (25k), 50,000 (50k), 250,000 (250k), 500,000 (0.5M) or 2,500,000 (2.5M). Each mouse received 400 μCi of PSMA-specific radiolabeled PET reagent 2-(3-{1-carboxy-5-[(6-[¹⁸F]fluoro-pyridine-3-carbonyl)-amino]-pentyl}-ureido)-pentanedioicacid ([¹⁸F]DCFPyL) (see, e.g., WO 2015/143029; Chen et al., Clin Cancer Res. 2011, 17(24):7645-7653, Szabo et al., Mol Imaging Biol. 2015 August; 17(4): 565-574), which binds to the active site of PSMA. PET scans were obtained 1 hour post injection of [¹⁸F]DCFPyL using a small animal PET scanner and corresponding CT scans were obtained using a small animal imaging device (see, e.g., exemplary methods described in WO 2015/143029). PET results were expressed in percentage of injected dose per cubic centimeter of tissue imaged (% ID/cc). PET and CT data were co-registered using AMIDE software (sourceforge.net). The images were reconstructed to minimize background signal in the kidney.

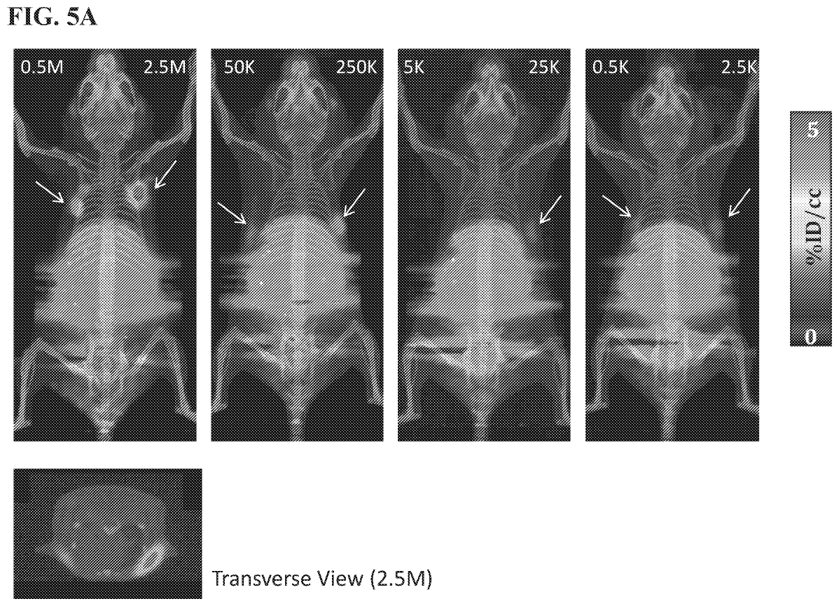

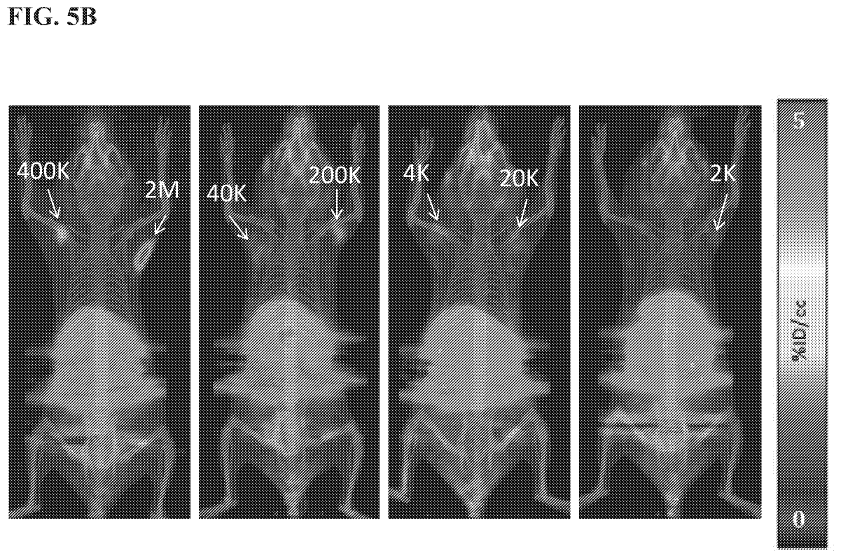

As shown in FIG. 5A, cells in each case were detectable by PET/CT, using the [¹⁸F]DCFPyL reagent, thereby demonstrating that CAR/PSMA^{(N9del)}-expressing T cells can bind the [¹⁸F]DCFPyL reagent in vivo. Detectable signal was observed by administration of as few as 1,000 anti-CD19 CAR/PSMA^{(N9del)} T cells. In additional experiments, following injection of about 1,000, 2,500, 5,000, 10,000 or more anti-CD19 CAR/PSMA^{(N9del)}-expressing T cells in 50 μL of Matrigel, the in vivo limit of detection was approximately 5,000 to 10,000 anti-CD19 CAR/PSMA^{(N9del)} T cells in 50 μL volume. An additional similar in vivo phantom imaging studies was carried out where 2,000 (2k), 4,000 (4k), 20,000 (20k),40,000 (40k), 200,000 (200k), 400,000 (400k) or 2,000,000 (2M) anti-CD19 CAR+ T cells expressing PSMA-N9del in 50 μL (50% Matrigel®) were injected into the shoulders of NOD/Scid/gc−/− (NSG) mice. The mice were injected with 14.8 MBq (400 μCi) of [¹⁸F]DCFPyL and imaged with a small animal PET/CT device 1 hour after injection. In this study, as few as 4,000 anti-CD19 CAR/PSMA^{(N9del)}-expressing T cells in a 50 μL volume were reliably detected using [¹⁸]DCFPyL and PET, as shown in FIG. 5B (white arrows indicating the location of the injected cells). In some embodiments, a limit of detection is (or the assay is capable of detecting) as low or few as, or as low or few as approximately, 4,000 anti-CD19 CAR/PSMA^{(N9del)} T cells in 50 μL volume, such as in a 50 μL volume of liquid, of a sample, and/or of an organ or tissue. The results are consistent with the utility of the modified PSMA variant (e.g., truncated PSMA, tPSMA) as a marker to detect CAR+ T cells in vivo with high sensitivity, by PET/CT.

### Example 5: In Vivo Detection of Administered PSMA-Expressing CAR+ T Cells in a Tumor Model Using a PSMA-Targeting Radiolabeled Agent ([¹⁸F]DCFPyL) by PET/CT

To assess detection of engineered cells in primary and metastatic tumor sites, a disseminated tumor model was developed and administered T cells expressing a CAR and PSMA variant. NSG mice were implanted subcutaneously (s.c.) with 5×10⁶ Nalm6-GFP-ffluc tumor cells (n=8/group). The Nalm6 subcutaneous implantation mouse model in some aspects can develop spontaneous metastatic lesions in the spleen, liver and bone marrow. On day 15 after tumor implantation, mice received a single intravenous (i.v.) injection of 2.5×10⁶ engineered primary human anti-CD19 CAR/PSMA^{(N9del)}-expressing T cells or anti-CD19 CAR/EGFRt T cells. As controls, mice were used that were not injected with any cells (no T cell) or that were injected with 2.5×10⁶ T cells that did not express a CAR (mock study group).

On day 24 after tumor implantation, the mice were subjected to bioluminescence imaging and PET/CT using the PSMA-targeting radiolabeled agent. Bioluminescence imaging to detect tumor cells was performed generally as described in Example 3 above. PET/CT was performed generally as described in Example 4 above. Specifically, each mouse received 400 μCi of the PSMA-specific radiolabeled PET reagent ([¹⁸F]DCFPyL). PET scans were obtained 1 hour post injection of [¹⁸F]DCFPyL.

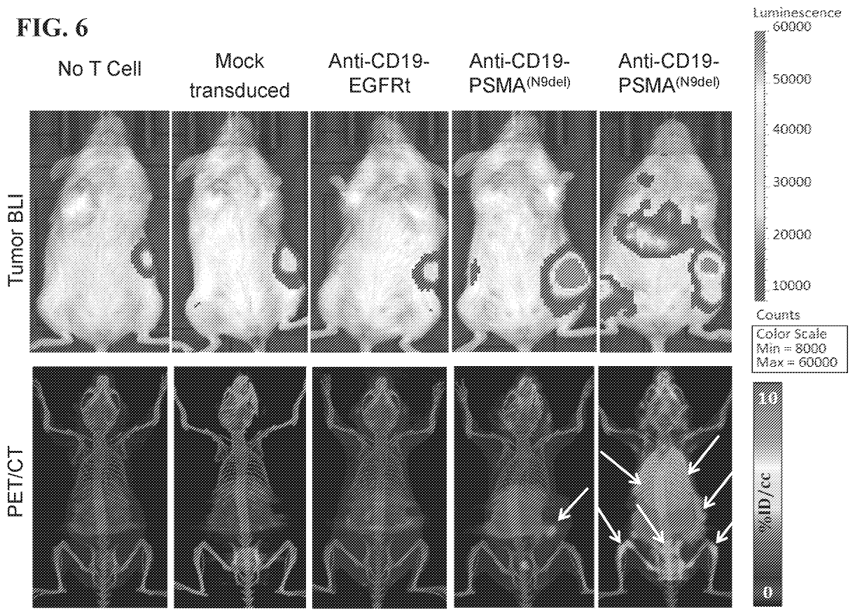

The top panels of FIG. 6 show bioluminescence imaging of tumors developed in exemplary mice in each test group. Bottom panels of FIG. 6 show the corresponding PET/CT scans for detection of the modified PSMA-expressing CAR+ cells using the PSMA-targeting agent in each of the mice shown on the respective portions of the top panel.

As shown in FIG. 6, no PET/CT signal was observed in mice that had not been administered T cells, or that had been administered mock T cells or anti-CD19 CAR+/EGFRt T cells. A PET/CT signal was observed in mice that had been administered the anti-CD19 CAR/PSMA^{(N9del)} T cells. This signal co-localized to the site of the tumor, as shown by correspondence of the tumor bioluminescence and PET/CT signal. In mice that were observed by bioluminescence signal to have developed spontaneous metastatic lesions, such as the mouse depicted in the far right panels in FIG. 6, the administered anti-CD19 CAR/PSMA^{(N9del)} T cells were detected via PET/CT reagent as co-localized with the bioluminescence signal indicative of metastasized lesions. The results demonstrated that engineered T cells co-expressing a modified PSMA containing an extracellular region of PSMA and a CAR could be detected via a PSMA-targeted PET agent, including at local and metastatic tumor sites.

### Example 6: In Vitro Detection of PSMA-Expressing CAR+ T Cells Using a PSMA-Targeting Radiolabeled Agent ([¹⁸F]DCFPyL) by PET

PSMA-expressing cells were detected in vitro using PSMA-targeting radiolabeled agent ([¹⁸F]DCFPyL). A varying number of engineered anti-CD19 CAR/PSMA^{(N9del)}-expressing T cells, either 40,000 (40K), 20,000 (20K), 10,000 (10K), 8,000 (8K), 6,000 (6K), 4,000 (4K), 2,000 (2K), 1,000 (1K), 800 (0.8K), 600 (0.6K), 400 (0.4K) or 200 (0.2K) cells, were added in 20 μL PBS to individual wells of a 384-well cell culture plates. A saturating dose (37 MBq) of [¹⁸F]DCFPyL was added to each well of the plate, and after 1 hour, the cells in each well were imaged by PET.

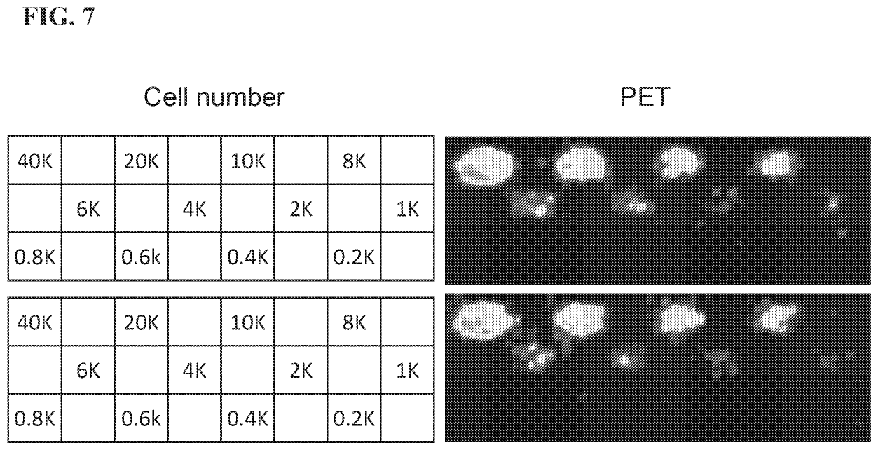

As shown in FIG. 7, at least as few as approximately 2,000 PSMA-expressing CAR T cells/20 μL were detectable in vitro in this study, using [¹⁸F]DCFPyL and PET. The results are consistent with the utility of the modified PSMA variant (e.g., truncated PSMA, tPSMA) as a marker to detect CAR+ T cells in vitro with high sensitivity, by PET/CT.

### Example 7: In Vivo Anti-Tumor Activity, in Vivo Detection and Localization of PSMA-Expressing CAR+ T Cells in Disseminated Tumor Model

To generate a disseminated xenograft model in which spontaneous metastases occur, NSG mice were implanted subcutaneously (s.c.) with 1×10⁶ Nalm6-GFP-ffluc tumor cells. On day 21 after tumor implantation, mice received a single intravenous (i.v.) injection of 5×10⁶ primary human T cells, of which 2×10⁶ cells were T cells engineered to express anti-CD19 CAR/PSMA^{(N9del)}. As controls, groups of mice was used in which mice were not injected with any cells (no T cell) or were injected with 5×10⁶ T cells that did not express a CAR (mock study group). Survival of mice was monitored over time for each group, and detection of tumor cells and CAR+ T cells was assessed by bioluminescence imaging (BLI) and PET/CT, respectively, generally as described in Examples 3 and 4.

A. Survival Curve

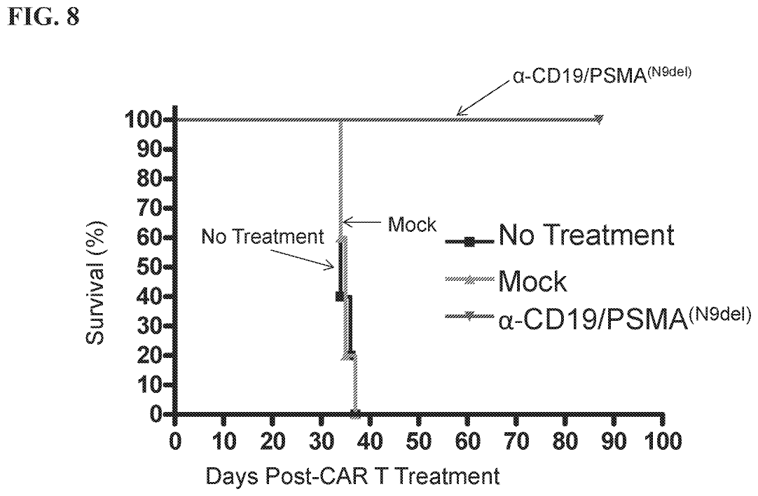

The survival of the mice in each group was monitored for up to 100 days after injection of the CD19+ Nalm-6 cells. The results are shown in FIG. 8. As shown, survival in all mice that had received adoptive transfer of anti-CD19 CAR/PSMA^{(N9del)} T cells was improved compared to that observed in mice administered mock transduced cells or left untreated.

B. In Vivo Detection of Tumor Cells and CAR+ T Cells

Bioluminescence imaging was carried out on mice on days 0 and 11 after injection of CAR+ T cells, and PET/CT imaging using the [¹⁸F]DCFPyL PSMA-targeting radiolabeled agent was carried out on days 5 and 12 after injection of CAR+ T cells.

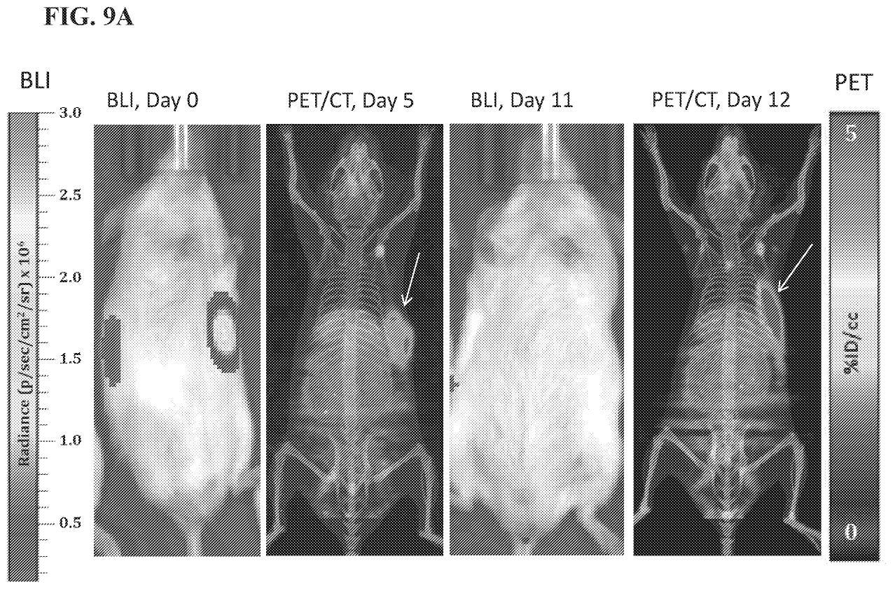

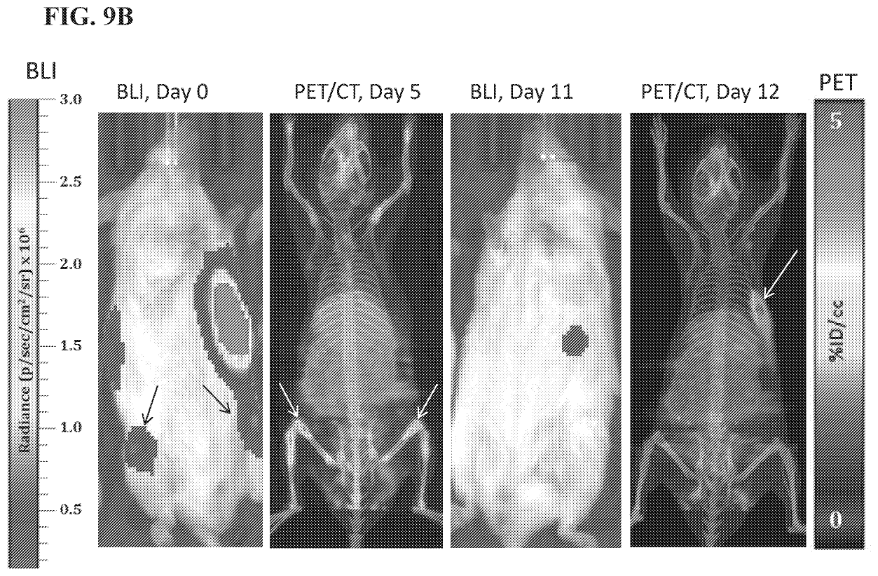

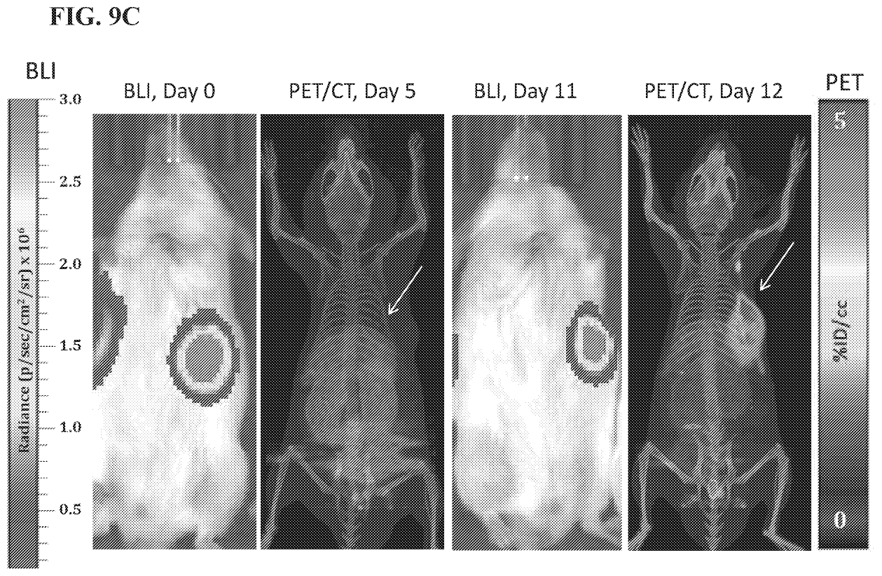

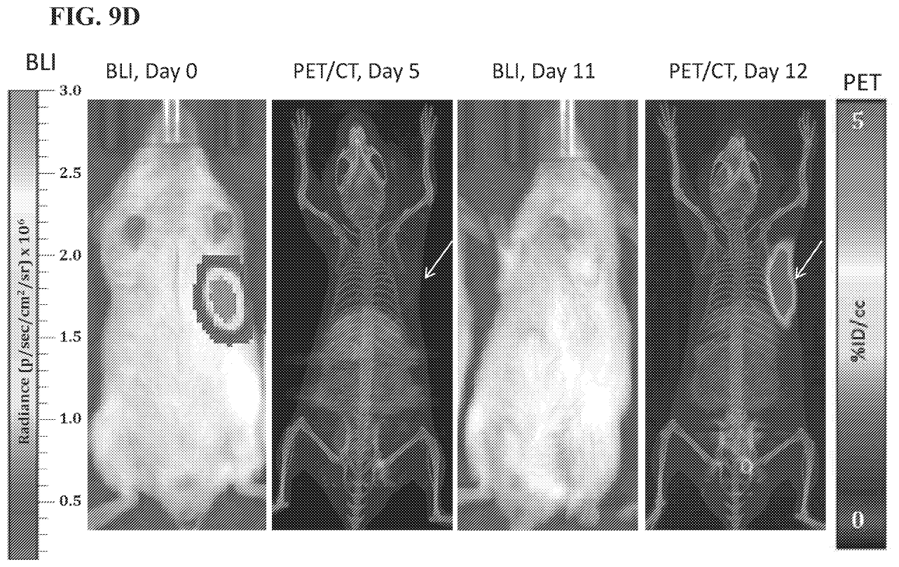

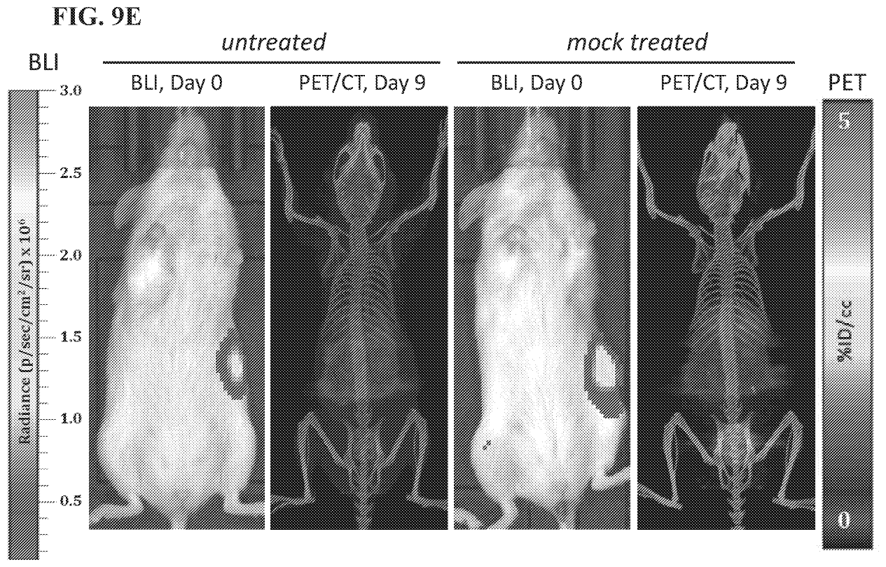

PET and BLI results from three exemplary mice are shown in FIGS. 9A-9C. The first and third image panels show the results of BLI, at day 0 and 11, respectively, and the second and fourth image panels show the results of the PET/CT scans, at days 5 and 12, respectively. The PET/CT signal generally co-localized to the site of the tumor, as shown by correspondence of the tumor bioluminescence and PET/CT signal. As shown in FIGS. 9D and 9E, similar results were observed in a fourth mouse similarly treated (FIG. 9D), and no substantial PET/CT signal was observed in mice that were not treated or in the mock study group (FIG. 9E). The results are consistent with the finding that that engineered T cells co-expressing PSMA containing an extracellular region of PSMA and a CAR could be detected via a PSMA-targeted PET agent, including at local and metastatic tumor sites.

C. Immunohistochemistry

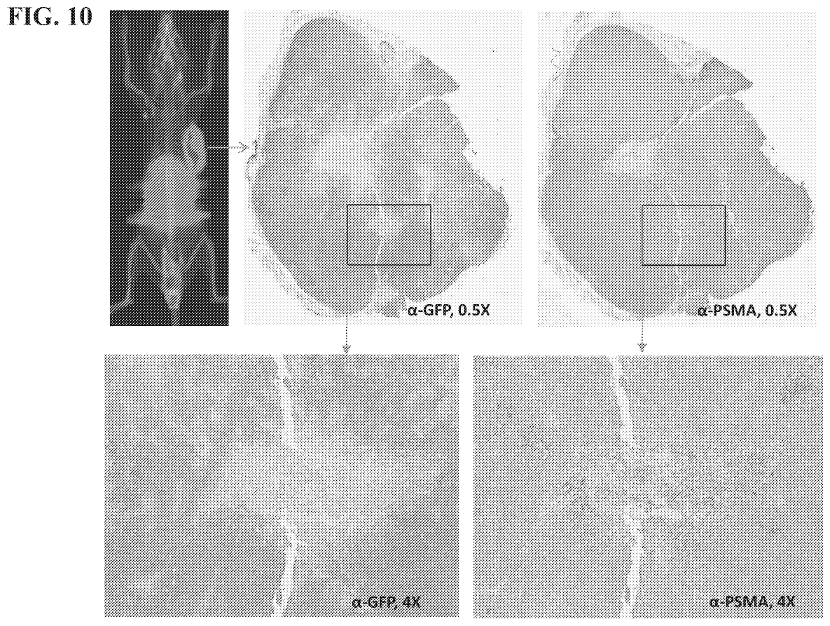

Tumor samples obtained from the mice after tumor implantation were subjected to immunohistochemistry, to determine the location of the CAR-expressing cells within the tumor. The obtained tumor samples were sectioned, and stained with an anti-CD3 antibody to detect T cells (not shown in FIG. 10), an anti-GFP antibody (a-GFP in FIG. 10) to detect the Nalm6-GFP-ffluc tumor cells or an anti-PSMA antibody (α-PSMA in FIG. 10) to detect the anti-CD19 CAR/PSMA^{(N9del)} T cells. As shown in FIG. 10, the results are consistent with the finding that CAR-expressing T cells are present within tumor sites in mice with disseminated tumors receiving an adoptive transfer of anti-CD19 CAR/PSMA^{(N9del)} T cells.

### Example 8: In Vivo Pharmacokinetics and Tumor Localization of PSMA-Expressing CAR+ T Cells in Tumor Model

The pharmacokinetics of administered PSMA-expressing CAR+ T cells in the tumor, peripheral blood and bone marrow in a mouse xenograft tumor model, were determined by PET/CT imaging and flow cytometry.

A. Pharmacokinetic Determination from PET/CT

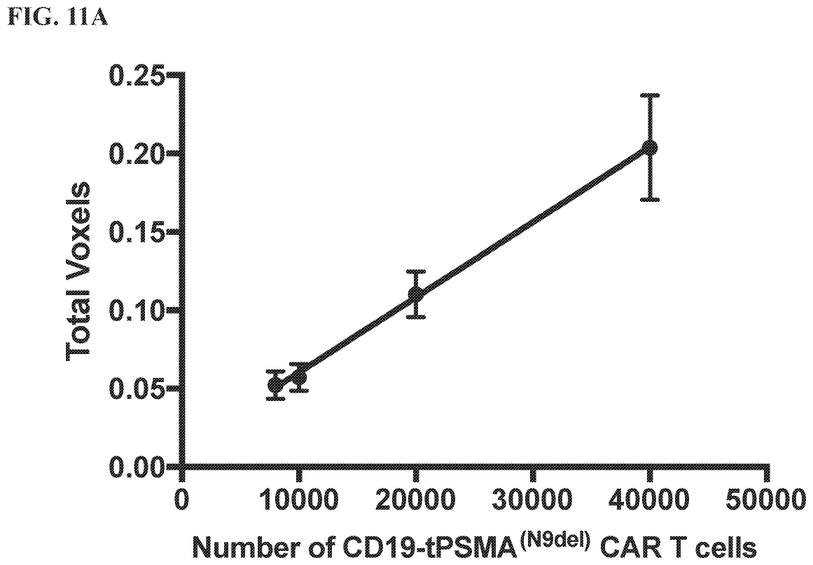

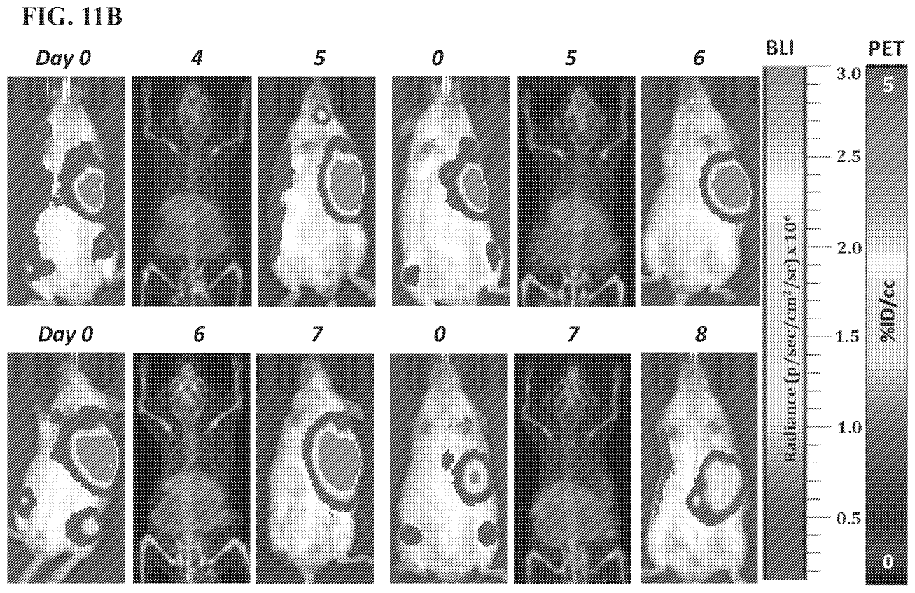

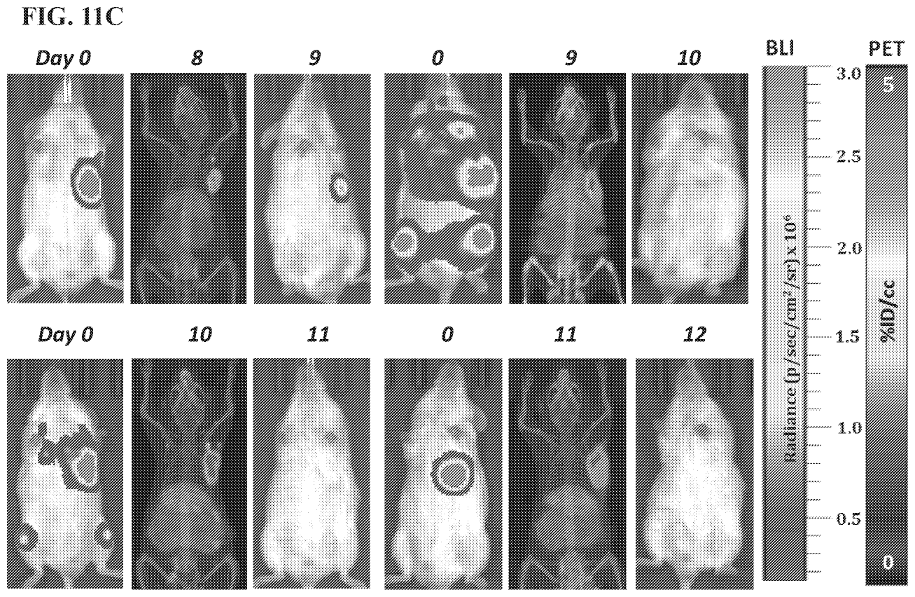

Nalm6-GFP-ffluc xenograft tumor model mice, generated generally as described in Example 7, were administered 2×10⁶ T cells engineered to express anti-CD19 CAR/PSMA^{(N9del)} one day after initial bioluminescence imaging (BLI) of the mice on day 0. On days 4, 5, 6, 7, 8, 9, 10, 11 and 12 after the initial BLI, the mice were assessed by PET/CT, after injection with 14.8 MBq (400 μCi) of [¹⁸F]DCFPyL, and BLI. The total number of CAR+ T cells located within tumor (in millions) was calculated by interpolating the signal intensity of the PET images compared to a standard curve depicted in FIG. 11A. The standard curve was determined by plotting the total voxels (unit of graphic information in three-dimensional space) from the PET images (n=8) from an in vitro phantom imaging experiment, against corresponding cell numbers. The resulting linear regression equation was y=5×10⁻⁶x+0.0122 (R²=0.9989). The error bars in FIG. 11A show the standard deviation. The percentage of tumor cells (by GFP signal) and CAR+ T cells (by anti-PSMA antibody staining) among live cells in the peripheral blood (PPB) or bone marrow (BM) samples were determined by flow cytometry.

The number of CAR+ T cells determined in the various locations from the mice depicted in FIGS. 11B and 11C are listed in Table 1 (days representing the dates of final BLI for each mouse). The results are consistent with the utility of the PSMA variants as a surrogate marker for detection and quantitation of administered CAR+ T cells and assessment of pharmacokinetics of the administered cells, both in vivo based on PET/CT and based on flow cytometry using an anti-PSMA antibody.

TABLE 1
CAR+ T Cell Pharmacokinetics in Mouse Tumor Model.

| | | | Total number | | |
|---|---|---|---|---|---|
| | Tumor | Tumor | of CAR T | CAR T | CAR T |
| Day | (PPB) | (BM) | (Million) | (PPB) | (BM) |
| D 5 | 0.38 | 0.42 | 0.11 | 10.3 | 1.16 |
| D 6 | 0 | 0 | 0.13 | 3.83 | 4.06 |
| D 7 | 0.03 | 0.05 | 0.35 | 18.5 | 5.38 |
| D 8 | 0 | 0 | 0.81 | 8.05 | 2.51 |
| D 9 | 0 | 0 | 1.44 | 2.28 | 0.21 |
| D 10 | 0 | 0 | 2.13 | 10.4 | 0.65 |
| D 11 | 0 | 0 | 2.88 | 5.96 | 1.05 |
| D 12 | 0 | 0 | 3.68 | 7.53 | 0.73 |

B. CAR+ T Cell Density in the Tumor

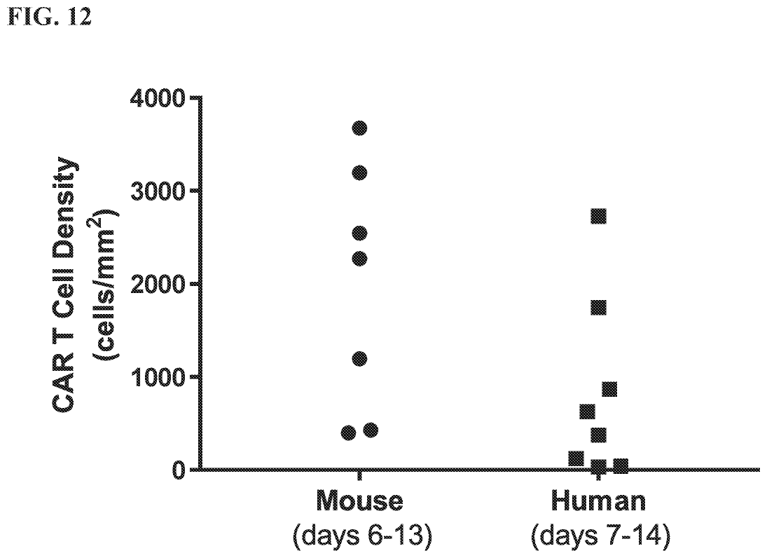

The density of administered CAR+ T cells present in tumor biopsy samples obtained between days 6 and 13 after administration was determined by counting the number of PSMA+ cells stained with an anti-PSMA antibody. As shown in FIG. 12, the CAR+ T cell density in the tumor was within a similar range to those observed from human tumor biopsy samples after administration of CAR+ T cells. The results are consistent with the utility of the PSMA variants as a surrogate marker for quantitation of CAR+ T cell density in the tumor.

C. Immunohistochemistry

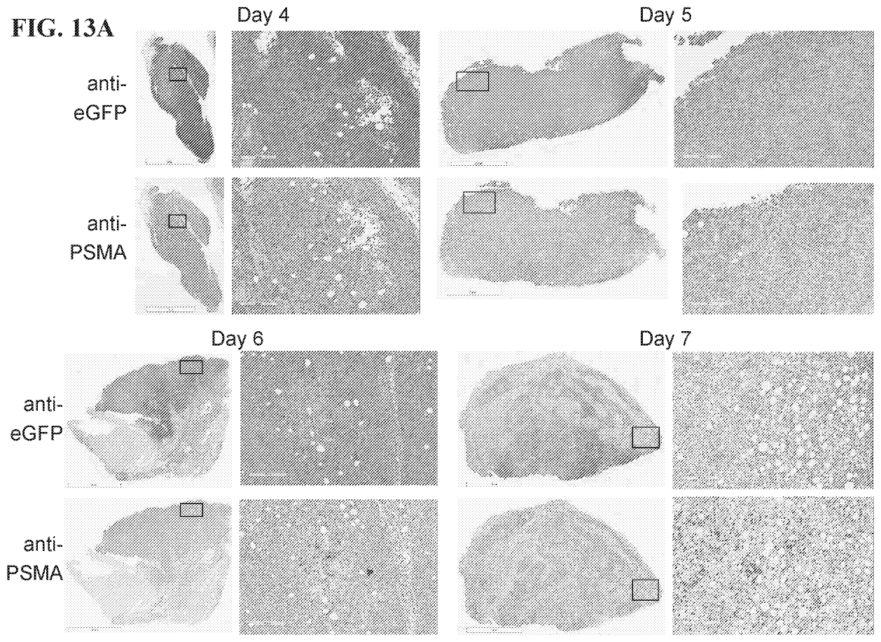

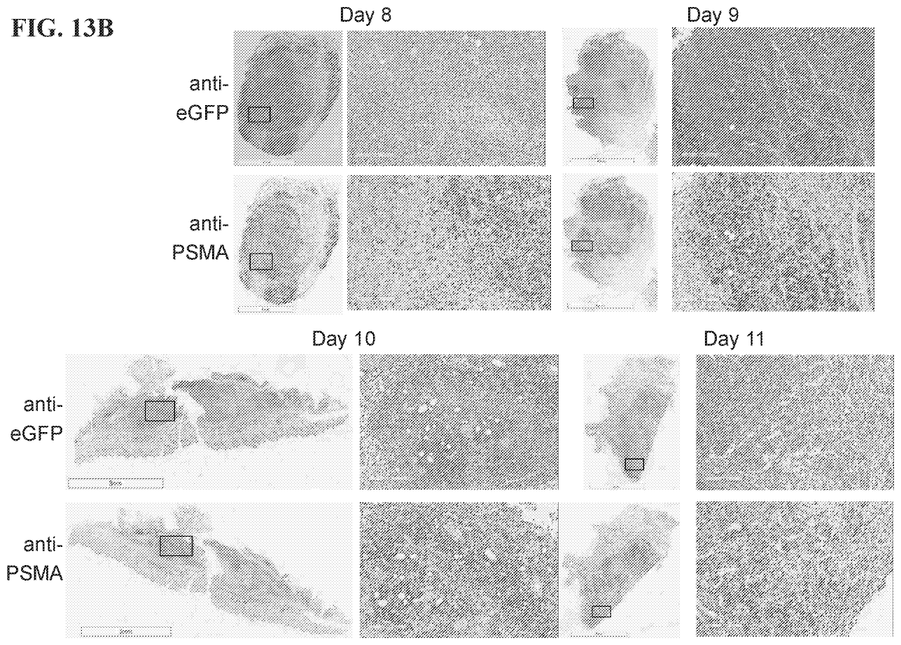

Tumor samples obtained from the mice, between days 4 and 11 after administration of CAR+ T cells were subjected to immunohistochemistry, to determine the presence of the CAR-expressing cells within the tumor. The obtained tumor samples were sectioned, and an anti-GFP antibody (α-GFP in FIGS. 13A-13B) to detect the Nalm6-GFP-ffluc tumor cells or an anti-PSMA antibody (α-PSMA in FIGS. 13A-13B) to detect the anti-CD19 CAR/PSMA^{(N9del)} T cells. The results are consistent with the utility of the PSMA variants as a surrogate marker for detection of administered CAR+ T cells, by immunohistochemistry using an anti-PSMA antibody.

### Example 9: Determination of the Number of Peripheral Blood and Bone Marrow CAR+ T Cells and Tumor-Localized CAR+ T Cells

The number of CAR+ T cells present in the peripheral blood and bone marrow and the number of CAR+ T cells localized to the tumor were determined using PET/CT and flow cytometry.

Five (5) Nalm6-GFP-ffluc xenograft tumor model mice, generated generally as described in Example 7, were administered 2×10⁶ T cells engineered to express anti-CD19 CAR/PSMA^{(N9del)} one day after initial bioluminescence imaging (BLI) of the mice on day 0. On day 10, 11 or 12 after the initial BLI, the mice were assessed by PET/CT, after injection with 14.8 MBq (400 μCi) of [¹⁸F]DCFPyL, or by BLI. The total number of CAR+ T cells located within the entire area of the tumor were calculated based on signal intensity of the PET images compared to a standard curve (see FIG. 11A). Further, the percentage of CAR+ T cells among live cells in peripheral blood (PPB) or within bone marrow (BM) samples was determined using flow cytometry by staining with an anti-PSMA antibody.

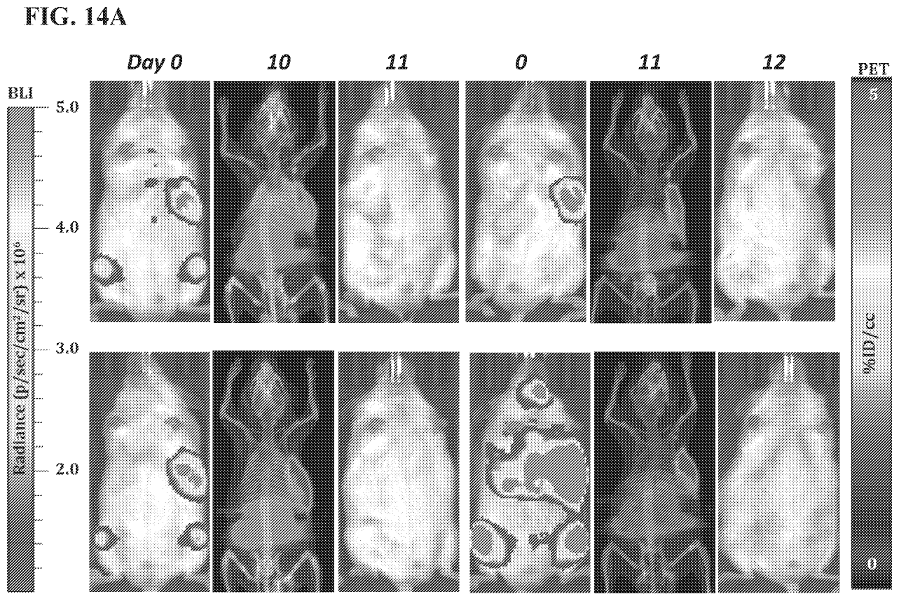

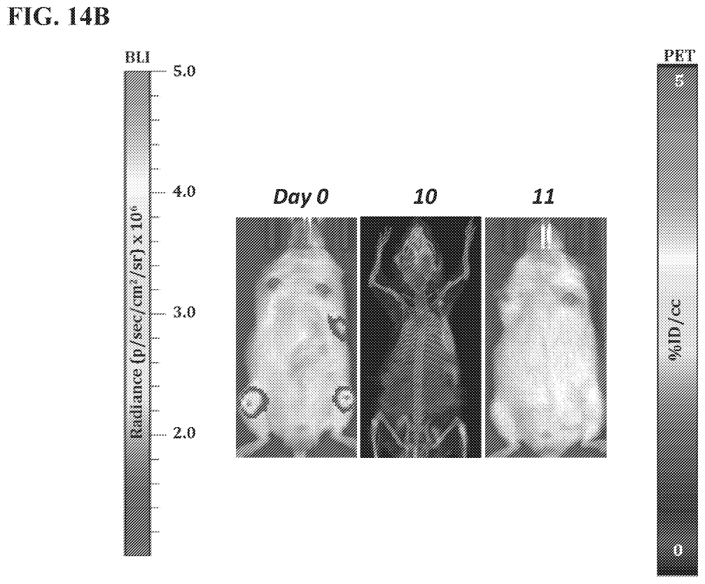

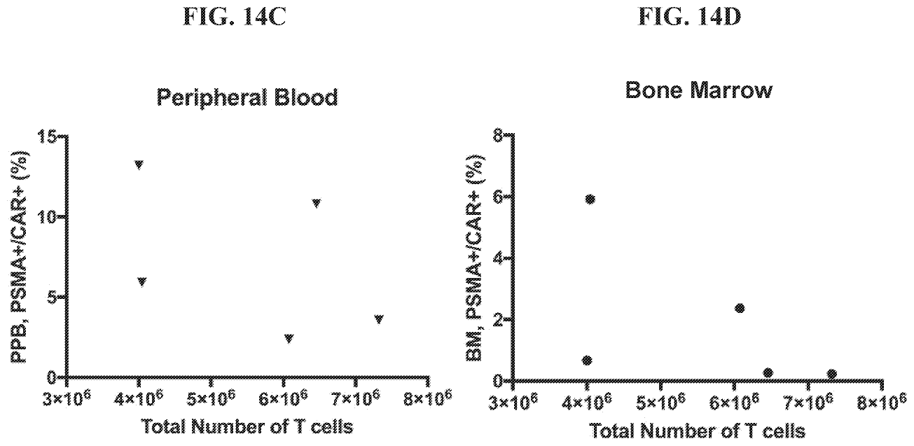

Representative BLI and PET/CT images are shown in FIGS. 14A-14B. The number of cells in the tumor determined from the PET/CT images compared to the percentage of CAR+ T cells among the live cells in PPB and BM for each of the mice shown in FIGS. 14A-14B are shown in FIGS. 14C (PPB) and 14D (BM). The results are consistent with the utility of the PSMA variants as a surrogate marker for detection and quantitation of administered CAR+ T cells, by PET/CT and flow cytometry using an anti-PSMA antibody.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | ESKYGPPCPPCP | spacer (IgG4hinge) |
| | | (aa) |
| | | Homo sapiens |
| 2 | GAATCTAAGTACGGACCGCCCTGCCCCCCTTGCCCT | spacer (IgG4hinge) |
| | | (nt) |
| | | Homo sapiens |
| 3 | ESKYGPPCPPCPGQPREPQVYTLPPSQEEMTKNQVSLTCLVKGFYPSDIAVEWE | Hinge-CH3 spacer |
| | SNGQPENNYKTTPPVLDSDGSFFLYSRLTVDKSRWQEGNVFSCSVMHEALHNHY | Homo sapiens |
| | TQKSLSLSLGK | |
| 4 | ESKYGPPCPPCPAPEFLGGPSVFLFPPKPKDTLMISRTPEVTCVVVDVSQEDPE | Hinge-CH2-CH3 |
| | VQFNWYVDGVEVHNAKTKPREEQFNSTYRVVSVLTVLHQDWLNGKEYKCKVSNK | spacer Homo |
| | GLPSSIEKTISKAKGQPREPQVYTLPPSQEEMTKNQVSLTCLVKGFYPSDIAVE | sapiens |
| | WESNGQPENNYKTTPPVLDSDGSFFLYSRLTVDKSRWQEGNVFSCSVMHEALHN | |
| | HYTQKSLSLSLGK | |
| 5 | RWPESPKAQASSVPTAQPQAEGSLAKATTAPATTRNTGRGGEEKKKEKEKEEQE | IgD-hinge-Fc |
| | ERETKTPECPSHTQPLGVYLLTPAVQDLWLRDKATFTCFVVGSDLKDAHLTWEV | Homo sapiens |
| | AGKVPTGGVEEGLLERHSNGSQSQHSRLTLPRSLWNAGTSVTCTLNHPSLPPQR | |
| | LMALREPAAQAPVKLSLNLLASSDPPEAASWLLCEVSGFSPPNILLMWLEDQRE | |
| | VNTSGFAPARPPPQPGSTTFWAWSVLRVPAPPSPQPATYTCVVSHEDSRTLLNA | |
| | SRSLEVSYVTDH | |
| 6 | LEGGGEGRGSLLTCGDVEENPGPR | T2A artificial |
| 7 | MLLLVTSLLLCELPHPAFLLIPRKVCNGIGIGEFKDSLSINATNIKHFKNCTSI | tEGFR artificial |
| | SGDLHILPVAFRGDSFTHTPPLDPQELDILKTVKEITGFLLIQAWPENRTDLHA | |
| | FENLEIIRGRTKQHGQFSLAVVSLNITSLGLRSLKEISDGDVIISGNKNLCYAN | |
| | TINWKKLFGTSGQKTKIISNRGENSCKATGQVCHALCSPEGCWGPEPRDCVSCR | |
| | NVSRGRECVDKCNLLEGEPREFVENSECIQCHPECLPQAMNITCTGRGPDNCIQ | |
| | CAHYIDGPHCVKTCPAGVMGENNTLVWKYADAGHVCHLCHPNCTYGCTGPGLEG | |
| | CPTNGPKIPSIATGMVGALLLLLVVALGIGLFM | |
| 8 | FWVLVVVGGVLACYSLLVTVAFIIFWV | CD28 (amino acids |
| | | 153-179 of |
| | | Accession No. |
| | | P10747) Homo |
| | | sapiens |
| 9 | IEVMYPPPYLDNEKSNGTIIHVKGKHLCPSPLFPGPSKPFWVLVVVGGVLACYS | CD28 (amino acids |
| | LLVTVAFIIFWV | 114-179 of |
| | | Accession No. |
| | | P10747) Homo |
| | | sapiens |
| 10 | RSKRSRLLHSDYMNMTPRRPGPTRKHYQPYAPPRDFAAYRS | CD28 (amino acids |
| | | 180-220 of |
| | | P10747) Homo |
| | | sapiens |
| 11 | RSKRSRGGHSDYMNMTPRRPGPTRKHYQPYAPPRDFAAYRS | CD28 (LL to GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) Homo |
| | | sapiens |
| 13 | RVKFSRSADAPAYQQGQNQLYNELNLGRREEYDVLDKRRGRDPEMGGKPRRKNP | CD3 zeta Homo |
| | QEGLYNELQKDKMAEAYSEIGMKGERRRGKGHDGLYQGLSTATKDTYDALHMQA | sapiens |
| | LPPR | |
| 14 | RVKFSRSAEPPAYQQGQNQLYNELNLGRREEYDVLDKRRGRDPEMGGKPRRKNP | CD3 zeta Homo |
| | QEGLYNELQKDKMAEAYSEIGMKGERRRGKGHDGLYQGLSTATKDTYDALHMQA | sapiens |
| | LPPR | |
| 15 | RVKFSRSADAPAYKQGQNQLYNELNLGRREEYDVLDKRRGRDPEMGGKPRRKNP | CD3 zeta Homo |
| | QEGLYNELQKDKMAEAYSEIGMKGERRRGKGHDGLYQGLSTATKDTYDALHMQA | sapiens |
| | LPPR | |
| 16 | RKVCNGIGIGEFKDSLSINATNIKHFKNCTSISGDLHILPVAFRGDSFTHTPPL | tEGFR artificial |
| | DPQELDILKTVKEITGFLLIQAWPENRTDLHAFENLEIIRGRTKQHGQFSLAVV | |
| | SLNITSLGLRSLKEISDGDVIISGNKNLCYANTINWKKLFGTSGQKTKIISNRG | |
| | ENSCKATGQVCHALCSPEGCWGPEPRDCVSCRNVSRGRECVDKCNLLEGEPREF | |
| | VENSECIQCHPECLPQAMNITCTGRGPDNCIQCAHYIDGPHCVKTCPAGVMGEN | |
| | NTLVWKYADAGHVCHLCHPNCTYGCTGPGLEGCPTNGPKIPSIATGMVGALLLL | |
| | LWALGIGLFM | |
| 17 | EGRGSLLTCGDVEENPGP | T2A artificial |
| 18 | PLGLWA | MMP cleavable |
| | | linker |
| 19 | GSGATNFSLLKQAGDVEENPGP | P2A |
| 20 | ATNFSLLKQAGDVEENPGP | P2A |
| 21 | QCTNYALLKLAGDVESNPGP | E2A |
| 22 | VKQTLNFDLLKLAGDVESNPGP | F2A |
| 23 | MWNLLHETDSAVATARRPRWLCAGALVLAGGFFLLGFLFGWFIKSSNEATNITP | PSMA WT (full |
| | KHNMKAFLDELKAENIKKFLYNFTQIPHLAGTEQNFQLAKQIQSQWKEFGLDSV | length) |
| | ELAHYDVLLSYPNKTHPNYISIINEDGNEIFNTSLFEPPPPGYENVSDIVPPFS | |
| | AFSPQGMPEGDLVYVNYARTEDFFKLERDMKINCSGKIVIARYGKVFRGNKVKN | |
| | AQLAGAKGVILYSDPADYFAPGVKSYPDGWNLPGGGVQRGNILNLNGAGDPLTP | |
| | GYPANEYAYRRGIAEAVGLPSIPVHPIGYYDAQKLLEKMGGSAPPDSSWRGSLK | |
| | VPYNVGPGFTGNFSTQKVKMHIHSTNEVTRIYNVIGTLRGAVEPDRYVILGGHR | |
| | DSWVFGGIDPQSGAAVVHEIVRSFGTLKKEGWRPRRTILFASWDAEEFGLLGST | |
| | EWAEENSRLLQERGVAYINADSSIEGNYTLRVDCTPLMYSLVHNLTKELKSPDE | |
| | GFEGKSLYESWTKKSPSPEFSGMPRISKLGSGNDFEVFFQRLGIASGRARYTKN | |
| | WETNKFSGYPLYHSVYETYELVEKFYDPMFKYHLTVAQVRGGMVFELANSIVLP | |
| | FDCRDYAVVLRKYADKIYSISMKHPQEMKTYSVSFDSLFSAVKNFTEIASKFSE | |
| | RLQDFDKSNPIVLRMMNDQLMFLERAFIDPLGLPDRPFYRHVIYAPSSHNKYAG | |
| | ESFPGIYDALFDIESKVDPSKAWGEVKRQIYVAAFTVQAAAETLSEVA | |
| 24 | MGNLLHETDSAVATARRPRWLCAGALVLAGGFFLLGFLFGWFIKSSNEATNITP | PSMA W2G (full |
| | KHNMKAFLDELKAENIKKFLYNFTQIPHLAGTEQNFQLAKQIQSQWKEFGLDSV | length) |
| | ELAHYDVLLSYPNKTHPNYISIINEDGNEIFNTSLFEPPPPGYENVSDIVPPFS | |
| | AFSPQGMPEGDLVYVNYARTEDFFKLERDMKINCSGKIVIARYGKVFRGNKVKN | |
| | AQLAGAKGVILYSDPADYFAPGVKSYPDGWNLPGGGVQRGNILNLNGAGDPLTP | |
| | GYPANEYAYRRGIAEAVGLPSIPVHPIGYYDAQKLLEKMGGSAPPDSSWRGSLK | |
| | VPYNVGPGFTGNFSTQKVKMHIHSTNEVTRIYNVIGTLRGAVEPDRYVILGGHR | |
| | DSWVFGGIDPQSGAAVVHEIVRSFGTLKKEGWRPRRTILFASWDAEEFGLLGST | |
| | EWAEENSRLLQERGVAYINADSSIEGNYTLRVDCTPLMYSLVHNLTKELKSPDE | |
| | GFEGKSLYESWTKKSPSPEFSGMPRISKLGSGNDFEVFFQRLGIASGRARYTKN | |
| | WETNKFSGYPLYHSVYETYELVEKFYDPMFKYHLTVAQVRGGMVFELANSIVLP | |
| | FDCRDYAVVLRKYADKIYSISMKHPQEMKTYSVSFDSLFSAVKNFTEIASKFSE | |
| | RLQDFDKSNPIVLRMMNDQLMFLERAFIDPLGLPDRPFYRHVIYAPSSHNKYAG | |
| | ESFPGIYDALFDIESKVDPSKAWGEVKRQIYVAAFTVQAAAETLSEVA | |
| 25 | SAVATARRPRWLCAGALVLAGGFFLLGFLFGWFIKSSNEATNITPKHNMKAFLD | PSMA deletion of |
| | ELKAENIKKFLYNFTQIPHLAGTEQNFQLAKQIQSQWKEFGLDSVELAHYDVLL | aa 1-9 |
| | SYPNKTHPNYISIINEDGNEIFNTSLFEPPPPGYENVSDIVPPFSAFSPQGMPE | |
| | GDLVYVNYARTEDFFKLERDMKINCSGKIVIARYGKVFRGNKVKNAQLAGAKGV | |
| | ILYSDPADYFAPGVKSYPDGWNLPGGGVQRGNILNLNGAGDPLTPGYPANEYAY | |
| | RRGIAEAVGLPSIPVHPIGYYDAQKLLEKMGGSAPPDSSWRGSLKVPYNVGPGF | |
| | TGNFSTQKVKMHIHSTNEVTRIYNVIGTLRGAVEPDRYVILGGHRDSWVFGGID | |
| | PQSGAAVVHEIVRSFGTLKKEGWRPRRTILFASWDAEEFGLLGSTEWAEENSRL | |
| | LQERGVAYINADSSIEGNYTLRVDCTPLMYSLVHNLTKELKSPDEGFEGKSLYE | |
| | SWTKKSPSPEFSGMPRISKLGSGNDFEVFFQRLGIASGRARYTKNWETNKFSGY | |
| | PLYHSVYETYELVEKFYDPMFKYHLTVAQVRGGMVFELANSIVLPFDCRDYAVV | |
| | LRKYADKIYSISMKHPQEMKTYSVSFDSLFSAVKNFTEIASKFSERLQDFDKSN | |
| | PIVLRMMNDQLMFLERAFIDPLGLPDRPFYRHVIYAPSSHNKYAGESFPGIYDA | |
| | LFDIESKVDPSKAWGEVKRQIYVAAFTVQAAAETLSEVA | |
| 26 | atgtggaatctccttcacgaaaccgactcggctgtggccaccgcgcgccgcccg | PSMA WT (nt) |
| | cgctggctgtgcgctggggcgctggtgctggcgggtggcttctttctcctcggc | |
| | ttcctcttcgggtggtttataaaatcctccaatgaagctactaacattactcca | |
| | aagcataatatgaaagcatttttggatgaattgaaagctgagaacatcaagaag | |
| | ttcttatataattttacacagataccacatttagcaggaacagaacaaaacttt | |
| | cagcttgcaaagcaaattcaatcccagtggaaagaatttggcctggattctgtt | |
| | gagetagcacattatgatgtcctgttgtcctacccaaataagactcatcccaac | |
| | tacatctcaataattaatgaagatggaaatgagattttcaacacatcattattt | |
| | gaaccacctcctccaggatatgaaaatgtttcggatattgtaccacctttcagt | |
| | gctttctctcctcaaggaatgccagagggcgatctagtgtatgttaactatgca | |
| | cgaactgaagacttctttaaattggaacgggacatgaaaatcaattgctctggg | |
| | aaaattgtaattgccagatatgggaaagttttcagaggaaataaggttaaaaat | |
| | gcccagctggcaggggccaaaggagtcattctctactccgaccctgctgactac | |
| | tttgctcctggggtgaagtcctatccagatggttggaatcttcctggaggtggt | |
| | gtccagcgtggaaatatcctaaatctgaatggtgcaggagaccctctcacacca | |
| | ggttacccagcaaatgaatatgcttataggcgtggaattgcagaggctgttggt | |
| | cttccaagtattcctgttcatccaattggatactatgatgcacagaagctccta | |
| | gaaaaaatgggtggctcagcaccaccagatagcagctggagaggaagtctcaaa | |
| | gtgccctacaatgttggacctggctttactggaaacttttctacacaaaaagtc | |
| | aagatgcacatccactctaccaatgaagtgacaagaatttacaatgtgataggt | |
| | actctcagaggagcagtggaaccagacagatatgtcattctgggaggtcaccgg | |
| | gactcatgggtgtttggtggtattgaccctcagagtggagcagctgttgttcat | |
| | gaaattgtgaggagctttggaacactgaaaaaggaagggtggagacctagaaga | |
| | acaattttgtttgcaagctgggatgcagaagaatttggtcttcttggttctact | |
| | gagtgggcagaggagaattcaagactccttcaagagcgtggcgtggcttatatt | |
| | aatgctgactcatctatagaaggaaactacactctgagagttgattgtacaccg | |
| | ctgatgtacagcttggtacacaacctaacaaaagagctgaaaagccctgatgaa | |
| | ggctttgaaggcaaatctctttatgaaagttggactaaaaaaagtccttcccca | |
| | gagttcagtggcatgcccaggataagcaaattgggatctggaaatgattttgag | |
| | gtgttcttccaacgacttggaattgcttcaggcagagcacggtatactaaaaat | |
| | tgggaaacaaacaaattcagcggctatccactgtatcacagtgtctatgaaaca | |
| | tatgagttggtggaaaagttttatgatccaatgtttaaatatcacctcactgtg | |
| | gcccaggttcgaggagggatggtgtttgagctagccaattccatagtgctccct | |
| | tttgattgtcgagattatgctgtagttttaagaaagtatgctgacaaaatctac | |
| | agtatttctatgaaacatccacaggaaatgaagacatacagtgtatcatttgat | |
| | tcacttttttctgcagtaaagaattttacagaaattgcttccaagttcagtgag | |
| | agactccaggactttgacaaaagcaacccaatagtattaagaatgatgaatgat | |
| | caactcatgtttctggaaagagcatttattgatccattagggttaccagacagg | |
| | cctttttataggcatgtcatctatgctccaagcagccacaacaagtatgcaggg | |
| | gagtcattcccaggaatttatgatgctctgtttgatattgaaagcaaagtggac | |
| | eettecaaggcctggggagaagtgaagagacagatttatgttgcagccttcaca | |
| | gtgcaggcagctgcagagactttgagtgaagtagcc | |
| 27 | ATGTGGAATCTCCTTCATGAAACAGACTCTGCTGTGGCCACAGCCAGAAGACCC | CpG-free PSMA |
| | AGATGGCTGTGTGCTGGGGCCCTGGTGCTGGCTGGTGGCTTCTTTCTCCTGGGC | |
| | TTCCTCTTTGGGTGGTTTATAAAATCCTCCAATGAAGCTACTAACATTACTCCA | |
| | AAGCATAATATGAAAGCATTTTTGGATGAATTGAAAGCTGAGAACATCAAGAAG | |
| | TTCTTATATAATTTTACACAGATACCACATTTAGCAGGAACAGAACAAAACTTT | |
| | CAGCTTGCAAAGCAAATTCAATCCCAGTGGAAAGAATTTGGCCTGGATTCTGTT | |
| | GAGCTAGCACATTATGATGTCCTGTTGTCCTACCCAAATAAGACTCATCCCAAC | |
| | TACATCTCAATAATTAATGAAGATGGAAATGAGATTTTCAACACATCATTATTT | |
| | GAACCACCTCCTCCAGGATATGAAAATGTTTCTGATATTGTACCACCTTTCAGT | |
| | GCTTTCTCTCCTCAAGGAATGCCAGAGGGAGATCTAGTGTATGTTAACTATGCA | |
| | AGAACTGAAGACTTCTTTAAATTGGAAAGGGACATGAAAATCAATTGCTCTGGG | |
| | AAAATTGTAATTGCCAGATATGGGAAAGTTTTCAGAGGAAATAAGGTTAAAAAT | |
| | GCCCAGCTGGCAGGGGCCAAAGGAGTCATTCTCTACTCTGACCCTGCTGACTAC | |
| | TTTGCTCCTGGGGTGAAGTCCTATCCAGATGGTTGGAATCTTCCTGGAGGTGGT | |
| | GTCCAGAGAGGAAATATCCTAAATCTGAATGGTGCAGGAGACCCTCTCACACCA | |
| | GGTTACCCAGCAAATGAATATGCTTATAGGAGAGGAATTGCAGAGGCTGTTGGT | |
| | CTTCCAAGTATTCCTGTTCATCCAATTGGATACTATGATGCACAGAAGCTCCTA | |
| | GAAAAAATGGGTGGCTCAGCACCACCAGATAGCAGCTGGAGAGGAAGTCTCAAA | |
| | GTGCCCTACAATGTTGGACCTGGCTTTACTGGAAACTTTTCTACACAAAAAGTC | |
| | AAGATGCACATCCACTCTACCAATGAAGTGACAAGAATTTACAATGTGATAGGT | |
| | ACTCTCAGAGGAGCAGTGGAACCAGACAGATATGTCATTCTGGGAGGTCACAGG | |
| | GACTCATGGGTGTTTGGTGGTATTGACCCTCAGAGTGGAGCAGCTGTTGTTCAT | |
| | GAAATTGTGAGGAGCTTTGGAACACTGAAAAAGGAAGGGTGGAGACCTAGAAGA | |
| | ACAATTTTGTTTGCAAGCTGGGATGCAGAAGAATTTGGTCTTCTTGGTTCTACT | |
| | GAGTGGGCAGAGGAGAATTCAAGACTCCTTCAAGAGAGGGGAGTGGCTTATATT | |
| | AATGCTGACTCATCTATAGAAGGAAACTACACTCTGAGAGTTGATTGTACACCC | |
| | CTGATGTACAGCTTGGTACACAACCTAACAAAAGAGCTGAAAAGCCCTGATGAA | |
| | GGCTTTGAAGGCAAATCTCTTTATGAAAGTTGGACTAAAAAAAGTCCTTCCCCA | |
| | GAGTTCAGTGGCATGCCCAGGATAAGCAAATTGGGATCTGGAAATGATTTTGAG | |
| | GTGTTCTTCCAAAGACTTGGAATTGCTTCAGGCAGAGCAAGGTATACTAAAAAT | |
| | TGGGAAACAAACAAATTCAGTGGCTATCCACTGTATCACAGTGTCTATGAAACA | |
| | TATGAGTTGGTGGAAAAGTTTTATGATCCAATGTTTAAATATCACCTCACTGTG | |
| | GCCCAGGTTAGAGGAGGGATGGTGTTTGAGCTAGCCAATTCCATAGTGCTCCCT | |
| | TTTGATTGTAGAGATTATGCTGTAGTTTTAAGAAAGTATGCTGACAAAATCTAC | |
| | AGTATTTCTATGAAACATCCACAGGAAATGAAGACATACAGTGTATCATTTGAT | |
| | TCACTTTTTTCTGCAGTAAAGAATTTTACAGAAATTGCTTCCAAGTTCAGTGAG | |
| | AGACTCCAGGACTTTGACAAAAGCAACCCAATAGTATTAAGAATGATGAATGAT | |
| | CAACTCATGTTTCTGGAAAGAGCATTTATTGATCCATTAGGGTTACCAGACAGG | |
| | CCTTTTTATAGGCATGTCATCTATGCTCCAAGCAGCCACAACAAGTATGCAGGG | |
| | GAGTCATTCCCAGGAATTTATGATGCTCTGTTTGATATTGAAAGCAAAGTGGAC | |
| | CCTTCCAAGGCCTGGGGAGAAGTGAAGAGACAGATTTATGTTGCAGCCTTCACA | |
| | GTGCAGGCAGCTGCAGAGACTTTGAGTGAAGTAGCCTAA | |
| 28 | PGGG-(SGGGG)5-P- wherein P is proline, G is glycine | linker |
| | and S is serine | |
| 29 | GSADDAKKDAAKKDGKS | linker |
| 30 | atgcttctcctggtgacaagccttctgctctgtgagttaccacacccagcattc | GMCSFR alpha |
| | ctcctgatccca | chain signal |
| | | sequence |
| 31 | MLLLVTSLLLCELPHPAFLLIP | GMCSFR alpha |
| | | chain signal |
| | | sequence |
| 32 | MALPVTALLLPLALLLHA | CD8 alpha signal |
| | | peptide |
| 33 | GFLG | linker |
| 34 | KLAKLAKKLAKLAK | peptide toxin |
| 35 | WQPDTAHHWATL | PSMA binding |
| | | peptide 1 |
| 36 | HNAYWHWPPSMT | PSMA binding |
| | | peptide 2 |
| 37 | GHLIPLRQPSR | PSMA binding |
| | | peptide 3 |
| 38 | YTSPHHSTTGHL | PSMA binding |
| | | peptide 4 |
| 39 | WTHHHSYPRPL | PSMA binding |
| | | peptide 5 |
| 40 | NSFPLMLMHHHP | PSMA binding |
| | | peptide 6 |
| 41 | KHMHWHPPALN | PSMA binding |
| | | peptide 7 |
| 42 | SLDSMSPQWHAD | PSMA binding |
| | | peptide 8 |
| 43 | SEFIHHWTPPPS | PSMA binding |
| | | peptide 9 |
| 44 | NGFSHHAPLMRY | PSMA binding |
| | | peptide 10 |
| 45 | HHEWTHHWPPP | PSMA binding |
| | | peptide 11 |
| 46 | AWPENPSRRPF | PSMA binding |
| | | peptide 12 |
| 47 | AGFOHHPSFYRF | PSMA binding |
| | | peptide 13 |
| 48 | KSLSRHDHIHHH | PSMA binding |
| | | peptide 14 |
| 49 | YRHWPIDYPPP | PSMA binding |
| | | peptide 15 |
| 50 | MIHTNHWWAQD | PSMA binding |
| | | peptide 16 |
| 51 | QRSPMMSRIRLP | PSMA binding |
| | | peptide 17 |
| 52 | MAVATARRPRWLCAGALVLAGGFTLLGFLFGWFIKSSNEATNITPKHNMKAFLD | PSMA N9del |
| | ELKAENIKKFLYNETQTPHLAGTEQNFQLAKQLQSQWKEFGLDSVELAHYDVLL | |
| | SYPNKTHPNYISIINEDGNEIENTSLFEPPPPGYENVSDIVPPFSAFSPQGMPE | |
| | GDLVYVNYARTEDFPKLERDMKINCSGKIVIARYGKVFRGNKVKNAQLAGAKGV | |
| | 1LYSDPADYTAPGVKSYPDGWNLPGGGVQRGNILNLNGAGDPLTPGYPANEYAY | |
| | RRGIAEAVGLPSIPVHPIGYYDAQKLLEKMGGSAPPDSSWRGSLKVPYNVGPGF | |
| | TGNFSTQKVKMHIHSTNEVTRIYNVIGTLRGAVEPDRYVILGGHRDSWVFGGID | |
| | PQSGAAVVHEIVRSFGTLKKEGWRPRRTILFASWDAEEFGLLGSTEWAEENSRL | |
| | LQERGVAYINADSSIEGNYTLRVDCTPLMYSLVHNLTKELKSPDEGFEGKSLYE | |
| | SWTKKSPSPEFSGMPRISKLGSGNDFEVFFQRLGIASGRARYTKNWETNKFSGY | |
| | PLYHSVYETYELVEKFYDPMFKYHLTVAQVRGGMVFELANSIVLPFDCRDYAVV | |
| | LRKYADKIYSISMKHPQEMKTYSVSFDSLFSAVKNFTEIASKFSERLQDFDKSN | |
| | PIVLRMMNDQLMFLERAFTDPLGLPDRPFYRHVIYAPSSHNKYAGESFPGIYDA | |
| | LFDIESKVDPSKAWGEVKRQIYVAAFTVQAAAPTLSEVA | |
| 53 | ATGGCCGTCGCAACCGCCCGCCGACCCCGCTGGCTGTGCGCCGGAGCCCTGGTG | PSMA N9del DNA |
| | CTGGCCGGCGGCTTCTTTCTGCTGGGCTTCCTGTTTGGCTGGTTTATCAAGAGC | |
| | TCCAACGAGGCCACCAATATCACACCTAAGCACAATATGAAGGCCTTCCTGGAC | |
| | GAGCTGAAGGCCGAGAATATCAAGAAGTTCCTGTATAACTTCACCCAGATCCCA | |
| | CACCTGGCCGGCACAGAGCAGAACTTTCAGCTGGCCAAGCAGATCCAGAGCCAG | |
| | TGGAAGGAGTTCGGCCTGGACTCCGTGGAGCTGGCCCACTACGATGTGCTGCTG | |
| | TCTTATCCAAATAAGACCCACCCCAACTATATCAGCATCATCAACGAGGATGGC | |
| | AATGAGATCTTCAACACATCTCTGTTTGAGCCCCCTCCACCCGGCTACGAGAAT | |
| | GTGAGCGACATCGTGCCTCCATTCTCTGCCTTTAGCCCACAGGGAATGCCTGAG | |
| | GGCGATCTGGTGTACGTGAATTATGCCAGAACCGAGGACTTCTTTAAGCTGGAG | |
| | AGAGATATGAAGATCAACTGCAGCGGCAAGATCGTGATCGCCAGATACGGCAAG | |
| | GTGTTTCGCGGCAATAAGGTGAAGAACGCACAGCTGGCCGGAGCAAAGGGCGTG | |
| | ATCCTGTACTCCGACCCCGCCGATTATTTCGCCCCTGGCGTGAAGTCCTATCCA | |
| | GACGGCTGGAATCTGCCAGGAGGAGGCGTGCAGAGGGGAAACATCCTGAACCTG | |
| | AATGGAGCAGGCGATCCTCTGACCCCAGGATACCCCGCCAACGAGTACGCCTAT | |
| | AGGAGAGGAATCGCAGAGGCAGTGGGCCTGCCTTCCATCCCAGTGCACCCCATC | |
| | GGCTACTATGACGCCCAGAAGCTGCTGGAGAAGATGGGAGGCTCTGCCCCACCT | |
| | GATTCTAGCTGGAGGGGCAGCCTGAAGGTGCCTTACAATGTGGGCCCAGGCTTC | |
| | ACCGGCAACTTTTCCACACAGAAGGTGAAGATGCACATCCACTCTACCAATGAG | |
| | GTGACACGGATCTATAACGTGATCGGCACCCTGAGGGGAGCAGTGGAGCCTGAC | |
| | AGATACGTGATCCTGGGCGGCCACAGAGACAGCTGGGTGTTTGGAGGAATCGAT | |
| | CCACAGTCCGGAGCAGCAGTCGTGCACGAGATCGTGAGGTCCTTCGGCACCCTG | |
| | AAGAAGGAGGGATGGCGCCCCAGGAGGACAATCCTGTTTGCCTCTTGGGATGCC | |
| | GAGGAGTTCGGCCTGCTGGGCTCCACAGAGTGGGCAGAGGAGAATTCTCGGCTG | |
| | CTGCAGGAGAGAGGCGTGGCCTACATCAATGCCGACTCCTCTATCGAGGGCAAC | |
| | TATACCCTGAGGGTGGATTGCACACCCCTGATGTACTCCCTGGTGCACAACCTG | |
| | ACCAAGGAGCTGAAGTCTCCTGACGAGGGCTTCGAGGGUAAGTCTCTGTATGAG | |
| | AGCTGGACAAAGAAGTCTCCAAGCCCCGAGTTTAGCGGCATGCCTAGGATCTCC | |
| | AAGCTGGGCTCTGGCAATGATTTCGAGGTGTTCTTTCAGCGCCTGGGAATCGCC | |
| | TCCGGCCGGGCAAGATACACCAAGAATTGGGAGACAAACAAGTTCTCTGGCTAG | |
| | CCACTGTATCACAGCGTGTACGAGACATATGAGCTGGTGGAGAAGTTCTACGAC | |
| | CCCATGTTTAAGTATCACCTGACAGTGGCACAGGTGCGGGGAGGAATGGTGTTT | |
| | GAGCTGGCCAATAGCATCGTGCTGCCATTCGACTGTAGGGATTATGCCGTGGTG | |
| | CTGCGCAAGTACGCCGACAAGATCTATTCCATCTCTATGAAGCACCCCCAGGAG | |
| | ATGAAGACCTACAGCGTGTCCTTCGATTCCCTGTTTTCTGCCGTGAAGAACTTC | |
| | ACAGAGATCGCCAGCAAGTTTTCCGAGAGACTGCAGGACTTCGATAAGAGCAAT | |
| | CCCATCGTGCTGCGGATGATGAAGGACCAGCTGATGTTCGTGGAGAGAGCCTTT | |
| | ATCGACCCTCTGGGGGTGCCTGATAGGCCATTGTACCGCCACGTGATGTATGCC | |
| | CCTAGCTCCCACAACAAGTACGCCGGCGAGTCCTTTCCAGGCATCTATGACGCC | |
| | CTGTTCGATATCGAGAGCAAGGTGGACCCCTCCAAGGCATGGGGAGAGGTGAAG | |
| | AGACAGATCTATGTCGCAGCATTCACTGTCCAGGCAGCAGCAGAAACCCTGTCA | |
| 54 | GGATCTGGAGCAACAAACTTCTCACTACTCAAACAAGCAGGTGACGTGGAGGAG | P2A DNA |
| | AATCCCGGACCC | |
| 55 | ESKYGPPCPSCP | Hinge |
| 56 | YGPPCPPCP | Hinge |
| 57 | KYGPPCPPCP | Hinge |
| 58 | EVVVKYGPPCPPCP | Hinge |
| 59 | RASQDISKYLN | FMC63 CDR L1 |
| 60 | SRLHSGV | FMC63 CDR L2 |
| 61 | GNTLPYTFG | FMC63 CDR L3 |
| 62 | DYGVS | FMC63 CDR H1 |
| 63 | VIWGSETTYYNSALKS | FMC63 CDR H2 |
| 64 | YAMDYWG | FMC63 CDR H3 |
| 65 | GSTSGSGKPGSGEGSTKG | Linker |
| 66 | X₁PPX₂P, X₁ is glycine, cysteine or arginine and X₂ is | spacer |
| | cysteine or threonine | |

1. An engineered cell, comprising
a modified prostate-specific membrane antigen (PSMA); and
a recombinant antigen receptor.

2-4. (canceled)

5. The engineered cell of claim 1, wherein the modified PSMA, is capable of being recognized by a PSMA-targeting molecule.

6-9. (canceled)

10. The engineered cell of claim 1, wherein the modified PSMA comprises one or more amino acid modifications compared to the amino acid sequence set forth in SEQ ID NO:23, and wherein the modified PSMA has at least 85% sequence identity to the amino acid sequence set forth in SEQ ID NO:23.

11-12. (canceled)

13. The engineered cell of claim 10, wherein the one or more amino acid modifications comprise one or more amino acid substitutions, deletions or insertions.

14. The engineered cell of claim 13, wherein the modified PSMA (i) exhibits reduced endogenous signaling; (ii) exhibits increased cell surface expression; or (iii) exhibits reduced cellular internalization compared to a wild-type or unmodified PSMA.

15. The engineered cell of claim 1, wherein the modified PSMA comprises an amino acid modification at position 2 with reference to positions in the sequence of amino acids set forth in SEQ ID NO:23.

16. The engineered cell of claim 15, wherein the modified PSMA comprises
i) the sequence of amino acids set forth in SEQ ID NO:24 or a fragment thereof, or
ii) a sequence of amino acids that exhibits at least 85% sequence identity to SEQ ID NO:24 and comprises at least one amino acid substitution compared to SEQ ID NO:24.

17. The engineered cell of claim 1, wherein the modified PSMA comprises a deletion of one or more N-terminal amino acid residues within the 19 N-terminal amino acid residues, compared to a wild-type or unmodified PSMA.

18. (canceled)

19. The engineered cell of claim 1, wherein the modified PSMA comprises a deletion of a contiguous amino acid sequence at the N-terminus starting from the amino acid residue at position 2, 3, 4, or 5 and up to position 6, 7, 8, 9, 10, 11, 12, 13, 14, 15, 16, 17 18 or 19, compared to a wild-type or unmodified PSMA, with reference to positions in the sequence of amino acids set forth in SEQ ID NO:23.

20. The engineered cell of claim 1, wherein the modified PSMA comprises
i) the sequence of amino acids set forth in SEQ ID NO: 52 or a fragment thereof; or
ii) a sequence of amino acids that exhibits at least 85% sequence identity to SEQ ID NOS: 52 and contains a methionine start codon.

21. The engineered cell of claim 1, wherein
i) the modified PSMA is encoded by the sequence of nucleic acids set forth in SEQ ID NO: 53 or a fragment thereof; or
ii) a sequence of nucleic acids that exhibits at least 85% sequence identity to SEQ ID NO: 53 and contains nucleotides encoding a methionine start codon.

22-24. (canceled)

25. The engineered cell of claim 5, wherein the PSMA-targeting molecule comprises a small molecule, a ligand, an antibody or antigen-binding fragment thereof, an aptamer, a peptide, or a conjugate thereof.

26. The engineered cell of claim 5, wherein the PSMA-targeting molecule comprises a small molecule.

27. The engineered cell of claim 26, wherein the small molecule is selected from the group consisting of 2 (3-{1-carboxy-5-[(6-fluoro-pyridine-3-carbonyl)-amino]-pentyl}-ureido)-pentanedioicacid (DCFPyL), N-[N-[(S)-1,3-dicarboxypropyl]carbamoyl]-4-fluorobenzyl-L-cysteine (DCFBC), (aminostyryl)pyridinium (ASP) dye, 2-(3-[1-carboxy-5-[(5-iodo-pyridine-3-carbonyl)-amino]-pentyl]-ureido)-pentanedioicacid (YC-VI-11), 2-[3-[1-carboxy-5-(4-iodo-benzoylamino)-pentyl]-ureido]-pentanedioicacid (DCIBzL or YC-7), 1-(3-carboxy-4-(3-hydroxy-6-oxo-6H-xanthen-9-yl)phenylamino)-9,16,24-trioxo-1-thioxo-2,8,17,23,25-pentaazaoctacosane-7,22,26,28-tetracarboxylicacid (YC-36), Glu-NH-CO-NH-Lys(Ahx)-HBED-CC (PSMA-HBED-CC), 9-(4-Fluoro-3-[hydroxymethyl]butyl)guanine (FHBG), Glu-urea-Lys-(Ahx)-[(HBED-CC)] (PSMA-11), PSMA-617, 2-(phosphonomethyl)pentanedioicacid, 2-PMPA, Fluorobenzoylphosphoramidate, (2S, 4S)-2-fluoro-4-(phosphonomethyl)pentanedioidacid (BAY1075553), N-[N-[(S)-1,3-dicarboxypropyl]carbamoyl]-S-methyl-L-cysteine (-DCMC), EuK-Subkff-DOTAGA, 2-[3-(1,3-dicarboxypropyl)ureido]pentanedioicacid (DUPA), PSMAN, N′-bis[2-hydroxy-5-(carboxyethyl)benzyl]ethylenedia-mine-N, N′-diaceticacid, MIP-1072, MIP-1095, MIP-1404, MIP-1405 and N-[[[(1S)-1-Carboxy-3-methylbutyl]amino]carbonyl]-L-glutamic acid (ZJ43), (S)-2-(4-iodobenzylphosphonomethyl)-pentanedioic acid (GPI-18431), 2-[(3-{4-[(2-amino-4-hydroxy-pteridin-6-ylmethyl)-amino]-benzoylamino}-3-carboxy-propyl)-hydroxy-phosphinoylmethyl]-pentanedioic acid (MPE), (2S,3′S)-{[(3′-Amino-3′-carboxy-propyl)-hydroxyphosphinoyl]methyl}-pentanedioic acid (EPE) and (2S)-2-{[(2-carboxy-ethyl)-hydroxy-phosphinoyl]methyl}-pentanedioic acid (SPE).

28. (canceled)

29. The engineered cell of claim 5, wherein the PSMA-targeting molecule comprises an antibody or antigen-binding fragment thereof having a binding site that specifically binds to PSMA.

30. The engineered cell of claim 29, wherein the antibody or antigen-binding fragment thereof is selected from the group consisting of J591, DFO-J591, CYT-356, J415, 3/A12, 3/F11, 3/E7, D2B, 107-1A4, YPSMA-1, YPSMA-2, 3E6, 2G7, 24.4E6, GCP-02, GCP-04, GCP-05, J533, E99, 1G9, 3C6, 4.40, 026, D7-Fc, D7-CH3, 4D4, A5, and antigen-binding fragments thereof.

31. The engineered cell of claim 5, wherein the PSMA-targeting molecule is or comprises an aptamer or a conjugate thereof, wherein the aptamer or conjugate thereof is selected from the group consisting of A9, A10, A10g, A10-3.2 and SZT101 or a conjugate thereof.

32. (canceled)

33. The engineered cell of claim 1, wherein the recombinant antigen receptor is capable of binding to a target antigen that is associated with, specific to, or expressed on a cell or tissue of a disease, disorder or condition.

34.-37. (canceled)

38. The engineered cell of claim 1, wherein the recombinant antigen receptor comprises a T cell receptor (TCR) or antigen-binding fragment thereof.

39. The engineered cell of claim 1, wherein the recombinant antigen receptor is a chimeric antigen receptor (CAR).

40-59. (canceled)

60. The engineered cell of claim 1, wherein the cell is a CD4+ T cell or a subtype thereof, a CD8+T cell or a subtype thereof, a NK cell, or an iPSC.

61-63. (canceled)

64. A polynucleotide comprising a first nucleic acid encoding a modified prostate-specific membrane antigen (PSMA) and a second nucleic acid encoding a recombinant antigen receptor.

65. The polynucleotide of claim 64, wherein the nucleic acid encoding the modified PSMA and the nucleic acid encoding the recombinant antigen receptor are separated by an internal ribosome entry site (IRES), or a nucleic acid encoding a self-cleaving peptide or a peptide that causes ribosome skipping.

66-67. (canceled)

68. A set of polynucleotides comprising a first polynucleotide comprising a nucleic acid encoding a modified prostate-specific membrane antigen (PSMA) and a second polynucleotide comprising a nucleic acid encoding a recombinant antigen receptor.

69. A composition comprising the set of polynucleotides of claim 68.

70-87. (canceled)

88. The polynucleotide of claim 64, wherein the modified PSMA comprises the sequence of amino acids set forth in SEQ ID NO: 52 or a fragment thereof; or a sequence of amino acids that exhibits at least 85% sequence identity to SEQ ID NO: 52 and contains a methionine start codon.

89. The polynucleotide of claim 64, wherein the modified PSMA
i) is encoded by the sequence of nucleic acids set forth in SEQ ID NO: 53 or a fragment thereof; or
ii) a sequence of nucleic acids that exhibits at least 85% sequence identity to SEQ ID NO: 53 and and contains a nucleic acid encoding a methionine start codon.

90-121. (canceled)

122. The polynucleotide of claim 64, wherein the polynucleotide comprises, in 5′ to 3′ order:
i) a nucleic acid encoding a signal peptide;
ii) a nucleic acid encoding the recombinant antigen receptor, wherein the recombinant antigen receptor is a chimeric antigen receptor (CAR) said CAR comprising an scFv, a spacer, a transmembrane domain, an intracellular region comprising a costimulatory signaling region, and an intracellular signaling domain of a CD3-zeta (CD3ζ) chain, or a signaling portion thereof;
iii) a nucleic acid sequence encoding a self-cleaving peptide or a peptide that causes ribosome skipping; and
iv) a nucleic acid encoding the modified PSMA, which comprises the sequence of amino acids set forth in SEQ ID NO: 52 or a fragment thereof, or a sequence of amino acids that exhibits at least 85% sequence identity to SEQ ID NO: 52.

123. A vector, comprising the polynucleotide of claim 64.

124-126. (canceled)

127. A set of vectors, comprising a first vector and a second vector, wherein the first vector comprises the first polynucleotide of any of claim 68 and the second vector comprises the second polynucleotide of any of claim 68.

128. A composition comprising the set of vectors of claim 127.

129. A method of producing an engineered cell, comprising introducing the polynucleotide of claim 64 into a cell.

130. (canceled)

131. An engineered cell, comprising the polynucleotide of claim 64.

132. (canceled)

133. A composition comprising the engineered cell of claim 1.

134-135. (canceled)

136. A method of treatment comprising administering the engineered cells of claim 1 to a subject.

137. The method of claim 136, further comprising:
administering to the subject a PSMA-targeting molecule, or a composition comprising a PSMA-targeting molecule.

138-174. (canceled)

175. The method of claim 136 further comprising detecting cells that express the modified PSMA, detecting the binding of the PSMA-targeting molecule to the PSMA or modified form thereof, or the presence of the PSMA-targeting molecule.

176. The method of claim 175, wherein said detecting is performed in vivo.

177. A method of detecting engineered cells, comprising:
(a) contacting the engineered cells of claim 1 a PSMA-targeting molecule; and
(b) detecting the binding of said PSMA-targeting molecule to the engineered cells.

178. (canceled)

179. A method of detecting the presence or absence of engineered cells in a subject, the method comprising:
(a) administering to a subject a PSMA-targeting molecule, said subject having been previously administered the engineered cells of claim 1; and
(b) detecting the binding of the PSMA-targeting molecule to the engineered cells in the subject.

180-213. (canceled)

214. A method of selecting, isolating or separating cells expressing a modified PSMA comprising:
(a) contacting a plurality of cells comprising the engineered cells of claim 1 with a PSMA-targeting molecule; and
(b) selecting, isolating or separating cells that are recognized or bound by the PSMA-targeting molecule.

215. A method of selecting, isolating or separating cells expressing a modified PSMA, comprising selecting, isolating or separating cells that are recognized or bound by a PSMA-targeting molecule, from a plurality of cells comprising the engineered cells of claim 1 that have been contacted with the PSMA-targeting molecule.

216-228. (canceled)

229. A kit, comprising:
(a) a composition comprising a therapeutically effective amount of the engineered cells of claim 1; and
(b) a composition comprising a PSMA-targeting molecule.

230-249. (canceled)

250. A PSMA-targeting molecule comprising a portion capable of binding to a PSMA or to a modified form thereof, wherein the PSMA-targeting molecule comprises an immunomodulatory agent or a therapeutic agent capable of modulating the tumor microenvironment (TME), wherein the therapeutic agent is linked to a portion of the PSMA targeting molecule by a releasable or cleavable linker responsive to one or more conditions or factors present in the TME.

251-261. (canceled)

262. A PSMA-targeting molecule comprising a portion capable of binding to a modified PSMA, wherein the PSMA-targeting molecule comprises a therapeutic agent capable of modulating the tumor microenvironment (TME), wherein the therapeutic agent is linked to a portion of the PSMA targeting molecule by a releasable or cleavable linker responsive to one or more conditions or factors present in the TME.

263-273. (canceled)

274. A method of treatment comprising administering the PSMA-targeting molecule of claim 250 to a subject.

275. (canceled)

276. A composition comprising the engineered cell of claim 131.

277. A method of treatment comprising administering the engineered cells of claim 131 to a subject.


SEQUENCE LISTING (66 sequences)

SEQ ID NO: 1 - protein, 12 aa, Homo sapiens
  Spacer (IgG4hinge)
ESKYGPPCPPCP

SEQ ID NO: 2 - DNA, 36 bp, Homo sapiens
  Spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28
FWVLVVVGGVLACYSLLVTVAFIIFWV

SEQ ID NO: 9 - protein, 66 aa, Homo sapiens
  CD28
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28
RSKRSRLLHSDYMNMTPRRPGPTRKHYQPYAPPRDFAAYRS

SEQ ID NO: 11 - protein, 41 aa, Homo sapiens
  CD28
RSKRSRGGHSDYMNMTPRRPGPTRKHYQPYAPPRDFAAYRS

SEQ ID NO: 12 - protein, 42 aa, Homo sapiens
  4-1BB
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence; identical to SEQ ID NO: 7
  tEGFR

SEQ ID NO: 17 - protein, 18 aa, Artificial Sequence
  T2A
EGRGSLLTCGDVEENPGP

SEQ ID NO: 18 - protein, 6 aa, Artificial Sequence
  MMP cleavable linker
PLGLWA

SEQ ID NO: 19 - protein, 22 aa, Artificial Sequence
  P2A
GSGATNFSLLKQAGDVEENPGP

SEQ ID NO: 20 - protein, 19 aa, Artificial Sequence
  P2A
ATNFSLLKQAGDVEENPGP

SEQ ID NO: 21 - protein, 20 aa, Artificial Sequence
  E2A
QCTNYALLKLAGDVESNPGP

SEQ ID NO: 22 - protein, 22 aa, Artificial Sequence
  F2A
VKQTLNFDLLKLAGDVESNPGP

SEQ ID NO: 23 - protein, 750 aa, Homo sapiens
  PSMA WT (full length)
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 24 - protein, 750 aa, Artificial Sequence
  PSMA W2G (full length)
MGNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 25 - protein, 741 aa, Artificial Sequence
  PSMA deletion of aa 1-9
SAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 26 - DNA, 2250 bp, Homo sapiens
atgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcc

SEQ ID NO: 27 - DNA, 2253 bp, Artificial Sequence
  CpG-free PSMA
atgtggaatctccttcatgaaacagactctgctgtggccacagccagaagacccagatggctgtgtgctggggccctggtgctggctggtggcttctttctcctgggcttcctctttgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttctgatattgtaccacctttcagtgctttctctcctcaaggaatgccagagggagatctagtgtatgttaactatgcaagaactgaagacttctttaaattggaaagggacatgaaaatcaattgctctgggaaaattgtaattgccagatatgggaaagttttcagaggaaataaggttaaaaatgcccagctggcaggggccaaaggagtcattctctactctgaccctgctgactactttgctcctggggtgaagtcctatccagatggttggaatcttcctggaggtggtgtccagagaggaaatatcctaaatctgaatggtgcaggagaccctctcacaccaggttacccagcaaatgaatatgcttataggagaggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcacagggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagaggggagtggcttatattaatgctgactcatctatagaaggaaactacactctgagagttgattgtacacccctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaaagacttggaattgcttcaggcagagcaaggtatactaaaaattgggaaacaaacaaattcagtggctatccactgtatcacagtgtctatgaaacatatgagttggtggaaaagttttatgatccaatgtttaaatatcacctcactgtggcccaggttagaggagggatggtgtttgagctagccaattccatagtgctcccttttgattgtagagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaa

SEQ ID NO: 28 - protein, 30 aa, Artificial Sequence
  linker
PGGGSGGGGSGGGGSGGGGSGGGGSGGGGP

SEQ ID NO: 29 - protein, 17 aa, Artificial Sequence
  linker
GSADDAKKDAAKKDGKS

SEQ ID NO: 30 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 31 - protein, 22 aa, Artificial Sequence
  GMCSFR alpha chain signal sequence
MLLLVTSLLLCELPHPAFLLIP

SEQ ID NO: 32 - protein, 18 aa, Artificial Sequence
  CD8 alpha signal peptide
MALPVTALLLPLALLLHA

SEQ ID NO: 33 - protein, 4 aa, Artificial Sequence
  linker
GFLG

SEQ ID NO: 34 - protein, 14 aa, Artificial Sequence
  peptide toxin
KLAKLAKKLAKLAK

SEQ ID NO: 35 - protein, 12 aa, Artificial Sequence
  PSMA binding peptide  1
WQPDTAHHWATL

SEQ ID NO: 36 - protein, 12 aa, Artificial Sequence
  PSMA binding peptide 2
HNAYWHWPPSMT

SEQ ID NO: 37 - protein, 11 aa, Artificial Sequence
  PSMA binding peptide 3
GHLIPLRQPSH

SEQ ID NO: 38 - protein, 12 aa, Artificial Sequence
  PSMA binding peptide 4
YTSPHHSTTGHL

SEQ ID NO: 39 - protein, 11 aa, Artificial Sequence
  PSMA binding peptide 5
WTHHHSYPRPL

SEQ ID NO: 40 - protein, 12 aa, Artificial Sequence
  PSMA binding peptide 6
NSFPLMLMHHHP

SEQ ID NO: 41 - protein, 11 aa, Artificial Sequence
  PSMA binding peptide 7
KHMHWHPPALN

SEQ ID NO: 42 - protein, 12 aa, Artificial Sequence
  PSMA binding peptide 8
SLDSMSPQWHAD

SEQ ID NO: 43 - protein, 12 aa, Artificial Sequence
  PSMA binding peptide 9
SEFIHHWTPPPS

SEQ ID NO: 44 - protein, 12 aa, Artificial Sequence
  PSMA binding peptide 10
NGFSHHAPLMRY

SEQ ID NO: 45 - protein, 11 aa, Artificial Sequence
  PSMA binding peptide 11
HHEWTHHWPPP

SEQ ID NO: 46 - protein, 11 aa, Artificial Sequence
  PSMA binding peptide 12
AWPENPSRRPF

SEQ ID NO: 47 - protein, 12 aa, Artificial Sequence
  PSMA binding peptide 13
AGFQHHPSFYRF

SEQ ID NO: 48 - protein, 12 aa, Artificial Sequence
  PSMA binding peptide 14
KSLSRHDHIHHH

SEQ ID NO: 49 - protein, 11 aa, Artificial Sequence
  PSMA binding peptide 15
YRHWPIDYPPP

SEQ ID NO: 50 - protein, 11 aa, Artificial Sequence
  PSMA binding peptide 16
MIHTNHWWAQD

SEQ ID NO: 51 - protein, 12 aa, Artificial Sequence
  PSMA binding peptide 17
QRSPMMSRIRLP

SEQ ID NO: 52 - protein, 741 aa, Artificial Sequence
  PSMA N9del
MAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 53 - DNA, 2223 bp, Artificial Sequence
  PSMA N9del DNA
atggccgtcgcaaccgcccgccgaccccgctggctgtgcgccggagccctggtgctggccggcggcttctttctgctgggcttcctgtttggctggtttatcaagagctccaacgaggccaccaatatcacacctaagcacaatatgaaggccttcctggacgagctgaaggccgagaatatcaagaagttcctgtataacttcacccagatcccacacctggccggcacagagcagaactttcagctggccaagcagatccagagccagtggaaggagttcggcctggactccgtggagctggcccactacgatgtgctgctgtcttatccaaataagacccaccccaactatatcagcatcatcaacgaggatggcaatgagatcttcaacacatctctgtttgagccccctccacccggctacgagaatgtgagcgacatcgtgcctccattctctgcctttagcccacagggaatgcctgagggcgatctggtgtacgtgaattatgccagaaccgaggacttctttaagctggagagagatatgaagatcaactgcagcggcaagatcgtgatcgccagatacggcaaggtgtttcgcggcaataaggtgaagaacgcacagctggccggagcaaagggcgtgatcctgtactccgaccccgccgattatttcgcccctggcgtgaagtcctatccagacggctggaatctgccaggaggaggcgtgcagaggggaaacatcctgaacctgaatggagcaggcgatcctctgaccccaggataccccgccaacgagtacgcctataggagaggaatcgcagaggcagtgggcctgccttccatcccagtgcaccccatcggctactatgacgcccagaagctgctggagaagatgggaggctctgccccacctgattctagctggaggggcagcctgaaggtgccttacaatgtgggcccaggcttcaccggcaacttttccacacagaaggtgaagatgcacatccactctaccaatgaggtgacacggatctataacgtgatcggcaccctgaggggagcagtggagcctgacagatacgtgatcctgggcggccacagagacagctgggtgtttggaggaatcgatccacagtccggagcagcagtggtgcacgagatcgtgaggtccttcggcaccctgaagaaggagggatggcgccccaggaggacaatcctgtttgcctcttgggatgccgaggagttcggcctgctgggctccacagagtgggcagaggagaattctcggctgctgcaggagagaggcgtggcctacatcaatgccgactcctctatcgagggcaactataccctgagggtggattgcacacccctgatgtactccctggtgcacaacctgaccaaggagctgaagtctcctgacgagggcttcgagggcaagtctctgtatgagagctggacaaagaagtctccaagccccgagtttagcggcatgcctaggatctccaagctgggctctggcaatgatttcgaggtgttctttcagcgcctgggaatcgcctccggccgggcaagatacaccaagaattgggagacaaacaagttctctggctacccactgtatcacagcgtgtacgagacatatgagctggtggagaagttctacgaccccatgtttaagtatcacctgacagtggcacaggtgcggggaggaatggtgtttgagctggccaatagcatcgtgctgccattcgactgtagggattatgccgtggtgctgcgcaagtacgccgacaagatctattccatctctatgaagcacccccaggagatgaagacctacagcgtgtccttcgattccctgttttctgccgtgaagaacttcacagagatcgccagcaagttttccgagagactgcaggacttcgataagagcaatcccatcgtgctgcggatgatgaacgaccagctgatgttcctggagagagcctttatcgaccctctgggcctgcctgataggccattctaccgccacgtgatctatgcccctagctcccacaacaagtacgccggcgagtcctttccaggcatctatgacgccctgttcgatatcgagagcaaggtggacccctccaaggcatggggagaggtgaagagacagatctatgtcgcagcattcactgtccaggcagcagcagaaaccctgtcagaagtcgca

SEQ ID NO: 54 - DNA, 66 bp, Artificial Sequence
  P2A DNA
ggatctggagcaacaaacttctcactactcaaacaagcaggtgacgtggaggagaatcccggaccc

SEQ ID NO: 55 - protein, 12 aa, Artificial Sequence
  Hinge
ESKYGPPCPSCP

SEQ ID NO: 56 - protein, 9 aa, Artificial Sequence
  Hinge
YGPPCPPCP

SEQ ID NO: 57 - protein, 10 aa, Artificial Sequence
  Hinge
KYGPPCPPCP

SEQ ID NO: 58 - protein, 14 aa, Artificial Sequence
  Hinge
EVVVKYGPPCPPCP

SEQ ID NO: 59 - protein, 11 aa, Artificial Sequence
  FMC63 CDR L1
RASQDISKYLN

SEQ ID NO: 60 - protein, 7 aa, Artificial Sequence
  FMC63 CDR L2
SRLHSGV

SEQ ID NO: 61 - protein, 9 aa, Artificial Sequence
  FMC63 CDR L3
GNTLPYTFG

SEQ ID NO: 62 - protein, 5 aa, Artificial Sequence
  FMC63 CDR H1
DYGVS

SEQ ID NO: 63 - protein, 16 aa, Artificial Sequence
  FMC63 CDR H2
VIWGSETTYYNSALKS

SEQ ID NO: 64 - protein, 7 aa, Artificial Sequence
  FMC63 CDR H3
YAMDYWG

SEQ ID NO: 65 - protein, 18 aa, Artificial Sequence
  Linker
GSTSGSGKPGSGEGSTKG

SEQ ID NO: 66 - protein, 5 aa, Artificial Sequence
  Spacer
  VARIANT 1: Xaa = Gly, Cys or Arg
  VARIANT 4: Xaa = Cys or Thr
XPPXP